(19) United States
(12) Patent Application Publication
(10) Pub. No.: US 2010/0129799 A1
(43) Pub. Date: May 27, 2010

(54) CANCER SUSCEPTIBILITY VARIANTS ON CHR8Q24.21

(75) Inventors: Júlíus Guômundsson, Reykjavik (IS); Patric Sulem, Reykjavik (IS); Augustine Kong, Reykjavik (IS); Andrei Manulescu, Reykjavik (IS); Laufey Amundadottir, Gaithersburg, MD (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 12/442,254

(22) PCT Filed: Oct. 26, 2007
(86) PCT No.: PCT/IS07/00019

§ 371 Date: Mar. 20, 2009

(30) Foreign Application Priority Data
Oct. 27, 2006 (IS) 8560

(51) Int. Cl. C12Q 1/68; C12M 1/34

(52) U.S. Cl. 435/6; 435/287.2

(57) ABSTRACT

A region on chromosome 8q24.21 has been demonstrated to play a major role in particular forms of cancer. It has been discovered that certain markers and haplotypes are indicative of a susceptibility to particular cancers, including prostate cancer. Diagnostic applications for identifying a susceptibility to cancer using these markers and haplotypes are described.

### BACKGROUND OF THE INVENTION

Cancer, the uncontrolled growth of malignant cells, is a major health problem of the modern medical era and is one of the leading causes of death in developed countries. In the United States, one in four deaths is caused by cancer (Jemal, A. et al., CA Cancer J. Clin. 52:23-47 (2002)).

The incidence of prostate cancer has dramatically increased over the last decades and prostate cancer is now a leading cause of death in the United States and Western Europe (Peschel, R. E. and J. W. Colberg, Lancet 4:233-41 (2003); Nelson, W. G. et al., N. Engl. J. Med. 349(4):366-81 (2003)). Prostate cancer is the most frequently diagnosed noncutaneous malignancy among men in industrialized countries, and in the United States, 1 in 8 men will develop prostate cancer during his life (Simard, J. et al., Endocrinology 143(6):2029-40 (2002)). Although environmental factors, such as dietary factors and lifestyle-related factors, contribute to the risk of prostate cancer, genetic factors have also been shown to play an important role. Indeed, a positive family history is among the strongest epidemiological risk factors for prostate cancer, and twin studies comparing the concordant occurrence of prostate cancer in monozygotic twins have consistently revealed a stronger hereditary component in the risk of prostate cancer than in any other type of cancer (Nelson, W. G. et al., N. Engl. J. Med. 349(4):366-81 (2003); Lichtenstein P. et. al., N. Engl. J. Med. 343(2):78-85 (2000)). In addition, an increased risk of prostate cancer is seen in 1^{st} to 5^{th} degree relatives of prostate cancer cases in a nation wide study on the familiality of all cancer cases diagnosed in Iceland from 1955-2003 (Amundadottir et. al., PLoS Medicine 1(3):e65 (2004)). The genetic basis for this disease, emphasized by the increased risk among relatives, is further supported by studies of prostate cancer among particular populations: for example, African Americans have among the highest incidence of prostate cancer and mortality rate attributable to this disease: they are 1.6 times as likely to develop prostate cancer and 2.4 times as likely to die from this disease than European Americans (Ries, L. A. G. et al., NIH Pub. No. 99-4649 (1999)).

An average 40% reduction in life expectancy affects males with prostate cancer. If detected early, prior to metastasis and local spread beyond the capsule, prostate cancer can be cured (e.g., using surgery). However, if diagnosed after spread and metastasis from the prostate, prostate cancer is typically a fatal disease with low cure rates. While prostate-specific antigen (PSA)-based screening has aided early diagnosis of prostate cancer, it is neither highly sensitive nor specific (Punglia et. al., N Engl J Med. 349(4):335-42 (2003)). This means that a high percentage of false negative and false positive diagnoses are associated with the test. The consequences are both many instances of missed cancers and unnecessary follow-up biopsies for those without cancer. As many as 65 to 85% of individuals (depending on age) with prostate cancer have a PSA value less than or equal to 4.0 ng/mL, which has traditionally been used as the upper limit for a normal PSA level (Punglia et. al., N Engl J Med. 349(4):335-42 (2003); Cookston, M. S., Cancer Control 8(2):133-40 (2001); Thompson, I. M. et. al., N Engl J Med. 350:2239-46 (2004)). A significant fraction of those cancers with low PSA levels are scored as Gleason grade 7 or higher, which is a measure of an aggressive prostate cancer. Id.

In addition to the sensitivity problem outlined above, PSA testing also has difficulty with specificity and predicting prognosis. PSA levels can be abnormal in those without prostate cancer. For example, benign prostatic hyperplasia (BPH) is one common cause of a false-positive PSA test. In addition, a variety of noncancer conditions may elevate serum PSA levels, including urinary retention, prostatitis, vigorous prostate massage and ejaculation. Id.

Subsequent confirmation of prostate cancer using needle biopsy in patients with positive PSA levels is difficult if the tumor is too small to see by ultrasound. Multiple random samples are typically taken but diagnosis of prostate cancer may be missed because of the sampling of only small amounts of tissue. Digital rectal examination (DRE) also misses many cancers because only the posterior lobe of the prostate is examined. As early cancers are nonpalpable, cancers detected by DRE may already have spread outside the prostate (Mistry K. J., Am. Board Fam. Pract. 16(2):95-101 (2003)).

Thus, there is clearly a great need for improved diagnostic procedures that would facilitate early-stage prostate cancer detection and prognosis, as well as aid in preventive and curative treatments of the disease. In addition, there is a need to develop tools to better identify those patients who are more likely to have aggressive forms of prostate cancer from those patients that are more likely to have more benign forms of prostate cancer that remain localized within the prostate and do not contribute significantly to morbidity or mortality. This would help to avoid invasive and costly procedures for patients not at significant risk.

Loci Associated with Various Forms of Prostate Cancer

The incidence of prostate cancer has dramatically increased over the last decades. Prostate cancer is a multifactoral disease with genetic and environmental components involved in its etiology. It is characterized by heterogeneous growth patterns that range from slow growing tumors to very rapid highly metastatic lesions.

Although genetic factors are among the strongest epidemiological risk factors for prostate cancer, the search for genetic determinants involved in the disease has been challenging. Studies have revealed that linking candidiate genetic markers to prostate cancer has been more difficult than identifying susceptibility genes for other cancers, such as breast, ovary and colon cancer. Several reasons have been proposed for this increased difficulty including: the fact that prostate cancer is often diagnosed at a late age thereby often making it difficult to obtain DNA samples from living affected individuals for more than one generation; the presence within high-risk pedigrees of phenocopies that are associated with a lack of distinguishing features between hereditary and sporadic forms; and the genetic heterogeneity of prostate cancer and the accompanying difficulty of developing appropriate statistical transmission models for this complex disease (Simard, J. et al., Endocrinology 143(6):2029-40 (2002)).

Various genome scans for prostate cancer-susceptibility genes have been conducted and several prostate cancer susceptibility loci have been reported. For example, HPC1 (1q24-q25), PCAP (1q42-q43), HCPX (Xq27-q28), CAPB (1p36), HPC20 (20q13), HPC2/ELAC2 (17p11) and 16q23 have been proposed as prostate cancer susceptibility loci (Simard, J. et al., Endocrinology 143(6):2029-40 (2002); Nwosu, V. et al., Hum. Mol. Genet. 10(20):2313-18 (2001)). In a genome scan conducted by Smith et al., the strongest evidence for linkage was at HPC1, although two-point analysis also revealed a LOD score of 1.5 at D4S430 and LOD scores 1.0 at several loci, including markers at Xq27-28 (Ostrander E. A. and J. L. Stanford, Am. J. Hum. Genet. 67:1367-75 (2000)). Another genome scan reported two-point LOD scores of 1.5 for chromosomes 10q, 12q and 14q using an autosomal dominant model of inheritance, and chromosomes 1q, 8q, 10q and 16p using a recessive model of inheritance. Id. Still another genome scan identified regions with nominal evidence for linkage on 2q, 12p, 15q, 16q and 16p. Id. A genome scan for prostate cancer predisposition loci using a small set of Utah high risk prostate cancer pedigrees and a set of 300 polymorphic markers provided evidence for linkage to a locus on chromosome 17p (Simard, J. et al., Endocrinology 143(6):2029-40 (2002)). Eight new linkage analyses were published in late 2003, which depicted remarkable heterogeneity. Eleven peaks with LOD scores higher than 2.0 were reported, none of which overlapped (see Actane consortium, Schleutker et. al., Wiklund et. al., Witte et. al., Janer et. al., Xu et. al., Lange et. al., Cunningham et. al.; all of which appear in Prostate, vol. 57 (2003)).

As described above, identification of particular genes involved in prostate cancer has been challenging. One gene that has been implicated is RNASEL, which encodes a widely expressed latent endoribonuclease that participates in an interferon-inducible RNA-decay pathway believed to degrade viral and cellular RNA, and has been linked to the HPC locus (Carpten, J. et al., Nat. Genet. 30:181-84 (2002); Casey, G. et al., Nat. Genet. 32(4):581-83 (2002)). Mutations in RNASEL have been associated with increased susceptibility to prostate cancer. For example, in one family, four brothers with prostate cancer carried a disabling mutation in RNASEL, while in another family, four of six brothers with prostate cancer carried a base substitution affecting the initiator methionine codon of RNASEL. Id. Other studies have revealed mutant RNASEL alleles associated with an increased risk of prostate cancer in Finnish men with familial prostate cancer and an Ashkenazi Jewish population (Rokman, A. et al., Am J. Hum. Genet. 70:1299-1304 (2002); Rennert, H. et al., Am J. Hum. Genet. 71:981-84 (2002)). In addition, the Ser217Leu genotype has been proposed to account for approximately 9% of all sporadic cases in Caucasian Americans younger than 65 years (Stanford, J. L., Cancer Epidemiol. Biomarkers Prev. 12(9):876-81 (2003)). In contrast to these positive reports, however, some studies have failed to detect any association between RNASEL alleles with inactivating mutations and prostate cancer (Wang, L. et al., Am. J. Hum. Genet. 71:116-23 (2002); Wiklund, F. et al., Clin. Cancer Res. 10(21):7150-56 (2004); Maier, C. et. al., Br. J. Cancer 92(6)1159-64 (2005)).

The macrophage-scavenger receptor 1 (MSR1) gene, which is located at 8p22, has also been identified as a candidate prostate cancer-susceptibility gene (Xu, J. et al., Nat. Genet. 32:321-25 (2002)). A mutant MSR1 allele was detected in approximately 3% of men with nonhereditary prostate cancer but only 0.4% of unaffected men. Id. However, not all subsequent reports have confirmed these initial findings (see, e.g., Lindmark, F. et al., Prostate 59(2):132-40 (2004); Seppala, E. H. et al., Clin. Cancer Res. 9(14):5252-56 (2003); Wang, L. et al., Nat. Genet. 35(2):128-29 (2003); Miller, D. C. et al., Cancer Res. 63(13):3486-89 (2003)). MSR1 encodes subunits of a macrophage-scavenger receptor that is capable of binding a variety of ligands, including bacterial lipopolysaccharide and lipoteichoic acid, and oxidized high-density lipoprotein and low-density lipoprotein in serum (Nelson, W. G. et al., N. Engl. J. Med. 349(4):366-81 (2003)).

The ELAC2 gene on Chr17 was the first prostate cancer susceptibility gene to be cloned in high risk prostate cancer families from Utah (Tavtigian, S. V., et al., Nat. Genet. 27(2):172-80 (2001)). A frameshift mutation (1641InsG) was found in one pedigree. Three additional missense changes: Ser217Leu; Ala541Thr; and Arg781His, were also found to associate with an increased risk of prostate cancer. The relative risk of prostate cancer in men carrying both Ser217Leu and Ala541Thr was found to be 2.37 in a cohort not selected on the basis of family history of prostate cancer (Rebbeck, T. R., et al., Am. J. Hum. Genet. 67(4):1014-19 (2000)). Another study described a new termination mutation (Glu216X) in one high incidence prostate cancer family (Wang, L., et al., Cancer Res. 61(17):6494-99 (2001)). Other reports have not demonstrated strong association with the three missense mutations, and a recent metaanalysis suggests that the familial risk associated with these mutations is more moderate than was indicated in initial reports (Vesprini, D., et al., Am. J. Hum. Genet. 68(4):912-17 (2001); Shea, P. R., et al., Hum. Genet. 111(4-5):398-400 (2002); Suarez, B. K., et al., Cancer Res. 61(13):4982-84 (2001); Severi, G., et al., J. Natl. Cancer Inst. 95(11):818-24 (2003); Fujiwara, H., et al., J. Hum. Genet. 47(12):641-48 (2002); Camp, N. J., et al., Am. J. Hum. Genet. 71(6):1475-78 (2002)).

Polymorphic variants of genes involved in androgen action (e.g., the androgen receptor (AR) gene, the cytochrome P-450c17 (CYP17) gene, and the steroid-5-reductase type II (SRD5A2) gene), have also been implicated in increased risk of prostate cancer (Nelson, W. G. et al., N. Engl. J. Med. 349(4):366-81 (2003)). With respect to AR, which encodes the androgen receptor, several genetic epidemiological studies have shown a correlation between an increased risk of prostate cancer and the presence of short androgen-receptor polyglutamine repeats, while other studies have failed to detect such a correlation. Id. Linkage data has also implicated an allelic form of CYP17, an enzyme that catalyzes key reactions in sex-steroid biosynthesis, with prostate cancer (Chang, B. et al., Int. J. Cancer 95:354-59 (2001)). Allelic variants of SRD5A2, which encodes the predominant isozyme of 5-reductase in the prostate and functions to convert testosterone to the more potent dihydrotestosterone, have been associated with an increased risk of prostate cancer and with a poor prognosis for men with prostate cancer (Makridakis, N. M. et al., Lancet 354:975-78 (1999); Nam, R. K. et al., Urology 57:199-204 (2001)).

In short, despite the effort of many groups around the world, the genes that account for a substantial fraction of prostate cancer risk have not been identified. Although twin studies have implied that genetic factors are likely to be prominent in prostate cancer, only a handful of genes have been identified as being associated with an increased risk for prostate cancer, and these genes account for only a low percentage of cases. Thus, it is clear that the majority of genetic risk factors for prostate cancer remain to be found. It is likely that these genetic risk factors will include a relatively high number of low-to-medium risk genetic variants. These low-to-medium risk genetic variants may, however, be responsible for a substantial fraction of prostate cancer, and their identification, therefore, a great benefit for public health. Furthermore, none of the published prostate cancer genes have been reported to predict a greater risk for aggressive prostate cancer than for less aggressive prostate cancer.

Extensive genealogical information for a population containing cancer patients has in a recent study been combined with powerful gene sharing methods to map a locus on chromosome 8q24.21, which has been demonstrated to play a major role in cancer (e.g., breast cancer, prostate cancer, lung cancer, melanoma). Various cancer patients and their relatives were genotyped with a genome-wide marker set including 1100 microsatellite markers, with an average marker density of 3-4 cM. (Amundadottir L. T., Nature Genet. 38(6):652-658 (2006)). Association was detected to a single LD block within the locus between positions 128.414 and 128.506 Mb (NCBI build 34) in Utah CEPH HapMap samples.

Breast cancer is a significant health problem for women in the United States and throughout the world. Although advances have been made in detection and treatment of the disease, breast cancer remains the second leading cause of cancer-related deaths in women, affecting more than 180,000 women in the United States each year. For women in North America, the life-time odds of getting breast cancer are now one in eight.

No universally successful method for the treatment or prevention of breast cancer is currently available. Management of breast cancer currently relies on a combination of early diagnosis (e.g., through routine breast screening procedures) and aggressive treatment, which may include one or more of a variety of treatments, such as surgery, radiotherapy, chemotherapy and hormone therapy. The course of treatment for a particular breast cancer is often selected based on a variety of prognostic parameters including an analysis of specific tumor markers. See, e.g., Porter-Jordan and Lippman, Breast Cancer 8:73-100 (1994).

Although the discovery of BRCA1 and BRCA2 were important steps in identifying key genetic factors involved in breast cancer, it has become clear that mutations in BRCA1 and BRCA2 account for only a fraction of inherited susceptibility to breast cancer (Nathanson, K. L. et al., Human Mol. Gen. 10(7):715-720 (2001); Anglican Breast Cancer Study Group. Br. J. Cancer 83(10):1301-08 (2000); and Syrjakoski K. et. al., J. Natl. Cancer Inst. 92:1529-31 (2000)). In spite of considerable research into therapies for breast cancer, breast cancer remains difficult to diagnose and treat effectively, and the high mortality observed in breast cancer patients indicates that improvements are needed in the diagnosis, treatment and prevention of the disease.

deCODE has demonstrated an increased risk of breast cancer in 1^{st} to 5^{th} degree relatives of breast cancer cases in a nation wide study of the familiality of all cancers diagnosed in Iceland from 1955-2003 (Amundadottir et. al., PLoS Med. 1(3):e65 (2004); Lichtenstein P. et. al., N. Engl. J. Med. 343(2):78-85 (2000)), where the authors show that breast cancer has one of the highest heritability of all cancers tested in a cohort of close to 45,000 twins.

It is estimated that only 5-10% of all breast cancers in women are associated with heriditary susceptibility due to mutations in autosomal dominant genes, such as BRCA1, BRCA2, p53, pTEN and STK11/LKB1 (Mincey, B. A. Oncologist 8:466-73 (2003)). One genetic locus, on Chromosome 8p, has been proposed as a locus for a breast cancer-susceptibility gene based on studies documenting allelic loss in this region in sporadic breast cancer (Seitz, S. et al., Br. J. Cancer 76:983-91 (1997); Kerangueven, F. et al., Oncogene 10:1023 (1995)). Studies have also suggested that a breast cancer-susceptibility gene may be located on 13q21 (Kainu, T. et al., Proc. Natl. Acad. Sci. USA 97:9603-08 (2000)). However, as with prostate cancer, identification of additional breast cancer-susceptibility genes has been difficult.

Lung cancer causes more deaths from cancer worldwide than any other form of cancer (Goodman, G. E., Thorax 57:994-999 (2002)). In the United States, lung cancer is the primary cause of cancer death among both men and women. In 2002, the death rate from lung cancer was an estimated 134,900 deaths, exceeding the combined total for breast, prostate and colon cancer. Lung cancer is also the leading cause of cancer death in all European countries and is rapidly increasing in developing countries. While environmental factors, such as lifestyle factors (e.g., smoking) and dietary factors, play an important role in lung cancer, genetic factors also contribute to the disease. For example, a family of enzymes responsible for carcinogen activation, degradation and subsequent DNA repair have been implicated in susceptibility to lung cancer. Studies have revealed that defects in both the p53 and RB/p16 pathway are essential for the malignant transformation of lung epithelial cells (Yokota, J. and T. Kohno, Cancer Sci. 95(3):197-204 (2004)). Other genes, such as K-ras, PTEN and MYO18B, are genetically altered less frequently than p53 and RB/p16 in lung cancer cells, suggesting that alterations in these genes are associated with further malignant progression or unique phenotypes in a subset of lung cancer cells. Molecular footprint studies that have been conducted at the sites of p53 mutations and RB/p16 deletions have further demonstrated that DNA repair activities and non-homologous end-joining of DNA double-strand breaks are important in the accumulation of genetic alterations in lung cancer cells. In addition, studies have identified candidate lung adenocarcinoma susceptibility genes, for example, drug carcinogen metabolism genes, such as NQ01 (NAD(P)H:quinone oxidoreductase) and GSTT1 (glutathione S-transferase T1), and DNA repair genes, such as XRCC1 (X-ray cross-complementary group 1) (Yanagitani, N. et al., Cancer Epidemiol. Biomarkers Prev. 12:366-71 (2003); Lin, P. et al., J. Toxicol. Environ. Health A. 58:187-97 (1999); Divine, K. K. et al., Mutat. Res. 461:273-78 (2001); Sunaga, N. et al., Cancer Epidemiol. Biomarkers Prev. 11:730-38 (2002)). A region of chromosome 19q13.3, which encompasses locus D19S246, has also been suggested as containing a gene(s) associated with lung adenocarcinoma (Yanagitani, N. et al., Cancer Epidemiol. Biomarkers Prev. 12:366-71 (2003)). In addition, an increased risk to familial members outside of the nuclear family has been shown by deCODE geneticists by analysing all lung cancer cases diagnosed in Iceland over 48 years. This increased risk could not be entirely accounted for by smoking indicating that genetic variants may predispose certain individuals to lung cancer (Jonson et al., JAMA 292(24):2977-83 (2004); Amundadottir et al., PLoS Med. 1(3):e65 (2004)).

The five-year survival rate among all lung cancer patients, regardless of the stage of disease at diagnosis, is only 13%. This contrasts with a five-year survival rate of 46% among cases detected while the disease is still localized. However, only 16% of lung cancers are discovered before the disease has spread. Early detection is difficult as clinical symptoms are often not observed until the disease has reached an advanced stage. Currently, diagnosis is aided by the use of chest x-rays, analysis of the type of cells contained in sputum and fiberoptic examination of the bronchial passages. Treatment regimens are determined by the type and stage of the cancer, and include surgery, radiation therapy and/or chemotherapy. In spite of considerable research into therapies for this and other cancers, lung cancer remains difficult to diagnose and treat effectively. Accordingly, there is a great need in the art for improved methods for detecting and treating such cancers.

The incidence of malignant melanoma is increasing more rapidly than any other type of human cancer in North America (Armstrong et al., Cancer Surv. 19-20:219-240 (1994)). Although melanoma is curable when identified at an early stage, it requires detection and removal of the primary tumor before it has spread to distant sites. Malignant melanomas have great propensity to metastasize and are notoriously resistant to conventional cancer treatments, such as chemotherapy and -irradiation. Once metastases have occurred the prognosis is very poor. Thus, early detection of melanoma is of vital importance in melanoma treatment and control.

Studies have demonstrated that genetic factors play an important role in the stepwise progression of normal pigment cells to atypical nevi to invasive primary melanoma and finally to cells with aggressive metastatic potential (Kim, C. J., et al., Cancer Control 9(1):49-53 (2002)). For example, genetic aberrations, such as rearrangements on chromosome 1, which harbors a tumor-suppressor gene, have been implicated in malignant melanomas. However, the molecular and biological mechanisms of how a normal melanocyte of adult skin transforms into a melanoma cell remains unclear.

Various studies have implicated genetic factors in melanoma. For example, elevated familial risk for early onset melanoma was noted by examination of a Utah population database (Cannon-Albright, L. A., et al., Cancer Res., 54(9):2378-85 (1994)). In addition, the Swedish Family-Cancer Database reported a familial standardized incidence ratios (SIR) of 2.54 and 2.98 for cutaneous malignant melanoma (CMM) in a individual with an affected parent or sib, respectively. For an offspring whose parent had multiple primary melanomas, the SIR rose to 61.78 (Hemminki, K., et al., J. Invest. Dermatol. 120(2):217-23 (2003)). In the Icelandic population-based study by Amundadottir et al. (PLoS Med. 1(3):e65 (2004)), comparable SIR-values were found. Although figures vary, it has been reported that about 10% of CMM cases are familial (Hansen, C. B., et al., Lancet Oncol. 5(5):314-19 (2004)). Given the known environmental risk factors for melanoma, shared environment in addition to genetics is likely to factor into these estimates. However, familial cases tend to have earlier ages of onset and a higher risk of multiple primary tumors, suggesting a genetic component (see, e.g., Tucker M., Oncogene 22(20):3042-52 (2003)). However, the molecular and biological mechanisms of how a normal melanocyte transforms into a melanoma cell remains unclear.

A series of linkage-based studies have implicated CDKN2a on Chr9p21 as a major CMM-susceptibility gene (Bataille, V., Eur. J. Cancer 39(10):1341-47 (2003)). CDK4 was identified as a pathway candidate shortly thereafter, however, mutations in CDK4 have only been observed in a few families worldwide (Zuo, L., et al., Nat. Genet. 12(1):97-99 (1996)). CDKN2a encodes the cyclin dependent kinase inhibitor p16, which inhibits CDK4 and CDK6, thereby preventing G1 to S cell cycle transit. An alternate transcript of CKDN2a produces p14ARF, which encodes a cell cycle inhibitor that acts through the MDM2-p53 pathway. It is likely that CDKN2a mutant melanocytes are deficient in cell cycle control or the establishment of senescence, either as a developmental state or in response to DNA damage (Ohtani, N., et al., J. Med. Invest. 51(3-4):146-53 (2004)). Overall penetrance of CDKN2a mutations in familial CMM cases is 67% by age 80. However, penetrance is increased in areas of high melanoma prevalence (Bishop, D. T., et al., J. Natl. Cancer Inst. 94(12):894-903 (2002)).

The Melanoma Genetics Consortium recently completed a genome-wide scan for CMM, using a set of predominantly Australian, high-risk families unlinked to 9p21 or CDK4 (Gillanders, E., et al., Am. J. Hum. Genet. 73(2):301-13 (2003)). The 10 cM resolution scan gave a non-parametric multipoint LOD score of 2.06 in the 1p22 region. Other locations on chromosomes 4, 7, 14, and 18 gave LODs in excess of 1.0. With additional markers to 1p22 and the application of an age-of-onset restriction, non-parametric LOD scores in excess of 5.0 were observed. Evidence suggests that a high-penetrance mutation of a tumor suppressor gene exists at this location, however the pattern of LOH is complex (Walker, G. J., et al., Genes Chromosomes Cancer, 41(1):56-64 (2004)).

Another genetic locus that has been implicated in CMM is that which encodes the Melanocortin 1 Receptor (MC1R). MC1R is a G-protein coupled receptor that is involved in promoting the switch from pheomelanin to eumelanin synthesis. Numerous well-characterized variants of the MC1R gene have been implicated in red-haired, pale-skinned and freckle-prone phenotypes. More than half of red-haired individuals carry at least one of these MC1R variants (Valverde, P., et al., Nat. Genet. 11(3):328-30 (1995); Palmer, J. S., et al., Am. J. Hum. Genet. 66(1):176-86 (2000)). Subsequently, it was shown that the same variants conferred risk for CMM with odds ratios of about 2.0 for a single variant and about 4.0 for compound heterozygotes. Recent studies have shown that the stronger variants of MC1R increase the penetrance of CDKN2a mutations and lower the age of onset (Box, N. F., et al., Am. J. Hum. Genet. 69(4):765-73 (2001); van der Velden, P. A., et al., Am. J. Hum. Genet., 69(4):774-79 (2001)).

A number of other candidate genes have been implicated in CMM. For example, a landmark study in cancer genomics identified somatic mutations in BRAF (the human B1 homolog of the v-raf murine sarcoma virus oncogene) in 60% of melanomas (Davies, H., et al., Nature 417(6892):949-54 (2002)). Mutations are also common in nevi, both typical and atypical, suggesting that mutation is an early event. Id. Germline mutations have not been reported, however, a germline SNP variant of BRAF has been implicated in CMM risk (Meyer, P., et al., J. Carcinog. 2(1):7 (2003)). Other candidate genes, which were identified through association studies and have been implicated in CMM risk include, e.g., XRCC3, XPD, EGF, VDR, NBS1, CYP2D6, and GSTM1 (Hayward, N. K., Oncogene, 22(20):3053-62 (2003)). However, such association studies frequently suffer from small sample sizes, reliance on single SNPs and potential population stratification.

Clearly, identification of markers and genes that are responsible for susceptibility to particular forms of cancer (e.g., prostate cancer, breast cancer, lung cancer, melanoma, colon cancer, testicular cancer) is one of the major challenges facing oncology today. Some of the pathways underlying cancer are shared in different forms of cancer. As a consequence, genetic risk factors identified for one particular form of cancer may also represent a risk factor for other cancer types. Diagnostic and therapeutic methods utilizing these risk factors may therefore have a common utility. Accordingly, therapeutic measures developed to target such risk factors may have implications for cancer in general, and not necessarily only the cancer for which the risk factor is originally identified. There is a need to identify means for the early detection of individuals that have a genetic susceptibility to cancer so that more aggressive screening and intervention regimens may be instituted for the early detection and treatment of cancer. Cancer genes may also reveal key molecular pathways that may be manipulated (e.g., using small or large molecule weight drugs) and may lead to more effective treatments regardless of the cancer stage when a particular cancer is first diagnosed.

### SUMMARY OF THE INVENTION

As described herein, it has been discovered that particular markers haplotypes in a specific DNA segment within chromosome 8q24.21 are indicative of susceptibility to particular cancers.

In a first aspect, the invention relates to a method for diagnosing a susceptibility to cancer in a human individual, comprising determining the presence or absence of at least one allele of at least one polymorphic marker in a nucleic acid sample obtained from the individual, wherein the at least one polymorphic marker is associated with SEQ ID NO:2, and wherein the presence of the at least one allele is indicative of a susceptibility to the cancer. In one embodiment, the at least one marker is associated with SEQ ID NO:1. In another embodiment, the at least one marker is located within a genomic region whose nucleotide sequence is set forth in SEQ ID NO:2. In an alternative embodiment, the at least one marker is located within a genomic region whose nucleotide sequence is set forth in SEQ ID NO:1. In one preferred embodiment, the at least one polymorphic marker comprises at least one marker selected from the group of markers set forth in Table 5A, 5B and 5C.

Due to the nature of linkage disequlibrium, the present invention may be practiced using a variety of polymorphic markers that are in linkage disequilibrium. Thus, in another embodiment, the at least one marker comprises at least one marker within Chr8q24.21 in strong linkage disequilibrium, as defined by |D′|>0.8 and/or r²>0.2, with one or more markers selected from the group consisting of the markers in Table 4A and 4B. In another embodiment, the at least one polymorphic marker is in linkage disequilibrium with HapC. In one preferred embodiment, the at least one marker is marker rs16901979 (SEQ ID NO: 73) and markers in linkage disequilibrium therewith. In another preferred embodiment, the at least one marker is selected from the markers set forth in Table 4A and 4B.

In some embodiments of the invention, the method of diagnosing a susceptibility to cancer further comprises assessing the frequency of at least one haplotype in the individual. The haplotype comprises in one such embodiment the markers rs1456314 allele G, rs17831626 allele T, rs7825414 allele G, rs6993569 allele G, rs6994316 allele A, rs6470494 allele T, rs1016342 allele C, rs1031588 allele G, rs1016343 allele T, rs1551510 allele G, rs1456306 allele C, rs1378897 allele G, rs1456305 allele T, rs7816535 allele G

In certain embodiments of the invention, susceptibility is represented by values for relative risk (RR). In other embodiments, the susceptibility is represented by an odds ratio (OR). In certain embodiments of the method diagnosing a susceptibility to cancer, the susceptibility is increased susceptibility, characterized by values for RR or OR of greater than one. In other embodiments, the susceptibility is decreased susceptibility, characterized by values for RR or OR of less than one. In particular embodiments of the invention, the increased susceptibility is characterized by a relative risk of at least 1.5, including a relative risk of at least 1.7, a relative risk of at least 2.0, a relative risk of at least 2.5, a relative risk of at least 3.0, a relative risk of at least 3.5, and a relative risk of at least 4.0. Other embodiments are characterized by relative risk of at least 1.75, 2.25, 2.75, 3.25, 3.75, and so on. Other values for the relative risk are however also within the scope of the present invention.

In certain other embodiments of the invention, certain alleles or haplotypes are found in decreased frequency in patients than in the population. Thus, certain alleles or haplotypes are found in decreased frequency in individuals diagnosed with, or at risk for, particular cancer (e.g., prostate cancer) than in the general population. Such markers are indicative of a protection against the cancer, or a decreased susceptibility of developing these disorders. Decreased susceptibility is in particular embodiments characterized by a relative risk of less than 0.7, including a relative risk of less than 0.6, a relative risk of less than 0.5, a relative risk of less than 0.4, a relative risk of less than 0.35, a relative risk of less than 0.3, and a relative risk of less than 0.25. Other values of relative risk characterizing the decreased susceptibility or decreased risk are however also possible and within scope of the invention, including, but not limited to, less than 0.8, less than 0.75, less than 0.65, less than 0.55, less than 0.45, less than 0.20, etc.

In particular embodiments of the methods of the invention, the at least one marker or haplotype comprises rs16901979 allele 1, with the at least one marker or haplotype conferring an increased susceptibility of the cancer. In another such embodiment, the at least one marker or haplotype is the marker rs16901979 allele 1. In other particular embodiments of the methods of the invention, the at least one marker or haplotype comprises rs16901979 allele 2, with the at least one marker or haplotype conferring an increased susceptibility of the cancer. In another such embodiment, the at least one marker or haplotype is the marker rs16901979 allele 2.

In certain embodiments of the methods of the invention, the cancer is selected from the group consisting of prostate cancer, colon cancer, breast cancer, testicular cancer, lung cancer and melanoma cancer. In a preferred embodiment, the cancer is prostate cancer.

The prostate cancer is in one embodiment an aggressive prostate cancer as defined by a combined Gleason score of 7(4+3)−10. The prostate cancer is in another embodiment a less aggressive prostate cancer as defined by a combined Gleason score of 2−7(3+4). In one embodiment, the at least one marker or haplotype is indicative of a more aggressive prostate cancer and/or a worse prognosis.

Another embodiment of the methods of the invention relates to the presence of the marker or haplotype being indicative of a different response rate of the subject to a particular treatment modality. In another embodiment, the presence of the at least one marker or haplotype is indicative of a predisposition to a somatic rearrangement of Chr8q24.21 in a tumor or its precursor. In one such embodiment, the somatic rearrangement is selected from the group consisting of an amplification, a translocation, an insertion and a deletion.

The methods, uses and kits of invention can in certain embodiments relate to individuals with a particular ancestry. Thus, in one embodiment of the invention, the individual is of a specific ancestry. In another embodiment, the ancestry is black African ancestry. Other ancestry of the individuals to be assessed by the methods of the invention are also possible, as described in further detail herein, and are also within scope of the present invention. In one embodiment, the ancestry is self-reported. In another embodiment, the ancestry is determined by detecting at least one allele of at least one polymorphic marker in a sample from the individual, wherein the presence or absence of the allele is indicative of the ancestry of the individual.

Another aspect of the present invention relates to a method of identification of a marker for use in assessing susceptibility to cancer, the method comprising
a. identifying at least one polymorphic marker within SEQ ID NO:2, or at least one polymorphic marker in linkage disequilibrium therewith;
b. determining the genotype status of a sample of individuals diagnosed with, or having a susceptibility to, prostate cancer; and
c. determining the genotype status of a sample of control individuals;
   wherein a significant difference in frequency of at least one allele in at least one polymorphism in individuals diagnosed with, or having a susceptibility to, prostate cancer, as compared with the frequency of the at least one allele in the control sample is indicative of the at least one polymorphism being useful for assessing susceptibility to cancer.

Linkage disequilibrium is in one particular embodiment characterized by numerical values of r² of greater than 0.2 and/or |D′| of greater than 0.8. In another embodiment, the at least one polymorphic marker is in linkage disequilibrium, as characterized by numerical values of r² of greater than 0.2 and/or |D′| of greater than 0.8 with HapC and/or marker rs16901979. In another embodiment, an increase in frequency of the at least one allele in the at least one polymorphism in individuals diagnosed with, or having a susceptibility to, cancer, as compared with the frequency of the at least one allele in the control sample is indicative of the at least one polymorphism being useful for assessing increased susceptibility to cancer. In yet another embodiment, a decrease in frequency of the at least one allele in the at least one polymorphism in individuals diagnosed with, or having a susceptibility to, exfoliation syndrome, as compared with the frequency of the at least one allele in the control sample is indicative of the at least one polymorphism being useful for assessing decreased susceptibility to, or protection against, cancer.

The present invention, in another aspect, relates to a method of genotyping a nucleic acid sample obtained from a human individual at risk for, or diagnosed with, cancer, comprising determining the presence or absence of at least one allele of at least one polymorphic marker in the sample, wherein the at least one marker is selected from the group consisting of the markers set forth in Table 4A and 4B, and markers in linkage disequilibrium therewith, and wherein the presence or absence of the at least one allele of the at least one polymorphic marker is indicative of a susceptibility of cancer. In one embodiment the at least one marker is rs16901979 (SEQ ID NO:73), and markers in linkage disequilibrium therewith. In another embodiment, linkage disequilibrium is determined by numerical values for r² of at least 0.2 and/or numerical values of |D′| of at least 0.8. In another embodiment, the genotyping comprises amplifying a segment of a nucleic acid that comprises the at least one polymorphic marker by Polymerase Chain Reaction (PCR), using a nucleotide primer pair flanking the at least one polymorphic marker. In yet another embodiment, genotyping is performed using a process selected from allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis. In one such embodiment, the process comprises allele-specific probe hybridization. In another embodiment, the process comprises nucleic acid sequencing. In another embodiment, the nucleic acid sequencing is DNA sequencing.

One embodiment of a method of genotyping according to the invention, comprises the steps of:
1) contacting copies of the nucleic acid with a detection oligonucleotide probe and an enhancer oligonucleotide probe under conditions for specific hybridization of the oligonucleotide probe with the nucleic acid; wherein
   a) the detection oligonucleotide probe is from 5-100 nucleotides in length and specifically hybridizes to a first segment of the nucleic acid whose nucleotide sequence is given by SEQ ID NO:2 that comprises at least one polymorphic site;
   b) the detection oligonucleotide probe comprises a detectable label at its 3′ terminus and a quenching moiety at its 5′ terminus;
   c) the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid; and
   d) a single base gap exists between the first segment and the second segment, such that when the oligonucleotide probe and the enhancer oligonucleotide probe are both hybridized to the nucleic acid, a single base gap exists between the oligonucleotides;
2) treating the nucleic acid with an endonuclease that will cleave the detectable label from the 3′ terminus of the detection probe to release free detectable label when the detection probe is hybridized to the nucleic acid; and
3) measuring free detectable label, wherein the presence of the free detectable label indicates that the detection probe specifically hybridizes to the first segment of the nucleic acid, and indicates the sequence of the polymorphic site as the complement of the detection probe.

In a particular embodiment, the copies of the nucleic acid are provided by amplification by Polymerase Chain Reaction (PCR). In some embodiments the susceptibility to be detected is increased susceptibility. In other embodiment, the susceptibility is decreased susceptibility. In particular embodiments, the cancer is selected from prostate cancer, colon cancer, breast cancer, lung cancer, testicular cancer and melanoma. In a preferred embodiment, the cancer is prostate cancer. In one such embodiment, the prostate cancer is an aggressive prostate cancer as defined by a combined Gleason score of 7(4+3)−10. In another such embodiment, the prostate cancer is a less aggressive prostate cancer as defined by a combined Gleason score of 2−7(3+4).

The methods of identification of markers for use in assessing a susceptibility to cancer, and the methods of genotyping, are in some embodiments practiced on individual is of a specific ancestry. In one such embodiment, the ancestry is black African ancestry. Other ancestry is also within the scope of the invention, as described in detail in the herein. the ancestry is in one embodiment self-reported. In another embodiment, the ancestry is determined by detecting at least one allele of at least one polymorphic marker in a sample from the individual, wherein the presence or absence of the allele is indicative of the ancestry of the individual.

Another aspect of the present invention relates to a method of assessing an individual for probability of response to a therapeutic agent for preventing and/or ameliorating symptoms associated with cancer, comprising: determining the presence or absence of at least one allele of at least one polymorphic marker in a nucleic acid sample obtained from the individual, wherein the at least one polymorphic marker is selected from the group consisting of the polymorphic markers listed in Table 5A, 5B and 5C, and markers in linkage disequilibrium therewith, wherein the presence of the at least one allele of the at least one marker is indicative of a probability of a positive response to a symptoms associated with exfoliation syndrome and/or glaucoma therapeutic agent. Another aspect of the present invention relates to a method of predicting prognosis of an individual diagnosed with, cancer, the method comprising determining the presence or absence of at least one allele of at least one polymorphic marker in a nucleic acid sample obtained from the individual, wherein the at least one polymorphic marker is selected from the group consisting of the polymorphic markers listed in Table 5A, 5B and 5C, and markers in linkage disequilibrium therewith, wherein the presence of the at least one allele is indicative of a worse prognosis of the cancer in the individual. Yet another aspect of the invention relates to a method of monitoring progress of a treatment of an individual undergoing treatment for cancer, the method comprising determining the presence or absence of at least one allele of at least one polymorphic marker in a nucleic acid sample obtained from the individual, wherein the at least one polymorphic marker is selected from the group consisting of the polymorphic markers listed in Table 5A, 5B and 5C, and markers in linkage disequilibrium therewith, wherein the presence of the at least one allele is indicative of the treatment outcome of the individual. In any of these aspects, the at least one polymorphic marker is in one embodiment rs16901979 (SEQ ID NO:73) and markers in linkage disequilibrium therewith. In another embodiment, linkage disequilibrium is defined by numerical values of r² of at least 0.2 and/or values of |D′| of at least 0.8. In one preferred embodiment, the cancer is prostate cancer. In one such embodiment, the prostate cancer is an aggressive prostate cancer as defined by a combined Gleason score of 7(4+3)−10. In another embodiment, the prostate cancer is a less aggressive prostate cancer as defined by a combined Gleason score of 2−7(3+4).

The methods of the present invention may be utilized as such. In some embodiments, the methods may also be utilized in combination with other useful in the methods described herein. In one such embodiment, the method further comprises assessing at least one biomarker in a sample from the individual. The biomarker may be any biological marker useful for aiding in any decision-making based on the methods as described herein. In one embodiment, the biomarker is PSA. In another embodiment, the sample is a blood sample or a cancer biopsy sample. Other sample types useful for practicing the invention are however also contemplated, and within scope of the invention, such as other body fluids or tissue samples from any human tissue type.

Other embodiments of the methods of the invention, further comprise analyzing non-genetic information to make risk assessment, diagnosis, or prognosis of the individual. The non-genetic information are in one embodiment selected from age, gender, ethnicity, socioeconomic status, previous disease diagnosis, medical history of subject, family history of cancer, biochemical measurements, and clinical measurements. In a preferred embodiment, the method further comprises calculating overall risk based on genetic and non-genetic information.

Another aspect of the present invention relates to a kit for assessing susceptibility to cancer in a human individual, the kit comprising reagents for selectively detecting at least one allele of at least one polymorphic marker in the genome of the individual, wherein the polymorphic marker is selected from the group consisting of the polymorphic markers within the segment whose sequence is set forth in SEQ ID NO:2, and markers in linkage disequilibrium therewith, and wherein the presence of the at least one allele is indicative of a susceptibility to cancer. In one embodiment, the kit comprises at least one polymorphic marker selected from the group of markers set forth in Table 5A, 5B and 5C, and markers in linkage disequilibrium therewith. In another embodiment, the at least one polymorphic markers is selected from the group of markers set forth in Table 4A and 4B. In one preferred embodiment, the at least one polymorphic marker is selected from rs16901979 (SEQ ID NO: 73), and markers in linkage disequilibrium therewith. In another embodiment, the at least one polymorphic marker is rs16901979. In one embodiment, linkage disequilibrium is defined by numerical values of r² of at least 0.2 and/or values of |D′| of at least 0.8. In another embodiment, the cancer is selected from prostate cancer, colon cancer, breast cancer, testicular cancer, lung cancer and melanoma cancer. In a preferred embodiment, the cancer is prostate cancer. In one such embodiment, the prostate cancer is an aggressive prostate cancer as defined by a combined Gleason score of 7(4+3)−10. In another embodiment, the prostate cancer is a less aggressive prostate cancer as defined by a combined Gleason score of 2−7(3+4).

The kits of the invention may be used in any of the methods of the invention, as described herein. Thus, kits comprising reagents for specifically detecting at least one allele of at least one polymorphic marker, as described herein, may be utilized to practice any of the methods described herein, as will be apparent to the skilled person.

In one embodiment of the kit of the invention, the reagents comprise at least one contiguous oligonucleotide that hybridizes to a fragment of the genome of the individual comprising the at least one polymorphic marker, a buffer and a detectable label. In one embodiment, the reagents comprise at least one pair of oligonucleotides that hybridize to opposite strands of a genomic nucleic acid segment obtained from the subject, wherein each oligonucleotide primer pair is designed to selectively amplify a fragment of the genome of the individual that includes one polymorphic marker, and wherein the fragment is at least 30 base pairs in size. In a preferred embodiment, the at least one oligonucleotide is completely complementary to the genome of the individual. In another embodiment, the oligonucleotide is about 18 to about 50 nucleotides in length. In yet another embodiment, the oligonucleotide is 20-30 nucleotides in length.

In one preferred embodiment of the kit of the invention, the kit comprises:
a. a detection oligonucleotide probe that is from 5-100 nucleotides in length;
b. an enhancer oligonucleotide probe that is from 5-100 nucleotides in length; and
c. an endonuclease enzyme;
   wherein the detection oligonucleotide probe specifically hybridizes to a first segment of the nucleic acid whose nucleotide sequence is given by SEQ ID NO: 2 that comprises at least one polymorphic site; and
   wherein the detection oligonucleotide probe comprises a detectable label at its 3′ terminus and a quenching moiety at its 5′ terminus;
   wherein the enhancer oligonucleotide is from 5-100 nucleotides in length and is complementary to a second segment of the nucleotide sequence that is 5′ relative to the oligonucleotide probe, such that the enhancer oligonucleotide is located 3′ relative to the detection oligonucleotide probe when both oligonucleotides are hybridized to the nucleic acid;
   wherein a single base gap exists between the first segment and the second segment, such that when the oligonucleotide probe and the enhancer oligonucleotide probe are both hybridized to the nucleic acid, a single base gap exists between the oligonucleotides; and
   wherein treating the nucleic acid with the endonuclease will cleave the detectable label from the 3′ terminus of the detection probe to release free detectable label when the detection probe is hybridized to the nucleic acid.

Another aspect of the invention relates to the use of an oligonucleotide probe in the manufacture of a diagnostic reagent for diagnosing and/or assessing susceptibility to cancer in a human individual, wherein the probe hybridizes to a segment of a nucleic acid whose nucleotide sequence is given by SEQ ID NO: 2 that comprises at least one polymorphic site, wherein the fragment is 15-500 nucleotides in length. In one embodiment, the polymorphic site is selected from the polymorphic markers set forth in Table 5A, 5B and 5C, and polymorphisms in linkage disequilibrium therewith. In another embodiment, the polymorphic site is rs16901979 (SEQ ID NO: 73). In one embodiment, the cancer is selected from prostate cancer, colon cancer, breast cancer, testicular cancer, lung cancer and melanoma cancer. In a preferred embodiment, the cancer is prostate cancer. In one such embodiment, the prostate cancer is an aggressive prostate cancer as defined by a combined Gleason score of 7(4+3)−10. In another embodiment, the prostate cancer is a less aggressive prostate cancer as defined by a combined Gleason score of 2−7(3+4).

In yet another aspect, the present invention relates to a computer-readable medium on which is stored:
a. an identifier for at least one polymorphic marker;
b. an indicator of the frequency of at least one allele of said at least one polymorphic marker in a plurality of individuals diagnosed with cancer; and
c. an indicator of the frequency of the least one allele of said at least one polymorphic markers in a plurality of reference individuals;
   wherein the at least one polymorphic marker is selected from the polymorphic markers set forth in Table 5A, 5B and 5C, and polymorphisms in linkage disequilibrium therewith.

In one embodiment, the polymorphic site is marker rs16901979 (SEQ ID NO:73), and markers in linkage disequilibrium therewith, as defined by numerical values of r² of at least 0.2 and/or values of |D′| of at least 0.8. In another embodiment, the cancer is selected from prostate cancer, colon cancer, breast cancer, testicular cancer, lung cancer and melanoma cancer. In a preferred embodiment, the cancer is prostate cancer. In one such embodiment, the prostate cancer is an aggressive prostate cancer as defined by a combined Gleason score of 7(4+3)−10. In another embodiment, the prostate cancer is a less aggressive prostate cancer as defined by a combined Gleason score of 2−7(3+4).

The computer-readable medium according to the invention may, in some embodiments, comprise information about the ancestry of the plurality of individuals. In another embodiment, the plurality of individuals diagnosed with cancer and the plurality of reference individuals is of a specific ancestry. In one embodiment, the ancestry is black African ancestry. In another embodiment, the ancestry is self-reported. In yet another embodiment, the ancestry is determined genetically, by genotyping a plurality of polymorphic markers to assess ancestry, as further described herein.

The invention furthermore relates to an apparatus for determining a genetic indicator for cancer in a human individual, comprising:
a computer readable memory; and
a routine stored on the computer readable memory;
   wherein the routine is adapted to be executed on a processor to analyze marker and/or haplotype information for at least one human individual with respect to at least one polymorphic marker selected from the markers set forth in Table 5A, 5B and 5C, and markers in linkage disequilibrium therewith, and generate an output based on the marker or haplotype information, wherein the output comprises a risk measure of the at least one marker or haplotype as a genetic indicator of cancer for the human individual. In one embodiment, the routine further comprises an indicator of the frequency of at least one allele of at least one polymorphic marker or at least one haplotype in a plurality of individuals diagnosed with cancer, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with cancer.

In one embodiment, the at least one polymorphic marker is rs16901979 (SEQ ID NO:73), and markers in linkage disequilibrium therewith, as defined by numerical values of r² of at least 0.2 and/or values of |D′| of at least 0.8. In another embodiment, the risk measure is characterized by an Odds Ratio (OR) or a Relative Risk (RR).

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 depicts the LD structure (HAPMAP) in the Chr8q24.21 LD Block C area. The LD structure for Caucasians (CEU) is shown in (A), while the LD structure for Africans from Yoruba (YRI) is shown in (B). The thick diagonal line indicates the location of LD block C (SEQ ID NO:1). Each marker is shown in a sequential order with equal distances between two consecutive markers.

FIG. 2 depicts the LD structure of the LD Block C′ (SEQ ID NO:2) in the Chr8q24.21 region. The LD block as defined overlaps LD Block C, representing a refined analysis of the region within which variants associated with prostate cancer as described herein are located. The LD structure for Caucasians (CEU) is shown in (A), while the LD structure for Africans from Yoruba (YRI) is shown in (B). The thick diagonal line indicates the location of LD block C. Each marker is shown in a sequential order with equal distances between two consecutive markers.

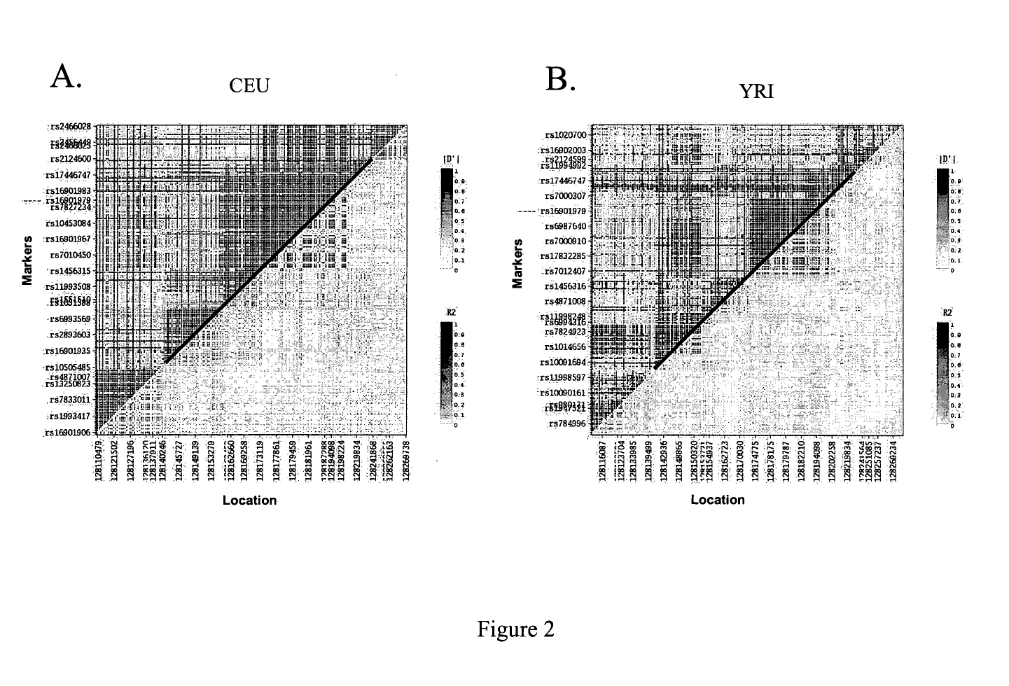

### DETAILED DESCRIPTION OF THE INVENTION

### Definitions

The following terms shall, in the present context, have the meaning as indicated:

A “polymorphic marker”, sometimes referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including single nucleotide polymorphisms (SNPs), microsatellites, insertions, deletions, duplications and translocations.

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome.

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “fragment” of a nucleotide or a protein, as described herein, comprises all or a part of the nucleotide or the protein.

An “animal”, as described herein, refers to any domestic animal (e.g., cats, dogs, etc.), agricultural animal (e.g., cows, horses, sheep, chicken, etc.), or test species (e.g., rabbit, mouse, rat, etc.), and also includes humans.

A “microsatellite”, as described herein, is a polymorphic marker that has multiple small repeats of bases that are 2-8 nucleotides in length (such as CA repeats) at a particular site, in which the number of repeat lengths varies in the general population.

An “indel”, as described herein, is a common form of polymorphism comprising a small insertion or deletion that is typically only a few nucleotides long.

A “haplotype,” as described herein, refers to a segment of genomic DNA within one strand of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles.

The term “susceptibility”, as described herein, encompasses both increased susceptibility and decreased susceptibility. Thus, particular polymorphic markers and/or haplotypes of the invention may be characteristic of increased susceptibility (i.e., increased risk) of glaucoma, as characterized by a relative risk (RR) of greater than one. Alternatively, the markers and/or haplotypes of the invention are characteristic of decreased susceptibility (i.e., decreased risk) of glaucoma, as characterized by a relative risk of less than one.

A “nucleic acid sample”, as described herein, is a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including as a blood sample, sample of amniotic fluid, sample of cerebrospinal fluid, or tissue sample from skin, muscle, buccal or conjunctival mucosa (buccal swab), placenta, gastrointestinal tract or other organs.

As used herein, “Chr8q24.21” and “8q24.21” refer to chromosomal band 8q24.21 which corresponds roughly to position 127,200,001-131,400,000 by in UCSC Build 34 (from the UCSC Genome browser Build 34 at www.genome.ucsc.edu).

As used herein, “LD Block C” refers to the LD block on Chr8q24.21 wherein association of variants to cancer, i.e. prostate cancer, breast cancer, lung cancer and melanoma is observed. NCBI Build 34 position of this LD block is from 128,032,278 to 128,094,256 by (SEQ ID NO:1).

As used herein, “LD Block C′” refers to the LD block on Chr8q24.21 wherein association of variants associated with cancer may preferably be detected. The NCBI Build 34 position of this LD block is from 128,029,113 to 128,126,447, and its sequence is set forth in SEQ ID NO:2

Bp. In NCBI Builds 35 and 36 the location of the region is from position 128,141,706 to 128,239,040. The sequence of the LD Block C′ region in Builds 34, 35 and 36 is identical over the entire span of 97,335 bp.

The term “African ancestry”, as described herein, refers to self-reported African ancestry of individuals.

The term “cancer therapeutic agent” refers to an agent that can be used to ameliorate or prevent symptoms associated with cancer (i.e., prostate cancer, lung cancer, breast cancer and/or melanoma).

The terms “associated with SEQ ID NO:2”, “associated with SEQ ID NO:1” “associated with LD Block C”, and “associated with LD Block C”, refer to those DNA segments (e.g. polymorphic markers) that are in linkage disequilibrium (LD) with the genomic segments represented by SEQ ID NO:2, SEQ ID NO:1, LD Block C and LD Block C′. In certain embodiments, such DNA segments are in LD with one or more markers within SEQ ID NO:2, SEQ ID NO:1, LD Block C or LD Block C′ as measured by values for |D′| greater than 0.8 and/or r² greater than 0.2.

### Association to Chr8q24.21

As discussed above, linkage to chromosome 8q24.21 and association to a linkage disequilibrium (LD) block within the linkage region has recently been reported. As described herein, it has now been surprisingly found that variants (markers and/or haplotypes) residing within another DNA segment of extensive LD (i.e., another LD block) in the chromosome 8q24.21 region are also associated with cancer. The association detected is independent of previously detected association in the region, a surprising result for the present inventors. In one embodiment of the invention, the association is detected by the haplotype HapC, comprising the markers rs1456314 allele G, rs17831626 allele T, rs7825414 allele G, rs6993569 allele G, rs6994316 allele A, rs6470494 allele T, rs1016342 allele C, rs1031588 allele G, rs1016343 allele T, rs1551510 allele G, rs1456306 allele C, rs1378897 allele G, rs1456305 allele T and rs7816535 allele G. As with other variants associated with human traits, a large number of surrogate variants (markers and/or haplotypes) can be described. One such surrogate marker for HapC is marker rs16901979. The region most likely to harbor surrogate variants is commonly defined as a region of extensive linkage disequilibrium, so called linkage disequilibrium blocks (LD block), as further described herein. In one embodiment, an LD block encompassing the variants associating with cancer is LD Block C characterized by the sequence set forth in SEQ ID NO:1. A further refinement of the signal originally detected by the inventors defines the LD Block C′ as the region between two recombination hotspots on chromosome 8q24.21, The hotspots are located at approximately position 128,029,113 and 128,126,447 on chromosome 8, and the region thus defined is as set forth in SEQ ID NO:2. Surrogate markers and/or haplotypes for HapC, rs16901979 can be found within either LD Block as defined (i.e, SEQ ID NO:1, and SEQ ID NO:2) and further described in detail herein.

In various embodiments of the invention, certain markers and/or SNPs, identified using the methods described herein, can be used for a diagnosis of increased susceptibility to cancer (e.g., prostate cancer), and also for a diagnosis of a decreased susceptibility to cancer (e.g., prostate cancer), i.e. for identification of variants that are protective against cancer (e.g., prostate cancer). The diagnostic assays presented below can be used to identify the presence or absence of these particular variants.

The Gleason score is the most frequently used grading system for prostate cancer (DeMarzo, A. M. et al., Lancet 361:955-64 (2003)). The system is based on the discovery that prognosis of prostate cancer is intermediate between that of the most predominant pattern of cancer and that of the second most predominate pattern. These predominant and second most prevalent patterns are identified in histological samples from prostate tumors and each is graded from 1 (most differentiated) to 5 (least differentiated) and the two scores are added. The combined Gleason grade, also known as the Gleason sum or score, thus ranges from 2 (for tumors uniformly of pattern 1) to 10 (for undifferentiated tumors). Most cases with divergent patterns, especially on needle biopsy, do not differ by more than one pattern.

The Gleason score is a prognostic indicator, with the major prognostic shift being between 6 and 7, as Gleason score 7 tumors behave much worse leading to more morbidity and higher mortality than tumors scoring 5 or 6. Score 7 tumors can further be subclassified into 3+4 or 4+3 (the first number is the predominant histologic subtype in the biopsied tumor sample and the second number is the next predominant histologic subtype), with the 4+3 score being associated with worse prognosis. A patient's Gleason score can also influence treatment options. For example, younger men with limited amounts of a Gleason score 5-6 on needle biopsy and low PSA concentrations may simply be monitored while men with Gleason scores of 7 or higher usually receive active management. In Table 1, the frequency of haplotype and the associated risk of aggressive prostate cancer (i.e., as indicated by a combined Gleason score of 7(4+3 only) to 10) and less aggressive prostate cancer (i.e., as indicated by a combined Gleason score of 2 to 7 (3+4 only)) are indicated. However, the Gleason score is not a perfect predictor of prognosis. Thus, patients with tumors with low Gleason scores may still have more aggressive prostate cancer (defined as tumors extending beyond the prostate locally or through distant metastasis).

In certain methods described herein, an individual who is at risk (increased susceptibility) for cancer (e.g., prostate cancer (aggressive or high Gleason grade prostate cancer, less aggressive or low Gleason grade prostate cancer) is an individual in whom an at-risk marker or haplotype is identified. In one embodiment, the strength of the association of a marker or haplotype is measured by relative risk (RR). RR is the ratio of the incidence of the condition among subjects who carry one copy of the marker or haplotype to the incidence of the condition among subjects who do not carry the marker or haplotype. This ratio is equivalent to the ratio of the incidence of the condition among subjects who carry two copies of the marker or haplotype to the incidence of the condition among subjects who carry one copy of the marker or haplotype.

In one embodiment, the invention is a method of diagnosing a susceptibility to prostate cancer (e.g., aggressive or high Gleason grade prostate cancer, less aggressive or low Gleason grade prostate cancer), comprising detecting a marker or haplotype associated with LD Block C (e.g., a marker or haplotype as set forth in Table 5, having a value of relative risk (RR) greater than one, indicating the marker is associated with increased susceptibility to disease/increased risk of disease and thus is an “at-risk” variant; a marker or haplotype with values of RR less than one indicate the marker is associated with decreased susceptibility to disease/decreased risk of disease and thus is a “protective” variant), wherein the presence of the marker or haplotype is indicative of a susceptibility to prostate cancer.

In another embodiment, the invention is a method of diagnosing a susceptibility to prostate cancer (e.g., aggressive or high Gleason grade prostate cancer, less aggressive or low Gleason grade prostate cancer), comprising detecting marker rs16901979. In one embodiment, the susceptibility is increased susceptibility, wherein the presence of the 1 allele at marker rs16901979 is indicative of an increased susceptibility to prostate cancer. In a further embodiment, the invention is a method of diagnosing increased susceptibility to prostate cancer in an individual whose ancestry comprises African ancestry, comprising detecting marker rs16901979, wherein the presence of the 1 allele at marker rs16901979 is indicative of an increased susceptibility to prostate cancer or an increased risk of prostate cancer. In particular embodiments, the marker or haplotype that is associated with a susceptibility to prostate cancer has a relative risk of at least 1.3, such as at least 1.5 or at least 1.7 or at least 2.0. In another embodiment, the prostate cancer is an aggressive prostate cancer, as defined by a combined Gleason score of 7(4+3) to 10 and/or an advanced stage of prostate cancer (e.g., Stages 2 to 4). In yet another embodiment, the prostate cancer is a less aggressive prostate cancer, as defined by a combined Gleason score of 2 to 7(3+4) and/or an early stage of prostate cancer (e.g., Stage 1). In another embodiment, the presence of a marker or haplotype associated with LD Block C, in conjunction with the subject having a PSA level greater than 4 ng/ml, is indicative of a more aggressive prostate cancer and/or a worse prognosis. In yet another embodiment, in patients who have a normal PSA level (e.g., less than 4 ng/ml), the presence of a marker or haplotype is indicative of a more aggressive prostate cancer and/or a worse prognosis.

In other embodiments, the invention is a method of diagnosing a decreased susceptibility to prostate cancer, comprising detecting a marker or haplotype associated with LD Block C, wherein the presence of that marker or haplotype is indicative of a decreased susceptibility to prostate cancer or of a protective marker or haplotype against prostate cancer. Thus, in one embodiment, the susceptibility is decreased susceptibility, wherein the presence of the 2 allele at marker rs16901979 is indicative of a decreased susceptibility to prostate cancer. In a further embodiment, the invention is a method of diagnosing decreased susceptibility to prostate cancer in an individual whose ancestry comprises African ancestry, comprising detecting marker rs16901979, wherein the presence of the s allele at marker rs16901979 is indicative of a decreased susceptibility to prostate cancer or a decreased risk of prostate cancer.

The segment on chromosome 8q24.21 of the present invention has also been found to play a role in other forms of cancer, e.g. breast cancer, colon cancer, lung cancer and melanoma. It has been discovered that particular markers and/or haplotypes in a specific DNA segment within the region are present at a higher than expected frequency in breast cancer subjects. Thus, in one embodiment, the invention is a method of diagnosing an increased susceptibility to cancer selected from breast cancer, lung cancer, colon cancer and melanoma, comprising detecting a marker or haplotype associated with the genomic segments whose sequence is set forth in SEQ ID NO:1 or SEQ ID NO:2, wherein the presence of the marker or haplotype is indicative of an increased susceptibility to the cancer (e.g., breast cancer, colon cancer, lung cancer and melanoma). In a particular embodiment, the marker or haplotype that is associated with a susceptibility to cancer (i.e., breast cancer, lung cancer and melanoma) has a relative risk of at least 1.3, such as at least 1.5, at least 1.7 or at least 2.0. In other embodiments, the invention is drawn to a method of diagnosing a decreased susceptibility to cancer (i.e., breast cancer, lung cancer and melanoma) comprising detecting a marker or haplotype associated with the genomic segments whose sequence is set forth in SEQ ID NO:1 or SEQ ID NO:2, wherein the presence of that marker or haplotype is indicative of a decreased susceptibility to cancer or of a protective marker or haplotype against breast cancer (protective against cancer (i.e., breast cancer, lung cancer and melanoma)). In a particular embodiment, the marker or haplotype that is associated with a decreased susceptibility to cancer (i.e., breast cancer, lung cancer and melanoma) has a relative risk of less than 0.9, such as less than 0.8, less than 0.7, less than 0.6 and less than 0.5. In another embodiment, the melanoma is malignant cutaneous melanoma.

### Assessment for Markers and Haplotypes

The genomic sequence within populations is not identical when individuals are compared.

Rather, the genome exhibits sequence variability between individuals at many locations in the genome. Such variations in sequence are commonly referred to as polymorphisms, and there are many such sites within each genome For example, the human genome exhibits sequence variations which occur on average every 500 base pairs. The most common sequence variant consists of base variations at a single base position in the genome, and such sequence variants, or polymorphisms, are commonly called Single Nucleotide Polymorphisms (“SNPs”). These SNPs are believed to have occurred in a single mutational event, and therefore there are usually two possible alleles possible at each SNPsite; the original allele and the mutated allele. Due to natural genetic drift and possibly also selective pressure, the original mutation has resulted in a polymorphism characterized by a particular frequency of its alleles in any given population. Many other types of sequence variants are found in the human genome, including microsatellites, insertions, deletions, inversions and copy number variations. A polymorphic microsatellite has multiple small repeats of bases (such as CA repeats, TG on the complimentary strand) at a particular site in which the number of repeat lengths varies in the general population. In general terms, each version of the sequence with respect to the polymorphic site represents a specific allele of the polymorphic site. These sequence variants can all be referred to as polymorphisms, occurring at specific polymorphic sites characteristic of the sequence variant in question. In general terms, polymorphisms can comprise any number of specific alleles. Thus in one embodiment of the invention, the polymorphism is characterized by the presence of two or more alleles in any given population. In another embodiment, the polymorphism is characterized by the presence of three or more alleles. In other embodiments, the polymorphism is characterized by four or more alleles, five or more alleles, six or more alleles, seven or more alleles, nine or more alleles, or ten or more alleles. All such polymorphisms can be utilized in the methods and kits of the present invention, and are thus within the scope of the invention.

In some instances, reference is made to different alleles at a polymorphic site without choosing a reference allele. Alternatively, a reference sequence can be referred to for a particular polymorphic site. The reference allele is sometimes referred to as the “wild-type” allele and it usually is chosen as either the first sequenced allele or as the allele from a “non-affected” individual (e.g., an individual that does not display a trait or disease phenotype).

Alleles for SNP markers as referred to herein refer to the bases A, C, G or T as they occur at the polymorphic site in the SNP assay employed. The allele codes for SNPs used herein are as follows: 1=A, 2=C, 3=G, 4=T. The person skilled in the art will however realise that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+strand or −strand).

Typically, a reference sequence is referred to for a particular sequence. Alleles that differ from the reference are sometimes referred to as “variant” alleles. A variant sequence, as used herein, refers to a sequence that differs from the reference sequence but is otherwise substantially similar. Alleles at the polymorphic genetic markers described herein are variants. Additional variants can include changes that affect a polypeptide. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences.

A haplotype refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, or five or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various genetic markers.

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), utilizing PCR, LCR, Nested PCR and other techniques for nucleic acid amplification. Specific methodologies available for SNP genotyping include, but are not limited to, TaqMan genotyping assays and SNPlex platforms (Applied Biosystems), mass spectrometry (e.g., MassARRAY system from Sequenom), minisequencing methods, real-time PCR, Bio-Plex system (BioRad), CEQ and SNPstream systems (Beckman), Molecular Inversion Probe array technology (e.g., Affymetrix GeneChip), and BeadArray Technologies (e.g., Illumina GoldenGate and Infinium assays). By these or other methods available to the person skilled in the art, one or more alleles at polymorphic markers, including microsatellites, SNPs or other types of polymorphic markers, can be identified.

In certain methods described herein, an individual who is at an increased susceptibility (i.e., increased risk) for any specific disease or trait under study, is an individual in whom at least one specific allele at one or more polymorphic marker or haplotype conferring increased susceptibility for the disease or trait is identified (i.e., at-risk marker alleles or haplotypes). In one aspect, the at-risk marker or haplotype is one that confers a significant increased risk (or susceptibility) of the disease or trait. In one embodiment, significance associated with a marker or haplotype is measured by a relative risk (RR). In another embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.2, including but not limited to: at least 1.2, at least 1.3, at least 1.4, at least 1.5, at least 1.6, at least 1.7, 1.8, at least 1.9, at least 2.0, at least 2.5, at least 3.0, at least 4.0, and at least 5.0. In a particular embodiment, a risk (relative risk and/or odds ratio) of at least 1.2 is significant. In another particular embodiment, a risk of at least 1.3 is significant. In yet another embodiment, a risk of at least 1.4 is significant. In a further embodiment, a relative risk of at least about 1.5 is significant. In another further embodiment, a significant increase in risk is at least about 1.7 is significant. However, other cutoffs are also contemplated, e.g. at least 1.15, 1.25, 1.35, and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention.

An at-risk polymorphic marker or haplotype of the present invention is one where at least one allele of at least one marker or haplotype is more frequently present in an individual at risk for the disease or trait (affected), compared to the frequency of its presence in a comparison group (control), and wherein the presence of the marker or haplotype is indicative of susceptibility to the disease or trait. The control group may in one embodiment be a population sample, i.e. a random sample from the general population. In another embodiment, the control group is represented by a group of individuals who are disease-free. Such disease-free control may in one embodiment be characterized by the absence of one or more specific disease-associated symptoms. In another embodiment, the disease-free control group is characterized by the absence of one or more disease-specific risk factors. Such risk factors are in one embodiment at least one environmental risk factor. Representative environmental factors are natural products, minerals or other chemicals which are known to affect, or contemplated to affect, the risk of developing the specific disease or trait. Other environmental risk factors are risk factors related to lifestyle, including but not limited to food and drink habits, geographical location of main habitat, and occupational risk factors. In another embodiment, the risk factors are at least one genetic risk factor.

As an example of a simple test for correlation would be a Fisher-exact test on a two by two table. Given a cohort of chromosomes, the two by two table is constructed out of the number of chromosomes that include both of the markers or haplotypes, one of the markers or haplotypes but not the other and neither of the markers or haplotypes.

In other embodiments of the invention, an individual who is at a decreased susceptibility (i.e., at a decreased risk) for a disease or trait is an individual in whom at least one specific allele at one or more polymorphic marker or haplotype conferring decreased susceptibility for the disease or trait is identified. The marker alleles and/or haplotypes conferring decreased risk are also said to be protective. In one aspect, the protective marker or haplotype is one that confers a significant decreased risk (or susceptibility) of the disease or trait. In one embodiment, significant decreased risk is measured as a relative risk of less than 0.9, including but not limited to less than 0.9, less than 0.8, less than 0.7, less than 0.6, less than 0.5, less than 0.4, less than 0.3, less than 0.2 and less than 0.1. In one particular embodiment, significant decreased risk is less than 0.7. In another embodiment, significant decreased risk is less than 0.5. In yet another embodiment, significant decreased risk is less than 0.3. In another embodiment, the decrease in risk (or susceptibility) is at least 20%, including but not limited to at least 25%, at least 30%, at least 35%, at least 40%, at least 45%, at least 50%, at least 55%, at least 60%, at least 65%, at least 70%, at least 75%, at least 80%, at least 85%, at least 90%, at least 95% and at least 98%. In one particular embodiment, a significant decrease in risk is at least about 30%. In another embodiment, a significant decrease in risk is at least about 50%. In another embodiment, the decrease in risk is at least about 70%. Other cutoffs or ranges as deemed suitable by the person skilled in the art to characterize the invention are however also contemplated, and those are also within scope of the present invention.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather, there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A. J., et al., Nature Genet. 29:217-222 (2001); May, C. A., et al., Nature Genet. 31:272-275 (2002)).

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrance of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD). Most capture the strength of association between pairs of biallelic sites. Two important pairwise measures of LD are r² (sometimes denoted Δ²) and |D′|. Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one preferred embodiment, the significant r² value can be at least 0.2. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). In certain embodiments, linkage disequilibrium is defined in terms of values for both the r² and |D′| measures. In one such embodiment, a significant linkage disequilibrium is defined as r²>0.1 and |D′|>0.8. In another embodiment, a significant linkage disequilibrium is defined as r²>0.2 and |D′|>0.9. Other combinations and permutations of values of r² and |D′| for determining linkage disequilibrium are also possible, and within the scope of the invention. Linkage disequilibrium can be determined in a single human population, as defined herein, or it can be determined in a collection of samples comprising individuals from more than one human population. In one embodiment of the invention, LD is determined in a sample from one or more of the HapMap populations (caucasian (CEU), african (YRI), japanese (JPT), chinese (CHB)), as defined (http://www.hapmap.org). In one such embodiment, LD is determined in the CEU population of the HapMap samples. In another embodiment, LD is determined in the YRI population. In yet another embodiment, LD is determined in samples from the Icelandic population.

If all polymorphisms in the genome were identical at the population level, then every single one of them would need to be investigated in association studies. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)).

There are two main methods for defining these haplotype blocks: blocks can be defined as regions of DNA that have limited haplotype diversity (see, e.g., Daly, M. et al., Nature Genet. 29:229-232 (2001); Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Some representative methods for identification of haplotype blocks are set forth, for example, in U.S. Published Patent Application Nos. 20030099964, 20030170665, 20040023237 and 20040146870. Haplotype blocks can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the present invention. One or more causative (functional) variants or mutations may reside within the region found to be associating to the disease or trait. Such variants may confer a higher relative risk (RR) or odds ratio (OR) than observed for the tagging markers used to detect the association. The present invention thus refers to the markers used for detecting association to the disease, as described herein, as well as markers in linkage disequilibrium with the markers. Thus, in certain embodiments of the invention, markers that are in LD with the markers and/or haplotypes of the invention, as described herein, may be used as surrogate markers. The surrogate markers have in one embodiment relative risk (RR) and/or odds ratio (OR) values smaller than for the markers or haplotypes initially found to be associating with the disease, as described herein. In other embodiments, the surrogate markers have RR or OR values greater than those initially determined for the markers initially found to be associating with the disease, as described herein. An example of such an embodiment would be a rare, or relatively rare (<10% allelic population frequency) variant in LD with a more common variant (>10% population frequency) initially found to be associating with the disease, such as the variants described herein. Identifying and using such markers for detecting the association discovered by the inventors as described herein can be performed by routine methods well known to the person skilled in the art, and are therefore within the scope of the present invention.

### Determination of Haplotype Frequency

The frequencies of haplotypes in patient and control groups can be estimated using an expectation-maximization algorithm (Dempster A. et al., J. R. Stat. Soc. B, 39:1-38 (1977)). An implementation of this algorithm that can handle missing genotypes and uncertainty with the phase can be used. Under the null hypothesis, the patients and the controls are assumed to have identical frequencies. Using a likelihood approach, an alternative hypothesis is tested, where a candidate at-risk-haplotype, which can include the markers described herein, is allowed to have a higher frequency in patients than controls, while the ratios of the frequencies of other haplotypes are assumed to be the same in both groups. Likelihoods are maximized separately under both hypotheses and a corresponding 1-df likelihood ratio statistic is used to evaluate the statistical significance.

To look for at-risk and protective markers and haplotypes within a linkage region, for example, association of all possible combinations of genotyped markers is studied, provided those markers span a practical region. The combined patient and control groups can be randomly divided into two sets, equal in size to the original group of patients and controls. The marker and haplotype analysis is then repeated and the most significant p-value registered is determined. This randomization scheme can be repeated, for example, over 100 times to construct an empirical distribution of p-values. In a preferred embodiment, a p-value of <0.05 is indicative of a significant marker and/or haplotype association.

### Haplotype Analysis

One general approach to haplotype analysis involves using likelihood-based inference applied to NEsted MOdels (Gretarsdottir S., et al., Nat. Genet. 35:131-38 (2003)). The method is implemented in the program NEMO, which allows for many polymorphic markers, SNPs and microsatellites. The method and software are specifically designed for case-control studies where the purpose is to identify haplotype groups that confer different risks. It is also a tool for studying LD structures. In NEMO, maximum likelihood estimates, likelihood ratios and p-values are calculated directly, with the aid of the EM algorithm, for the observed data treating it as a missing-data problem.

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. The information measure for haplotype analysis is described in Nicolae and Kong (Technical Report 537, Department of Statistics, University of Statistics, University of Chicago; Biometrics, 60(2):368-75 (2004)) as a natural extension of information measures defined for linkage analysis, and is implemented in NEMO.

For single marker association to a disease, the Fisher exact test can be used to calculate two-sided p-values for each individual allele. Usually, all p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1273-1288 (1998)), DNA pooling (ibid) for sibships so that it can be applied to general familial relationships, and present both adjusted and unadjusted p-values for comparison. The differences are in general very small as expected. To assess the significance of single-marker association corrected for multiple testing we can carry out a randomization test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(f/pᵢ)/(f/pᵢ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

### Linkage Disequilibrium Using NEMO

LD between pairs of markers can be calculated using the standard definition of D′ and r² (Lewontin, R., Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and r² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

### Risk Assessment and Diagnostics

As described herein, certain polymorphic markers and haplotypes comprising such markers are found to be useful for risk assessment of cancer (e.g., prostate cancer (e.g., aggressive prostate cancer, lung cancer, colon cancer, breast cancer, melanoma). Risk assessment can involve the use of the markers for diagnosing a susceptibility to cancer. Particular alleles of polymorphic markers are found more frequently in individuals with cancer, than in individuals without diagnosis of cancer. Therefore, these marker alleles have predictive value for detecting cancer, or a susceptibility to cancer, in an individual. Tagging markers within haplotype blocks or LD blocks comprising at-risk markers, such as the markers of the present invention, can be used as surrogates for other markers and/or haplotypes within the haplotype block or LD block. Markers with values of r² equal to 1 are perfect surrogates for the at-risk variants, i.e. genotypes for one marker perfectly predicts genotypes for the other. Markers with smaller values of r² than 1, can also be surrogates for the at-risk variant, or alternatively represent variants with relative risk values as high as or possibly even higher than the at-risk variant. The at-risk variant identified may not be the functional variant itself, but is in this instance in linkage disequilibrium with the true functional variant. The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation genotype surrogate markers in linkage disequilibrium with the markers and/or haplotypes as described herein. The tagging or surrogate markers in LD with the at-risk variants detected, also have predictive value for detecting association to the cancer, or a susceptibility to the cancer, in an individual. These tagging or surrogate markers that are in LD with the markers of the present invention can also include other markers that distinguish among haplotypes, as these similarly have predictive value for detecting susceptibility to cancer.

The markers and haplotypes of the invention, e.g., the markers presented in Table 4A, 4B, 5A, 5B, 5C, may be useful for risk assessment and diagnostic purposes for, either alone or in combination. Thus, even in cases where the increase in risk by individual markers is relatively modest, i.e. on the order of 10-30%, the association may have significant implications. Thus, relatively common variants may have significant contribution to the overall risk (Population Attributable Risk is high), or combination of markers can be used to define groups of individual who, based on the combined risk of the markers, is at significant combined risk of developing the disease.

Thus, in one embodiment of the invention, a plurality of variants (genetic markers, biomarkers and/or haplotypes) is used for overall risk assessment. These variants are in one embodiment selected from the variants as disclosed herein. Other embodiments include the use of the variants of the present invention in combination with other variants known to be useful for diagnosing a susceptibility to cancer. In such embodiments, the genotype status of a plurality of markers and/or haplotypes is determined in an individual, and the status of the individual compared with the population frequency of the associated variants, or the frequency of the variants in clinically healthy subjects, such as age-matched and sex-matched subjects. Methods known in the art, such as multivariate analyses or joint risk analyses, may subsequently be used to determine the overall risk conferred based on the genotype status at the multiple loci. Assessment of risk based on such analysis may subsequently be used in the methods and kits of the invention, as described herein.

As described in the above, the haplotype block structure of the human genome has the effect that a large number of variants (markers and/or haplotypes) in linkage disequilibrium with the variant originally associated with a disease or trait may be used as surrogate markers for assessing association to the disease or trait. The number of such surrogate markers will depend on factors such as the historical recombination rate in the region, the mutational frequency in the region (i.e., the number of polymorphic sites or markers in the region), and the extent of LD (size of the LD block) in the region. These markers are usually located within the physical boundaries of the LD block or haplotype block in question as defined using the methods described herein, or by other methods known to the person skilled in the art. However, sometimes marker and haplotype association is found to extend beyond the physical boundaries of the haplotype block as defined. Such markers and/or haplotypes may in those cases be also used as surrogate markers and/or haplotypes for the markers and/or haplotypes physically residing within the haplotype block as defined. As a consequence, markers and haplotypes in LD (typically characterized by r² greater than 0.1, such as r² greater than 0.2, including r² greater than 0.3, also including r² greater than 0.4) with the markers and haplotypes described herein are also within the scope of the invention, even if they are physically located beyond the boundaries of the haplotype block as defined. The invention thus relates to markers that are described herein (e.g., Table 4A, 4B, 5A, 5B, 5C), but may also include other markers that are in strong LD (e.g., characterized by r² greater than 0.1 or 0.2 and/or |D′|>0.8) with one or more of the markers listed herein.

For the SNP markers described herein, the opposite allele to the allele found to be in excess in patients (at-risk allele) with a particular cancer (e.g., prostate cancer) is found in decreased frequency in patients with the cancer. These markers and haplotypes in LD and/or comprising such markers, are thus protective for the cancer, i.e. they confer a decreased risk or susceptibility of individuals carrying these markers and/or haplotypes developing the cancer. In other embodiments, haplotypes comprising at least two polymorphic markers may be found in decreased frequency in individuals with the particular cancer, and are thus protective for the cancer. Such markers and haplotypes are useful for diagnosing a decreased susceptibility to the cancer in an individual.

Certain variants of the present invention, including certain haplotypes comprise, in some cases, a combination of various genetic markers, e.g., SNPs and microsatellites. Detecting haplotypes can be accomplished by methods known in the art and/or described herein for detecting sequences at polymorphic sites. Furthermore, correlation between certain haplotypes or sets of markers and disease phenotype can be verified using standard techniques. A representative example of a simple test for correlation would be a Fisher-exact test on a two by two table.

In specific embodiments, a marker allele or haplotype found to be associated with cancer, (e.g., marker alleles as listed in Tables 1, 2, 3, 4A and 4B) is one in which the marker allele or haplotype is more frequently present in an individual at risk for cancer (affected), compared to the frequency of its presence in a healthy individual (control), wherein the presence of the marker allele or haplotype is indicative of cancer or a susceptibility to cancer. In other embodiments, at-risk markers in linkage disequilibrium with one or more markers found to be associated with cancer are tagging markers that are more frequently present in an individual at risk for cancer (affected), compared to the frequency of their presence in a healthy individual (control), wherein the presence of the tagging markers is indicative of increased susceptibility to cancer. In a further embodiment, at-risk markers alleles (i.e. conferring increased susceptibility) in linkage disequilibrium with one or more markers found to be associated with cancer (e.g., marker alleles as listed in Table 4A, 4B, 5A, 5B and 5C), are markers comprising one or more allele that is more frequently present in an individual at risk for cancer, compared to the frequency of their presence in a healthy individual (control), wherein the presence of the markers is indicative of increased susceptibility to.

### Study Population

In a general sense, the methods and kits of the invention can be utilized from samples containing genomic DNA from any source, i.e. any individual. In preferred embodiments, the individual is a human individual. The individual can be an adult, child, or fetus. The present invention also provides for assessing markers and/or haplotypes in individuals who are members of a target population. Such a target population is in one embodiment a population or group of individuals at risk of developing the disease, based on other genetic factors, biomarkers, biophysical parameters (e.g., weight, BMD, blood pressure), or general health and/or lifestyle parameters (e.g., history of disease or related diseases, previous diagnosis of disease, family history of disease).

The invention provides for embodiments that include individuals from specific age subgroups, such as those over the age of 40, over age of 45, or over age of 50, 55, 60, 65, 70, 75, 80, or 85. Other embodiments of the invention pertain to other age groups, such as individuals aged less than 85, such as less than age 80, less than age 75, or less than age 70, 65, 60, 55, 50, 45, 40, 35, or age 30. Other embodiments relate to individuals with age at onset of the disease in any of the age ranges described in the above. It is also contemplated that a range of ages may be relevant in certain embodiments, such as age at onset at more than age 45 but less than age 60. Other age ranges are however also contemplated, including all age ranges bracketed by the age values listed in the above. The invention furthermore relates to individuals of either gender, males or females.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Stacey, S. N., et al., Nat. Genet. May 27, 2007 (Epub ahead of print; Helgadottir, A., of al., Science 316:1491-93 (2007); Steinthorsdottir, V., et al., Nat. Genet. 39:770-75 (2007); Gudmundsson, J., et al., Nat. Genet. 39:631-37 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

The markers of the present invention found to be associated with cancer (e.g., prostate cancer) are believed to show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, Asian populations, Central/South Asian populations, East Asian populations, Middle Eastern populations, African populations, Hispanic populations, and Oceanian populations. European populations include, but are not limited to, Swedish, Norwegian, Finnish, Russian, Danish, Icelandic, Irish, Kelt, English, Scottish, Dutch, Belgian, French, German, Spanish, Portugues, Italian, Polish, Bulgarian, Slavic, Serbian, Bosnian, Chech, Greek and Turkish populations. The invention furthermore in other embodiments can be practiced in specific human populations that include Bantu, Mandenk, Yoruba, San, Mbuti Pygmy, Orcadian, Adygel, Russian, Sardinian, Tuscan, Mozabite, Bedouin, Druze, Palestinian, Balochi, Brahui, Makrani, Sindhi, Pathan, Burusho, Hazara, Uygur, Kalash, Han, Dai, Daur, Hezhen, Lahu, Miao, Orogen, She, Tujia, Tu, Xibo, Yi, Mongolan, Naxi, Cambodian, Japanese, Yakut, Melanesian, Papuan, Karitianan, Surui, Colmbian, Maya and Pima.

In one preferred embodiment, the invention relates to populations that include black African ancestry such as populations comprising persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. In another example, self-reported persons of black African ancestry may have at least one parent of black African ancestry or at least one grandparent of black African ancestry.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)). In one embodiment, genetic ancestry is estimated using a set of microsatellite markers selected from the about 2000 microsatellites genotyped in a previously described study (Pritchard, J. K. et al., Genetics 15:945-59 (2000)), using a multi-ethnic cohort. In one such embodiment, a cohort of 35 European Americans, 88 African Americans, 34 Chinese, and 29 Mexican Americans is used, as described herein. One particular embodiment useful for estimating genetic ancestry from this set comprises 30 unlinked microsatellite markers. The selected set shows the most significant differences between European Americans, African Americans, and Asians of the 2000 markers described my Prichard et al., and also has good quality and yield. Thus, in one embodiment, genetic ancestry is determined by genotyping the set of microsatellite markers consisting of D1S2630, D1S2847, D1S466, D1S493, D2S166, D3S1583, D3S4011, D3S4559, D4S2460, D4S3014, D5S1967, DG5S802, D6S1037, D8S1719, D8S1746, D9S1777, D9S1839, D9S2168, D10S1698, D11S1321, D11S4206, D12S1723, D13S152, D14S588, D17S1799, D17S745, D18S464, D19S113, D20S878 and D22S1172. Appropriate primer pairs for amplifying a segment comprising marker DG5S802 is set forth in SEQ ID NO:4 and SEQ ID NO:5. The skilled person will realize that other combinations of microsatellite markers, or other types of polymorphic markers (e.g., SNPs) may also be used to estimate genetic ancestry.

In certain embodiments, the invention relates to markers and/or haplotypes identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Utility of Genetic Testing

The person skilled in the art will appreciate and understand that the variants described herein in general do not, by themselves, provide an absolute identification of individuals who will develop a particular cancer, e.g., prostate cancer. The variants described herein do however indicate increased and/or decreased likelihood that individuals carrying the at-risk or protective variants of the invention will develop a particular form of cancer, accompanied by symptoms associated with the cancer. This information is however extremely valuable in itself, as outlined in more detail in the below, as it can be used to, for example, initiate preventive measures at an early stage, perform regular physical and/or mental exams to monitor the progress and/or appearance of symptoms, or to schedule exams at a regular interval to identify early signs of the cancer, so as to be able to apply treatment at an early stage.

The knowledge about a genetic variant that confers a risk of developing cancer offers the opportunity to apply a genetic-test to distinguish between individuals with increased risk of developing the cancer (i.e. carriers of the at-risk variant) and those with decreased risk of developing the cancer (i.e. carriers of the protective variant). The core values of genetic testing, for individuals belonging to both of the above mentioned groups, are the possibilities of being able to diagnose the cancer at an early stage and provide information to the clinician about prognosis/aggressiveness of the cancer in order to be able to apply the most appropriate treatment. For example, the application of a genetic test for cancer (e.g., prostate cancer (aggressive or high Gleason grade prostate cancer, less aggressive or low Gleason grade prostate cancer)) can provide an opportunity for the detection of the disease at an earlier stage which may lead to the application of therapeutic measures at an earlier stage, and thus can minimize the deleterious effects of the symptoms and serious health consequences conferred by cancer. Some advantages of genetic tests for cancer include:

### 1. To Aid Early Detection

The application of a genetic test for prostate cancer can provide an opportunity for the detection of the disease at an earlier stage which leads to higher cure rates, if found locally, and increases survival rates by minimizing regional and distant spread of the tumor. For prostate cancer, a genetic test will most likely increase the sensitivity and specificity of the already generally applied Prostate Specific Antigen (PSA) test and Digital Rectal Examination (DRE). This can lead to lower rates of false positives (thus minimize unnecessary procedures such as needle biopsies) and false negatives (thus increasing detection of occult disease and minimizing morbidity and mortality due to PCA).

### 2. To Determine Aggressiveness

Genetic testing can provide information about pre-diagnostic prognostic indicators and enable the identification of individuals at high or low risk for aggressive tumor types that can lead to modification in screening strategies. For example, an individual determined to be a carrier of a high risk allele for the development of aggressive prostate cancer will likely undergo more frequent PSA testing, examination and have a lower threshold for needle biopsy in the presence of an abnormal PSA value.

Furthermore, identifying individuals that are carriers of high or low risk alleles for aggressive tumor types will lead to modification in treatment strategies. For example, if prostate cancer is diagnosed in an individual that is a carrier of an allele that confers increased risk of developing an aggressive form of prostate cancer, then the clinician would likely advise a more aggressive treatment strategy such as a prostatectomy instead of a less aggressive treatment strategy.

As is known in the art, Prostate Specific Antigen (PSA) is a protein that is secreted by the epithelial cells of the prostate gland, including cancer cells. An elevated level in the blood indicates an abnormal condition of the prostate, either benign or malignant. PSA is used to detect potential problems in the prostate gland and to follow the progress of prostate cancer therapy. PSA levels above 4 ng/ml are indicative of the presence of prostate cancer (although as known in the art and described herein, the test is neither very specific nor sensitive).

In one embodiment, the method of the invention is performed in combination with (either prior to, concurrently or after) a PSA assay. In a particular embodiment, the presence of a marker or haplotype, in conjunction with the subject having a PSA level greater than 4 ng/ml, is indicative of a more aggressive prostate cancer and/or a worse prognosis. As described herein, particular markers and haplotypes are associated with high Gleason (i.e., more aggressive) prostate cancer. In another embodiment, the presence of a marker or haplotype, in a patient who has a normal PSA level (e.g., less than 4 ng/ml), is indicative of a high Gleason (i.e., more aggressive) prostate cancer and/or a worse prognosis. A “worse prognosis” or “bad prognosis” occurs when it is more likely that the cancer will grow beyond the boundaries of the prostate gland, metastasize, escape therapy and/or kill the host.

In one embodiment, the presence of a marker or haplotype is indicative of a predisposition to a somatic rearrangement of Chr8q24.21 (e.g., one or more of an amplification, a translocation, an insertion and/or deletion) in a tumor or its precursor. The somatic rearrangement itself may subsequently lead to a more aggressive form of prostate cancer (e.g., a higher histologic grade, as reflected by a higher Gleason score or higher stage at diagnosis, an increased progression of prostate cancer (e.g., to a higher stage), a worse outcome (e.g., in terms of morbidity, complications or death)). As is known in the art, the Gleason grade is a widely used method for classifying prostate cancer tissue for the degree of loss of the normal glandular architecture (size, shape and differentiation of glands). A grade from 1-5 is assigned successively to each of the two most predominant tissue patterns present in the examined tissue sample and are added together to produce the total or combined Gleason grade (scale of 2-10). High numbers indicate poor differentiation and therefore more aggressive cancer.

Aggressive prostate cancer is cancer that grows beyond the prostate, metastasizes and eventually kills the patient. As described herein, one surrogate measure of aggressivity is a high combined Gleason grade. The higher the grade on a scale of 2-10 the more likely it is that a patient has aggressive disease.

As used herein and unless noted differently, the term “stage” is used to define the size and physical extent of a cancer (e.g., prostate cancer). One method of staging various cancers is the TNM method, wherein in the TNM acronym, T stands for tumor size and invasiveness (e.g., the primary tumor in the prostate); N relates to nodal involvement (e.g., prostate cancer that has spread to lymph nodes); and M indicates the presence or absense of metastates (spread to a distant site).

The present invention furthermore relates to risk assessment for cancer (e.g., prostate cancer), including diagnosing whether an individual is at risk for developing the cancer. The polymorphic markers of the present invention can be used alone or in combination, as well as in combination with other factors, including other genetic or non-genetic risk factors or biomarkers (e.g., PSA), for risk assessment of an individual for a particular cancer (e.g., prostate cancer). Many factors may affect the predisposition of an individual towards developing risk of developing cancer are known to the person skilled in the art and can be utilized in such assessment. These include, but are not limited to, age, gender, smoking history and smoking status, physical activity, waist-to-hip circumference ratio, family history of cancer, previously diagnosed cancer, obesity, hypertriglyceridemia, low HDL cholesterol, hypertension, elevated blood pressure, cholesterol levels, HDL cholesterol, LDL cholesterol, triglycerides, apolipoprotein Al and B levels, fibrinogen, ferritin, C-reactive protein and leukotriene levels. Methods known in the art can be used for such overall risk assessment, including multivariate analyses or logistic regression.

### Methods of the Invention

Methods for the diagnosis of susceptibility to cancer (e.g., prostate cancer (e.g., aggressive prostate cancer), breast cancer, lung cancer, melanoma) are described herein and are encompassed by the invention. Kits for assaying a sample from a subject to detect susceptibility to cancer (e.g., prostate cancer (e.g., aggressive prostate cancer), breast cancer, lung cancer, melanoma) are also encompassed by the invention.

### Diagnostic and Screening Assays

In certain embodiments, the present invention pertains to methods of diagnosing, or aiding in the diagnosis of, cancer or a susceptibility to cancer, by detecting particular alleles at genetic markers that appear more frequently in cancer subjects or subjects who are susceptible to cancer. In a particular embodiment, the invention is a method of diagnosing a susceptibility to prostate cancer (e.g., aggressive prostate cancer), breast cancer, colon cancer, lung cancer and/or melanoma by detecting one or more particular polymorphic markers (e.g., the markers or haplotypes described herein). The present invention describes methods whereby detection of particular markers or haplotypes is indicative of a susceptibility to cancer (e.g., prostate cancer (e.g., aggressive prostate cancer), breast cancer, lung cancer, melanoma). Such prognostic or predictive assays can also be used to determine prophylactic treatment of a subject prior to the onset of symptoms associated with such cancers.

In addition, in certain other embodiments, the present invention pertains to methods of diagnosing, or aiding in the diagnosis of, a decreased susceptibility to cancer, by detecting particular genetic marker alleles or haplotypes that appear less frequently in cancer. In a particular embodiment, the invention is a method of diagnosing a decreased susceptibility to prostate cancer (e.g., aggressive prostate cancer), breast cancer, lung cancer and/or melanoma by detecting one or more particular genetic markers (e.g., the markers or haplotypes described herein). The present invention describes methods whereby detection of particular markers or haplotypes is indicative of a decreased susceptibility to cancer (e.g., prostate cancer (e.g., aggressive prostate cancer), breast cancer, colon cancer, lung cancer, melanoma), or of a protective marker or haplotype against the cancer.

As described and exemplified herein, particular markers or haplotypes associated with the Chr8q24.21 LD Block C (SEQ ID NO:1) and LD Block C′ (SEQ ID NO:2) are associated with cancer (e.g., prostate cancer (e.g., aggressive prostate cancer), breast cancer, lung cancer, colon cancer, melanoma). In one embodiment, the marker or haplotype is one that confers a significant risk or susceptibility to prostate cancer, breast cancer, lung cancer, colon cancer and/or melanoma. In another embodiment, the invention pertains to methods of diagnosing a susceptibility to cancer (e.g., prostate cancer (e.g., aggressive prostate cancer), breast cancer, lung cancer, melanoma) in a subject, by screening for a marker or haplotype associated with SEQ ID NO:2 (e.g., markers as set forth in 5A, 5B and 5C, and markers in linkage disequilibrium therewith) that is more frequently present in a subject having, or who is susceptible to, cancer (affected), as compared to the frequency of its presence in a healthy subject (control). In certain embodiments, the marker or haplotype has a p value <0.05. In other embodiments, the significance of association is characterized by smaller p-values, such as <0.01, <0.001, <0.0001, <0.00001, <0.000001, <0.0000001, <0.00000001 or <0.000000001.

In these embodiments, the presence of the marker or haplotype is indicative of a susceptibility to cancer (e.g., prostate cancer (e.g., aggressive prostate cancer), breast cancer, colon cancer, lung cancer, melanoma). These diagnostic methods involve detecting the presence or absence of a marker or haplotype that is associated with cancer, as described herein. The haplotypes described herein include combinations of various genetic markers (e.g., SNPs, microsatellites). The detection of the particular genetic markers that make up the particular haplotypes can be performed by a variety of methods described herein and/or known in the art. For example, genetic markers can be detected at the nucleic acid level (e.g., by direct nucleotide sequencing) or at the amino acid level if the genetic marker affects the coding sequence of a protein encoded by a cancer-associated nucleic acid, e.g. a nucleic acid whose sequence is as set forth in SEQ ID NO:1 or SEQ ID NO:2 (e.g., by protein sequencing or by immunoassays using antibodies that recognize such a protein). The marker alleles or haplotypes of the present invention correspond to fragments of a genomic DNA sequence associated with cancer (e.g. prostate cancer). Such fragments encompass the DNA sequence of the polymorphic marker or haplotype in question, but may also include DNA segments in strong LD (linkage disequilibrium) with the marker or haplotype. In one embodiment, such segments comprises genomic segments in LD with the marker or haplotype as determined by a value of r² greater than 0.2 and/or |D′|>0.8). Examples of such segments in LD with the variants of the invention are set forth in SEQ ID NO:1 and SEQ IN NO:2.

In one embodiment, diagnosis of a susceptibility to cancer (e.g., prostate cancer (e.g., aggressive prostate cancer), breast cancer, lung cancer, melanoma) can be accomplished using hybridization methods, such as Southern analysis, Northern analysis, and/or in situ hybridizations (see Current Protocols in Molecular Biology, Ausubel, F. et al., eds., John Wiley & Sons, including all supplements). A biological sample from a test subject or individual (a “test sample”) of genomic DNA, RNA, or cDNA is obtained from a subject suspected of having, being susceptible to, or predisposed for cancer (the “test subject”). The subject can be an adult, child, or fetus. The test sample can be from any source that contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined. The presence of a specific marker allele can be indicated by sequence-specific hybridization of a nucleic acid probe specific for the particular allele. The presence of more than specific marker allele or a specific haplotype can be indicated by using several sequence-specific nucleic acid probes, each being specific for a particular allele. In one embodiment, a haplotype can be indicated by a single nucleic acid probe that is specific for the specific haplotype (i.e., hybridizes specifically to a DNA strand comprising the specific marker alleles characteristic of the haplotype). A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to cancer (e.g., prostate cancer (e.g., aggressive prostate cancer), breast cancer, lung cancer, melanoma), a hybridization sample is formed by contacting the test sample containing a cancer-associated nucleic acid, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length that is sufficient to specifically hybridize under stringent conditions to appropriate mRNA or genomic DNA. For example, the nucleic acid probe can be all or a portion of SEQ ID NO:1 or all or a portion of SEQ ID NO:2, optionally comprising at least one allele contained in the haplotypes described herein, or the probe can be the complementary sequence of such a sequence. In a particular embodiment, the nucleic acid probe is a portion of SEQ ID NO:1 or a portion of SEQ ID NO:2, optionally comprising at least one allele contained in the haplotypes described herein, or the probe can be the complementary sequence of such a sequence. Other suitable probes for use in the diagnostic assays of the invention are described herein.

Hybridization can be performed by methods well known to the person skilled in the art (see, e.g., Current Protocols in Molecular Biology, Ausubel, F. et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe. The process can be repeated for any markers of the present invention, or markers that make up a haplotype of the present invention, or multiple probes can be used concurrently to detect more than one marker alleles at a time. It is also possible to design a single probe containing more than one marker alleles of a particular haplotype (e.g., a probe containing alleles complementary to 2, 3, 4, 5 or all of the markers that make up a particular haplotype). Detection of the particular markers of the haplotype in the sample is indicative that the source of the sample has the particular haplotype (e.g., a haplotype) and therefore is susceptible to cancer (e.g., prostate cancer).

In one preferred embodiment, a method utilizing a detection oligonucleotide probe comprising a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In another hybridization method, Northern analysis (see Current Protocols in Molecular Biology, Ausubel, F. et al., eds., John Wiley & Sons, supra) is used to identify the presence of a polymorphism associated with cancer or a susceptibility to cancer (e.g., prostate cancer (e.g., aggressive prostate cancer), breast cancer, lung cancer, melanoma). For Northern analysis, a test sample of RNA is obtained from the subject by appropriate means. As described herein, specific hybridization of a nucleic acid probe to RNA from the subject is indicative of a particular allele complementary to the probe. For representative examples of use of nucleic acid probes, see, for example, U.S. Pat. Nos. 5,288,611 and 4,851,330.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P., et al., Bioconjug. Chem. 5:3-7 (1994)). The PNA probe can be designed to specifically hybridize to a molecule in a sample suspected of containing one or more of the genetic markers of a haplotype that is associated with cancer (e.g., prostate cancer (e.g., aggressive prostate cancer), breast cancer, lung cancer, melanoma). Hybridization of the PNA probe is diagnostic for cancer or a susceptibility to cancer.

In one embodiment of the invention, a test sample containing genomic DNA obtained from the subject is collected and the polymerase chain reaction (PCR) is used to amplify a fragment comprising one or more markers or haplotypes of the present invention. As described herein, identification of a particular marker allele or haplotype associated with cancer can be accomplished using a variety of methods (e.g., sequence analysis, analysis by restriction digestion, specific hybridization, single stranded conformation polymorphism assays (SSCP), electrophoretic analysis, etc.). In another embodiment, diagnosis is accomplished by expression analysis using quantitative PCR (kinetic thermal cycling). This technique can, for example, utilize commercially available technologies, such as TaqMan® (Applied Biosystems, Foster City, Calif.). The technique can assess the presence of an alteration in the expression or composition of a polypeptide or splicing variant(s) that is encoded by a nucleic acid associated with cancer (e.g., a nucleic acid whose sequence comprises all or a fragment of the sequence set forth in SEQ ID NO:1 or SEQ ID NO:2). Further, the expression of the variant(s) can be quantified as physically or functionally different.

In another method of the invention, analysis by restriction digestion can be used to detect a particular allele if the allele results in the creation or elimination of a restriction site relative to a reference sequence. A test sample containing genomic DNA is obtained from the subject. PCR can be used to amplify particular regions of SEQ ID NO:1 or SEQ ID NO:2 in the test sample from the test subject. Restriction fragment length polymorphism (RFLP) analysis can be conducted, e.g., as described in Current Protocols in Molecular Biology, supra. The digestion pattern of the relevant DNA fragment indicates the presence or absence of the particular allele in the sample.

Sequence analysis can also be used to detect specific alleles at polymorphic sites associated with SEQ ID NO:1 or SEQ ID NO:2. Therefore, in one embodiment, determination of the presence or absence of a particular marker alleles or haplotypes comprises sequence analysis of a test sample of DNA or RNA obtained from a subject or individual. PCR or other appropriate methods can be used to amplify a portion of SEQ ID NO:1 or SEQ ID NO:2, and the presence of a specific allele can then be detected directly by sequencing the polymorphic site (or, alternatively, multiple polymorphic sites in a haplotype) of the genomic DNA in the sample.

Allele-specific oligonucleotides can also be used to detect the presence of a particular allele at a polymorphic site associated with cancer, through the use of dot-blot hybridization of amplified oligonucleotides with allele-specific oligonucleotide (ASO) probes (see, for example, Saiki, R. et al., Nature, 324:163-166 (1986)). An “allele-specific oligonucleotide” (also referred to herein as an “allele-specific oligonucleotide probe”) is an oligonucleotide of approximately 10-50 base pairs or approximately 15-30 base pairs, that specifically hybridizes to a region of SEQ ID NO:1 or SEQ ID NO:2, and which contains a specific allele at a polymorphic site (e.g., a polymorphism described herein). An allele-specific oligonucleotide probe that is specific for one or more particular polymorphisms associated with SEQ ID NO:1 or SEQ ID NO:2 can be prepared using standard methods (see, e.g., Current Protocols in Molecular Biology, supra). PCR can be used to amplify the desired region of SEQ ID NO:1 or SEQ ID NO:2. The DNA containing the amplified LD Block C region can be dot-blotted using standard methods (see, e.g., Current Protocols in Molecular Biology, supra), and the blot can be contacted with the oligonucleotide probe. The presence of specific hybridization of the probe to the amplified region can then be detected. Specific hybridization of an allele-specific oligonucleotide probe to DNA from the subject is indicative of a specific allele at a polymorphic site associated with cancer (e.g., prostate cancer) (see, e.g., Gibbs, R. et al., Nucleic Acids Res., 17:2437-2448 (1989) and WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tₘ) of 64° C. and 74° C. when in complex with complementary DNA or RNA, respectively, as opposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from a subject, can be used to identify polymorphisms in a cancer-associated nucleic acid. For example, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art (see, e.g., U.S. Pat. No. 5,143,854, PCT Patent Publication Nos. WO 90/15070 and 92/10092). These arrays can generally be produced using mechanical synthesis methods or light directed synthesis methods that incorporate a combination of photolithographic methods and solid phase oligonucleotide synthesis methods (Fodor, S. of al., Science, 251:767-773 (1991); Pirrung et al., U.S. Pat. No. 5,143,854 (see also published PCT Application No. WO 90/15070); and Fodor. S. et al., published PCT Application No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is allowed to hybridize with the array. Detection of hybridization is a detection of a particular allele in the nucleic acid of interest. Hybridization and scanning are generally carried out by methods described herein and also in, e.g., published PCT Application Nos. WO 92/10092 and WO 95/11995, and U.S. Pat. No. 5,424,186, the entire teachings of each of which are incorporated by reference herein. In brief, a target nucleic acid sequence, which includes one or more previously identified polymorphic markers, is amplified by well-known amplification techniques (e.g., PCR). Typically this involves the use of primer sequences that are complementary to the two strands of the target sequence, both upstream and downstream, from the polymorphic site. Asymmetric PCR techniques can also be used. Amplified target, generally incorporating a label, is then allowed to hybridize with the array under appropriate conditions that allow for sequence-specific hybridization. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphic site, arrays can include multiple detection blocks, and thus be capable of analyzing multiple, specific polymorphisms (e.g., multiple polymorphisms of a particular haplotype (e.g., an haplotype)). In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional descriptions of use of oligonucleotide arrays for detection of polymorphisms can be found, for example, in U.S. Pat. Nos. 5,858,659 and 5,837,832, the entire teachings of both of which are incorporated by reference herein.

Other methods of nucleic acid analysis can be used to detect a particular allele at a polymorphic site associated with cancer (e.g., a polymorphic site associated with the genomic segment on Chr8q24.21 whose nucleic acid sequence is represented by the sequence set forth in SEQ ID NO:1 and SEQ ID NO:2). Representative methods include, for example, direct manual sequencing (Church and Gilbert, Proc. Natl. Acad. Sci. USA, 81: 1991-1995 (1988); Sanger, F., et al., Proc. Natl. Acad. Sci. USA, 74:5463-5467 (1977); Beavis, et al., U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V., et al., Proc. Natl. Acad. Sci. USA, 86:232-236 (1989)), mobility shift analysis (Orita, M., et al., Proc. Natl. Acad. Sci. USA, 86:2766-2770 (1989)), restriction enzyme analysis (Flavell, R., et al., Cell, 15:25-41 (1978); Geever, R., et al., Proc. Natl. Acad. Sci. USA, 78:5081-5085 (1981)); heteroduplex analysis; chemical mismatch cleavage (CMC) (Cotton, R., et al., Proc. Natl. Acad. Sci. USA, 85:4397-4401 (1985)); RNase protection assays (Myers, R., et al., Science, 230:1242-1246 (1985); use of polypeptides that recognize nucleotide mismatches, such as E. coli mutS protein; and allele-specific PCR.

In another embodiment of the invention, diagnosis of cancer or a susceptibility to cancer (e.g., prostate cancer (e.g., aggressive prostate cancer), breast cancer, lung cancer, melanoma) can be made by examining expression and/or composition of a polypeptide encoded by cancer-associated nucleic acid in those instances where the genetic marker(s) or haplotype described herein results in a change in the composition or expression of the polypeptide. Thus, diagnosis of a susceptibility to cancer (e.g., prostate cancer (e.g., aggressive prostate cancer), breast cancer, lung cancer, melanoma) can be made by examining expression and/or composition of one of these polypeptides, or another polypeptide encoded by cancer-associated nucleic acid, in those instances where the genetic marker or haplotype described herein results in a change in the composition or expression of the polypeptide. The haplotypes and markers described herein that show association to cancer may play a role through their effect on one or more of these nearby genes. Possible mechanisms affecting these genes include, e.g., effects on transcription, effects on RNA splicing, alterations in relative amounts of alternative splice forms of mRNA, effects on RNA stability, effects on transport from the nucleus to cytoplasm, and effects on the efficiency and accuracy of translation.

The c-myc gene on Chr8q24.21 encodes the c-MYC protein that was identified over 20 years ago as the cellular counterpart of the viral oncogene v-myc of the avian myelocytomatosis retrovirus (Vennstrom et al., J. Virology 42:773-79 (1982)). The c-MYC protein is a transcription factor that is rapidly induced upon treatment of cells with mitogenic stimuli. c-MYC regulates the expression of many genes by binding E-boxes (CACGTG) in a heterodimeric complex with a protein named MAX. Many of the genes regulated by c-MYC are involved in cell cycle control. c-MYC promotes cell-cycle progression, inhibits cellular differentiation and induces apoptosis. c-MYC also has a negative effect on double strand DNA repair (Karlsson, A, et al., Proc. Natl. Acad. Sci. USA 100(17):9974-79 (2003)). c-MYC also promotes angiogenesis (Ngo, C. V., et al., Cell Growth Differ. 11(4):201-10 (2000); Baudino T. A., et al., Genes Dev. 16(19):2530-43 (2002)).

The c-myc gene is highly tumorigenic in vitro and in vivo. c-MYC synergizes with proteins that inhibit apoptosis such as BCL, BCL-X_{L} or with the loss of p53 or ARF in lymphomagenesis in transgenic mice (Strasser et al., Nature 348:331-333 (1990); Blyth, K., et al., Oncogene 10:1717-23 (1990); Elson, A., et al., Oncogene 11:181-90 (1995); Eischen, C. M., et al., Genes Dev. 13:2658-69 (1999)).

Amplification and overexpression of the c-myc gene is seen in prostate cancer and is often associated with aggressive tumors, hormone independence and a poor prognosis (Jenkins, R. B., of al., Cancer Res. 57(3):524-31 (1997); El Gedaily, A., et al., Prostate 46(3):184-90 (2001); Saramaki, O., et al., Am. J. Pathol. 159(6):2089-94 (2001); Bubendorf, L., et al., Cancer Res. 59(4):803-06 (1999)). c-myc and the Chr8q24.21 region is furthermore gained in prostate, breast and lung tumors and in melanoma (Blancato J., et al., Br. J. Cancer 90(8):1612-9 (2004); Kubokura, H., et al., Ann. Thorac. Cardiovasc. Surg. 7(4):197-203 (2001); Treszl, A., et al., Cytometry 60B(1):37-46 (2004); Kraehn, G. M., et al., Br. J. Cancer 84(1):72-79 (2001)). In addition, many other tumor types show a gain of this region including colon, liver, ovary, stomach, intestinal and bladder cancer. Combining all tumor types shows that Chr8q24.21 is the most frequently gained chromosomal region with gain in approximately 17% of all tumor types (www.progenetix.com).

The oncogene is involved in Burkitt's lymphoma as a result of translocations that juxtapose c-myc to immunoglobulin enhancers, thereby activating expression of the gene (Dalla-Favera, R., et al., Proc. Natl. Acad. Sci. USA 79(24):7824-27 (1982); Taub, R., et al., Proc. Natl. Acad. Sci. USA 79(24):7837-41 (1982). It is also involved in cervical cancer by Human papillomavirus (HPV) integration close to the gene. In most cases, HPV integrations occur in a region spanning 500 kb centromeric and 200 kb telomeric of the c-myc gene (Ferber, J. M., et al., Cancer Genetics Cytogenetics 154:1-9 (2004); Ferber, M. J., et al., Oncogene 22:7233-7242 (2003)).

Two fragile sites, FRA8C and FRA8D, lie centromeric and telomeric to c-myc, respectively, on Chr8q24.21. Fragile sites are prone to breakage in the presence of agents that arrest DNA synthesis. Replication of fragile sites is thought to occur late in S-phase and upon induction even later. The involvement of fragile sites in chromosomal amplification, translocation and/or viral insertion may relate to the late replication of these sites and that a break is initiated at or close to stalled replication forks (Hellman, A., et al., Cancer Cell 1:89-97 (2002)).

It is possible that markers or haplotypes described here within LD Block C (SEQ ID NO:1) or LD Block C′ (SEQ ID NO:2) or in strong LD with LD Block C (SEQ ID NO:1) or LD Block C′ (SEQ ID NO:2) (e.g., as measured by r² greater than 0.2 and/or |D′|>0.8) could affect the stability of the region leading to gene amplifications of the c-myc gene or other nearby genes. That is, a person could inherit a particular variant form of the region as set forth in SEQ ID NO:1 or SEQ ID NO:2 from one or both parents and therefore be more likely to have a somatic mutational event later in one or more cells leading to progression of cancer to a more aggressive form. Thus, in one embodiment, identification of a marker or haplotype of the invention (e.g., a marker or haplotype associated with SEQ ID NO:2 or SEQ ID NO:1) may be used to diagnose a susceptibility to a somatic mutational event, which can lead to progression of cancer to a more aggressive form.

In one embodiment, the marker or haplotype does not comprise a marker that is located within the c-myc open reading frame (i.e., chr8:128,705,092-128,710,260 by in NCBI Build 34). In another embodiment, the marker or haplotype does not comprise a marker that is located within the c-myc promoter or open reading frame. In yet another embodiment, the marker or haplotype does not comprise a marker that is located within the c-myc promoter, enhancer or open reading frame. In still other embodiments, the marker or haplotype does not comprise a marker that is located within 1 kb, 2 kb, 5 kb, 10 kb, 15 kb, 20 kb or 25 kb of the c-myc open reading frame.

A variety of methods can be used to make such a detection, including enzyme linked immunosorbent assays (ELISA), Western blots, immunoprecipitations and immunofluorescence. A test sample from a subject is assessed for the presence of an alteration in the expression and/or an alteration in composition of the polypeptide encoded by a Chr8q24.21-associated nucleic acid and/or a nucleic acid associated with LD Block C (SEQ ID NO:1) or LD Block C′ (SEQ ID NO:2). An alteration in expression of a polypeptide encoded by such a nucleic acid can be, for example, an alteration in the quantitative polypeptide expression (i.e., the amount of polypeptide produced). An alteration in the composition of a polypeptide encoded by the nucleic acid is an alteration in the qualitative polypeptide expression (e.g., expression of a mutant polypeptide or of a different splicing variant). In one embodiment, diagnosis of a susceptibility to cancer (e.g., prostate cancer (e.g., aggressive prostate cancer), breast cancer, colon cancer lung cancer, melanoma) is made by detecting a particular splicing variant encoded by a cancer-associated nucleic acid as described herein (e.g., a Chr8q24.21-associated nucleic acid, a LD Block C (SEQ ID NO:1)-associated nucleic acid, and/or a LD Block C′ (SEQ ID NO:2)-associated nucleic acid), or a particular pattern of splicing variants.

Both such alterations (quantitative and qualitative) can also be present. An “alteration” in the polypeptide expression or composition, as used herein, refers to an alteration in expression or composition of a polypeptide in a test sample, as compared to the expression or composition of the polypeptide in a control sample. A control sample is a sample that corresponds to the test sample (e.g., is from the same type of cells), and is from a subject who is not affected by, and/or who does not have a susceptibility to, cancer (e.g., a subject that does not possess a marker or haplotype as described herein). Similarly, the presence of one or more different splicing variants in the test sample, or the presence of significantly different amounts of different splicing variants in the test sample, as compared with the control sample, can be indicative of a susceptibility to cancer (e.g., prostate cancer (e.g., aggressive prostate cancer), breast cancer, colon cancer, lung cancer, melanoma). An alteration in the expression or composition of the polypeptide in the test sample, as compared with the control sample, can be indicative of a specific variant (e.g., marker allele or haplotype) in the instance where the variant alters a splice site relative to the reference in the control sample. Various means of examining expression or composition of a polypeptide encoded by a nucleic acid are known to the person skilled in the art and can be used, including spectroscopy, colorimetry, electrophoresis, isoelectric focusing, and immunoassays (e.g., David et al., U.S. Pat. No. 4,376,110) such as immunoblotting (see, e.g., Current Protocols in Molecular Biology, particularly chapter 10, supra).

For example, in one embodiment, an antibody (e.g., an antibody with a detectable label) that is capable of binding to a polypeptide encoded by a nucleic acid associated with cancer as described herein can be used. Antibodies can be polyclonal or monoclonal. An intact antibody, or a fragment thereof (e.g., Fv, Fab, Fab′, F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a labeled secondary antibody (e.g., a fluorescently-labeled secondary antibody) and end-labeling of a DNA probe with biotin such that it can be detected with fluorescently-labeled streptavidin.

In one embodiment of this method, the level or amount of polypeptide encoded by a nucleic acid associated with cancer (e.g., prostate cancer) in a test sample is compared with the level or amount of the polypeptide in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic for a particular allele responsible for causing the difference in expression. Alternatively, the composition of the polypeptide in a test sample is compared with the composition of the polypeptide in a control sample. In another embodiment, both the level or amount and the composition of the polypeptide can be assessed in the test sample and in the control sample.

In another embodiment, the diagnosis of a susceptibility to cancer (e.g., prostate cancer (e.g., aggressive prostate cancer), breast cancer, colon cancer, lung cancer, melanoma) is made by detecting at least one marker or haplotype of the invention (e.g., the markers as set forth in Table 5A, 5B and 5C, and markers or haplotypes in linkage disequilibrium therewith) in combination with an additional protein-based, RNA-based or DNA-based assay (e.g., other cancer diagnostic assays including, but not limited to: PSA assays, carcinoembryonic antigen (CEA) assays, BRCA1 assays BRCA2 assays). Such cancer diagnostic assays are known in the art, and include also other genetic risk factors for cancer known to the skilled person. The methods of the invention can also be used in combination with an analysis of a subject's family history and risk factors (e.g., environmental risk factors, lifestyle risk factors).

As is known in the art, and as described herein, PSA testing has aided early diagnosis of prostate cancer, but it is neither highly sensitive nor specific (Punglia et al., N. Engl. J. Med. 349(4):335-42 (2003)). Accordingly, PSA testing alone leads to a high percentage of false negative and false positive diagnoses, which results in both many instances of missed cancers and unnecessary follow-up biopsies for those without cancer. In one embodiment, the diagnosis of prostate cancer or a susceptibility to prostate cancer is made by detecting at least one Chr8q24.21-associated allele and/or LD Block C-associated allele in combination with a PSA assay.

### Kits

Kits useful in the methods of the invention comprise components useful in any of the methods described herein, including for example, hybridization probes, restriction enzymes (e.g., for RFLP analysis), allele-specific oligonucleotides, antibodies that bind to an altered polypeptide encoded by a nucleic acid of the invention as described herein (e.g., a genomic segment comprising at least one polymorphic marker and/or haplotype of the present invention) or to a non-altered (native) polypeptide encoded by a nucleic acid of the invention as described herein, means for amplification of a nucleic acid associated with cancer, means for analyzing the nucleic acid sequence of a nucleic acid associated with cancer, means for analyzing the amino acid sequence of a polypeptide encoded by a nucleic acid associated with cancer, etc. The kits can for example include necessary buffers, nucleic acid primers for amplifying nucleic acids of the invention (e.g., one or more of the polymorphic markers associated with cancer, e.g., the markers set forth in Tables 5A, 5B and 5C), and reagents for allele-specific detection of the fragments amplified using such primers and necessary enzymes (e.g., DNA polymerase). Additionally, kits can provide reagents for assays to be used in combination with the methods of the present invention, e.g., reagents for use with other cancer diagnostic assays.

In one embodiment, the invention is a kit for assaying a sample from a subject to aid in the detection of a particular cancer (e.g., prostate cancer (e.g., aggressive prostate cancer), lung cancer, colon cancer, breast cancer, melanoma) or a susceptibility to cancer (e.g., prostate cancer, lung cancer, colon cancer, breast cancer, melanoma) in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism, wherein the polymorphism is selected from the group consisting of the polymorphisms as set forth in Table 5A, 5B and 5C, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking polymorphisms (e.g., SNPs or microsatellites) that are indicative of cancer. In another embodiment, the kit comprises one or more labeled nucleic acids capable of allele-specific detection of one or more specific polymorphic markers or haplotypes associated with cancer, and reagents for detection of the label. Suitable labels include, e.g., a radioisotope, a fluorescent label, an enzyme label, an enzyme co-factor label, a magnetic label, a spin label, an epitope label.

In particular embodiments, the polymorphic marker or haplotype to be detected by the reagents of the kit comprises one or more markers, two or more markers, three or more markers, four or more markers or five or more markers selected from the group consisting of the markers set forth in Table 5A, Table 5B and Table 5C. In another embodiment, the marker or haplotype to be detected comprises the markers set forth in Table 4A and Table 4B. In another embodiment, the marker or haplotype to be detected comprises at least one marker from the group of markers in strong linkage disequilibrium, as defined by values of r² greater than 0.2, to at least one of the group of markers consisting of the markers listed in Table 4A, and Table 4B. In a preferred embodiment, the marker or haplotype to be detected comprises rs16901979, and markers in linkage disequilibrium therewith. In another preferred embodiment, the marker or haplotype to be detected comprises HapC (rs1456314 allele G, rs17831626 allele T, rs7825414 allele G, rs6993569 allele G, rs6994316 allele A, rs6470494 allele T, rs1016342 allele C, rs1031588 allele G, rs1016343 allele T, rs1551510 allele G, rs1456306 allele C, rs1378897 allele G, rs1456305 allele T, and rs7816535 allele G).

In one preferred embodiment, the kit for detecting the markers of the invention comprises a detection oligonucleotide probe, that hybridizes to a segment of template DNA containing a SNP polymorphisms to be detected, an enhancer oligonucleotide probe and an endonuclease. As explained in the above, the detection oligonucleotide probe comprises a fluorescent moiety or group at its 3′ terminus and a quencher at its 5′ terminus, and an enhancer oligonucleotide, is employed, as described by Kutyavin et al. (Nucleic Acid Res. 34:e128 (2006)). The fluorescent moiety can be Gig Harbor Green or Yakima Yellow, or other suitable fluorescent moieties. The detection probe is designed to hybridize to a short nucleotide sequence that includes the SNP polymorphism to be detected. Preferably, the SNP is anywhere from the terminal residue to −6 residues from the 3′ end of the detection probe. The enhancer is a short oligonucleotide probe which hybridizes to the DNA template 3′ relative to the detection probe. The probes are designed such that a single nucleotide gap exists between the detection probe and the enhancer nucleotide probe when both are bound to the template. The gap creates a synthetic abasic site that is recognized by an endonuclease, such as Endonuclease IV. The enzyme cleaves the dye off the fully complementary detection probe, but cannot cleave a detection probe containing a mismatch. Thus, by measuring the fluorescence of the released fluorescent moiety, assessment of the presence of a particular allele defined by nucleotide sequence of the detection probe can be performed.

The detection probe can be of any suitable size, although preferably the probe is relatively short. In one embodiment, the probe is from 5-100 nucleotides in length. In another embodiment, the probe is from 10-50 nucleotides in length, and in another embodiment, the probe is from 12-30 nucleotides in length. Other lengths of the probe are possible and within scope of the skill of the average person skilled in the art.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

Certain embodiments of the detection probe, the enhancer probe, and/or the primers used for amplification of the template by PCR include the use of modified bases, including modified A and modified G. The use of modified bases can be useful for adjusting the melting temperature of the nucleotide molecule (probe and/or primer) to the template DNA, for example for increasing the melting temperature in regions containing a low percentage of G or C bases, in which modified A with the capability of forming three hydrogen bonds to its complementary T can be used, or for decreasing the melting temperature in regions containing a high percentage of G or C bases, for example by using modified G bases that form only two hydrogen bonds to their complementary C base in a double stranded DNA molecule. In a preferred embodiment, modified bases are used in the design of the detection nucleotide probe. Any modified base known to the skilled person can be selected in these methods, and the selection of suitable bases is well within the scope of the skilled person based on the teachings herein and known bases available from commercial sources as known to the skilled person.

In one of such embodiments, the presence of the marker or haplotype is indicative of a susceptibility (increased susceptibility or decreased susceptibility) to cancer (e.g. prostate cancer (e.g., aggressive prostate cancer), lung cancer, colon cancer, breast cancer, melanoma). In another embodiment, the presence of the marker or haplotype is indicative of response to a therapeutic agent for cancer. In another embodiment, the presence of the marker or haplotype is indicative of cancer prognosis in an individual. In yet another embodiment, the presence of the marker or haplotype is indicative of progress of cancer treatment. Such treatment may include intervention by surgery, medication or by other means (e.g., lifestyle changes).

Diagnosis of Disease Associated with Variants of the Invention

Although the methods of the invention have been generally described in the context of diagnosing susceptibility to cancer (e.g. prostate cancer), the methods can also be used to diagnose cancer associated with the polymorphic markers of the present invention. For example, an individual having cancer or who is at risk for developing cancer can be assessed to determine whether the presence in the individual of a polymorphism or haplotype of the present invention could have been a contributing factor to the diagnosed cancer in the individual. In one embodiment, identification of a cancer associated with the markers and/or haplotypes of the present invention facilitates treatment planning. For example, preventive treatment to minimize the occurrence of the individual developing cancer can be administered. Such preventive treatment can also include assessment of (i) whether the individual is heterozygous or homozygous for the at-risk variant; (ii) the individual's age, and (iii) the individual's sex, since the variants of the present invention have been shown to be associated with lower age at onset of coronary artery disease and myocardial infarction. In other embodiments of the invention, therapies can be designed and therapeutics selected to target the appropriate gene or protein associated with the polymorphism and/or haplotype of the present invention.

In one embodiment of the invention, diagnosis of a cancer associated with the markers and/or haplotypes of the present invention is made by detecting a polymorphism or haplotype of the present invention. Particular polymorphisms are described herein. In particular embodiments, the polymorphic marker or haplotype to be detected comprises one or more markers, two or more markers, three or more markers, four or more markers or five or more markers selected from the group consisting of the markers set forth in Table 5A, Table 5B and Table 5C. In another embodiment, the marker or haplotype to be detected comprises the markers set forth in Table 4A and Table 4B. In another embodiment, the marker or haplotype to be detected comprises at least one marker from the group of markers in strong linkage disequilibrium, as defined by values of r² greater than 0.2, to at least one of the group of markers consisting of the markers listed in Table 4A, and Table 4B. In a preferred embodiment, the marker or haplotype to be detected comprises rs16901979, and markers in linkage disequilibrium therewith. In another preferred embodiment, the marker or haplotype to be detected comprises HapC (rs1456314 allele G, rs17831626 allele T, rs7825414 allele G, rs6993569 allele G, rs6994316 allele A, rs6470494 allele T, rs1016342 allele C, rs1031588 allele G, rs1016343 allele T, rs1551510 allele G, rs1456306 allele C, rs1378897 allele G, rs1456305 allele T, and rs7816535 allele G).

A test sample of genomic DNA, RNA, or cDNA, is obtained from a subject having cancer to determine whether the disease is associated with one or more polymorphisms of the invention. The DNA, RNA or cDNA sample is then examined to determine whether a specific allele of a polymorphism or a specific haplotype of the invention is found to be present in the sample. If the nucleic acid sample is found to contain the specific allele of the polymorphism or the haplotype, then the presence of the allele or haplotype is indicative of the cancer being associated with the polymorphism and/or haplotype.

Methods known to the person skilled in the art, as described in further detail herein in the methods and kits of the present invention, can be used to detect the polymorphism.

### Therapeutic Agents

Variants of the present invention (e.g., the markers and/or haplotypes of the invention, e.g., the markers listed in Tables 5A, 5B and 5C, and markers in linkage disequlibrium therewith, e.g., the markers listed in Table 4A and 4B) can be used to identify novel therapeutic targets for cancer (e.g., prostate cancer). For example, genes containing, or in linkage disequilibrium with, variants (markers and/or haplotypes) associated with cancer, or their products, as well as genes or their products that are directly or indirectly regulated by or interact with these variant genes or their products, can be targeted for the development of therapeutic agents to treat cancer, or prevent or delay onset of symptoms associated with cancer. In one embodiment, the gene is c-myc. Therapeutic agents may comprise one or more of, for example, small non-protein and non-nucleic acid molecules, proteins, peptides, protein fragments, nucleic acids (DNA, RNA), PNA (peptide nucleic acids), or their derivatives or mimetics which can modulate the function and/or levels of the target genes or their gene products.

The nucleic acids and/or variants of the invention, or nucleic acids comprising their complementary sequence, may be used as antisense constructs to control gene expression in cells, tissues or organs. The methodology associated with antisense techniques is well known to the skilled artisan, and is described and reviewed in AntisenseDrug Technology: Principles, Strategies, and Applications, Crooke, ed., Marcel Dekker Inc., New York (2001). In general, antisense nucleic acid molecules are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Lavery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002)

The variants described herein can be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (markers and/or haplotypes) can be inhibited or blocked. In one embodiment, the antisense molecules are designed to specifically bind a particular allelic form (i.e., one or several variants (alleles and/or haplotypes)) of the target nucleic acid, thereby inhibiting translation of a product originating from this specific allele or haplotype, but which do not bind other or alternate variants at the specific polymorphic sites of the target nucleic acid molecule.

As antisense molecules can be used to inactivate mRNA so as to inhibit gene expression, and thus protein expression, the molecules can be used to treat a disease such as cancer, including prostate cancer (e.g., aggressive prostate cancer), lung cancer, colon cancer, breast cancer, melanoma. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants of the present invention (e.g., the nucleotide sequence of markers set forth in Table 5A, 5B and 5C, and markers in linkage disequilibrium therewith, e.g., the markers set forth in Table 4A and 4B) can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Lavery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. 9:R440-442 (1999).

A genetic defect leading to increased predisposition or risk for development of a disease, including cancer, or a defect causing the disease, may be corrected permanently by administering to a subject carrying the defect a nucleic acid fragment that incorporates a repair sequence that supplies the normal/wild-type nucleotide(s) at the site of the genetic defect. Such site-specific repair sequence may concompass an RNA/DNA oligonucleotide that operates to promote endogenous repair of a subject's genomic DNA. The administration of the repair sequence may be performed by an appropriate vehicle, such as a complex with polyethelenimine, encapsulated in anionic liposomes, a viral vector such as an adenovirus vector, or other pharmaceutical compositions suitable for promoting intracellular uptake of the adminstered nucleic acid. The genetic defect may then be overcome, since the chimeric oligonucleotides induce the incorporation of the normal sequence into the genome of the subject, leading to expression of the normal/wild-type gene product. The replacement is propagated, thus rendering a permanent repair and alleviation of the symptoms associated with the disease or condition.

The present invention provides methods for identifying compounds or agents that can be used to treat cancer. Thus, the variants of the invention are useful as targets for the identification and/or development of therapeutic agents. Such methods may include assaying the ability of an agent or compound to modulate the activity and/or expression of a nucleic acid that includes at least one of the variants (markers and/or haplotypes) of the present invention, or the encoded product of the nucleic acid. This in turn can be used to identify agents or compounds that inhibit or alter the undesired activity or expression of the encoded nucleic acid product. Assays for performing such experiments can be performed in cell-based systems or in cell-free systems, as known to the skilled person. Cell-based systems include cells naturally expressing the nucleic acid molecules of interest, or recombinant cells that have been genetically modified so as to express a certain desired nucleic acid molecule.

Variant gene expression in a patient can be assessed by expression of a variant-containing nucleic acid sequence (for example, a gene containing at least one variant of the present invention, which can be transcribed into RNA containing the at least one variant, and in turn translated into protein), or by altered expression of a normal/wild-type nucleic acid sequence due to variants affecting the level or pattern of expression of the normal transcripts, for example variants in the regulatory or control region of the gene. Assays for gene expression include direct nucleic acid assays (mRNA), assays for expressed protein levels, or assays of collateral compounds involved in a pathway, for example a signal pathway. Furthermore, the expression of genes that are up- or down-regulated in response to the signal pathway can also be assayed. One embodiment includes operably linking a reporter gene, such as luciferase, to the regulatory region of the gene(s) of interest.

Modulators of gene expression can in one embodiment be identified when a cell is contacted with a candidate compound or agent, and the expression of mRNA is determined. The expression level of mRNA in the presence of the candidate compound or agent is compared to the expression level in the absence of the compound or agent. Based on this comparison, candidate compounds or agents for treating cancer can be identified as those modulating the gene expression of the variant gene. When expression of mRNA or the encoded protein is statistically significantly greater in the presence of the candidate compound or agent than in its absence, then the candidate compound or agent is identified as a stimulator or up-regulator of expression of the nucleic acid. When nucleic acid expression or protein level is statistically significantly less in the presence of the candidate compound or agent than in its absence, then the candidate compound is identified as an inhibitor or down-regulator of the nucleic acid expression.

The invention further provides methods of treatment using a compound identified through drug (compound and/or agent) screening as a gene modulator (i.e. stimulator and/or inhibitor of gene expression).

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

### Methods of Assessing Probability of Response to Therapeutic Agents, Methods of Monitoring Progress of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site or haplotype is indicative of a different, e.g. a different response rate, to a particular treatment modality. This means that a patient diagnosed with cancer (e.g. prostate cancer (e.g., aggressive prostate cancer), breast cancer, lung cancer, colon cancer, melanoma), and carrying a certain allele at a polymorphic or haplotype of the present invention (e.g., the at-risk and protective alleles and/or haplotypes of the invention) would respond better to, or worse to, a specific therapeutic, drug and/or other therapy used to treat the cancer. Therefore, the presence or absence of the marker allele or haplotype could aid in deciding what treatment should be used for a the patient. For example, for a newly diagnosed patient, the presence of a marker or haplotype of the present invention may be assessed (e.g., through testing DNA derived from a blood sample, as described herein). If the patient is positive for a marker allele or haplotype at (that is, at least one specific allele of the marker, or haplotype, is present), then the physician recommends one particular therapy, while if the patient is negative for the at least one allele of a marker, or a haplotype, then a different course of therapy may be recommended (which may include recommending that no immediate therapy, other than serial monitoring for progression of the disease, be performed). Thus, the patient's carrier status could be used to help determine whether a particular treatment modality should be administered. The value lies within the possibilities of being able to diagnose the disease at an early stage, to select the most appropriate treatment, and provide information to the clinician about prognosis/aggressiveness of the disease in order to be able to apply the most appropriate treatment.

The present invention also relates to methods of monitoring progress or effectiveness of a treatment for a particular cancer (e.g. prostate cancer (e.g., aggressive prostate cancer), breast cancer, lung cancer, colon cancer, melanoma). This can be done based on the genotype and/or haplotype status of the markers and haplotypes of the present invention, i.e., by assessing the absence or presence of at least one allele of at least one polymorphic marker as disclosed herein, or by monitoring expression of genes that are associated with the variants (markers and haplotypes) of the present invention. The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype and/or haplotype status of at least one risk variant for the cancer as presented herein is determined before and during treatment to monitor its effectiveness.

Alternatively, biological networks or metabolic pathways related to the markers and haplotypes of the present invention can be monitored by determining mRNA and/or polypeptide levels. This can be done for example, by monitoring expression levels or polypeptides for several genes belonging to the network and/or pathway, in samples taken before and during treatment. Alternatively, metabolites belonging to the biological network or metabolic pathway can be determined before and during treatment. Effectiveness of the treatment is determined by comparing observed changes in expression levels/metabolite levels during treatment to corresponding data from healthy subjects.

In a further aspect, the markers of the present invention can be used to increase power and effectiveness of clinical trials. Thus, individuals who are carriers of at least one at-risk variant of the present invention, i.e. individuals who are carriers of at least one allele of at least one polymorphic marker or haplotype conferring increased risk of developing cancer may be more likely to respond to a particular treatment modality. In one embodiment, individuals who carry at-risk variants for gene(s) in a pathway and/or metabolic network for which a particular treatment (e.g., small molecule drug) is targeting, are more likely to be responders to the treatment. In another embodiment, individuals who carry at-risk variants for a gene, which expression and/or function is altered by the at-risk variant, are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., the markers and haplotypes of the present invention, are statistically significantly likely to show positive response to the therapeutic agent, i.e. experience alleviation of symptoms associated with cancer when taking the therapeutic agent or drug as prescribed.

In a further aspect, the markers and haplotypes of the present invention can be used for targeting the selection of pharmaceutical agents for specific individuals. Personalized selection of treatment modalities, lifestyle changes or combination of the two, can be realized by the utilization of the at-risk variants of the present invention. Thus, the knowledge of an individual's status for particular markers of the present invention, can be useful for selection of treatment options that target genes or gene products affected by the at-risk variants of the invention. Certain combinations of variants may be suitable for one selection of treatment options, while other gene variant combinations may target other treatment options. Such combination of variant may include one variant, two variants, three variants, or four or more variants, as needed to determine with clinically reliable accuracy the selection of treatment module.

In addition to the diagnostic and therapeutic uses of the variants of the present invention, the variants (markers and haplotypes) can also be useful markers for human identification, and as such be useful in forensics, paternity testing and in biometrics. The specific use of SNPs for forensic purposes is reviewed by Gill (Int. J. Legal Med. 114:204-10 (2001)). Genetic variations in genomic DNA between individuals can be used as genetic markers to identify individuals and to associate a biological sample with an individual. Genetic markers, including SNPs and microsatellites, can be useful to distinguish individuals. The more markers that are analyzed, the lower the probability that the allelic combination of the markers in any given individual is the same as in an unrelated individual (assuming that the markers are unrelated, i.e. that the markers are in perfect linkage equilibrium). Thus, the variants used for these purposes are preferably unrelated, i.e. they are inherited independently. Thus, preferred markers can be selected from available markers, such as the markers of the present invention, and the selected markers may comprise markers from different regions in the human genome, including markers on different chromosomes.

In certain applications, the SNPs useful for forensic testing are from degenerate codon positions (i.e., the third position in certain codons such that the variation of the SNP does not affect the amino acid encoded by the codon). In other applications, such for applications for predicting phenotypic characteristics including race, ancestry or physical characteristics, it may be more useful and desirable to utilize SNPs that affect the amino acid sequence of the encoded protein. In other such embodiments, the variant (SNP or other polymorphic marker) affects the expression level of a nearby gene, thus leading to altered protein expression.

### Nucleic Acids and Polypeptides

The nucleic acids and polypeptides described herein can be used in methods and kits of the present invention, as described in the above.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S. and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988).

In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence set forth in SEQ ID NO:1 or SEQ ID NO:2, or a nucleotide sequence comprising, or consisting of, the complement of the nucleotide sequence of SEQ ID NO:1 or SEQ ID NO:2, wherein the nucleotide sequence comprises at least one polymorphic allele contained in the markers and haplotypes described herein. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled) and used as a probe for screening a cDNA library derived from human cells. The cDNA can be derived from mRNA and contained in a suitable vector. Corresponding clones can be isolated, DNA can obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify cancer (e.g., prostate cancer) or a susceptibility to cancer (e.g., prostate cancer), and as probes, such as to hybridize and discover related DNA sequences or to subtract out known sequences from a sample (e.g., subtractive hybridization). The nucleic acid sequences can further be used to derive primers for genetic fingerprinting, to raise anti-polypeptide antibodies using immunization techniques, and/or as an antigen to raise anti-DNA antibodies or elicit immune responses.

### Antibodies

Polyclonal antibodies and/or monoclonal antibodies that specifically bind one form of a gene product but not to the other form of the gene product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorpic marker of the invention, can be used to identify individuals that require modified treatment modalities.

Antibodies can furthermore be useful for assessing expression of variant proteins in disease states, such as in active stages of a cancer (e.g., prostate cancer), or in an individual with a predisposition to cancer related to the function of the protein, in particular prostate cancer. Antibodies specific for a variant protein of the present invention that is encoded by a nucleic acid that comprises at least one polymorphic marker or haplotype as described herein can be used to screen for the presence of the variant protein, for example to screen for a predisposition to cancer (e.g., prostate cancer) as indicated by the presence of the variant protein.

Antibodies can be used in other methods. Thus, antibodies are useful as diagnostic tools for evaluating proteins, such as variant proteins of the invention, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention is now illustrated by the following Examples, which are not intended to be limiting in any way.

### Example 1

### Identification of the LD Block C Region Associated with Prostate Cancer

### Patients Involved in the Genetics Study

A population based list of all prostate cancer patients that were diagnosed with prostate cancer in Iceland from 1955 to 2005 form the basis for this study. Patients have been invited to join the study since 2001 on an ongoing basis. As of October 2006, blood samples from 1564 prostate cancer patients have been collected. Of that collection 1455 prostate cancer patients in addition to 4182 control individuals were genotyped with the Illumina 317K Bead chip.

### Statistical Methods for Association and Haplotype Analysis

For single marker association to the disease, Fisher exact test was used to calculate a two-sided P-value for each individual allele. When presenting the results, we used allelic frequencies rather than carrier frequencies for SNPs and haplotypes. Haplotype analyses were performed using a computer program we developed at deCODE called NEMO (NEsted MOdels) (Gretarsdóttir, et al., Nat. Genet. 2003 October; 35(2):131-8). NEMO was used both to study marker-marker association and to calculate linkage disequilibrium (LD) between markers, and for case-control haplotype analysis. With NEMO, haplotype frequencies are estimated by maximum likelihood and the differences between patients and controls are tested using a generalized likelihood ratio test. The maximum likelihood estimates, likelihood ratios and P-values are computed with the aid of the EM-algorithm directly for the observed data, and hence the loss of information due to the uncertainty with phase and missing genotypes is automatically captured by the likelihood ratios, and under most situations, large sample theory can be used to reliably determine statistical significance. The relative risk (RR) of an allele or a haplotype, i.e., the risk of an allele compared to all other alleles of the same marker, is calculated assuming the multiplicative model (Terwilliger, J. D. & Ott, J. A haplotype-based ‘haplotype relative risk’ approach to detecting allelic associations. Hum. Hered. 42, 337-46 (1992) and Falk, C. T. & Rubinstein, P. Haplotype relative risks: an easy reliable way to construct a proper control sample for risk calculations. Ann. Hum. Genet. 51 (Pt 3), 227-33 (1987)), together with the population attributable risk (PAR).

In the haplotype analysis, it may be useful to group haplotypes together and test the group as a whole for association to the disease. This is possible to do with NEMO. A model is defined by a partition of the set of all possible haplotypes, where haplotypes in the same group are assumed to confer the same risk while haplotypes in different groups can confer different risks. A null hypothesis and an alternative hypothesis are said to be nested when the latter corresponds to a finer partition than the former. NEMO provides complete flexibility in the partition of the haplotype space. In this way, it is possible to test multiple haplotypes jointly for association and to test if different haplotypes confer different risk. As a measure of LD, we use two standard definitions of LD, D′ and R² (Lewontin, R., Genetics, 49:49-67 (1964) and Hill, W. G. and A. Robertson, Theor. Appl. Genet., 22:226-231 (1968)) as they provide complementary information on the amount of LD. For the purpose of estimating D′ and R², the frequencies of all two-marker allele combinations are estimated using maximum likelihood methods and the deviation from linkage disequilibrium is evaluated using a likelihood ratio test. The standard definitions of D′ and R² are extended to include microsatellites by averaging over the values for all possible allele combinations of the two markers weighted by the marginal allele probabilities.

The number of possible haplotypes that can be constructed out of the dense set of markers genotyped over the whole genome is very large and even though the number of haplotypes that are actually observed in the patient and control cohort is much smaller, testing all of those haplotypes for association to the disease is a formidable task. It should be noted that we do not restrict our analysis to haplotypes constructed from a set of consecutive markers, as some markers may be very mutable and might split up an otherwise well conserved haplotype constructed out of surrounding markers

### Results

As described herein, a region on chromosome 8q24.21 (LD block C) was identified that confers an increased risk for particular cancers (e.g., prostate cancer (e.g., aggressive prostate cancer). Particular haplotypes and markers associated with an increased risk of prostate cancer are depicted in Table 1. As indicated in Table 1, the haplotype (HapC) involves the following markers (e.g., SNPs) and alleles: rs1456314 3 allele, rs17831626 4 allele, rs7825414 3 allele, rs6993569 3 allele, rs6994316 1 allele, rs6470494 4 allele, rs1016342 2 allele, rs1031588 3 allele, rs1016343 4 allele, rs1551510 3 allele, rs1456306 2 allele, rs1378897 3 allele, rs1456305 4 allele, rs7816535 3 allele. By examining results for the Caucasian (CEU) samples in the HapMap project allele 1 of the SNP marker rs16901979 was shown to be strongly correlated with HapC (D′=1, r²=1). The genotyping of rs16901979 in the Icelandic samples confirmed its correlation with HapC (D′=0.98, r2=0.70 in controls). The markers and haplotypes are located in what we call LD block C between positions 128,032,278 and 128,094,256 by (NCBI Build 34) and positions of the individual markers are indicated in Table 2). Allele codes for SNPs are as follows: 1=A, 2=C, 3=G, 4=T. Note that an increased risk is seen in aggressive prostate cancer as compared to all prostate cancer. The aggressive phenotype is determined by prostate cancer with combined Gleason grades of 7 or higher AND/OR stage T3 or higher AND/OR node positive AND/OR metastasis positive disease AND/OR death because of prostate cancer. Note that it is sufficient to have one of these criteria to be determined aggressive prostate cancer. These clinical parameters are well known surrogates for increased aggressiveness of the disease.

TABLE 1
Association of SNP markers on Chr8q24 to prostate cancer (PrCa) in Iceland

| Affected | # of Individuals | | | | Allele frequency | | | |
|---|---|---|---|---|---|---|---|---|
| Phenotype | Affected | Controls | p-value | RR | Affected | Controls | Allele or haplotype | PAR |
| PrCa | 1455 | 4182 | 7.86E−09 | 1.72 | 0.072 | 0.043 | 1 rs16901979 | 0.059 |
| PrCa | 1455 | 4182 | 3.89E−12 | 2.05 | 0.063 | 0.032 | HapC | 0.064 |
| Aggressive PrCa | 745 | 4182 | 4.82E−07 | 1.82 | 0.076 | 0.043 | 1 rs16901979 | 0.067 |
| Aggressive PrCa | 745 | 4182 | 3.69E−10 | 2.26 | 0.069 | 0.032 | HapC | 0.076 |

Alleles for the marker rs16901979 and SNPs in same LD block at 8q24.21 are shown and the corresponding numbers of cases and controls (N), allelic frequencies of variants in affected and control individuals, the odds-ratio (OR) and two-sided P values. Affected individuals are those diagnosed with prostate cancer (ICD10 = C61); or those diagnosed with prostate cancer (ICD10:C61) and have aggressive cancer as determined by a combined phenotype including cancers with Gleason combined grade of 7 or higher or cancers with stage T3 or higher or node positive disease or metastasis positive disease or men that have died because of prostate cancer. Controls are unaffected population based controls. Allele codes for SNPs are as follows: 1 = A, 2 = C, 3 = G, 4 = T. PAR is population attributable risk that can be explained by these variants.
Results for allele 1 of rs16901979 are generated by using genotypes for rs16901979 as well as other correlated SNPs in HapC to increase genotype yield.
HapC: 3 rs1456314 4 rs17831626 3 rs7825414 3 rs6993569 1 rs6994316 4 rs6470494 2 rs1016342 3 rs1031588 4 rs1016343 3 rs1551510 2 rs1456306 3 rs1378897 4 rs1456305 3 rs7816535.

As shown in table 1, both marker rs16901979 1 allele and the 14 marker HapC haplotype give significant association to prostate cancer. The population frequency of the haplotype is slightly lower than that of the rs16901979 1 allele, and the corresponding relative risk somewhat higher. The population attributable risk of these variants in Iceland is 5.9-6.4% in all prostate cancer and slightly higher or 6.7-7.6% in the aggressive prostate cancer patients.

The opposite allele of SNP marker rs16901979 or allele 2 shows significant protection to prostate cancer in carriers. These results are shown in Table 2.

TABLE 2
Variants on Chr8q24 that confer a protection
to prostate cancer in Iceland

| | # of Individuals | | | | Allele frequency | | |
|---|---|---|---|---|---|---|---|
| Affected Phenotype | Affected | Controls | p-value | RR | Affected | Controls | Allele |
| Prostate Cancer | 1446 | 4182 | 1.63E−08 | 0.59 | 0.928 | 0.957 | 2 rs16901979 |
| Aggressive PrCa | 745 | 4182 | 4.82E−07 | 0.55 | 0.924 | 0.957 | 2 rs16901979 |

Alleles for the marker rs16901979 at 8q24.21 are shown and the corresponding numbers of cases and controls (N), allelic frequencies of variants in affected and control individuals, the odds-ratio (OR) and two-sided P values. Affected individuals are those diagnosed with prostate cancer (ICD10 = C61); or those diagnosed with prostate cancer (ICD10:C61) and have aggressive cancer as determined by a combined phenotype including cancers with Gleason combined grade of 7 or higher or with stage T3 or higher or node positive disease or metastasis positive disease or have died because of prostate cancer. Controls are unaffected population based controls. Allele codes for SNPs are as follows: 1 = A, 2 = C, 3 = G, 4 = T

Table 3 lists SNP markers in HapC in the LD Block C region. Location of the SNPs is given with respect to NCBI Build34 of the human genome assembly. The relative position of the markers in basepair position is indicated.

TABLE 3
Genomic location of SNPs
in HapC and marker rs16901979

| rs name | BUILD34 |
|---|---|
| rs1456314 | 128032278 |
| rs17831626 | 128037011 |
| rs7825414 | 128038109 |
| rs6993569 | 128040685 |
| rs6994316 | 128041127 |
| rs6470494 | 128044492 |
| rs1016342 | 128049043 |
| rs1031588 | 128049865 |
| rs1016343 | 128049885 |
| rs1551510 | 128050067 |
| rs1456306 | 128073088 |
| rs1378897 | 128079247 |
| rs16901979 | 128081505 |
| rs1456305 | 128083840 |
| rs7816535 | 128094256 |

SNP markers in HapC and rs16901979 with genomic locations (base pairs) in NCBI Build 34.

To find this association of LD block C, as manifested by the marker and haplotypes tested and indicated in Table 1, we performed a genome-wide scan using Infinium HumanHap300 SNP chips from Illumina for assaying approximately 317,000 single nucleotide polymorphisms (SNPs) on a single chip. We genotyped 1455 patients and 4182 controls in this manner. The controls represent an unaffected Icelandic population sample. Association analysis was done with single markers, two markers and haplotypes representing all consecutive markers within each and every LD block in the human genome. The SNP marker rs16901979 1 allele was found to give highly significant association to prostate cancer. The marker rs16901979 is not on the Illumina Hap300 chip and was genotyped independently. Two marker haplotypes of markers within LD block C also gave highly significant association to prostate cancer. When haplotypes consisting of all consecutive SNP markers within each LD block throughout the whole genome were tested (LD block haplotypes) for their association to prostate cancer, the most significant haplotype was on Chr8q24 at location 128.414-128.506 Mb (NCBI Build34). This finding is already described by us (Amundadottir et. al. Nat. Genet. 2006 June; 38(6):652-8). Much to our surprise the second best LD block haplotype was located only a few hundred thousand basepairs away on Chr8q24 or at 128.032-128.095 Mb (NCBI Build34). We can call this haplotype and LD block haplotype C (HapC) and LD block C. Due to the LD block (haplotype block) structure of the human genome, we proceeded to investigate the LD block C, within which rs16901979 resides.

There are 30 SNPs that are in strong LD with rs16901979 and would therefore be just as good in detecting the association to prostate cancer described herein. The LD between these SNPs and rs16901979 as measured by r² is greater than 0.5. These markers are listed in Table 4A. Table 4B lists markers in LD with rs16901979 in the different HapMap populations with values of r² to rs 16901979 of at least 0.2 in that population.

TABLE 4A
SNPs that are correlated with
rs16901979

| rs name | BUILD34 |
|---|---|
| rs10453084 | 128069367 |
| rs10505483 | 128081783 |
| rs1551512 | 128080714 |
| rs16901948 | 128063689 |
| rs16901949 | 128063741 |
| rs16901950 | 128063831 |
| rs16901952 | 128063858 |
| rs16901953 | 128064817 |
| rs16901959 | 128066118 |
| rs16901966 | 128066840 |
| rs16901967 | 128066865 |
| rs16901969 | 128068685 |
| rs16901970 | 128069303 |
| rs16901984 | 128087549 |
| rs6470498 | 128072308 |
| rs6983561 | 128063468 |
| rs6987640 | 128069616 |
| rs6987723 | 128069447 |
| rs6988257 | 128068044 |
| rs6989838 | 128085960 |
| rs6990420 | 128065313 |
| rs7001069 | 128067234 |
| rs7010450 | 128065267 |
| rs7013255 | 128087075 |
| rs7817677 | 128082092 |
| rs7824451 | 128071049 |
| rs7824785 | 128071298 |
| rs7826388 | 128066364 |
| rs7830341 | 128066518 |
| rs7844219 | 128075403 |

All SNPs are listed with a r² > 0.5 with rs16901979. SNP names are listed with genomic locations (base pair start) in NCBI Build 34.

TABLE 4B
Markers in strong linkage disequilibrium to marker rs16901979. Markers that are correlated to rs16901979 by
values of r² of at least 0.2 in the Caucasian CEPH population CEU, African Yuroba
population YRI, Japanese population JPT and Chinese population CHB are shown. Shown are values for the
degree of LD of each marker with rs16901979 (D′, r² and p-value), as well as the location of the markers
in NCBI Builds 34, 35, 36 and SEQ ID NO: 2 (LD Block C′), as well a reference to their SEQ ID NO.
CEU HapMap

| | | | | | | | Position | |
|---|---|---|---|---|---|---|---|---|
| | | | | Position | | Position | SEQ ID | SEQ ID |
| Marker | D′ | R2 | p | (B34) | Position B35 | (B36) | NO: 2 | NO |
| rs12682344 | 1 | 1 | 1.18E−07 | 128063373 | 128175966 | 128175966 | 34261 | 43 |
| rs6983561 | 1 | 1 | 1.18E−07 | 128063469 | 128176062 | 128176062 | 34357 | 21 |
| rs16901948 | 1 | 1 | 1.18E−07 | 128063690 | 128176283 | 128176283 | 34578 | 9 |
| rs16901949 | 1 | 1 | 1.18E−07 | 128063742 | 128176335 | 128176335 | 34630 | 10 |
| rs16901950 | 1 | 1 | 1.18E−07 | 128063832 | 128176425 | 128176425 | 34720 | 11 |
| rs16901952 | 1 | 1 | 1.24E−07 | 128063859 | 128176452 | 128176452 | 34747 | 12 |
| rs16901953 | 1 | 1 | 1.18E−07 | 128064818 | 128177411 | 128177411 | 35706 | 13 |
| rs7010450 | 1 | 1 | 1.18E−07 | 128065268 | 128177861 | 128177861 | 36156 | 28 |
| rs6990420 | 1 | 0.74026 | 1.28E−06 | 128065314 | 128177907 | 128177907 | 36202 | 26 |
| rs16901959 | 1 | 1 | 1.18E−07 | 128066119 | 128178712 | 128178712 | 37007 | 14 |
| rs7826388 | 1 | 1 | 1.18E−07 | 128066365 | 128178958 | 128178958 | 37253 | 33 |
| rs7830341 | 1 | 1 | 1.18E−07 | 128066519 | 128179112 | 128179112 | 37407 | 34 |
| rs16901966 | 1 | 1 | 1.18E−07 | 128066841 | 128179434 | 128179434 | 37729 | 15 |
| rs16901967 | 1 | 1 | 1.18E−07 | 128066866 | 128179459 | 128179459 | 37754 | 16 |
| rs7001069 | 1 | 1 | 1.18E−07 | 128067235 | 128179828 | 128179828 | 38123 | 27 |
| rs6988257 | 1 | 1 | 1.18E−07 | 128068045 | 128180638 | 128180638 | 38933 | 24 |
| rs16901969 | 1 | 1 | 1.18E−07 | 128068686 | 128181279 | 128181279 | 39574 | 17 |
| rs16901970 | 1 | 1 | 1.18E−07 | 128069304 | 128181897 | 128181897 | 40192 | 18 |
| rs10453084 | 1 | 1 | 1.18E−07 | 128069368 | 128181961 | 128181961 | 40256 | 6 |
| rs6987723 | 1 | 1 | 1.18E−07 | 128069448 | 128182041 | 128182041 | 40336 | 23 |
| rs6987640 | 1 | 1 | 1.18E−07 | 128069617 | 128182210 | 128182210 | 40505 | 22 |
| rs7824451 | 1 | 1 | 1.18E−07 | 128071050 | 128183643 | 128183643 | 41938 | 31 |
| rs7824785 | 1 | 1 | 1.18E−07 | 128071299 | 128183892 | 128183892 | 42187 | 32 |
| rs6470498 | 1 | 1 | 1.18E−07 | 128072309 | 128184902 | 128184902 | 43197 | 20 |
| rs7844219 | 1 | 1 | 1.18E−07 | 128075404 | 128187997 | 128187997 | 46292 | 35 |
| rs1551512 | 1 | 1 | 1.18E−07 | 128080715 | 128193308 | 128193308 | 51603 | 8 |
| rs10505483 | 1 | 1 | 1.18E−07 | 128081784 | 128194377 | 128194377 | 52672 | 7 |
| rs7817677 | 1 | 1 | 1.18E−07 | 128082093 | 128194686 | 128194686 | 52981 | 30 |
| rs6989838 | 1 | 1 | 1.18E−07 | 128085961 | 128198554 | 128198554 | 56849 | 25 |
| rs7013255 | 1 | 1 | 1.18E−07 | 128087076 | 128199669 | 128199669 | 57964 | 29 |
| rs16901984 | 1 | 1 | 1.45E−07 | 128087550 | 128200143 | 128200143 | 58438 | 19 |

| | | | | | | | | |
|---|---|---|---|---|---|---|---|---|
| | | | | | | | Position | |
| | | | | Location | | Location | SEQ ID | SEQ ID |
| M1 | D′ | r2 | p | (B34) | Location B35 | (B36) | NO: 2 | NO |

YRI HapMap

| | | | | | | | | |
|---|---|---|---|---|---|---|---|---|
| rs1456315 | 0.583779 | 0.237876 | 9.12E−07 | 128060526 | 128173119 | 128173119 | 31414 | 46 |
| rs13254738 | 0.767679 | 0.260913 | 4.36E−08 | 128060932 | 128173525 | 128173525 | 31820 | 45 |
| rs7006390 | 0.923431 | 0.652818 | 8.36E−21 | 128060956 | 128173549 | 128173549 | 31844 | 63 |
| rs1073997 | 0.86296 | 0.720149 | 1.90E−22 | 128061776 | 128174369 | 128174369 | 32664 | 41 |
| rs7012442 | 0.966424 | 0.933975 | 3.18E−32 | 128062320 | 128174913 | 128174913 | 33208 | 65 |
| rs6983561 | 0.962864 | 0.782622 | 1.44E−25 | 128063469 | 128176062 | 128176062 | 34357 | 21 |
| rs16901948 | 1 | 0.507692 | 7.86E−19 | 128063690 | 128176283 | 128176283 | 34578 | 9 |
| rs16901949 | 1 | 0.507692 | 7.86E−19 | 128063742 | 128176335 | 128176335 | 34630 | 10 |
| rs16901950 | 1 | 0.507692 | 7.86E−19 | 128063832 | 128176425 | 128176425 | 34720 | 11 |
| rs16901952 | 1 | 0.507692 | 2.04E−18 | 128063859 | 128176452 | 128176452 | 34747 | 12 |
| rs16901953 | 1 | 0.507692 | 7.86E−19 | 128064818 | 128177411 | 128177411 | 35706 | 13 |
| rs7010450 | 1 | 0.507692 | 1.27E−18 | 128065268 | 128177861 | 128177861 | 36156 | 28 |
| rs12544977 | 0.93185 | 0.31233 | 2.91E−10 | 128065876 | 128178469 | 128178469 | 36764 | 42 |
| rs16901959 | 1 | 0.507692 | 7.86E−19 | 128066119 | 128178712 | 128178712 | 37007 | 14 |
| rs7826337 | 0.95706 | 0.604515 | 1.40E−19 | 128066163 | 128178756 | 128178756 | 37051 | 69 |
| rs7826388 | 1 | 0.294728 | 6.60E−12 | 128066365 | 128178958 | 128178958 | 37253 | 33 |
| rs7830341 | 1 | 0.489879 | 2.94E−18 | 128066519 | 128179112 | 128179112 | 37407 | 34 |
| rs16901966 | 1 | 0.507692 | 7.86E−19 | 128066841 | 128179434 | 128179434 | 37729 | 15 |
| rs16901967 | 1 | 0.507692 | 7.86E−19 | 128066866 | 128179459 | 128179459 | 37754 | 16 |
| rs7000910 | 0.954702 | 0.55904 | 4.30E−18 | 128067194 | 128179787 | 128179787 | 38082 | 62 |
| rs7001069 | 1 | 0.507692 | 7.86E−19 | 128067235 | 128179828 | 128179828 | 38123 | 27 |
| rs7006409 | 0.932011 | 0.31983 | 6.19E−10 | 128068018 | 128180611 | 128180611 | 38906 | 64 |
| rs6988257 | 1 | 0.507692 | 7.86E−19 | 128068045 | 128180638 | 128180638 | 38933 | 24 |
| rs16901969 | 1 | 0.507692 | 7.86E−19 | 128068686 | 128181279 | 128181279 | 39574 | 17 |
| rs16901970 | 1 | 0.507692 | 7.86E−19 | 128069304 | 128181897 | 128181897 | 40192 | 18 |
| rs10453084 | 1 | 0.507692 | 7.86E−19 | 128069368 | 128181961 | 128181961 | 40256 | 6 |
| rs6987723 | 1 | 0.489879 | 2.94E−18 | 128069448 | 128182041 | 128182041 | 40336 | 23 |
| rs6987640 | 1 | 0.507692 | 7.86E−19 | 128069617 | 128182210 | 128182210 | 40505 | 22 |
| rs7824451 | 1 | 0.507692 | 7.86E−19 | 128071050 | 128183643 | 128183643 | 41938 | 31 |
| rs7824785 | 1 | 0.507692 | 7.86E−19 | 128071299 | 128183892 | 128183892 | 42187 | 32 |
| rs6470498 | 1 | 0.507692 | 7.86E−19 | 128072309 | 128184902 | 128184902 | 43197 | 20 |
| rs7827234 | 1 | 0.610001 | 1.92E−21 | 128074695 | 128187288 | 128187288 | 45583 | 70 |
| rs7844219 | 0.964897 | 0.930545 | 3.58E−30 | 128075404 | 128187997 | 128187997 | 46292 | 35 |
| rs6470499 | 1 | 0.934871 | 5.99E−34 | 128079983 | 128192576 | 128192576 | 50871 | 56 |
| rs1551512 | 1 | 0.934871 | 5.99E−34 | 128080715 | 128193308 | 128193308 | 51603 | 8 |
| rs10505483 | 1 | 1 | 7.04E−38 | 128081784 | 128194377 | 128194377 | 52672 | 7 |
| rs7817677 | 1 | 0.525988 | 2.02E−19 | 128082093 | 128194686 | 128194686 | 52981 | 30 |
| rs6989838 | 1 | 1 | 7.04E−38 | 128085961 | 128198554 | 128198554 | 56849 | 25 |
| rs7013255 | 1 | 1 | 7.04E−38 | 128087076 | 128199669 | 128199669 | 57964 | 29 |
| rs16901984 | 1 | 0.525043 | 6.71E−18 | 128087550 | 128200143 | 128200143 | 58438 | 19 |
| rs7000307 | 1 | 0.334526 | 3.19E−13 | 128089665 | 128202258 | 128202258 | 60553 | 61 |
| rs7840773 | 1 | 0.334526 | 3.19E−13 | 128091923 | 128204516 | 128204516 | 62811 | 71 |
| rs7824364 | 1 | 0.334526 | 3.19E−13 | 128091954 | 128204547 | 128204547 | 62842 | 68 |

JPT HapMap

| | | | | | | | | |
|---|---|---|---|---|---|---|---|---|
| rs16901932 | 0.871703 | 0.309945 | 0.000085 | 128028107 | 128140700 | 128140700 | | 48 |
| rs16901935 | 0.668518 | 0.254464 | 0.000841 | 128033134 | 128145727 | 128145727 | 4022 | 49 |
| rs6470494 | 0.654239 | 0.236706 | 0.000813 | 128044493 | 128157086 | 128157086 | 15381 | 55 |
| rs1016343 | 1 | 0.52 | 3.40E−11 | 128049886 | 128162479 | 128162479 | 20774 | 37 |
| rs7841060 | 1 | 0.52 | 3.40E−11 | 128053066 | 128165659 | 128165659 | 23954 | 72 |
| rs7006390 | 1 | 0.921053 | 4.71E−17 | 128060956 | 128173549 | 128173549 | 31844 | 63 |
| rs1073997 | 1 | 1 | 7.61E−17 | 128061776 | 128174369 | 128174369 | 32664 | 41 |
| rs7012442 | 1 | 1 | 1.14E−18 | 128062320 | 128174913 | 128174913 | 33208 | 65 |
| rs12682344 | 1 | 1 | 1.14E−18 | 128063373 | 128175966 | 128175966 | 34261 | 43 |
| rs6983561 | 1 | 1 | 9.10E−18 | 128063469 | 128176062 | 128176062 | 34357 | 21 |
| rs16901948 | 1 | 1 | 1.14E−18 | 128063690 | 128176283 | 128176283 | 34578 | 9 |
| rs16901949 | 1 | 1 | 1.14E−18 | 128063742 | 128176335 | 128176335 | 34630 | 10 |
| rs16901950 | 1 | 1 | 1.14E−18 | 128063832 | 128176425 | 128176425 | 34720 | 11 |
| rs16901952 | 1 | 1 | 1.14E−18 | 128063859 | 128176452 | 128176452 | 34747 | 12 |
| rs16901953 | 1 | 1 | 1.14E−18 | 128064818 | 128177411 | 128177411 | 35706 | 13 |
| rs7010450 | 1 | 1 | 1.14E−18 | 128065268 | 128177861 | 128177861 | 36156 | 28 |
| rs6990420 | 1 | 0.736842 | 1.81E−14 | 128065314 | 128177907 | 128177907 | 36202 | 26 |
| rs16901959 | 1 | 1 | 1.14E−18 | 128066119 | 128178712 | 128178712 | 37007 | 14 |
| rs7826337 | 1 | 0.238919 | 3.93E−07 | 128066163 | 128178756 | 128178756 | 37051 | 69 |
| rs7826388 | 1 | 1 | 1.14E−18 | 128066365 | 128178958 | 128178958 | 37253 | 33 |
| rs7830341 | 1 | 1 | 9.10E−18 | 128066519 | 128179112 | 128179112 | 37407 | 34 |
| rs16901966 | 1 | 1 | 1.14E−18 | 128066841 | 128179434 | 128179434 | 37729 | 15 |
| rs16901967 | 1 | 1 | 1.14E−18 | 128066866 | 128179459 | 128179459 | 37754 | 16 |
| rs7000910 | 1 | 0.238919 | 3.93E−07 | 128067194 | 128179787 | 128179787 | 38082 | 62 |
| rs7001069 | 1 | 1 | 9.10E−18 | 128067235 | 128179828 | 128179828 | 38123 | 27 |
| rs6988257 | 1 | 1 | 9.10E−18 | 128068045 | 128180638 | 128180638 | 38933 | 24 |
| rs16901969 | 1 | 1 | 1.14E−18 | 128068686 | 128181279 | 128181279 | 39574 | 17 |
| rs16901970 | 1 | 1 | 1.14E−18 | 128069304 | 128181897 | 128181897 | 40192 | 18 |
| rs10453084 | 1 | 1 | 1.14E−18 | 128069368 | 128181961 | 128181961 | 40256 | 6 |
| rs6987723 | 1 | 1 | 1.14E−18 | 128069448 | 128182041 | 128182041 | 40336 | 23 |
| rs6987640 | 1 | 1 | 9.10E−18 | 128069617 | 128182210 | 128182210 | 40505 | 22 |
| rs7824451 | 1 | 1 | 9.10E−18 | 128071050 | 128183643 | 128183643 | 41938 | 31 |
| rs7824785 | 1 | 1 | 9.10E−18 | 128071299 | 128183892 | 128183892 | 42187 | 32 |
| rs6470498 | 1 | 1 | 1.14E−18 | 128072309 | 128184902 | 128184902 | 43197 | 20 |
| rs7827234 | 1 | 0.238919 | 3.93E−07 | 128074695 | 128187288 | 128187288 | 45583 | 70 |
| rs7844219 | 1 | 1 | 1.14E−18 | 128075404 | 128187997 | 128187997 | 46292 | 35 |
| rs6470499 | 1 | 1 | 9.10E−18 | 128079983 | 128192576 | 128192576 | 50871 | 56 |
| rs1551512 | 1 | 1 | 9.10E−18 | 128080715 | 128193308 | 128193308 | 51603 | 8 |
| rs10505483 | 1 | 1 | 1.26E−17 | 128081784 | 128194377 | 128194377 | 52672 | 7 |
| rs7817677 | 1 | 1 | 1.14E−18 | 128082093 | 128194686 | 128194686 | 52981 | 30 |
| rs6989838 | 1 | 1 | 9.10E−18 | 128085961 | 128198554 | 128198554 | 56849 | 25 |
| rs7013255 | 1 | 1 | 9.10E−18 | 128087076 | 128199669 | 128199669 | 57964 | 29 |
| rs16901984 | 1 | 1 | 8.21E−15 | 128087550 | 128200143 | 128200143 | 58438 | 19 |
| rs16901997 | 1 | 0.387755 | 5.84E−07 | 128128090 | 128240683 | 128240683 | | 50 |
| rs283710 | 0.634312 | 0.209223 | 0.002854 | 128246180 | 128358773 | 128358773 | | 52 |
| rs283719 | 0.657568 | 0.254825 | 0.000865 | 128266367 | 128378960 | 128378960 | | 53 |
| rs7017081 | 0.576954 | 0.264459 | 0.003929 | 128275936 | 128388529 | 128388529 | | 66 |
| rs6984136 | 0.570098 | 0.235283 | 0.002359 | 128276727 | 128389320 | 128389320 | | 58 |
| rs1995613 | 0.570098 | 0.235283 | 0.002359 | 128278532 | 128391125 | 128391125 | | 51 |

CHB HapMap

| | | | | | | | | |
|---|---|---|---|---|---|---|---|---|
| rs10216560 | 0.562661 | 0.293676 | 0.001256 | 127962278 | 128074871 | 128074871 | | 38 |
| rs6470494 | 0.649644 | 0.224207 | 0.00107 | 128044493 | 128157086 | 128157086 | 15381 | 55 |
| rs1016342 | 1 | 0.246652 | 2.77E−08 | 128049044 | 128161637 | 128161637 | 19932 | 36 |
| rs1031589 | 1 | 0.235197 | 5.23E−08 | 128049571 | 128162164 | 128162164 | 20459 | 40 |
| rs1016343 | 0.77074 | 0.361993 | 9.71E−06 | 128049886 | 128162479 | 128162479 | 20774 | 37 |
| rs1031587 | 1 | 0.235197 | 5.23E−08 | 128050061 | 128162654 | 128162654 | 20949 | 39 |
| rs1551510 | 1 | 0.22807 | 1.07E−07 | 128050067 | 128162660 | 128162660 | 20955 | 47 |
| rs4871008 | 1 | 0.224138 | 9.74E−08 | 128050130 | 128162723 | 128162723 | 21018 | 54 |
| rs6981122 | 1 | 0.224138 | 9.74E−08 | 128051049 | 128163642 | 128163642 | 21937 | 57 |
| rs13252298 | 1 | 0.206124 | 3.66E−07 | 128051745 | 128164338 | 128164338 | 22633 | 44 |
| rs7841060 | 0.77074 | 0.361993 | 9.71E−06 | 128053066 | 128165659 | 128165659 | 23954 | 72 |
| rs6997559 | 1 | 0.235197 | 5.23E−08 | 128054167 | 128166760 | 128166760 | 25055 | 60 |
| rs7006390 | 1 | 0.895105 | 2.06E−21 | 128060956 | 128173549 | 128173549 | 31844 | 63 |
| rs1073997 | 1 | 1 | 2.42E−25 | 128061776 | 128174369 | 128174369 | 32664 | 41 |
| rs7012442 | 1 | 1 | 2.42E−25 | 128062320 | 128174913 | 128174913 | 33208 | 65 |
| rs12682344 | 1 | 1 | 2.42E−25 | 128063373 | 128175966 | 128175966 | 34261 | 43 |
| rs6983561 | 1 | 1 | 2.42E−25 | 128063469 | 128176062 | 128176062 | 34357 | 21 |
| rs16901948 | 1 | 1 | 2.42E−25 | 128063690 | 128176283 | 128176283 | 34578 | 9 |
| rs16901949 | 1 | 1 | 2.42E−25 | 128063742 | 128176335 | 128176335 | 34630 | 10 |
| rs16901950 | 1 | 1 | 2.42E−25 | 128063832 | 128176425 | 128176425 | 34720 | 11 |
| rs16901952 | 1 | 1 | 4.86E−25 | 128063859 | 128176452 | 128176452 | 34747 | 12 |
| rs16901953 | 1 | 1 | 2.42E−25 | 128064818 | 128177411 | 128177411 | 35706 | 13 |
| rs7010450 | 1 | 1 | 2.42E−25 | 128065268 | 128177861 | 128177861 | 36156 | 28 |
| rs6990420 | 1 | 0.729647 | 5.85E−18 | 128065314 | 128177907 | 128177907 | 36202 | 26 |
| rs12544977 | 1 | 0.325 | 4.30E−10 | 128065876 | 128178469 | 128178469 | 36764 | 42 |
| rs16901959 | 1 | 1 | 2.42E−25 | 128066119 | 128178712 | 128178712 | 37007 | 14 |
| rs7826337 | 1 | 0.40625 | 7.40E−12 | 128066163 | 128178756 | 128178756 | 37051 | 69 |
| rs7826388 | 1 | 1 | 2.42E−25 | 128066365 | 128178958 | 128178958 | 37253 | 33 |
| rs7830341 | 1 | 1 | 2.42E−25 | 128066519 | 128179112 | 128179112 | 37407 | 34 |
| rs16901966 | 1 | 1 | 2.42E−25 | 128066841 | 128179434 | 128179434 | 37729 | 15 |
| rs16901967 | 1 | 1 | 2.42E−25 | 128066866 | 128179459 | 128179459 | 37754 | 16 |
| rs7000910 | 1 | 0.40625 | 7.40E−12 | 128067194 | 128179787 | 128179787 | 38082 | 62 |
| rs7001069 | 1 | 1 | 2.42E−25 | 128067235 | 128179828 | 128179828 | 38123 | 27 |
| rs7006409 | 1 | 0.3125 | 1.46E−09 | 128068018 | 128180611 | 128180611 | 38906 | 64 |
| rs6988257 | 1 | 1 | 2.42E−25 | 128068045 | 128180638 | 128180638 | 38933 | 24 |
| rs16901969 | 1 | 1 | 2.42E−25 | 128068686 | 128181279 | 128181279 | 39574 | 17 |
| rs16901970 | 1 | 1 | 2.42E−25 | 128069304 | 128181897 | 128181897 | 40192 | 18 |
| rs10453084 | 1 | 1 | 2.42E−25 | 128069368 | 128181961 | 128181961 | 40256 | 6 |
| rs6987723 | 1 | 1 | 2.42E−25 | 128069448 | 128182041 | 128182041 | 40336 | 23 |
| rs6987640 | 1 | 1 | 2.42E−25 | 128069617 | 128182210 | 128182210 | 40505 | 22 |
| rs7824451 | 1 | 1 | 2.42E−25 | 128071050 | 128183643 | 128183643 | 41938 | 31 |
| rs7824785 | 1 | 1 | 2.42E−25 | 128071299 | 128183892 | 128183892 | 42187 | 32 |
| rs6470498 | 1 | 1 | 2.42E−25 | 128072309 | 128184902 | 128184902 | 43197 | 20 |
| rs7827234 | 1 | 0.40625 | 7.40E−12 | 128074695 | 128187288 | 128187288 | 45583 | 70 |
| rs7844219 | 1 | 1 | 2.42E−25 | 128075404 | 128187997 | 128187997 | 46292 | 35 |
| rs6470499 | 1 | 1 | 2.42E−25 | 128079983 | 128192576 | 128192576 | 50871 | 56 |
| rs1551512 | 1 | 1 | 2.42E−25 | 128080715 | 128193308 | 128193308 | 51603 | 8 |
| rs10505483 | 1 | 1 | 2.42E−25 | 128081784 | 128194377 | 128194377 | 52672 | 7 |
| rs7817677 | 1 | 1 | 2.42E−25 | 128082093 | 128194686 | 128194686 | 52981 | 30 |
| rs6989838 | 1 | 1 | 2.42E−25 | 128085961 | 128198554 | 128198554 | 56849 | 25 |
| rs7013255 | 1 | 1 | 2.42E−25 | 128087076 | 128199669 | 128199669 | 57964 | 29 |
| rs16901984 | 1 | 1 | 2.38E−24 | 128087550 | 128200143 | 128200143 | 58438 | 19 |
| rs7816535 | 1 | 0.283608 | 3.74E−09 | 128094257 | 128206850 | 128206850 | 65145 | 67 |
| rs6990483 | 0.510459 | 0.260568 | 0.000619 | 128655867 | 128768460 | 128768460 | | 59 |

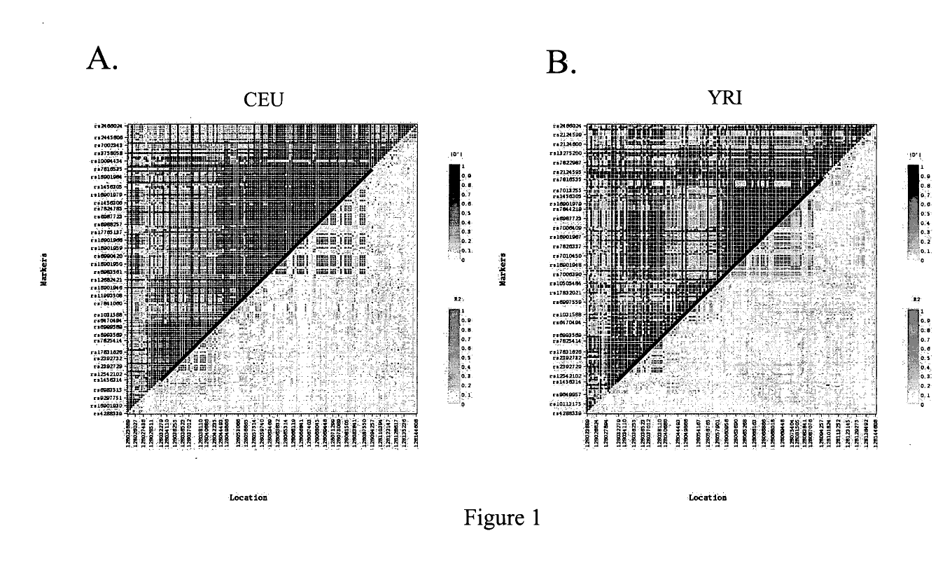

The LD structure in the LD Block C area of the markers and haplotypes that associates with prostate cancer is shown in FIG. 1. The structure was derived from HAPMAP data release 19. This LD block (LD block C) is located between markers rs1456314, located at 128,032,278 bp, and rs7816535, located at 128,094,256 by (NCBI build 34), and is almost 65 kb in length. The LD structure is seen as a block of DNA that has a high r² and |D′| between markers as indicated in the figure. Markers are represented equidistantly, i.e. with the same distance between any two markers.

It is possible that other markers residing within the LD block C region also are associated with prostate cancer, since such markers are in linkage disequilibrium with rs16901979. Table 5A provides a list of publicly known SNP markers in the LD Block C spanned by LD block C (positions 128032278-128094256 by in NCBI Build 34). The Table 5B provides a list of all public SNP markers in LD block C′ (corresponding to the sequence set forth in SEQ ID NO:2), and Table 5C provides a list of all microsatellite markers within that region.

TABLE 5A
All known SNPs within LD
block C (SEQ ID NO: 1)

| rs name | BUILD34 |
|---|---|
| rs1456314 | 128032278 |
| rs1551516 | 128032477 |
| rs3940781 | 128032803 |
| rs16901935 | 128033133 |
| rs12542102 | 128034109 |
| rs11987219 | 128035269 |
| rs11988135 | 128035997 |
| rs2124598 | 128036063 |
| rs2392726 | 128036067 |
| rs2392727 | 128036184 |
| rs2392728 | 128036231 |
| rs2392729 | 128036254 |
| rs1014656 | 128036271 |
| rs2392730 | 128036282 |
| rs2392731 | 128036328 |
| rs1014655 | 128036427 |
| rs2392732 | 128036522 |
| rs2893603 | 128036545 |
| rs9656965 | 128036836 |
| rs17831626 | 128037011 |
| rs16901938 | 128037088 |
| rs28451013 | 128037234 |
| rs28385355 | 128037374 |
| rs11785961 | 128037423 |
| rs13267056 | 128037675 |
| rs7824674 | 128037681 |
| rs7824679 | 128037698 |
| rs7824923 | 128037726 |
| rs7843300 | 128037819 |
| rs7824957 | 128037848 |
| rs7825394 | 128038037 |
| rs10308400 | 128038109 |
| rs7825414 | 128038109 |
| rs11994932 | 128038486 |
| rs13248907 | 128038933 |
| rs13282331 | 128038957 |
| rs13282364 | 128038997 |
| rs12548394 | 128039290 |
| rs10089648 | 128039437 |
| rs13363429 | 128039437 |
| rs11985630 | 128040216 |
| rs6993569 | 128040685 |
| rs6994316 | 128041127 |
| rs12550334 | 128041512 |
| rs11998124 | 128042068 |
| rs6999589 | 128042234 |
| rs11998248 | 128042343 |
| rs7005114 | 128043346 |
| rs11319438 | 128043465 |
| rs6991325 | 128043514 |
| rs1902431 | 128043664 |
| rs6470494 | 128044492 |
| rs7843737 | 128044654 |
| rs10095770 | 128045158 |
| rs6987760 | 128046899 |
| rs10306042 | 128047254 |
| rs6470495 | 128047254 |
| rs4581008 | 128047453 |
| rs7461435 | 128047550 |
| rs4620246 | 128047550 |
| rs12544839 | 128047970 |
| rs12544844 | 128048146 |
| rs10956351 | 128048147 |
| rs1016342 | 128049043 |
| rs1551511 | 128049402 |
| rs1031589 | 128049570 |
| rs1378896 | 128049642 |
| rs1031588 | 128049865 |
| rs1016343 | 128049885 |
| rs1031587 | 128050060 |
| rs1551510 | 128050066 |
| rs4871008 | 128050129 |
| rs11363193 | 128050445 |
| rs6981122 | 128051048 |
| rs7001504 | 128051071 |
| rs13252298 | 128051744 |
| rs7841060 | 128053065 |
| rs4540377 | 128053502 |
| rs6997559 | 128054166 |
| rs11554368 | 128055276 |
| rs7007694 | 128055754 |
| rs17831913 | 128055754 |
| rs13257371 | 128055830 |
| rs4571699 | 128055857 |
| rs1840709 | 128056043 |
| rs3037753 | 128056058 |
| rs7837848 | 128056120 |
| rs3837167 | 128056513 |
| rs3837166 | 128056618 |
| rs11993508 | 128056664 |
| rs17832021 | 128056764 |
| rs3999809 | 128056896 |
| rs3999810 | 128056955 |
| rs28663168 | 128056997 |
| rs3999811 | 128057050 |
| rs3999812 | 128057095 |
| rs3999806 | 128057100 |
| rs3857883 | 128057194 |
| rs2392733 | 128057260 |
| rs1456317 | 128057294 |
| rs1456316 | 128057436 |
| rs16901946 | 128057513 |
| rs28735726 | 128057517 |
| rs9656813 | 128057552 |
| rs9656814 | 128057565 |
| rs9656815 | 128057591 |
| rs10505484 | 128057900 |
| rs11994653 | 128057931 |
| rs6470496 | 128058617 |
| rs6470497 | 128058918 |
| rs7844454 | 128059089 |
| rs7826322 | 128059141 |
| rs12677860 | 128059213 |
| rs12677861 | 128059215 |
| rs7000321 | 128059607 |
| rs12682421 | 128059739 |
| rs7000967 | 128059948 |
| rs7001895 | 128060267 |
| rs1456315 | 128060525 |
| rs5013678 | 128060567 |
| rs7463708 | 128060643 |
| rs13254738 | 128060931 |
| rs7006390 | 128060955 |
| rs1073997 | 128061775 |
| rs7012407 | 128062181 |
| rs7012442 | 128062319 |
| rs7016828 | 128062820 |
| rs7016830 | 128062823 |
| rs12682344 | 128063372 |
| rs6983561 | 128063468 |
| rs16901948 | 128063689 |
| rs1869931 | 128063724 |
| rs16901949 | 128063741 |
| rs16901950 | 128063831 |
| rs16901952 | 128063858 |
| rs7005144 | 128064232 |
| rs16901953 | 128064817 |
| rs4871009 | 128065004 |
| rs7010450 | 128065267 |
| rs6990420 | 128065313 |
| rs6994359 | 128065511 |
| rs17832285 | 128065581 |
| rs7825340 | 128065717 |
| rs12544977 | 128065875 |
| rs16901959 | 128066118 |
| rs7826337 | 128066162 |
| rs7826388 | 128066364 |
| rs7830341 | 128066518 |
| rs7830381 | 128066713 |
| rs16901966 | 128066840 |
| rs16901967 | 128066865 |
| rs7000910 | 128067193 |
| rs7001069 | 128067234 |
| rs17765137 | 128067402 |
| rs11781162 | 128067484 |
| rs11774449 | 128067620 |
| rs7006076 | 128067826 |
| rs7005862 | 128067930 |
| rs7006251 | 128067931 |
| rs7006409 | 128068017 |
| rs6988257 | 128068044 |
| rs16901969 | 128068685 |
| rs16901970 | 128069303 |
| rs10453084 | 128069367 |
| rs6987723 | 128069447 |
| rs6987640 | 128069616 |
| rs12675027 | 128069785 |
| rs13259163 | 128070111 |
| rs13256837 | 128070171 |
| rs13259247 | 128070181 |
| rs13256863 | 128070189 |
| rs13257829 | 128070218 |
| rs13259520 | 128070224 |
| rs10106712 | 128070449 |
| rs3956788 | 128070480 |
| rs7824451 | 128071049 |
| rs7824785 | 128071298 |
| rs13268116 | 128071384 |
| rs6470498 | 128072308 |
| rs1011829 | 128072331 |
| rs6999725 | 128072340 |
| rs28686871 | 128073068 |
| rs1456306 | 128073088 |
| rs13260084 | 128073418 |
| rs12544678 | 128074274 |
| rs7827234 | 128074694 |
| rs7844219 | 128075403 |
| rs6982324 | 128075644 |
| rs10098867 | 128075845 |
| rs28667456 | 128076744 |
| rs28368838 | 128076745 |
| rs12676925 | 128076746 |
| rs12216801 | 128076898 |
| rs10956352 | 128077241 |
| rs17866275 | 128077520 |
| rs7464849 | 128077876 |
| rs4341134 | 128077876 |
| rs12549692 | 128078335 |
| rs7837051 | 128078437 |
| rs1378898 | 128078955 |
| rs4593501 | 128078994 |
| rs1378897 | 128079247 |
| rs2392734 | 128079338 |
| rs13259145 | 128079368 |
| rs6470499 | 128079982 |
| rs7842760 | 128080058 |
| rs16901976 | 128080672 |
| rs1551512 | 128080714 |
| rs16901979 | 128081504 |
| rs10505483 | 128081783 |
| rs7817677 | 128082092 |
| rs10505482 | 128082437 |
| rs1456305 | 128083840 |
| rs17184796 | 128085047 |
| rs12678505 | 128085094 |
| rs16901983 | 128085630 |
| rs6989838 | 128085960 |
| rs7341677 | 128086973 |
| rs7013255 | 128087075 |
| rs11274077 | 128087187 |
| rs16901984 | 128087549 |
| rs10956353 | 128087878 |
| rs10098156 | 128088379 |
| rs6995291 | 128088912 |
| rs7000307 | 128089664 |
| rs12545722 | 128089822 |
| rs16901985 | 128090565 |
| rs13253958 | 128090894 |
| rs7840773 | 128091922 |
| rs7824364 | 128091953 |
| rs28645998 | 128094025 |
| rs7816535 | 128094256 |

SNP names in LD Block C with genomic locations (base pair start) in NCBI Build 34. Based on db SNP 125.

TABLE 5B
SNP markers within the region defined by SEQ ID NO: 2 (LD block C′).
Shown are marker names, position in NCBI builds 34, 35 and 36, SEQ ID NO: 2
and SEQ ID NO: 1, as well as SEQ ID of the marker, as applicable.

| | | | Pos SEQ ID | Pos SEQ ID | |
|---|---|---|---|---|---|
| Marker | Pos Build 34 | Pos Build 35 & 36 | NO: 2 | NO: 1 | SEQ ID NO |
| rs1031586 | 128029114 | 128141707 | 2 | | |
| rs17764031 | 128030209 | 128142802 | 1097 | | |
| rs35569027 | | 128142871 | 1166 | | |
| rs10091694 | 128030343 | 128142936 | 1231 | | |
| rs1456314 | 128032279 | 128144872 | 3167 | 280 | |
| rs1551516 | 128032478 | 128145071 | 3366 | 479 | |
| rs3940781 | 128032804 | 128145397 | 3692 | 805 | |
| rs16901935 | 128033134 | 128145727 | 4022 | 1135 | 49 |
| rs35664936 | | 128146550 | 4845 | 1958 | |
| rs12542102 | 128034110 | 128146703 | 4998 | 2111 | |
| rs35732005 | | 128146827 | 5122 | 2235 | |
| rs36032667 | | 128146879 | 5174 | 2287 | |
| rs11987219 | 128035270 | 128147863 | 6158 | 3271 | |
| rs11988135 | 128035998 | 128148591 | 6886 | 3999 | |
| rs2124598 | 128036064 | 128148657 | 6952 | 4065 | |
| rs2392726 | 128036068 | 128148661 | 6956 | 4069 | |
| rs2392727 | 128036185 | 128148778 | 7073 | 4186 | |
| rs2392728 | 128036232 | 128148825 | 7120 | 4233 | |
| rs2392729 | 128036255 | 128148848 | 7143 | 4256 | |
| rs1014656 | 128036272 | 128148865 | 7160 | 4273 | |
| rs2392730 | 128036283 | 128148876 | 7171 | 4284 | |
| rs2392731 | 128036329 | 128148922 | 7217 | 4330 | |
| rs1014655 | 128036428 | 128149021 | 7316 | 4429 | |
| rs2392732 | 128036523 | 128149116 | 7411 | 4524 | |
| rs2893603 | 128036546 | 128149139 | 7434 | 4547 | |
| rs34138989 | | 128149265 | 7560 | 4673 | |
| rs9656965 | 128036837 | 128149430 | 7725 | 4838 | |
| rs17831626 | 128037012 | 128149605 | 7900 | 5013 | |
| rs16901938 | 128037089 | 128149682 | 7977 | 5090 | |
| rs28451013 | 128037235 | 128149828 | 8123 | 5236 | |
| rs28385355 | 128037375 | 128149968 | 8263 | 5376 | |
| rs35755774 | | 128149994 | 8289 | 5402 | |
| rs11785961 | 128037424 | 128150017 | 8312 | 5425 | |
| rs13267056 | 128037676 | 128150269 | 8564 | 5677 | |
| rs7824674 | 128037682 | 128150275 | 8570 | 5683 | |
| rs7824679 | 128037699 | 128150292 | 8587 | 5700 | |
| rs7824923 | 128037727 | 128150320 | 8615 | 5728 | |
| rs35346020 | | 128150398 | 8693 | 5806 | |
| rs7843300 | 128037820 | 128150413 | 8708 | 5821 | |
| rs7824957 | 128037849 | 128150442 | 8737 | 5850 | |
| rs7825394 | 128038038 | 128150631 | 8926 | 6039 | |
| rs10308400 | 128038110 | 128150703 | 8998 | 6111 | |
| rs7825414 | 128038110 | 128150703 | 8998 | 6111 | |
| rs35954086 | | 128150967 | 9262 | 6375 | |
| rs11994932 | 128038487 | 128151080 | 9375 | 6488 | |
| rs13248907 | 128038934 | 128151527 | 9822 | 6935 | |
| rs13282331 | 128038958 | 128151551 | 9846 | 6959 | |
| rs13282364 | 128038998 | 128151591 | 9886 | 6999 | |
| rs35832881 | | 128151824 | 10119 | 7232 | |
| rs12548394 | 128039291 | 128151884 | 10179 | 7292 | |
| rs35409776 | | 128151899 | 10194 | 7307 | |
| rs10089648 | 128039438 | 128152031 | 10326 | 7439 | |
| rs13363429 | 128039438 | 128152031 | 10326 | 7439 | |
| rs34895815 | | 128152438 | 10733 | 7846 | |
| rs11985630 | 128040217 | 128152810 | 11105 | 8218 | |
| rs6993569 | 128040686 | 128153279 | 11574 | 8687 | |
| rs35520562 | | 128153449 | 11744 | 8857 | |
| rs6994316 | 128041128 | 128153721 | 12016 | 9129 | |
| rs34629453 | | 128153986 | 12281 | 9394 | |
| rs12550334 | 128041513 | 128154106 | 12401 | 9514 | |
| rs35090438 | | 128154122 | 12417 | 9530 | |
| rs11998124 | 128042069 | 128154662 | 12957 | 10070 | |
| rs6999589 | 128042235 | 128154828 | 13123 | 10236 | |
| rs11998248 | 128042344 | 128154937 | 13232 | 10345 | |
| rs35837627 | | 128155023 | 13318 | 10431 | |
| rs35450468 | | 128155214 | 13509 | 10622 | |
| rs34658436 | | 128155215 | 13510 | 10623 | |
| rs34985891 | | 128155403 | 13698 | 10811 | |
| rs7005114 | 128043347 | 128155940 | 14235 | 11348 | |
| rs11319438 | 128043466 | 128156059 | 14354 | 11467 | |
| rs6991325 | 128043515 | 128156108 | 14403 | 11516 | |
| rs1902431 | 128043665 | 128156258 | 14553 | 11666 | |
| rs34371194 | | 128156400 | 14695 | 11808 | |
| rs6470494 | 128044493 | 128157086 | 15381 | 12494 | 55 |
| rs7843737 | 128044655 | 128157248 | 15543 | 12656 | |
| rs10095770 | 128045159 | 128157752 | 16047 | 13160 | |
| rs41388647 | | 128158164 | 16459 | 13572 | |
| rs6987760 | 128046900 | 128159493 | 17788 | 14901 | |
| rs10306042 | 128047255 | 128159848 | 18143 | 15256 | |
| rs6470495 | 128047255 | 128159848 | 18143 | 15256 | |
| rs34872094 | | 128159883 | 18178 | 15291 | |
| rs4581008 | 128047454 | 128160047 | 18342 | 15455 | |
| rs4620246 | 128047551 | 128160144 | 18439 | 15552 | |
| rs7461435 | 128047551 | 128160144 | 18439 | 15552 | |
| rs36002700 | | 128160340 | 18635 | 15748 | |
| rs12544839 | 128047971 | 128160564 | 18859 | 15972 | |
| rs12544844 | 128048147 | 128160740 | 19035 | 16148 | |
| rs10956351 | 128048148 | 128160741 | 19036 | 16149 | |
| rs36012530 | | 128160820 | 19115 | 16228 | |
| rs34375527 | | 128160828 | 19123 | 16236 | |
| rs35719509 | | 128160838 | 19133 | 16246 | |
| rs1016342 | 128049044 | 128161637 | 19932 | 17045 | 36 |
| rs1551511 | 128049403 | 128161996 | 20291 | 17404 | |
| rs1031589 | 128049571 | 128162164 | 20459 | 17572 | 40 |
| rs1378896 | 128049643 | 128162236 | 20531 | 17644 | |
| rs1031588 | 128049866 | 128162459 | 20754 | 17867 | |
| rs1016343 | 128049886 | 128162479 | 20774 | 17887 | 37 |
| rs1031587 | 128050061 | 128162654 | 20949 | 18062 | 39 |
| rs1551510 | 128050067 | 128162660 | 20955 | 18068 | 47 |
| rs4871008 | 128050130 | 128162723 | 21018 | 18131 | 54 |
| rs11363193 | 128050446 | 128163039 | 21334 | 18447 | |
| rs35534753** | | 128163334 | 21629 | 18742 | |
| rs6981122 | 128051049 | 128163642 | 21937 | 19050 | 57 |
| rs7001504 | 128051072 | 128163665 | 21960 | 19073 | |
| rs13252298 | 128051745 | 128164338 | 22633 | 19746 | 44 |
| rs35201402 | | 128165598 | 23893 | 21006 | |
| rs7841060 | 128053066 | 128165659 | 23954 | 21067 | 72 |
| rs35024323 | | 128165800 | 24095 | 21208 | |
| rs4540377 | 128053503 | 128166096 | 24391 | 21504 | |
| rs6997559 | 128054167 | 128166760 | 25055 | 22168 | 60 |
| rs11554368 | 128055277 | 128167870 | 26165 | 23278 | |
| rs1802259 | | 128168243 | 26538 | 23651 | |
| rs17831913 | 128055755 | 128168348 | 26643 | 23756 | |
| rs7007694 | 128055755 | 128168348 | 26643 | 23756 | |
| rs13257371 | 128055831 | 128168424 | 26719 | 23832 | |
| rs4571699 | 128055858 | 128168451 | 26746 | 23859 | |
| rs1840709 | 128056044 | 128168637 | 26932 | 24045 | |
| rs3037753 | 128056059 | 128168652 | 26947 | 24060 | |
| rs7837848 | 128056121 | 128168714 | 27009 | 24122 | |
| rs3837167 | 128056513 | 128169106 | 27401 | 24514 | |
| rs3837166 | 128056618 | 128169211 | 27506 | 24619 | |
| rs11993508 | 128056665 | 128169258 | 27553 | 24666 | |
| rs17832021 | 128056765 | 128169358 | 27653 | 24766 | |
| rs3999809 | 128056897 | 128169490 | 27785 | 24898 | |
| rs3999810 | 128056956 | 128169549 | 27844 | 24957 | |
| rs28663168 | 128056998 | 128169591 | 27886 | 24999 | |
| rs3999811 | 128057051 | 128169644 | 27939 | 25052 | |
| rs3999812 | 128057096 | 128169689 | 27984 | 25097 | |
| rs3999806 | 128057101 | 128169694 | 27989 | 25102 | |
| rs3857883 | 128057195 | 128169788 | 28083 | 25196 | |
| rs2392733 | 128057261 | 128169854 | 28149 | 25262 | |
| rs1456317 | 128057295 | 128169888 | 28183 | 25296 | |
| rs3999807*** | | 128169888 | 28183 | 25296 | |
| rs1456316 | 128057437 | 128170030 | 28325 | 25438 | |
| rs16901946 | 128057514 | 128170107 | 28402 | 25515 | |
| rs28735726 | 128057518 | 128170111 | 28406 | 25519 | |
| rs9656813 | 128057553 | 128170146 | 28441 | 25554 | |
| rs9656814 | 128057566 | 128170159 | 28454 | 25567 | |
| rs9656815 | 128057592 | 128170185 | 28480 | 25593 | |
| rs10505484 | 128057901 | 128170494 | 28789 | 25902 | |
| rs11994653 | 128057932 | 128170525 | 28820 | 25933 | |
| rs6470496 | 128058618 | 128171211 | 29506 | 26619 | |
| rs6470497 | 128058919 | 128171512 | 29807 | 26920 | |
| rs7844454 | 128059090 | 128171683 | 29978 | 27091 | |
| rs7826322 | 128059142 | 128171735 | 30030 | 27143 | |
| rs12677860 | 128059214 | 128171807 | 30102 | 27215 | |
| rs12677861 | 128059216 | 128171809 | 30104 | 27217 | |
| rs7000321 | 128059608 | 128172201 | 30496 | 27609 | |
| rs12682421 | 128059740 | 128172333 | 30628 | 27741 | |
| rs7000967 | 128059949 | 128172542 | 30837 | 27950 | |
| rs7001895 | 128060268 | 128172861 | 31156 | 28269 | |
| rs1456315 | 128060526 | 128173119 | 31414 | 28527 | 46 |
| rs5013678 | 128060568 | 128173161 | 31456 | 28569 | |
| rs7463708 | 128060644 | 128173237 | 31532 | 28645 | |
| rs34446482 | | 128173246 | 31541 | 28654 | |
| rs13254738 | 128060932 | 128173525 | 31820 | 28933 | 45 |
| rs7006390 | 128060956 | 128173549 | 31844 | 28957 | 63 |
| rs1073997 | 128061776 | 128174369 | 32664 | 29777 | 41 |
| rs7012407 | 128062182 | 128174775 | 33070 | 30183 | |
| rs7012442 | 128062320 | 128174913 | 33208 | 30321 | 65 |
| rs7016828 | 128062821 | 128175414 | 33709 | 30822 | |
| rs7016830 | 128062824 | 128175417 | 33712 | 30825 | |
| rs12682344 | 128063373 | 128175966 | 34261 | 31374 | 43 |
| rs6983561 | 128063469 | 128176062 | 34357 | 31470 | 21 |
| rs16901948 | 128063690 | 128176283 | 34578 | 31691 | 9 |
| rs1869931 | 128063725 | 128176318 | 34613 | 31726 | |
| rs16901949 | 128063742 | 128176335 | 34630 | 31743 | 10 |
| rs16901950 | 128063832 | 128176425 | 34720 | 31833 | 11 |
| rs16901952 | 128063859 | 128176452 | 34747 | 31860 | 12 |
| rs7005144 | 128064233 | 128176826 | 35121 | 32234 | |
| rs16901953 | 128064818 | 128177411 | 35706 | 32819 | 13 |
| rs4871009 | 128065005 | 128177598 | 35893 | 33006 | |
| rs7010450 | 128065268 | 128177861 | 36156 | 33269 | 28 |
| rs6990420 | 128065314 | 128177907 | 36202 | 33315 | 26 |
| rs35365584 | | 128177908 | 36203 | 33316 | |
| rs6994359 | 128065512 | 128178105 | 36400 | 33513 | |
| rs17832285 | 128065582 | 128178175 | 36470 | 33583 | |
| rs7825340 | 128065718 | 128178311 | 36606 | 33719 | |
| rs12544977 | 128065876 | 128178469 | 36764 | 33877 | 42 |
| rs16901959 | 128066119 | 128178712 | 37007 | 34120 | 14 |
| rs7826337 | 128066163 | 128178756 | 37051 | 34164 | 69 |
| rs7826388 | 128066365 | 128178958 | 37253 | 34366 | 33 |
| rs7830341 | 128066519 | 128179112 | 37407 | 34520 | 34 |
| rs7830381 | 128066714 | 128179307 | 37602 | 34715 | |
| rs16901966 | 128066841 | 128179434 | 37729 | 34842 | 15 |
| rs16901967 | 128066866 | 128179459 | 37754 | 34867 | 16 |
| rs7000910 | 128067194 | 128179787 | 38082 | 35195 | 62 |
| rs7001069 | 128067235 | 128179828 | 38123 | 35236 | 27 |
| rs17765137 | 128067403 | 128179996 | 38291 | 35404 | |
| rs11781162 | 128067485 | 128180078 | 38373 | 35486 | |
| rs11774449 | 128067621 | 128180214 | 38509 | 35622 | |
| rs7006076 | 128067827 | 128180420 | 38715 | 35828 | |
| rs7005862 | 128067931 | 128180524 | 38819 | 35932 | |
| rs7006251 | 128067932 | 128180525 | 38820 | 35933 | |
| rs7006409 | 128068018 | 128180611 | 38906 | 36019 | 64 |
| rs6988257 | 128068045 | 128180638 | 38933 | 36046 | 24 |
| rs16901969 | 128068686 | 128181279 | 39574 | 36687 | 17 |
| rs16901970 | 128069304 | 128181897 | 40192 | 37305 | 18 |
| rs10453084 | 128069368 | 128181961 | 40256 | 37369 | 6 |
| rs6987723 | 128069448 | 128182041 | 40336 | 37449 | 23 |
| rs6987640 | 128069617 | 128182210 | 40505 | 37618 | 22 |
| rs12675027 | 128069786 | 128182379 | 40674 | 37787 | |
| rs13259163 | 128070112 | 128182705 | 41000 | 38113 | |
| rs13256837 | 128070172 | 128182765 | 41060 | 38173 | |
| rs13259247 | 128070182 | 128182775 | 41070 | 38183 | |
| rs13256863 | 128070190 | 128182783 | 41078 | 38191 | |
| rs13257829 | 128070219 | 128182812 | 41107 | 38220 | |
| rs13259520 | 128070225 | 128182818 | 41113 | 38226 | |
| rs10106712 | 128070450 | 128183043 | 41338 | 38451 | |
| rs3956788 | 128070481 | 128183074 | 41369 | 38482 | |
| rs7824451 | 128071050 | 128183643 | 41938 | 39051 | 31 |
| rs7824785 | 128071299 | 128183892 | 42187 | 39300 | 32 |
| rs13268116 | 128071385 | 128183978 | 42273 | 39386 | |
| rs6470498 | 128072309 | 128184902 | 43197 | 40310 | 20 |
| rs1011829 | 128072332 | 128184925 | 43220 | 40333 | |
| rs6999725 | 128072341 | 128184934 | 43229 | 40342 | |
| rs34965819 | | 128185532 | 43827 | 40940 | |
| rs28686871 | 128073069 | 128185662 | 43957 | 41070 | |
| rs1456306 | 128073089 | 128185682 | 43977 | 41090 | |
| rs13260084 | 128073419 | 128186012 | 44307 | 41420 | |
| rs35270158 | | 128186496 | 44791 | 41904 | |
| rs12544678 | 128074275 | 128186868 | 45163 | 42276 | |
| rs7827234 | 128074695 | 128187288 | 45583 | 42696 | 70 |
| rs34512164 | | 128187312 | 45607 | 42720 | |
| rs7844219 | 128075404 | 128187997 | 46292 | 43405 | 35 |
| rs6982324 | 128075645 | 128188238 | 46533 | 43646 | |
| rs10098867 | 128075846 | 128188439 | 46734 | 43847 | |
| rs34171547 | | 128189021 | 47316 | 44429 | |
| rs34646118 | | 128189070 | 47365 | 44478 | |
| rs28667456 | 128076745 | 128189338 | 47633 | 44746 | |
| rs28368838 | 128076746 | 128189339 | 47634 | 44747 | |
| rs12676925 | 128076747 | 128189340 | 47635 | 44748 | |
| rs34425474 | | 128189416 | 47711 | 44824 | |
| rs12216801 | 128076899 | 128189492 | 47787 | 44900 | |
| rs10956352 | 128077242 | 128189835 | 48130 | 45243 | |
| rs17866275 | 128077521 | 128190114 | 48409 | 45522 | |
| rs4341134 | 128077877 | 128190470 | 48765 | 45878 | |
| rs7464849 | 128077877 | 128190470 | 48765 | 45878 | |
| rs12549692 | 128078336 | 128190929 | 49224 | 46337 | |
| rs7837051 | 128078438 | 128191031 | 49326 | 46439 | |
| rs1378898 | 128078956 | 128191549 | 49844 | 46957 | |
| rs4593501 | 128078995 | 128191588 | 49883 | 46996 | |
| rs1378897 | 128079248 | 128191841 | 50136 | 47249 | |
| rs2392734 | 128079339 | 128191932 | 50227 | 47340 | |
| rs13259145 | 128079369 | 128191962 | 50257 | 47370 | |
| rs6470499 | 128079983 | 128192576 | 50871 | 47984 | 56 |
| rs7842760 | 128080059 | 128192652 | 50947 | 48060 | |
| rs16901976 | 128080673 | 128193266 | 51561 | 48674 | |
| rs1551512 | 128080715 | 128193308 | 51603 | 48716 | 8 |
| rs16901979 | 128081505 | 128194098 | 52393 | 49506 | 73 |
| rs10505483 | 128081784 | 128194377 | 52672 | 49785 | 7 |
| rs7817677 | 128082093 | 128194686 | 52981 | 50094 | 30 |
| rs35045168 | | 128194764 | 53059 | 50172 | |
| rs10505482 | 128082438 | 128195031 | 53326 | 50439 | |
| rs1456305 | 128083841 | 128196434 | 54729 | 51842 | |
| rs35997767 | | 128196523 | 54818 | 51931 | |
| rs17184796 | 128085048 | 128197641 | 55936 | 53049 | |
| rs12678505 | 128085095 | 128197688 | 55983 | 53096 | |
| rs16901983 | 128085631 | 128198224 | 56519 | 53632 | |
| rs34933805 | | 128198512 | 56807 | 53920 | |
| rs6989838 | 128085961 | 128198554 | 56849 | 53962 | 25 |
| rs7341677 | 128086974 | 128199567 | 57862 | 54975 | |
| rs7013255 | 128087076 | 128199669 | 57964 | 55077 | 29 |
| rs11274077 | 128087187 | 128199780 | 58075 | 55188 | |
| rs16901984 | 128087550 | 128200143 | 58438 | 55551 | 19 |
| rs34826080* | | 128200278 | 58573 | 55686 | |
| rs10956353 | 128087879 | 128200472 | 58767 | 55880 | |
| rs10098156 | 128088380 | 128200973 | 59268 | 56381 | |
| rs6995291 | 128088913 | 128201506 | 59801 | 56914 | |
| rs34537182 | | 128201519 | 59814 | 56927 | |
| rs7000307 | 128089665 | 128202258 | 60553 | 57666 | 61 |
| rs12545722 | 128089823 | 128202416 | 60711 | 57824 | |
| rs16901985 | 128090566 | 128203159 | 61454 | 58567 | |
| rs34720002 | | 128203299 | 61594 | 58707 | |
| rs35610862 | | 128203459 | 61754 | 58867 | |
| rs13253958 | 128090895 | 128203488 | 61783 | 58896 | |
| rs7840773 | 128091923 | 128204516 | 62811 | 59924 | 71 |
| rs7824364 | 128091954 | 128204547 | 62842 | 59955 | 68 |
| rs28645998 | 128094026 | 128206619 | 64914 | 62027 | |
| rs7816535 | 128094257 | 128206850 | 65145 | 62258 | 67 |
| rs16901988 | 128095149 | 128207742 | 66037 | | |
| rs7821533 | 128095346 | 128207939 | 66234 | | |
| rs7821299 | 128095370 | 128207963 | 66258 | | |
| rs34074310 | | 128208988 | 67283 | | |
| rs12335235 | 128100703 | 128213296 | 71591 | | |
| rs34933261 | | 128214191 | 72486 | | |
| rs2124596 | 128101759 | 128214352 | 72647 | | |
| rs2124595 | 128101824 | 128214417 | 72712 | | |
| rs4624993 | 128101824 | 128214417 | 72712 | | |
| rs10111496 | 128102406 | 128214999 | 73294 | | |
| rs13364435 | 128102406 | 128214999 | 73294 | | |
| rs35623297 | | 128215361 | 73656 | | |
| rs34035606 | | 128215465 | 73760 | | |
| rs10101884 | 128102897 | 128215490 | 73785 | | |
| rs6470500 | 128103187 | 128215780 | 74075 | | |
| rs6987409 | 128106750 | 128219343 | 77638 | | |
| rs35775018 | | 128219607 | 77902 | | |
| rs36080589 | | 128219616 | 77911 | | |
| rs17446747 | 128107241 | 128219834 | 78129 | | |
| rs17446776 | 128108261 | 128220854 | 79149 | | |
| rs10094434 | 128110394 | 128222987 | 81282 | | |
| rs7017820 | 128110462 | 128223055 | 81350 | | |
| rs7018238 | 128110470 | 128223063 | 81358 | | |
| rs7018243 | 128110480 | 128223073 | 81368 | | |
| rs11286339 | 128110587 | 128223180 | 81475 | | |
| rs13253040 | 128111938 | 128224531 | 82826 | | |
| rs13252570 | 128111939 | 128224532 | 82827 | | |
| rs13252598 | 128111964 | 128224557 | 82852 | | |
| rs2466040 | 128112253 | 128224846 | 83141 | | |
| rs7822987 | 128113252 | 128225845 | 84140 | | |
| rs7822995 | 128113277 | 128225870 | 84165 | | |
| rs2516280 | 128115044 | 128227637 | 85932 | | |
| rs13257144 | 128115979 | 128228572 | 86867 | | |
| rs3100857 | 128116021 | 128228614 | 86909 | | |
| rs34946000 | | 128228837 | 87132 | | |
| rs35282781 | | 128228987 | 87282 | | |
| rs28824784 | 128116644 | 128229237 | 87532 | | |
| rs34195077 | | 128230348 | 88643 | | |
| rs2392724 | 128118125 | 128230718 | 89013 | | |
| rs2925465 | 128118125 | 128230718 | 89013 | | |
| rs4427134 | 128118125 | 128230718 | 89013 | | |
| rs36021065 | | 128230793 | 89088 | | |
| rs2392725 | 128118217 | 128230810 | 89105 | | |
| rs2954005 | 128118217 | 128230810 | 89105 | | |
| rs4593502 | 128118217 | 128230810 | 89105 | | |
| rs2893602 | 128118242 | 128230835 | 89130 | | |
| rs4552862 | 128118242 | 128230835 | 89130 | | |
| rs7459511 | 128118242 | 128230835 | 89130 | | |
| rs35636657 | | 128230862 | 89157 | | |
| rs2445605 | 128118533 | 128231126 | 89421 | | |
| rs7843464 | 128118533 | 128231126 | 89421 | | |
| rs17374770 | 128119309 | 128231902 | 90197 | | |
| rs17446916 | 128119563 | 128232156 | 90451 | | |
| rs11341635 | 128119823 | 128232416 | 90711 | | |
| rs35532717 | | 128232429 | 90724 | | |
| rs5894869 | 128120238 | 128232831 | 91126 | | |
| rs33965163 | | 128232832 | 91127 | | |
| rs3037747 | 128120240 | 128232833 | 91128 | | |
| rs3758058 | 128122147 | 128234740 | 93035 | | |
| rs13275200 | 128123145 | 128235738 | 94033 | | |
| rs34106304 | | 128237228 | 95523 | | |
| rs34285962 | | 128238577 | 96872 | | |
| rs13269873 | 128126198 | 128238791 | 97086 | | |
| rs10111439 | 128126446 | 128239039 | 97334 | | |

*(—/TATT polymorphism)
**(A/AGAA polymorphism)
***(—/GTTT polymorphism)

TABLE 5C
Microsatellite markers within the SEQ ID NO: 2 (LD Block C′)
region. Shown is the marker name, start and end positions of a
400 bp genomic segment centered around each microsatellite
marker in NCBI Build 34, and the corresponding location in
SEQ ID NO: 2

| | | | Start SEQ | End SEQ |
|---|---|---|---|---|
| Marker name | Start B34 | End B34 | ID NO: 2 | ID NO: 2 |
| DG8S502 | 128029888 | 128030288 | 776 | 1176 |
| DG8S815 | 128043386 | 128043621 | 14274 | 14509 |
| DG8S345 | 128055889 | 128056158 | 26777 | 27046 |
| DG8S1334 | 128056004 | 128056111 | 26892 | 26999 |
| DG8S625 | 128064928 | 128065358 | 35816 | 36246 |
| DG8S551 | 128071376 | 128071525 | 42264 | 42413 |
| DG8S911 | 128071591 | 128071748 | 42479 | 42636 |
| DG8S1179 | 128075716 | 128076097 | 46604 | 46985 |
| DG8S1508 | 128087019 | 128087367 | 57907 | 58255 |
| DG8S1088 | 128094303 | 128094579 | 65191 | 65467 |
| DG8S733 | 128097441 | 128097570 | 68329 | 68458 |
| DG8S402 | 128099742 | 128100125 | 70630 | 71013 |
| DG8S1431 | 128108504 | 128108934 | 79392 | 79822 |
| DG8S1230 | 128119990 | 128120400 | 90878 | 91288 |

Genes and predicted genes that map to LD block C on chromosome 8q24.21 of the human genome include a retrotransposed gene that is an intronless copy of the SRRM1 gene on Chr1p36 (Genbank Accession No. BC017315), as well as predicted genes (e.g., NT_{—}008046.701, chr8_{—}1173.1, chr8.129.001.a, vugee.bDec03, kloger, keebly). The underlying variation in markers or haplotypes associated with the LD block C region and cancer may affect expression of genes within the LD block C, but also nearby genes, such as NSE2, POU5FLC20, c-MYC, PVT1, and/or other known, unknown or predicted genes in the area. Furthermore, such variation may affect RNA or protein stability or may have structural consequences, such that the region is more prone to somatic rearrangement in haplotype carriers. This is in accordance with LD Block C being amplified in a large percentage of cancers, including, but not limited to, prostate cancer (www.progenetix.com). In fact, Chr8q21-24 is the most frequently gained chromosomal region in all cancers combined (about 17%) and in prostate cancer (about 20%) (www.progenetix.com). Thus, the underlying variation could affect uncharacterized genes directly linked to the haplotypes described herein, or could influence neighbouring genes not directly linked to the haplotypes described herein.

TABLE 6
Key to sequence ID's provide herein.

| SEQ ID NO | Marker/Sequence |
|---|---|
| 1 | LD Block C |
| 2 | LD Block C′ |
| 3 | DG5S802 |
| 4 | DG5S803-F |
| 5 | DG5S803-R |
| 6 | rs10453084 |
| 7 | rs10505483 |
| 8 | rs1551512 |
| 9 | rs16901948 |
| 10 | rs16901949 |
| 11 | rs16901950 |
| 12 | rs16901952 |
| 13 | rs16901953 |
| 14 | rs16901959 |
| 15 | rs16901966 |
| 16 | rs16901967 |
| 17 | rs16901969 |
| 18 | rs16901970 |
| 19 | rs16901984 |
| 20 | rs6470498 |
| 21 | rs6983561 |
| 22 | rs6987640 |
| 23 | rs6987723 |
| 24 | rs6988257 |
| 25 | rs6989838 |
| 26 | rs6990420 |
| 27 | rs7001069 |
| 28 | rs7010450 |
| 29 | rs7013255 |
| 30 | rs7817677 |
| 31 | rs7824451 |
| 32 | rs7824785 |
| 33 | rs7826388 |
| 34 | rs7830341 |
| 35 | rs7844219 |
| 36 | rs1016342 |
| 37 | rs1016343 |
| 38 | rs10216560 |
| 39 | rs1031587 |
| 40 | rs1031589 |
| 41 | rs1073997 |
| 42 | rs12544977 |
| 43 | rs12682344 |
| 44 | rs13252298 |
| 45 | rs13254738 |
| 46 | rs1456315 |
| 47 | rs1551510 |
| 48 | rs16901932 |
| 49 | rs16901935 |
| 50 | rs16901997 |
| 51 | rs1995613 |
| 52 | rs283710 |
| 53 | rs283719 |
| 54 | rs4871008 |
| 55 | rs6470494 |
| 56 | rs6470499 |
| 57 | rs6981122 |
| 58 | rs6984136 |
| 59 | rs6990483 |
| 60 | rs6997559 |
| 61 | rs7000307 |
| 62 | rs7000910 |
| 63 | rs7006390 |
| 64 | rs7006409 |
| 65 | rs7012442 |
| 66 | rs7017081 |
| 67 | rs7816535 |
| 68 | rs7824364 |
| 69 | rs7826337 |
| 70 | rs7827234 |
| 71 | rs7840773 |
| 72 | rs7841060 |
| 73 | rs16901979 |

### Discussion

As described herein, a locus on chromosome 8q24.21 has been demonstrated to play a role in cancer (e.g., prostate cancer (e.g., aggressive prostate cancer). Particular markers and haplotypes (e.g., HapC, haplotypes containing one or more of the markers depicted in Table 3) are present at a higher than expected frequency in subjects having prostate cancer. Based on the haplotypes described herein, which are associated with a propensity for particular forms of cancer, genetic susceptibility assays (e.g., a diagnostic screening test) can be used to identify individuals at risk for cancer.

The markers and haplotypes described herein do have a higher relative risk in the aggressive prostate cancer as compared to the whole prostate cancer group (Table 1), thereby indicating an increased risk for aggressive, fast growing prostate cancer. Given that a significant percentage of prostate cancer is a non-aggressive form that will not spread beyond the prostate and cause morbidity or mortality, and treatments of prostate cancer including prostatectomy, radiation, and chemotherapy all have side effects and significant cost, it would be valuable to have diagnostic markers, such as those described herein, that show greater risk for aggressive prostate cancer as compared to the less aggressive form(s).

### Example 2

### Verification of Association with Prostate Cancer in Several Cohorts

Additional analysis further supports the presence of the variants associated with prostate cancer on chromosome 8q24. As depicted in Table 7, both the rs16901979 1 allele and HapC were found to be associated with prostate cancer in two cohorts of Caucasian ancestry and one cohort of African ancestry.

Replication study of association of single markers and haplotypes within LD Block C with prostate cancer. The cohorts are comprised of samples of Caucasian origin from Chicago, U.S. (Northwestern University) and Spain (Zaragoza University Hospital), and an African American cohort from Michigan, U.S. (University of Michigan). Allele codes for SNPs are as follows: 1=A, 2=C, 3=G, 4=T, and X=any allele

TABLE 7
Association of SNP markers on Chr8q24 to prostate cancer (PrCa) in Spain and the United States

| | # of Individuals | | | | Allele frequency | | | |
|---|---|---|---|---|---|---|---|---|
| Affected Phenotype | Affected | Controls | p-val | RR | Affected | Controls | Allele | PAR |
| PrCa-Chicago | 419 | 237 | 0.006 | 2.39 | 0.054 | 0.023 | 1 rs16901979 | 0.061 |
| PrCa-Spain | 385 | 892 | 0.005 | 1.71 | 0.066 | 0.040 | 1 rs16901979 | 0.054 |
| PrCa-JHU | 373 | 372 | 0.005 | 1.35 | 0.500 | 0.420 | 1 rs16901979 | 0.240 |

Alleles for the marker rs16901979 and SNPs in same LD block at 8q24.21 are shown and the corresponding numbers of PrCa cases and controls (N), allelic frequencies of variants in affected and control individuals, the odds-ratio (OR) and two-sided P values. Affected individuals are those diagnosed with prostate cancer (ICD10 = C61). Controls are unaffected population based controls. Allele codes for SNPs are as follows: 1 = A, 2 = C, 3 = G, 4 = T. PAR is population attributable risk. The prostate cancer patients and controls from Spain and Chicago are of Caucasian ethnicity and the cohort from John Hopkins University (JHU) is of African American ethnicity. 1 rs16901979: using information from other SNPs to increase genotype yield.

The population frequency of the at-risk variants (approximated by the frequency in controls) in the Caucasian samples is comparable to that of the Icelandic population. However, it is noted that the population frequency in African Americans is much greater. The population attributable risk in the African American cohort is thus much higher or about 24% as compared to about 5-6% in the two Caucasian origin cohorts. This suggests that a greater percentage of prostate cancer in African Americans can be explained by the at-risk variants of the present invention than in Caucasians.

### Materials and Methods

The Caucasian U.S. study population consisted of 419 prostate cancer patients (ICD10 C61), who underwent surgery at the Urology Department of Northwestern Memorial Hospital, Chicago, and 237 population based controls enrolled at the Department of Human Genetics, University of Chicago. Medical records were examined to retrieve clinical information including stage and biopsy Gleason score. The mean age at diagnosis was 59 years for patients. Both patients and controls were of self-reported European American ethnicity. This was confirmed by the estimation of genetic ancestry using 30 microsatellite markers distributed randomly throughout the genome (see below). The mean and median portion of European ancestry in this cohort were both greater than 0.99 (see methods described below for details). The study protocols were approved by the Institutional Review Boards of Northwestern University and the University of Chicago. All subjects gave written informed consent.

The Spanish study population consisted of 390 prostate cancer patients, recruited from the Oncology Department of Zaragoza Hospital from June 2005 to June 2006. Patients were recruited by Dr. Jose I. Mayordomo and collaborating oncolologists at the Division of Medical Oncology, University Hospital in Zaragoza, Spain. During the 12-month interval when the study samples were collected, 700 patients were eligible. Of these, about 600 (˜85%) patients were approached of whom 440 enrolled (73% participation rate). All patients were of Caucasian ethnicity. The median time interval from prostate cancer diagnosis to collection of blood samples was 5 months (mean 7 months, range 1-67 months). Clinical information including age at onset, grade and stage was collected from medical records. The mean age at onset for the patients is 69 years (median 71 years) and the range is from 45-83 years. The 892 Spanish controls were approached at the University Hospital in Zaragoza, Spain for all other diseases than cancer, questioned to rule out prior cancers, before drawing the blood sample. Study protocols were approved by the Institutional Review Boards of Zaragoza University Hospital. All subjects gave written informed consent.

### Evaluation of Genetic Ancestry

The program Structure (Pritchard, J. K. et al., Genetics 115:945-59 (2000)) was used to estimate the genetic ancestry of individuals. Structure infers the allele frequencies of K ancestral populations on the basis of multilocus genotypes from a set of individuals and a user-specified value of K, and assigns a proportion of ancestry from each of the inferred K populations to each individual. The analysis of the data set was run with K=3, with the aim of identifying the proportion of African and European ancestry in each individual. The statistical significance of the difference in mean European ancestry between African American patients and controls was evaluated by reference to a null distribution derived from 10000 randomized datasets.

To evaluate genetically estimated ancestry of the study cohorts from the US, 30 unlinked microsatellite markers were selected from about 2000 microsatellites genotyped in a previously described (Pritchard, J. K. et al., Genetics 115:945-59 (2000)) multi-ethnic cohort of 35 European Americans, 88 African Americans, 34 Chinese, and 29 Mexican Americans. Of the 2000 microsatellite markers the selected set showed the most significant differences between European Americans, African Americans, and Asians, and also had good quality and yield: D152630, D1S2847, D1S466, D1S493, D2S166, D3S1583, D3S4011, D3S4559, D4S2460, D4S3014, D5S1967, DG5S802, D6S1037, D8S1719, D8S1746, D9S1777, D9S1839, D9S2168, D10S1698, D11S1321, D11S4206, D12S1723, D13S152, D14S588, D17S1799, D17S745, D18S464, D19S113, D20S878 and D22S1172. The following primer pairs were used for DG5S802: DG5S802-F: CAAGTTTAGCTGTGATGTACAGGTTT (SEQ ID NO: 46) and DG5S802-R: TTCCAGAACCAAAGCCAAAT (SEQ ID NO: 47).

Discussion

In summary, significant association of prostate cancer risk to the 1 allele of rs16901979 has been demonstrated in three cohorts of European ancestry from Iceland, Spain and Chicago. The corresponding population attributable risk (PAR) ranges from about 5-6% when considering all prostate cancer in these populations and is slightly increased in aggressive prostate cancer or to about 7-8%. The association was replicated between prostate cancer and the 1 allele of rs16901979 in an African American cohort with a relative risk of 1.35 (P=0.005). Due to the increased frequency of these variants in the African American sample, the PAR in this population is much higher as compared to the Caucasian samples or about 24%.

The variants described herein were identified through a genome wide association analysis. Over 300,000 SNPs were typed in 1455 prostate cancer patients and 4182 controls. Our attention focused to this region as it is the second most significant haplotype contained within an individual LD block in the whole genome (LD block haplotype.

Of importance is the greatly increased frequency of this haplotype in African Americans as compared to Caucasians which may explain the higher incidence of prostate cancer in this group.

1. A. method for diagnosing a susceptibility to cancer in a human individual, comprising determining the presence or absence of at least one allele of at least one polymorphic marker in a nucleic acid sample obtained from the individual, wherein the at least one polymorphic marker is associated with SEQ ID NO:2, and wherein the presence of the at least one allele is indicative of a susceptibility to the cancer.

2. The method of claim 1, wherein the at least one polymorphic marker comprises at least one marker selected from the group consisting of the markers set forth in Table 4A, 4B, 5A, 5B and 5C.

3. The method of claim 2, wherein the at least one marker comprises at least one marker within Chr8q24.21 in strong linkage disequilibrium, as defined by |D′|>0.8 and/or r2>0.2, with one or more markers selected from the group consisting of the markers in Table 4A and 4B.

4. The method of claim 1, wherein the at least one polymorphic marker is in linkage disequilibrium with HapC.

5. The method of claim 1, wherein the at least one marker is marker rs16901979 (SEQ ID NO: 73), and or markers in linkage disequilibrium therewith.

6. (canceled)

7. The method of claim 1, further comprising assessing the frequency of at least one haplotype in the individual.

8. The method of claim 7, wherein the at least one haplotype comprises at least one marker selected from markers rs1456314 allele G, rs17831626 allele T, rs7825414 allele G, rs6993569 allele G, rs6994316 allele A, rs6470494 allele T, rs1016342 allele C, rs1031588 allele G, rs1016343 allele T, rs1551510 allele G, rs1456306 allele C, rs1378897 allele G, rs1456305 allele T, and rs7816535 allele G.

9-18. (canceled)

19. The method of claim 1, wherein the cancer is selected from the group consisting of prostate cancer, colon cancer, breast cancer, testicular cancer, lung cancer and melanoma cancer.

20. The method of claim 19, wherein the cancer is prostate cancer.

21-26. (canceled)

27. The method of claim 1, wherein the individual is of a specific ancestry.

28. The method of claim 27, wherein the ancestry is black African ancestry.

29. (canceled)

30. The method of claim 27, wherein the ancestry is determined by detecting at least one allele of at least one polymorphic marker in a sample from the individual, wherein the presence or absence of the allele is indicative of the ancestry of the individual.

31. A method of identification of a marker for use in assessing susceptibility to cancer, the method comprising
a) identifying at least one polymorphic marker within SEQ ID NO:2, or at least one polymorphic marker in linkage disequilibrium therewith;
b) determining the genotype status of a sample of individuals diagnosed with, or having a susceptibility to, prostate cancer; and
c) determining the genotype status of a sample of control individuals;
wherein a significant difference in frequency of at least one allele in at least one polymorphism in individuals diagnosed with, or having a susceptibility to, prostate cancer, as compared with the frequency of the at least one allele in the control sample is indicative of the at least one polymorphism being useful for assessing susceptibility to cancer.

32. (canceled)

33. The method of claim 31, wherein the at least one polymorphic marker is in linkage disequilibrium, as characterized by numerical values of r² of greater than 0.2 and/or |D′| of greater than 0.8 with HapC and/or marker rs16901979.

34-47. (canceled)

48. The method of claim 31, wherein the cancer is selected from prostate cancer, colon cancer, breast cancer, lung cancer, testicular cancer and melanoma.

49-51. (canceled)

52. The method of claim 31, wherein the individual is of a specific ancestry.

53. The method of claim 52, wherein the ancestry is black African ancestry.

54. (canceled)

55. The method of claim 52, wherein the ancestry is determined by detecting at least one allele of at least one polymorphic marker in a sample from the individual, wherein the presence or absence of the allele is indicative of the ancestry of the individual.

56. A method of assessing an individual for probability of response to a therapeutic agent for preventing and/or ameliorating symptoms associated with cancer, comprising: determining the presence or absence of at least one allele of at least one polymorphic marker in a nucleic acid sample obtained from the individual, wherein the at least one polymorphic marker is selected from the group consisting of the polymorphic markers listed in Table 4A, 4B, 5A, 5B and 5C, and markers in linkage disequilibrium therewith, wherein the presence of the at least one allele of the at least one marker is indicative of a probability of a positive response to a symptoms associated with exfoliation syndrome and/or glaucoma therapeutic agent.

57. A method of predicting prognosis of an individual diagnosed with, cancer, the method comprising determining the presence or absence of at least one allele of at least one polymorphic marker in a nucleic acid sample obtained from the individual, wherein the at least one polymorphic marker is selected from the group consisting of the polymorphic markers listed in Table 4A, 4B, 5A, 5B and 5C, and markers in linkage disequilibrium therewith, wherein the presence of the at least one allele is indicative of a worse prognosis of the cancer in the individual.

58. A method of monitoring progress of a treatment of an individual undergoing treatment for cancer, the method comprising determining the presence or absence of at least one allele of at least one polymorphic marker in a nucleic acid sample obtained from the individual, wherein the at least one polymorphic marker is selected from the group consisting of the polymorphic markers listed in Table 4A, 4B, 5A, 5B and 5C, and markers in linkage disequilibrium therewith, wherein the presence of the at least one allele is indicative of the treatment outcome of the individual.

59-68. (canceled)

69. A kit for assessing susceptibility to cancer in a human individual, the kit comprising reagents for selectively detecting at least one allele of at least one polymorphic marker in the genome of the individual, wherein the polymorphic marker is selected from the group consisting of the polymorphic markers within the segment whose sequence is set forth in SEQ ID NO:2, and markers in linkage disequilibrium therewith, and wherein the presence of the at least one allele is indicative of a susceptibility to cancer.

70. The kit of claim 69, wherein the at least one polymorphic marker is selected from the group consisting of the markers set forth in Table 4A, 4B, 5A, 5B and 5C, and markers in linkage disequilibrium therewith.

71. (canceled)

72. The kit of claim 69, wherein the at least one polymorphic marker is rs16901979 (SEQ ID NO: 73).

73-90. (canceled)

91. A computer-readable medium on which is stored:
a) an identifier for at least one polymorphic marker;
b) an indicator of the frequency of at least one allele of said at least one polymorphic marker in a plurality of individuals diagnosed with cancer; and
c) an indicator of the frequency of the least one allele of said at least one polymorphic markers in a plurality of reference individuals;
wherein the at least one polymorphic marker is selected from the group consisting of the polymorphic markers set forth in Table 4A, 4B, 5A, 5B and 5C, and polymorphisms in linkage disequilibrium therewith.

92. The medium according to claim 91, wherein the polymorphic site is rs16901979 (SEQ ID NO: 73), and markers in linkage disequilibrium therewith, as defined by numerical values of r² of at least 0.2 and/or values of |D′| of at least 0.8.

93. The medium of claim 91, wherein the cancer is selected from prostate cancer, colon cancer, breast cancer, testicular cancer, lung cancer and melanoma cancer.

94-96. (canceled)

97. The medium claim 91, further comprising information about the ancestry of the plurality of individuals.

98-100. (canceled)

101. An apparatus for determining a genetic indicator for cancer in a human individual, comprising:
a computer readable memory; and
a routine stored on the computer readable memory;
wherein the routine is adapted to be executed on a processor to analyze marker and/or haplotype information for at least one human individual with respect to at least one polymorphic marker selected from the group consisting of the markers set forth in Table 4A, 4B, 5A, 5B and 5C, and markers in linkage disequilibrium therewith, and generate an output based on the marker or haplotype information, wherein the output comprises a risk measure of the at least one marker or haplotype as a genetic indicator of cancer for the human individual.

102. The apparatus of claim 101, wherein the routine further comprises an indicator of the frequency of at least one allele of at least one polymorphic marker or at least one haplotype in a plurality of individuals diagnosed with cancer, and an indicator of the frequency of at the least one allele of at least one polymorphic marker or at least one haplotype in a plurality of reference individuals, and wherein a risk measure is based on a comparison of the at least one marker and/or haplotype status for the human individual to the indicator of the frequency of the at least one marker and/or haplotype information for the plurality of individuals diagnosed with cancer.

103. The apparatus of claim 101, wherein the at least one polymorphic marker is rs16901979 (SEQ ID NO:73), and markers in linkage disequilibrium therewith, as defined by numerical values of r² of at least 0.2 and/or values of |D′| of at least 0.8.

104. (canceled)


SEQUENCE LISTING (73 sequences)

SEQ ID NO: 1 - DNA, 63001 bp, Homo sapiens
gataacatgcagtacttttaagtaatactgtatatagagaagtagatcgaaataggtaaactattaacataaattttcctgtagtggaatcccaattaaagcaataatgtagagcaataagaagcaaaatctgcttggatccaaatcatttctgaactttgtaactttggtcgtgttgctttctgtgaatcagtttcttcatttaaaaaagttgttatgaggatagaacatgctagtatttataaaatgctcataaattcttggcacaaagtaagctatagactcaaaaatggtgattccagacctcaagaaataagaacttgaatttcagagaaatcttcccttattatccctgaggtccactccatagcttgatcaaagaagtgggatactatgtagtagactagtgtggcagcaccttaaacccacaaataagtttggctgttgtaaagagaagggaaaccaaaattatttcaggacctactatgtgcagagtttttatctgctatatacataggcatatgctattccatcagtggttcagagggcataaacagttaataaagctatctcagggatgattaaattgagagttgctcagggtcatacaatttttgagtcaggaattaaacccagggctatttcattccaaggcccatgttactttctggtaaaacaaataaccatgaaatataatatattttctttgaatccctaatagtgggtgctattatacccccaattacttaaattgatcctgtacattgccaagagattaaattataaattattgagttcaaaaagttaatttcttcctccaaagagatggcatcttacagtttaattactctgacagctttttttttttattttttctcctagtgaattatcaacagaaactacctagagaaacctgtaacttcagatcttctgattaattgattttctttgggtgctgcttgacagtaacagaatggtccatggggacagtttttcagtgaatgttttcaacaatgataggaacacagagctaagcagagtgtcaagagacatttagtgcagtagagacatctcatctgtttttctgcagaacttgcataaaattagcatctgattattatcacctcagaatgaaccggaacccgtaaagatgtctttgatcgtaaaagtggggaggagaccctgatggaagaagagaagacttcagcctcacaatagaaaagactgtgtttatgcacctaaatctaacttagacaccatctaattcaagaaacaggcacatttagggaaataaagaaaagaggcagaaagagatgaagcaccttggctgaatcagagaaattaggggttaatgttccagactgatgacttgaaggagggtgtgcattttcatgcaagggaccattctgtagaagaagcagcacattcacccttaaatgagcaaagagaatcccaatcactctccctctcaccctccccttttccttcatttctctagctctttatttccaagaaggccagcccttctcttcagccacatggtatttggacagcctaggctcaatccctgcagggaaagctcaatcagccagctgagccagtcactgaacaaccaagtctggtaagaacaggggtgaagtatttaagaagcctatcgtggctggagggctaaagccttgttctcaaatcagctttataggtaataaaactgacattaaagcaaaaacaaataagagtaataaggcttcttctgtgtgtttcttgggttttgtttctcatttgattccaaatggggagacaagggtatatttatcctacccttcccccagcctcaacaggttaaatgggttaatacatgaaaagcatcaatcacagtgtctggagcattatcagtatcagtaaatgttaactattactttgaggctggcccatgtggctcctggcattacaggcaccagaggaattccagggagaccaggaggattatagtctgggggagtatggaggatgtgctactgtgtggcaggtgctttgcaaactgcattaaaatagtcttgatattttcagaatctaacgggatcccatatactatgattctgaaattcacttgcgcttctttagattttgcatctacatgatgcatagtaacagttcttaatttggtttattcctagcttatctatggttgtcattatttcaagagcctagtaaagttgaaatcaattttagaactacaaaaaaaaaaaaaaaggaagagatgtgagtagggtctcactgtgttgcacgtaaatgcattgacatggaagatttaaaacacctgatgagttttattctgtttctttattctctattaaaggtagtactgtgcaacattccctaccctctatttttgacactgacgttgggcttattttgattgtctaagaggtcctgtgctacaagctggcagggcttgctgggatgaagtgacaagcaaagatttgctatatggtatagccaggggcagagacaggttttctgggggcatgagacttctacagtggggagacactattaagaaatagaatacaaaattatgactacaaaatgagaggccccaaagcttatgtttcattagcttcacagggagctcacttctgcccagagccaaggactgtggacatgacctggtaatggcatcaaatgattccctttattgcctcccctggggcagaaatggtggcagcaaagcaacttccaatcacaactatcttctttcatttgacacaggaaggggaagctccctctttaatctctaatagaaacattaaacagcaggccatcacaatttctagctaaagagttttcataggacctttgcaaaggttgcattcctcccttcccttgtggcagtatgcataatgtaccttttaggaatataaattcagtgtcttggaacacatgatttagaatcagtgttcagttaccaactatcaaataaaaaaataaatccagcatatgactaacggaaagaagtcaatctgaaatggttacatattatatgattccaactatatgacattttgggaaaggcaagactatggagacagtgaaaaggtcagtggttgccaggggttagaaggaagggagagatgaatcggggaagcacagaggatttttagggcagcaaaactgttctgtatgatactataatggttgatacatgtcattgtacatttgtcaaaacacatatgtcttggtccatttaggctgctctaagaaaatatcttagactgggtggcttataaacaacagcaattggtttcttacagttctgaagtccacgatgaaggcactggcagattcagtgtctggtgcaggcgatcctcttgctttaacttcactggtggaaggggcaaagcagctgtctagggcttcttacataaaggtattaatctcattcatgagggctctgccctcatgacctagtcacctctaaaaggtcccacctcttaaaaccatcacactgggctttaggtttagtggggatgaattttgtggggaacccaaataacagaccaaaattcactgggaattgttttggggagacataaacattcagaccacagaagcacagcacatacaacaccaagagtaaatgtttatgtaaactgcagatttttagtgatgtcacggagaaaaacttctcctcgactctcttaggtttagtgcttgggaaccagtaaattaaactaactaaagacaagttaacaagagaaaaagcacaagtttttattgatatttacatgcataggagttcatggaaaagaaatgaaattcaaagatgcaattagactcacaggggttatataccattttaacaaaggaaaggtttggggcttcaaatgatgctaaattgtggggaagtgactaggaaagatacagggaaacaaaggaaagataagggttattttactaaggtctgtttatgtaaacttgtctgactctcaatctccagtggtaagagtggctctcctcttcccagttcaggagagggagacaccttcacaagaggaaatgtatgccctgattttaatctgataagaagagggcagagaacccttcctacatttggttgttttcaattgccttcagctcaaaatggtccttacgtggaagtgacatatttttatggtggtggtatattctgatctctttcaataataattgtgtgtcaatgagtctcattgattataatagatataccgctctggtgcaggatttgatagtaagagaggttgtacttgcgtggaggcaggatgagtaggtgaatgaactttctatattctccactcaatttttgtttgaatttaatattgctctaaacataaaatctgttcaataaataaagtaaaaaacaaaacaataaaataaagtaaaaaaaacaaatctagactgaggtaagtagaagctttattggaaaagattattacagagggggaagggactccaactataaaacagcaagcttctcagagctcaggcagaaaaaggcttttcttttatacgaaggagagaacaaggctagaaagaaccagatgtaagtaagtgaggtggcctgattggatagtagatcagagaacgttttaccctcaggccagccgcttcctggaaggagccattgagaaggagctaaattctggctcgacttagaatgcatcagagttcaagagtctggggaaaagaaagaaccgtaaccaaaattttttcaggtcaggttaataggcattctgttctaatagatcactggggacaataaatttagctaatcatttatgaagccaagaacaggagtttggagcgtctgtatctggctttgtcataggtaaacaatgccggcaatccttgagcctcatctaactcatatcgggaagggtggtcctttggaataagctattctctgaaacagtttagtctaagtacaatgtatttcaccagcaaataccttgattgtgccttggaaggaaggggttggtgatggtaagtctggaaaaggccagacaaagagagggagaattctggtccctgaagagacatcccatatgctctcgggtgagattgtgagaaatgctggaagaatgaagtggatgactaccaagcccatcttatttcaatgtacatagataatacttcattttgcaaaaatgaaaagtatgggacaaatactaaatcagacagggcactgggataagaggcgcgaattaggatatgtagtcatttactgtactaatttgcatgaatagaaatgcagagagagctctataaaagaaaaatattttgtgtaatctattagtatagaaactcgtaattaatttactaagatataacgatttattcgtcatgttaggcaacgtattttcaatgtcatcttatggagaattgtaaagattttcttgtatgttaaaggtctttggtcaccatcattattcacacgattgttattcacctggttatcattattcctcatcattaaggacactagtaatcttttaaacctttctggctctttctggggaaaagggcaggcttaatacaggcattcctcattttagttagcttcactagcttcactttattgtgcctcacagatactatgtttttcacaaattgaaggtttgtggcaaccctgcctcaagcaaacctatcagcaccattcttccaacaacatgggctcatttcctgtttccacgtcacatttgggtaattctgacaatatttcaaaatgtattattattatagctgttatgatagtcagtgagcagtgatctttgatgttactcttgtacttgattggggaaccatgaaccacaaccatattaggcagcaagcttaattgataaatgttatgtgtagtctaaccgctcctccaaatggccattcccccatctctttctctctgctcaggcctccctattccctgagacacaaaaatatttaaattatgctaattaataacccaacaatagcctctaagtgttcaaataacaagaagagtcccacgtctctcactttaaatcaaaaccttggcataattaaggttactgaggagggcatatagaaagctgagacagaccaaaagctagacttcttatgcagaacagttaaccatgttgtgaatgcaaaagaaacgttcttgaaagaaataaaaaatagaagatgctaccctctataattctatgaaggttcagagaggtgaggaagctgcaaaggagaagttggaagctagcagaagttggttcatgagatttaaggaaataagccacctctataatataaaagtacaaggagaagcagcaagtgctgatgtagaaggtgcagcaagttatccagaagatgtagctaagatcattgatgaaggtggctacagtaagtaacagatttttaatgtagaccaaacaaccttatgttgaaagaagacaccatctaggactttcatacctatagaggagaagtcaatgcctggcttcaaagcttcaaaagataagctgactctcttgttaggggctattgtatctggtggctttaagtcaaagccaatgttcatttaccattctgaaaatcaaagagtccttcagaattatgctaaatctactctgcctgtgctctgtaagcagaacaaactggatagcagcacgtctgtttatagcatggcttactcaatcttttaagcccactgttgaaaattaatgctcagaaaaataagatttatttcaaaatattactgcttattgacaatgcacctagttgcccaagagctctgatggagttgtacaaggagattaatgttgttttcatgcctgctaatacaacattcattccaaagcctatggaccaatgagcaatttggaatttcaaatcttattatttaaaaaatacattttgtaaggctatagtttctatagatagtgattcctctgacggatctcagcaaagtacagaacaacatggctttgaactgtgctggtccacctatacatagatttttatctgcctctgccacccctgagacagcaaggcccacacctcctcctcctcagtctactcaacgtgaagatgatgaggatgaagacctttatgatgatccaccccacttaataaatagtaaatatatgttttcttccttatgattttcttaatacattttcttttctctagcttactttattctaaaaatccattatataatacatatgacataaaaaatatgttaattgacggtttatgttatccataaggcttctgttctacattaggccattagtttagtttttgggaagtcaaagttatatacaaattttcaactgtgcaggtggcccaaatccctaactcccacattactcaagggtccactgtaagttgaaaactcctggaaaggattcatcactgtagatattagggctttcatgattcatggggggaagtcaaaatttcaatattaacaggggtatgggagaagttgattccaacccttatcgatgactttgaagggttcaaagatttagtggtggaaggagctgcagatgtggtagaaatagcaagagaggcctggcgtggtagctcacgcctgtaatcccagcactttgggaggccaacgtggtggatcacaaggtcaggaaatcgagaccatcttggtcaacatggtgaaacctcatctttactaaaatacaaaaaattagccgggtatggtgacgcatgcctataatcccagctacttgggaggctgaggcaggggaatcgcttgaaaccaggaggtggaggttgcagtgagctgagatcacgccactgttctccagcctggccacagagcaagactccatccaaaaaaaaaaaaaaaaagaagaaagtaagaaagaaagagcaagagaactagaagtgggtcctgaagatgtgactgaattgctacaatctcatggtaaaacttgaatgaataaggagttgctttgtctgaatcagcaaagaaagtggttgcctgagatggaatctacttctggtgcacaggctttgaacattgttgaaataaagacaaaggatttagaatattgtacataaacttaattgataaagtggtagcagagttcaagaggattgactccaaatttgaaagaagttttattgtagataaaacgttaaatagcagcacattctacagagaaacctttcaggaaaggaagagtcaatttatgtggcaaacttcattgttgtcatattttcagaaattgccacagacacaccagctattagcaactaccaccctgttcagtcagcagccatcaacactgaggcaaaatcttccaccagcaaaatgaatatgagttgctgaagactcagacatctttagcattttttagcaataaagttttaactaagatagtacatttttaagacatatgctattgcacattttatacactacagtatagggtaaacataacttttatatgtattggaaacaaaaaaaatttatatgactcactttattgtaatattggctttaccgtggtgatctggacacagaatccacagtatctctgaggaatgcctgtagtttgaatatgagtgatgcaaagaaggatttttagctattgtagtatctctggatgtatttcccacaatctttttgggttatagttgctcttgtatctgttttgcctttcccaatagtgattgcacttagcagaatttgtgctaggaaacattgttattgttattgatacaaaagtaattttctgtaaagaatagatcattttgtctgattcattattttagtggctctctgaaaagtatttttcttcatttcacttctgaaacagaatctagaaaaaaaaaataccatcagctgagacatttagaaacatctgtactttcacacaatgcatagacaacaaacccttacagaattatttttaggaactctattgaaattagatattttctaaaagtatttgctaaaagagaaatgcattttagcttacatcatattgtttaattttaaatctatcatattgtgacaataacaagaataaaaacaatttcaccctcagtgtaaaacttacagattttgttgccgagaaacttcaaaacatacctctgaatagtaagaagtgtagtaaaatttaaatgaaatgttgggccaaatatagaatgattgaaaaatcacccttgaattataaaaatttgtgctcatcttgtgaatactttaaatgtcttgctcatggtaaggacttcgtgctatctttgtacaattaacatatagggcaaatttcaataccaatgacagtagatgtaatttcacatatcaagtagtttccctaataatttaggacttttttcactgacttcttgtcatatatgattctgtcattacttttttatatagctcaaaatatggctgatggagtgcttgtgcccttccatggttttttcatttggtatcactagtattatatttatttattctttttttccaggacatttgcaagacattcgtcaagttttgaggtttatttaaggtataactagataatgtattatgtaaatcagcctaaccagcacgcataaactaaaccatgtcacaggatgccaatagtgacagacggagtgctgatgcctctggaaatctcttcacattgtggagtttctcctcttctcttgggatatcttctctattttctgtgacagatgcctatttgctagatcaactgagagcatcagtgcctataggtaatgaaatacaaataaaactcaatttgtgttatggtctgaatgtttatattcctccaaaattcatatgtcgaaatccggtccccagtgtgttggtattaagagtcgaggcttttgggaagtgacagggtcatgagagtggagcacttgtgaacaggattagtggtcttatgaaagatggagcgtctgtttttcccttcccccatgggagtgggaaaatacggatagaagccactatccatggggaacaagcctccaccaaaaactgaatctgctggcaccttgatcttggacttcccagcctccagaacgctgaacaacacatttctgttgtttgtaaattacctaatctaagacagcagcctgagtggactaggacaattggtttcttttttgtttaggtagcaataactagaaacagaatttctacagaggcaacacacacaaaaataggtgtgattttatcaaattaaaatacttctgcacagtaaaagaaacaattaccaaagtggaaaaaaaataacctatggggtgggagaaaatatttgtaaaccatgtatatgataaagcgttaatatccaaaatgtatgagaaattcctccaagtcaatagcaaacaaaccaatagaaaaccagaagcaaaagaaaaaagaaaataagttaaccaatttgaaaaatagacaaagaaactgaatagacatctctcgaaagaagacatacaaatggtcaacagatatctgaaaaagtgtggatatcactaatcaggaaaatgcaagttaatatcacaagataccacctcacagctgttaggataactattagcaaaaaaaacaaaagcggccccttgctttgtgtctgccagaatccattatggctgccactatagttctccaggagtctggatagcagtcccggctgggaatgtctggagtctgcaggccccagtcactatggacagtttttccttcagtattggggttctaagaggcagaaattagaaataggaatgcagatggttgcagagtgtggttgggggttagagcggcgccctggaaaaaaaaaaaaaaaaaagatgagtgttggaaatgatgtgcagaaaagggaaccccagtatattattagtggaaatgtaaattggtgcagcctctatgaaataatgtatatttcaatacataataatgtatattccaaaattgtttttatagagtagattttagatgttctcaccacaaataaatgataagtatgtgaaatgatgcatgtgttagcctgttttaatcattccacaatgtacacaagtataacatttacattataccacataaatatatactctatatatatactatatatatatatactactgtttgtccatttaagtaattttttacaaataattgttctaaataataagattatcttataaaggagacagtaggtttgttttgctccagatggtagagttaatccctgaagctagaagatgcttagggtgaagtttgtgctcaaaatatttttctaaaaatagagcagactgcctcaaaggtgaggtatgttgcagaaaaaagtcactgtaaggtatgtgtgggatgctgtttggggataatcagggactcaggatagggaactaaggagtttaggttgtctgctgtatagaggaaactagaggagatagtttattcagccatcattaattcattcattcagtaaacattactgaacacctgtgtgccaggtgctgtgattgacactggagatttaaagttgaataatatctagtcactggggccctgatgtcaaggcacatgctctagtggaagagaggcacacgtagctaagagtgtgtggaaagtgttatgaggaaagtatgttcaggatagtgtagaggctgaggaagaggattcccatctcacctgctgcgggcaaagaaaggtggatggtattcattagggattatcacaggagggatcaactgaactaatccttaaaatataatacagtaggagccagcccacaaaatgctaatagaggcagtggggagggtggtggaaaaaaaaaataggtatgagtcaaaggagagaattccagacagatgaaagctgtaaaaatagtttagttttactgtaaaaaggtcaaagccagaagtaaggggctgcaaagtccatttaagctgagaactttattacagctcctgttccaagcccatgctctgtgcgactgcagatgggatattctaataccacttgtggaaatcaatctttttggacaaagaccaagagagacccatgttgtgattcactctctatggcaacccaaaaggaagggtgaatgcctgcagatgagagttgcctcttcatgccagccacttaataggctatggaaaagggaagagcagtccccatggtgggcctaataggaatcataatctaactgattatcacagcagtgactctttcaaatctggcctaactgaagctagcaccacaccaggatctctgctgggcacacaccaatcacccaggaggtcagtatcatccccattttacagatttgaaagctgaggcacaaggtaaataacagttatgcagaagtcttgcttcagtgtctaacttcctcatctcactttattctgtttttcaataggagaagagatataatctcatgatgaaaagtgccatctaaagtggcaccttagacacagtaagcaaactggattggaagtgaagaagtcagtctcaagatacttgacaatgtctcctatttgtagattgtttagcaaaatgctttcacctgagttatttcatttgttgctcacaaccaatctgtctggtaggaaaggcagatgatatgttcacctccagataagaaaaatgaaattcagagaggccaaggggcttgctcaagattccacagtgaagaatctacttgcaggaattttaacaaaggttttcaaattcagaagtccacaggatgactgcccttgaaatccagtggtaacatataataaggtgtctttgagcaggtaagagaggaccaaccttttctaaagcctttctcctcttgctctctgtgcgccaacctgagcagcctcctgagagattctggaacatgctagaatttttcttgctttagggccacagttcatgtcactccttcttcctggaatgttcttcacaagcactgtgtggctccttcttatccttcaaatatctgctgaaatgttcccttcttagaaaggacttgctgaccaccctgatggaagtgccctctccagctcacctctttactgactctgtttttattccttttgcaagcacattttataatctatatgtagtgtttaatgcaacagtttgctaaggactgaaatttgcattcctccagcaatcaaaacccactgcgatggtttgtggaggtgggacttttagggggtaattaggtttagatgaggttgtgagggtggggcccctcataatggaattagtgttcttataagaagagacagagagaacgaagctctctctctttgtgtgtgtgtgtctctctctctctaagacatgggaggacacaatgagaagacaactgtctacaagccaggaaaagggacgtcaccaggaaccgagcctgctgacatcttgatcttagactttccaacctcccaaactgttagaaatcattatctgttgtttaagccacccagactgtgatattttgccatgatgcctgaactgactaatatacagtttacctgtacattgtttgttcctctagattataagctctccaaggcagggaccatatagttctttaaaagataatgtcatggctataagtttactgctatttccccatcacatgggaaaaagaattgtcagagttgtcagccattagagtttatttaccttaagggtgtctgacgaaatgatctgattatctccttaacttcctaaatagctcaaacttcataatgtttgaatttggaaatatctgttgattcaagagagacagattacatggtctctcttgaactttggacatctcttccacaaacttccaaatattaggccatctattaccagaccttctgaacttaaacaaatcttactttcaaccccactttagccaccttcaccatggcctaacttttgcccttgttatttgttatccatagttttctaaacttagaatccttaatcactggcattatagttgtttacttcaacctccaacacttacaggtccctcaaatcctcatttattcaacccttgttttttgtcatcttataaactgcaaattcttgaaaatcacatgtcctctctataaatcagatcccttcatatcatgttttcaatctgagtttggctccatgattaatcttccaaatcaaaggtttacaaactgtggcctgtggaataaatctggtctgcacctgttttagtaaataaagttttactggaacacagtcatactcatccatttacatattttttatatccattttcatgttacaatggcacaggtgactagtggcaacagaatctgtgtggctcacaaagcctagaacattttctatctgtcctttatagaaagaagttaccaacccctaatcaaaatcaataatctatgaagtagaaagtaaaagatgatcttttggtgctgacatgaacatattatgattaaaaaaacactttattgaggtttgattagcatacagaagctgtacataacacatacaacttgaagggtttggtgatcagtatgcaatcatgaaaccatcactatgatgaatgtcataaacatatccatcacccccaaaggtttcctctgccttatttatttatctatttataagaatacttaacataagatttgctaccctcttagcaaatttagcaactctttgagtatgcaatataatatagttaactatagacactatgctgcacatagatttctaggacttatttaacttgcgtagcaaaaactttgttctctttgcccaatacctccctgtttccccctccttcatcccctggtaaccatcattctactgtctgcttctatgagtttgactgttttagatttctcatatgagtgggatcatttagtatttgtccttcgatgtctggcttatttcacttagcataatgttcaccaggtttatcaatgttgttgcaaatgacaagattcctttttttattattaaggctagataatattccattgcaaatatataccacattttctttgtctattcatctgtagatgaacattcaggttgctttcatgtcttgggtattgcgagtaatgttgcaatggacatagaagaacaggtatctctttgacatactaatttcatgcctttgggtaaatacccagaagtgaaattactaaatcacatggtagttctattttttggtttttgaggagcctccatacagttttccataatggctgtactaatttacattctcaccaacagtgtaaaaaggttcctttttctccacttcttcaccaacatttaaatttcatctttttgataatagccattctgacagatctgaggtgatatttaattgtggtttcaatttgcatttccccaatgattagtgatagggatattgggctttgttattaataacccactggccatttatgtgtcttcttttgagaaatatctgttcaagtcatttgactatttttaatgtaatcacttgttttcttattattgagttgtttaatttctctctgtattttggatattagagccccttaacagatgtattatttgcacatatttttctcttaacctatgggttgtctctttattttgtaaattgtttcctttgctgtgcgtaagctttttagtttgatgcaatacaataactaatagattaatggtctctttttgtttttgttgcctgtgcttttagggtcatggtcaaaaaaatctttgtccagatcagtgtgtggagctttcctcttacgttttttatttctaatagttttatagtttcagatcttatatttaagtcttcaacccactttgagttgattcttgtatatgggatgtgatgtgttcaatttcattcttcttcatgaggacatccagttttttcaacaccatttgttgagaattcaataaagttgcagtatataaaatcaacgtaaaaaatcagtaggtttttcatacactgacaatgaactatctggaaaagaaattaagaaaataatcccattttaataccatagcaaaatacttagtggtaaatttaaccaagcaagaatatggaaatatctatattctaaaaactataaaacattgatcaaagaaattgaagattacacaaataaatggaaatatgtctcatatccataagttgaaaaccgatattgttgaaatgtccatactaaatgtgatctaaagagtccatgtgatttctataaaaaatccaacgtcatcttttatcgaaattgaaaaaaaaatgctaaaatttatatggaaccagagaagacctgaataaacaaagcaatcttgagccataagaacaaagttggaggcatcacactacctgatttcaaaatatattgcaaaactatagtaatcaaaatggcatagtactggtgaacaaacagatacaaaaaccaatggaataggatagagagcccaggaataaatccacaaattaaaaaccaactgatttttgacaaagatgccaagaatggggaagggagagtttctttcattatttattctctgtattcttttcatctcaggaaaaatatccagtttcctcaattgtatatccattcactcacccgataatttcttcattcacttatttgtttattcatttaatctcaattgtttgttcattctgtaaatattcagatttctttttatgcattttctcagaattggaagcataatactgaacaaaataactataaatctcagcctcccactcccatatttacagtttgattagggaggcacatttagatatgcagtgataattgctttgcttagagaaattcaaggtgatagaatgcatggtgacacctaacccagactggtagagaaagggaattcttccactgggaatgacatgattatctaaataagtaggctcaatcaggtcaggaaagggcctgaaagactatttcaagcagagggaaggtatttgccaaggccagggtgtgtagtggagagaatgggcagtggcagagaattatgaagtgttccaatgactaaaagtaaagtaacaagttccttgtccaagagcttggattgtatcctaactgaaatgagtatacactaagtgtttgaagaagagggatgaaatggtcaagttttcattacacaaaaataacctgttcctttcattttatgtttatttatttttttaattttctgactgctcctttctggaaatctcaaatttatatttgccaaatattgtcacattttcgatggagaatacaaactaagaatgggttagggaactgagtcagaaagtccctgttgtacaattcaatcatgtttttctaaggatgtgcttttggacattatggaaactatcttaggctctcacttggatcctagaaaagaaggcacctgttaaaaggaatgtccagccccacctaatttggcagctgcccctccaagctaatgacattagggatgtagtggattcaagaggctgatgatgccatctggtcaactcatgtgactatttctatcagctttatttctgcaacatcctgtgcccacagaggggacacaaaatcgctttataattccttcaccatgtgaagatacagatacacccagaaccttaaaggcaagaatatgattgaaatgtcaaatggggacttggtgatctaaattatgtcccccaaaagccaatgtcttgccaccaccagtgccctatgggtggagtttctaaacagattactcaaaacacaaactttcaaaaagggaaagtcataaccctctagtcatcagggcaattacggaataacattgctggagtaaggttttctcaatgcccaagagatgagctggcaatgccacaacaatgtccaattcttagtgggtccaagaccatgtgttacatttccctcccatgattactcacagcttcacagttctgctgtcctcttcgcctctctgccacctcttaactgcacctttgacctcctacccctaagattcaaccctgtgagattacttgtcttttcatctacactctggtcactctgacccccattctttagattcaggattttctcttttcctggcttactctccagcacaagtagaaaaatattgtgcttcattggaaaatgcatgttgtttgaatcacactctttcagattatacaattgtagtctttcattatctttgagccattttataatgctgtaaactaatattaatgcatataattcttgtctatagacatgtaaattgtgtccaggggtgatttaattgacttttcttccccattgtggaaaaggctagtagttttgcctccatttgcccatttatttctatattcctgatctgtaaatgcatttctgggttttcccttggactcattctaacatctcttctttttcctcattaattaattaaattaaatctttaacacctcttcatatttttgtcagtatgagagtataaaaaaaaatctttttttgaaaattatcttttaataggtgacaaaaagaataaacactgaacaagaagtcctgagctcctactacaatccaaagtgaactagttgcagtgccataggatgatgtttctcctgagacttctttactaggatcatttagactgttataaatgcagattcctgggcctctccttagacccactgaatcagacactctaagaggagggtcagaaatctccatcttagcaagttaatgtccaggagagttagagactcactatgacgtatgataaaaagtagaaggaaggactttccagcaccttaaactacttggaaagatagattaatgtctagatgaaacttgatgagactttagactaatatgttacatatacatcaggagatgcatgtataaaccaaatccaaataacccaaagcaaaatttcttttagaaatagtgataaataaatgagtgaggagtttgtcactcacatcccccaggtaaaacatacctttttagcctaaataaatgctatagtttgaatgtgttccctccaaaattcaggtactgatagttaacagcagccgatatgatggtattacacaaaatagtaggttctttaggaggttgttaggccatgagtgcctcccccaagaatgggatgaaggacctcataaaagaggcttctcacagcattgtgacctctagccctcccaccttccataagtgaggacacagtgttcctcccctctggaggatgcagcaacaaagtgtcatcttggaagcagaggagccatcaccagacaacagaaccagccaacagcttgatcctggacatctcattttctccagaactgggataaaataaattcctgttttttaataaatttcccaatttcaggtattttgtgatagcagcaaaaacagactaagacaactagtatgaaaatatacattaacaaataaaattaatcataatatatgtttgcttttaaaagaaaataaataagccaatatgctttctgttgattgattgatttactaaacattgattggccatctccactggggatatggcatttaagagatctctttgatcttagtacttttactgctttttaaataggatcaaatacacccaaggtaaaaaatagaacacactatacgttacattttggaactgttagaaattcctttgaagctaaaattactgctatcatttgacaacttttacccctaaaataatgtggtgctcaccagcttgcttaagttacagcacttgctgtcttctcagatacaatatcagaaacttataatccaagaaaaatctaaatggcaagtgtgagttaatggaagcctcataaagcaagaggtgttttggaagtgtatggaagacatcaataaatgatatgtataacatcaagtgcaaaagtgtgtgctaggaaagttcaaaaaagaaaaaaaatatggtaaggtaagaccagagattggggagtatatagcttttgggaattcaggaaatgctaacgtctatggagaatttgcataggtgaaagatcagattggaagcctttctctgtggaagcattgtggatctaagtccagaagtgatcctgagccacctactatggaaaggtgtcagtgagcaagagactgtctgacaaaggtggaagctgagcagacttctactgcgcatcgcctatgtacaggccagattccaagggctgatattacactgctagtttgatctttctcagatagctgggtagagagggaaaagtttcaccccaaataccagatgcctccaaacatctagatgcttggtgttatgtatttcaaacagcaggtttgataaagctgtttttactctccagttggatgttggttgtcaaggctgtcattaacttctgggagttcccaaatcctcagagagagagaaataagcagttctgcctgcaatcagaagctggctttgagccccacatggcaacatgcctgtatttaattagaagtggcttagttcttgggatttactgtggattgagtctacgaagctggaaggttttatcctgagtattccactgtctcctcatgtggattttatcaaatttctcctttaaccatataagcgctttcaaagttgacatatcacacttagttatgaggggaacctagaagtatgattgtgacgtgcattgttgagagaccatgctttgtaagtgttaataattaaaattattttgaacactaattatgctgaagactggattttctactccttcagatgttttccagtggacatattttgcccttttaataaattgattgcgaacttatcttcatttcacctttatgatgttataccttttcatttttgtcttctcatagctagggtctcctggtccccaaatgtagacacacatcttacaccaatcccagagccattttgtataagagccaccatggatttaaccagctttagctccagtatttgaacataatgttcagcatcatcacctggccaccaaatcaaaactgagcaccctttaatccatcaacaagttctctgcagccatgcaaggttatgaaatgggcacagacatcaatatacagtctttgtgtttaagaggttcatggtctacctgagaaatgcatctttaaacctaaagtagacgctctgtttattccataaatgatttttaagcatcaatggtatatcaagcactgtactggcttctgggctataataaatatataaagaccacaagtttgaatttcatgacattgaactataatgtttaaatgttataataatcatagtaaatgtccttgaggagctacggaagattcctgcatgaagcagaaacaagaagctgaagaaaaaacaactggctttgggggctatataaatataaccctcaaattaaaaactcaatagtttgattgattaccaaatcaatacagccagaaaataaatgtattaccccaaaagcttgagtgaagaaagacttctgtaagttactagaaagcgctaaggaagaaataaaagaatgatattcctgagagccaggtgctttctgtaggacacacagatacagaatgagggaaaacagaaaattctatggttgtggattcaaaatagaggcatcaccatgtctatctcatcagggtttaaccagaggaaaccaaaccagtaagatatctgtattaagagatttcttgcaaagaattgacctatgtgattgtgggcactggctaggtaaatcctaagtccacagagcaggcaggaagaaaacaggctgggacttgtaggcacaggatgaagctgcaatactcatgtggatgctgctcttcttcagggaagacttggctctgctctcaaggactttcaggtgattaaatcaggcccactcacattatctaaaataatctcccttactcaaaaccaagtgattatggactttaatcacatctataaaatatcattatagtaacacctaaattagtgtttgaataactgagagttgtaactgatatggtttggctatgtccccacccaaatctcatcttgaattatagttcttataatccccatgtgttgtgggaggaaccaggtggagataattgaatcataggggcagtttctcccattctgttctcatgatagtgagttagttctcaggagatctcatggttttataaagggcttccctctttgctccactttcattctccttcttcctgctgccatgtgaagaaggacatgtttgcttccccttctgccatgattgtaagtttcctgcggcctccccagccacgctgaactgtgagtcaattaaatctcttttctgtataaattacccagtcttgggcagttctttatagcagcatgagaatggactaatacagtaactttaccaagtggacacataaaactgatcattacaatgtacagtgaatatttggtgagttaatagatatattcataactgaatgaaagaggatggtgattcctacttcagggtggtattatgagagttaaaagggttagcatagatagaacacttttctatgattgatctaaggttggcttttagggactacattatacatatgggttttgtcttcatgtttttgcttgcatttctctctctgcccactcttggggctatttggatgtcacctcttcttcttctgagctccagtctatgtattccctgttccatcatcacaaccactgaagtctacactcctcattagattcagggaacccagatgccagccaaagacatccctttccctcatgacccagttgaaaaaattctccacccttctgttaattctagaatatttaggagtaaagatcttttccctcagatatctgtcaattcccggccttatatctcaaagccccttttcatccaggtttcctagatcatttcttgtcgtccctgtgctataactcattaacccaggcataaaactcatcagctactaatgtcctcttccatcccagactcccctcacaccaataattcttgaatagagcaacccaaattgagaattttaagtaaaattgaaggtaaatttaattgcaggaggacatacaacattaacctttaaatagagatcctaattcttaaaaaaaaaagtcctcattgatctcggggccgtttcacgcaggtgcttttctaaatcatcaaggttatcttcagcttcactttcagtctccttcttcggctctggcgctgccactggctcttcctgtgctaatgtggttgtggcagctgcaatggctgcaggggtcacagcagctgcagcagctgcagccacagccttttccttcttgtgttttttgtgcttcttgtgcttcttatcctttttgtgtttcctgtccttctttttcttctttttctttccatctccttcttgatctgaatttcttggcagtgatgggctcggtgttgggcttttggcctttttgaccggtacagctggtgaccagtttgtagacggtgactgagactggatgggggatggagatgctgggggctttttagctgctggctcaggagacccagagacagatcgggaggatgagaccctccttacagactgtgggcttggggaagcagcctttttttatctttttaggttccggagtcctggagactctcctaatgggcctagtacttggagacggggactgccttctttggggtgatgacgacgctcctcttcgaacgggtggaggacttgaggtctgaggagctcgaggccgtgatgagggcgaatgccatttgtttggatgcggtgatcgggcctcccgggtagagcggcttggggaagaccctttcctatgcttggatgatagtgaaggtgaacgtctcttggtgactggggagcttctttggaaggtggagaatgggagacccgccgctttggtggtggaaatggtgatgctcttcgtttaggagggggaggaggtgaagccgttcttctctttggaggtggagaaggagagtatctcctctgtattggaggagagtatcttctaggagaaggtgagcgccgacgtaggggaggagaaggagtccttcgtcgtggtggtggtggtgtgggagtcctgagccgtcgaggatgaggggcgggagaaggagaccgtcgccttctggtgggtggtggggatggacttctcctccgtctaccatgaggggaagtctctttttggtgctttcgtggtgatggagaagcactccgggaaggggaatgtctccgccgcttgccaacctcaccattcttcacatgggatctcttgggtcgttcatcttctgaggagaaggaggaaccagagtcagatgaagactgctggttttgtcgtctgtattggcgtctctgctgagatcaatgaggaaggcccaagtgtccccacagtcttagggggaaatgtttgttatgatgtaaattttatttggtttgtacgcagttcaatttcaaaattgctaaaatgtgtttgagctttagactataacatttgttgtaataattgctaggttgaagttcaacatgtaaaaaaagggggcatggatttacattgcaaaaggtgtccacagtgtattagtgacattctttcattgacagctgacataattcattgagtgaaatattttaagccaaaaaaaattccctttttaaaaaagggggtttaaatactgttgacacttttatggttcctttaaatgctctggctattcccagaggggtttttttgtttgtttttttggttttgatttgctttttgtttttctttcttcttcttacatttttttccatttgagtcttagctcccatttaagttatgcttctgaccttgtatggtctgtaagcttgcccagaaataagaccactgttttgaactaccacaaaagtataaatgaatattttaatgccacagtctttcctgttgcctgtggagtctctgctgaaatgaatcaggattcgagctctaggataagacagaaaatgaaagcatgttgtttgccaggacactgtgggtttatattgatgtgtaacaacttgatttggaacactggactctcattctgttcttctggttttgtttttttgttttgtttttttcttttgtaaaggccatgaactagtcccagaaaggattccttcagttacatacaatttgtttaatgaaatgtcatggctctgttcatatttttgtcttgttcttccaattggtgtatacaactttcagagcctcttgtatttggaaggctggaagggcccagactttggaatagtgtctcggtttcactgtttttgttttgattttttttttttattttttttaaactaaagctatataaagcttgtggattaaacagaataaatttctaaatttaaaaatttaaaaaaaaaaaagtctattgtcttccctcccctaccctaagcaatatgcaatagtggctcttcaatagtcccagactcttcttctcttcctggactgcccatctcctgatcaacccttaatttctcttccttctctcacccttcttttcaggattgaattaatgaatcctttcttctcactcatgcagagtaagtttctgcctccctgggtctttctgtttactgaccgcaacaacttcagattatacctcttctactccaagtgctttcaaagaaagtcctctgccaagacaaattcattacgttttttccctctacctgtttgcctttattctcttttgtatttcatcttctcatctagattgaataatctttgagagcacagatgtttatttatatttttcctttccatttctactcagcatgaggtgtccattgaacaaacttgatgaatttttattgcttaatatcttgctagaggtggggagagaggttgggggcggttaaggaactatcagctagcctaggagatattagagctgcagagatttggctatcttgttcaacgttatatccctagggattagtacataggcttgcaaatagcaggtatgaataaaaaattattgaatgagtaaatgaatttaaaatataagttacttaggcggtatcttcaggcatatctgtgtttatgtggtattcaatggcccacaaatgtctacatcctaattcctaagatctgtaaacattaatttgcatgacaaaagagactttacagatgtgattaaatgaaaggattttgacatgcagataatatcctgtattcttcatgtggaaccaatgtatttacaagggtccttataagtaaaacagagaagcaggaaaatgagggtcgcaaaaaaaaaaaaaaaaacaaacatgaagacagagaagaggttagagtgatgttggctttagagatggaaggagtcacaagctgtcttaaaggaataagacaagctgtcttaaaggaattgttataaaggaatagctgaagctgggtaatttattttaaaaaggtttattttgctcactattctcatgtctggaaaagtttaatattgggtagctgcatctggcaagggcctcaggctgtttccactcatgtcagaacgtaaaggggagctggtgtgtttagagatcacgtggggagagaggaagcaagagagagggaggaggggccaggctttttttaaacaaccagctctttttttaaaaaaaaattatactttaagttctggtatacatgtgcagaatgtgcaggtttgctacataggaatacacatgccatagtggtttgctgcacccattaacctgtcttctacattaggtatttctcctaatgctatcccttccttaacccccaaaccctgacaggccctggtgtgtgatgttcccctccctgtgtccatgtgttgtcattgttcatctcccacttatgaatgataacatgcagtgttgggttttctgtccttgtgatagtttgctgagaatgatgatttccagcttcatccatgtccctgcaaaggacatgaacttatcctttttatggttgcatagtattccatggtgtatatgtgccacattttctttatccactctatcattgatgggcatttgggttggttccaagtctttgctattatgaacagtgctgcagtaaacatacgtgtgcatgtgtctttgtagtacagtgatttataatcctttgggtatatacacagtaatggaattgctgagtcaaatggtatttctggttctagatccttgaggaattgcaacattgtcttccacaatggaacaaccagttctcttaagaataaaagtgagaactcacttccctggccccagagagagcaaaagcaattcatccccatcacccaaacacctcccattaggccctacctccaacattgggatcaaatttcaacatgaggtttttaggggacaaacatccaaactatgtcacaagacaattcatgtaagcagcctctaaaagatggaagaggcaaggaaacagattctcccctaaagcccacagaaggaaagcagccctgccaacttcttgattttaacccagtaagacccgttttggactttggacaccaagagctataagatgattatgttgttttaagccattaagcttgaggcaatttgttacagcagcaattggaaactaatacagatcacattctaattcaattagtattgttcccagttctctggacctcagatttctttcctgaaaaacattaaaaataatacctgaaagttttgcacacgagtgcagagtgcctatttactagagagatcagcatttgtttaggctctgaatagatttgaggatgaaattaaatagcataaataaagttcctagtgatgcttctgataaaaaaatatctccttcaaaatgccagaggcaggtcctaaaaacccacaaagcaggtgaactggcaaaagactgtaaaaagcaaagtagaggttcctcttcaaagactttcctctccatctaattaggaataaatagtaacttatcttagaaacaaaatttattcaaagacctgtgctaacattctgaaatatctgctagccgtaataaataaatcgatgtactttatgttcttagctcccacaatttaacctaaatatttgccctggcatgcttatactggtccaagcaagcattaggtcatagcctgttcctcttctttattttaaggtgtttttacctttgtcagcatgccacaagttacttcctccttcctttgttctcctctgcctttgactcttttaaagagtcctaagttgctagccaatcaggacaaatacagaatgtgaggtcccgtttcagccaatggaaagtggacacagcaggaaggtggatgggtcaggttataaatgaccctgtctcctttgttcggtgtactcttgtggcaaaactgctggcaagtgtaccctttctgcaataggtaaaaactgccttgctgaggaaattaaatttatgttcaagtgctatttctttatggcaccggggaacaagcatttctaacaagactatgtaatttaatttcaggaacctaaaaaagtgggatgaagaactgaggttgctaataaatctatacaacttataagtaaatatttaatttactaacatataataataaagacatcattgtaagacaatgttaaaacattttacacattttaaatgtgcaatagtaaatccttcactattcagggattatttggaatcccttgtcaccagaagctcttaaggaaataacttctacttcgttgcaaatatgttcttggcttagttgaggtaatgcaaatactagaatacttgtttgtttaacagcttattcttccctgaagctgttcctccagtccctgccagtgggatcttatgtctccaggagtacttaacacccctaatagccccatcttttaagcctccctgggacctgccctcgcagtacctcttatacctactccacttcctcctcatggcctcctgcagaatgccattctaaaattaggttctattttcctcgcccgcattctcttttgcaaagcctccaaaaaatttactttgcttctctgcgcctgctttatctctattttctacactcgctccttctttttctaattatctataataggcgtcacaaaatttgcatttgttggaaccaaaatttccatggttgcctcaaaatatacagatgtaaatttgcatataattaaattttgcataagggaaactctcatttggggagatatgcaatgcccaataaatggcagtttccttcaatgtccccaggccagcctcccagtctgtgtgtttccccctggctgcagctaccaggactctgctctggggatttacggacaagggtatcaagttttaattaaactaaccctctcaaactgaatgagtggcttaaaatcttcctgtaaagaaaccgcaaaataataatgctggcattgagaagtaagaaaagagcgagccagcacccccaccccccaaatcctgtgacaaggtgtatttttgtgttttgttttttttctttggcagcattatgggggaaaagcaatgatgatctaatgagatctgataagaagttagcccaaaacaaggaaattgttgagggttctctttgaagtatggatttatacccaccaaccttagctgcgaaccttacctcaagtgttacctgtgccttgagatgtttcctggtcatagtactaagctatcataatgagcaagacattcaataagcaagtgtgatggctatgaggacagatcttaacaggtttttttttctggaaggcttaaaatcatgcattactcaatctaatacttcacgaaatttcagtaaaacctaatgataatatagaagcttgtgttgtagttttgtaatcaacagcaaaacataaaatttaaaaaaaacatacattactggggctgtatcctgctacaataataaggctgacataatagatggagaacaatatggtaacaagccaaaatgtattacttcatccacaaatagtatcgtgctatatatagacagacttgttaaaaatttaaagaaatacacaatcaattacacaatagaaaatttgctatatggtgcatggtggcatgcaactacaaatgtttctaacatgtttctcttcataggattttctgaattttcatttaatattcaagcacatcaaaaacaccttttcaggtgtgatcctatacagcaaagctgtcctcacaaacaatagttgactaaataaacacatggctttatggaagaaatgtgtaagtatagccattgttggagcagatgctctgcttaaaaagaaaaaaaataagttaaagttatagatctcaccatgcttatttactgcttaagtcatagccaatttattgcaccaaagttgaagttcaaagcataaagaatactatatataatgcaattaatgaggttgatgtccctaaaacaagagagaattagtaaatgttttacaatagttttcatgagatgggaaatgacaatagaaatctttgttacaatgcagattttattgtggaaatgatctcatggcaaagtttttaaagaggctgcagaacaataaagagagataacacattttgcttttatgaaaaagccgatgttccaaatttgctgaccttctctgtaaataagaagtgactgtcagtagccagcagatgtgtttataaaaataacccacttgatctgtccctgcaaggtaagagtggcattttcaaagtgcatgagagaactgctctcaagagaaaatcatactgtaaagacagcatttcaaaaacatttatttggaaatatttaaatgatgtgatctatttatttatttggtcaaaaatgcccaacttgcctaacattattttattccttaaaacacagatcaagcaacagatcctacagttagactcttctgatcaccctgagagttagttacccctttctttggtttcttctctttaacgtcctgcatatatgctgccattgtgtttatagcacaatatcagagtgtgttatttatacgtgcatctccctcattagattgggagtttcttgatgtcagaacacagcaattgtcccaccagaataaaaagaatattgccaaaagatgctctataaatgtttggccattggtttaaaaaacaaataatggaccaatgggctcaaaagcaaactggctaatataaaaataataataacaaccacaataataaaataagggctaacacttaaacggttgtgtactcactatgtactaggcactgatcaaagtactttgtacatattttcttatttaatattcgctaccatcatattacaatatactgttattaaccccaatgtatagatgtaggtgaagaaacttgtcacaaatcatacagctagttgtctgagatgcaatccatgtgatttgttcacagagctcaggttctgtgaagcgggtaaaaacaaaatttggcatccagtttcaaaaggagaattgcaaactaatagaacatatagcacaaaatgattatatcaatagaatgctaattgcatatcaaggatatttggtataatacaaattattctaccttaaacatatggaaatttgtggtccatgatgttgtagattctatcttcccactctgcattttcaaaggcatatggtattgactcattcgattaattgttggatagtctttattatagactaaatcatagaataaatacatggatacatgcacgaatattatatctcaagggctttacatagttcattatctcacttcatagtcaaaacaaacctactgatagttccaatgcaaagcctagaacgctttggcttagagaggcccaagtcttttctcagtgctgcactgctggtacgtggcgtggtcccctctcttctctcagtacacactacccatgcagactatcactctcagtcttgtttatctcaaatacagagggtataactaactggaatgtatccagaacagtgaggccaaagtgtggggaagctccttaaccatgctgctgcatgaggaacagctggagagactgagaacatgaggcctaaagaggagactcagggagatgggatcacaatcttcaaatatttaaaagacatcaaggggaaaagagattaaacaaggtaatgtagctctagagagcaaatccaagagtgttgagtggaagtgaaagggaggctggtttcagtcagatagtaggaagaactttctagtatttggtactacaatgggaaagactattttgtgagatttttttaaatttttttttaattatactttaagttctagggtacatgtgcacaacgtgcaggtttgttacatacgtatacatgtgccatgttggtgtgctgtacccattaactcatcatttaacattaggtatatctcctaatgctatccctccccgctccccccaccccacaacaggccccggtgtgtgatgttccccttcctgtgtccaagtgttctcactgttcaattcccacctatgagtgagaacatgcggtgtttggttttttgtccttgcaatagtttgctgagaatgatggtttccagcttcatccatgttcctacaaaggacatgaactcatcattttttatggctgcatagtattccatggtgtatatgtgccacattttcttaatccagtctatcattgttggacatttgggttggttccaagtctttgctattgtgaatagtgcctcaataaacatacatgtgcatgtgtctttatagcagcatgacttaaaatcctttgggtatatacccagtaatgggatggctgggtcaaatggtatttctagttctagatccctgaggaataaatgaccaactattgagaaattgcagggtagtccctacatgagggttaggtagaattgacctgctttctgcctcataaattttagaaaattaataagataatttattacggggtggtgtttgttccctcagtactttatcatctatgttgataatgttaataattaattgcataattaacaaatagcaaattattgtgggggtgtgtgtgtgtgtgtgtgtgtgtgtttagacagggtcttgctgtgtcacccaggctggagtgcagtggcgtgatctcggctcactgcaacctgtgccttccaggttcaagccatcatcctgcctcagcctccctagtagctgggattacaggcgcctgccaccatgcccagctaatttttgtatttttaatagaaatgggatttcaccatgttggctaggctagtcttgaactcctgacatcaggtgatccatccgactcatttcccaaagtgctgggattacaggcatgggccatcatgcctggcccgcaaattgttgttatttataactcttcaatccaaatcatcagtgtctatgttgtttccttaactatcaaatgatgataataatagtaccttcttcataagatagttgaaaggtttttaatatccatatggtactgagaatgatgcctgaaacatagtaactaccccatttttattatatttctgttaataataatacataccattattgctcttgcataccatattgctcttgcataccatatatgctcttgctatatgctacacacagtatttcatttaggcctcactatgtccctgatgtaggcattaatatctttattttgcaaatgagaaaacagtctgtaccttgtatgccatgctgctattgtttatctgtttgaatctcaagcaaatctgcttgataattggtaccaaaataagcctttttctgggtaaggaatctgatattgtgttttaaaaaacacacatttaatcctggggctgctgcattactcctgctgccccatcctactgtgatcaaaggcacatacatgagatggtgagttgtccccttgccaatgagggtttggtaagaaaggaaagtgcagtacttctttgtttctgaattgcaagtatgtgtgggttagagggggaggctgaatatgaaggtcctgggacagcccaccaggtatcccatgagactttgcaaaggaaaaggaggtgagtgacagcccagggtccaataggatagaaggaaaagccaggccatggagttcctcagacctgctttctaagggcaactctaccacctcagcaagccattgaacttctctgagctcagtcctttcatttataaaatggggtgacagtgctcacatgccaggaatacaaagggattgaaagataaaacacgtaattaagcacctgttgttacacatctgtcagggaccccaataaggtcagctgtcttcctgttgacttctgttcttggtggttctccaagatcataccttccatcaacatttaccgtcactcccccaccccatgcccaatactgaacagtggagggacgcttcacctacagttataatgttgaaacttcaacccaaagcaagtactgttaggatctctggaaactttccctcaaataagggatttgaatgggacaagaagaagttttacagatagccaatggagatgatttaatggggttatgatagaaacgagaaagtaaaacaaacccatgctttaaagtctaccatttcaggtccatattttcgcttgaaaattgagattcctattaaacaatgacatttacaccaaaaagtagaggagttggttgaaggacagggtaatgccaggaggaattgggaatttgagagtcaagtcaaaggactgaaatactcagaatactaagggcacctcagggctctaccaaggacacgtagaagctttgaatttgcagcaccaccctaatttaacgagctacctcagcacgtagtggagccttggaaaacagatgtcacaaactctcattagattgtcaaacattttccagcatttcctctcccatcatagctggttatcaagatatatagacacacacgtgcatacacataaataccttgataagttactagagaaagcagaaaaatgtctgacagtttaatgagatttgggtgaaagaaaattctatatttcattgttttccaggcactagaaataattcatcaatgtttctaagactcattcagcgtggctgcattttttaaaatattttcataaattttgaggagcaaataccattattaggcactaaaaaggttgaagtctaatagattagccgcttcatcctccttcactcagctcagcattcgttcaactggctcttactggttaacatccacacgcctcctgactggctactcagtgccgatgacatttccttcacacacagggctggttttaagatacattgaggtgacatcaggtggcctgtaaagtggtcattttaggatatcctattcaaagacatctgtggaagtgtggaccaatttattgatgaataacagtgaaggggtttccaccagcaagtaacataattttttacaatgatgatgctgaagtagaaagagtttctagtcagggactggacaaatcaatttgcagacgatttttaggaagaaaaacattgcaacagtaaattgtaattgataacttctagagccactttaagtactgctattttaggattctgaggggaagaaagtgttctgcaaagcaataagcaaagtgatttgttccaagccccaaatttaagcagtttgagaggtaaaaagagtcattaccaatgtgggtatagaacatgtgctaggcaaatctctttcacatacatgtggggaggtaataaaattataatttgaggccgggcacggtggctcatgcctgtaatcccagcactttgggaggctgaggtgggtggatcacgagatcaagagattgagaccatcctggccaacatggtaaaacctcgtctctactaaaaatacaaaaaattagctgggcatggtggcacacacctgtagtcccagctgctcgggaggctgaggtgggagaatcacttgaacctgggaggcagaggttgcattgagttgaaatcacgccactgcactccagcctggtgacagagcaagactccatctcaaaaacaaacaaaaattatagtatgaaataggcattaaaatattgtgtattttagaggagactgaggattggaggctgaagaattactctaaattaatcagcttgtgtacttcagagctaagatagctctttgggttctaaattctgtgatcttctttttgatttctcttggagcaataatgaaggcaaaacatcaataaacataacaaactgggtaagggagaccattgagaaggactaaggacaccttcaaagttctgagtgagtttaaaaagaagaatgatgaaaactttgatagaaataggaaaaaaagtagaggaacttgtttggcttgaaacttcttaatgtttaggctaattatattgaagatgacagtggtcattgagaaaacaaaatccccaaagcaattttggaataagagccaacatttaatacttaccagacaactattctaagtattttactatattcactcatagcaactctaaaaagcaggtagtattaacagagaaaatgaggcacagtgaggttaaatagctggtccgaggctacacagctaatcagtgggagagttgggacttagacccagaggtccagttttgaagtccacacttttagccattacactacaatggaaagaaatttagaagatatacacagaaaactataggcacatagattaggggttagtagaatgctctgggcagttaaaggaactcttcttaaaggaggtaaagcttgaatgagactgttagtaagctatttttcactcattggtgaatgatgttttgtgcagtgtgtttttttccccatagaaaaataagaaagaaaagaaaattgagaactctctctataaaaatgtgtaacatatctcatattccaagagatccttttggtagtattaatttttatctgctcacagtactggcttcattatttggagttaaaaattaactcaaccagataaaaaaatcagtgctgtgtatttgtttatctttcaaatctgtgttctaattttaaaaagttatttaacagaacgaagctatcagctaagacaatggcaaagccgtaaacaaacataggttgcgtttatgcgaatggtcaggtccaaagtagatgcagaatatgccaggttcactaattttaatccctattcagcccaggactatgtaccataagattactgctagtgttttctgaaaatgatgtatcaaggcattttctgtagaaatacgaaacagtgacatacagtagggagagctggattgaggcagagtagtatagatggaagtttcctgaaagcattttggggaaacatcttttgggtatggttcttggatgaagagttgatttattagtactggaagggtgtatgggagagaggaagtgagaggttatgagagaatgaccctcccgtgatggtgagtgggagaattattgcagtatgtacgttagcattgctatgtggtgaagttcttgggatttcctggggtccgtgctggacagcatgcttagccaccagtcacatgtgggcactgagcactgacaatgtgggtagtctgaactgggatatgctgtaagtgtaaaatacaaactggactccaaagatttagtatgaaaaaaagaatttgaaatatctcattaatgatgtgtatttggtttcatattgaaaacaactttggtattatatattgagttaaataaaatgtcattaaaattaaattttacttaaactaaaatttaaaattctatctttacctttttttttttttttttttttttttgaggtgccgtttcactcttgttgtccaggcttgagtgcaatggtgcgatcttggctcaccacaacctccgtctcctgggttcaagcgattctcctgcctcagcctccctggtagctgggattgcaggcacgcaccaccacgcctggctaattttttatttttagtagagacagggtttctccatgttggtcaggctggtctcaaactcccaacctcaggtgatccgtccacctcggcctcccaaagggctgggattacaggtgtgagccaccgtgcctggcctatctttacctttttaaggtagtgactagcaactttaagattcatatgtggctcatgctgtatttctattatggaactgccttatgactttcaatgggtaggatggacacatccttggtgggatggagaaatctatcatagcagctggtcttgaaggtgggtggggatatgataataacttaggtggggaggctcaggaggactcacagaataactggcaaccctgcccctgtctgtaaaaaccccatcctggaggaaataagttaggaaaaggttttgcattttgtggaatgaaaagtctgttgcatctagcttgagacagagcaaaaagagttgattgtcagcttcatgaagaccagggggtctaaaagacccagggatcaacaaccaatgagagcagcatggaggccaagaaccaggcaaaatgctgattccaggactgggattcaggatgatttccttctatgcaataatctgctccttgaaagggtatctaattgggcattgcttttacttgctgctttcaattcttttatgttctttcctagtaaatattttttcttaatttcattgcagctcgtatttatcctgggaacagagagaaatgtttcacaagcttaagccagtcttttaaaaggagaatggcaggactccaaaaacagacatgctgatatgtactggggaatttttaagtgctgaaacctccaagacaaaagagactgtgtctttattgttctctgaattactcgtacccagctcggtacctggaacatgatagggatcccatagtggtttgatgaataaattagtgactccaagagtaaagtaatcctcaggaggacaaaggcagatagcttcccttccctatcagaatgtacttctcttaaagcttttcttggtataattcttggagaattttgccttacagaagtcaaatcacataccaaagtgaaaactggatcttctacaaataatggaagaatcaactctatcaaaacaacaattatacatatgatcaatggaggggttgtcacgagccaggctaagagctttacatatattatctcattctgtctatgccagagaatcaactatgacatatgtaacattaaatctcattttatagatgcaaaaactggggtgtaaagaagtcaaagaatcagccagaatgtacagaattagcaaaggtggaactgggatttgaattcagacagtctgactccagacgccatctccgaattatgcataattatatttcaattattaacattcataaattgaaatatgagggataatgtaccttttcatgaaagctttgctcgttgtgtggatgagtgtgtgtacatgtaactgcttatgtgtgctatcactgaggtagaagacatctctctctctctctctctctctctctctctgttttggtctacttttagtaagacttgtatttgattgagttcagaagtttgattatctttttaactaacctgtttgttttaattatattaaaaattagtcactttcaacatatttgcataggtaattgttagggtgatcttttggatgatcagatgtaatatactactactacacacagacacccagacacacacacacacacacacacacacacgcatgcagacacatccttgagctcaaagagctttctcaggactacatactttacatttacaacaagtcatttagaaaactatcaatcctattcaaatctcagcaaaacagagataccaggctctgcctttctctttaactgctctttttttgctgtagacaaagctgtttcttgcactgctacatatataaaaaagtgacaaatccttaactgtcaaacaagagaaatagtttgataaatataataattccataagatggcacattatacactactaaattgtaaggacagtaaagttactgttaagtaccaaaaagtcatgattaaatgttaagtgaaaaaatagaatatagctagatttgaatttgaatattcaatctgtataccagtatgtatagaaggaagagtatataccaaatagtaagagtatctatctgttttataatttgatataatacaaattattctaccttaaacatatgagaatttgtggtccatgatgttgtagattctatcttctcaccctgcatttccgaagacatatggtattggctcattagactatttgttgaatagtctttattctattatcatagaaaaaataaatgagtgcatatatccatatacaaaatagaggtctgttcttcctgtatatatttatactaaaaaaactgagacttttttttacagttgtatatatacaaacatatttgtttatttatatacacatatataaatcaattttatgtacatgtgggtatacatacatccatgcatataactctgaagtgctgactctctaaagaaagcccaggtattggtcagaattcatgctcggctcaggagtatagaattaagagatacaaacctcaaaaaagagggaaccgaatcttcaaatctgagccaccttacaagaatttttaaggtaactgttttaagtgtaaacattatggcaatgtaatagtataatttgtatggcacaagatggagtcctggtggccagaactgagtatgggaatagtcagtgctaatctttgtgcaaagcacaaaggaaaattggtatacaaggcctgagagagaggtcaaggaagcaaatactataaagtcccagaaggcagttggataggacaaggggatcggaagagcaggatcaagatcagagagcagggaagatggggctagctaatctagggaatggagggagaagggatacccagaagcaggattcagggggttagagcacttacaccatgctcatggccatgcccatggacaggagaagcatatgatgggtgcacatggtggagctggtctaaagaagagggggattcatatcacagtaaaattaagtgcaatttatccacagagcagaggaggttgtgaagagcaggcataggctatttttcatgtctgaatgctctcaagacctcttagtgttgtaggtagatgacgcacaataaatattccttgatttgaattggcataaaaggtcaaatcagaaagccagaagttctttaaggtttcaaactagattctaagaagtccaaaggcatctcagaggtcatctcagggagaaaaggggaagcaatgaggtagaactctgaacctccactctacctaaacaaggcagccctgctgtgctccgagtcatgtatggcgacatcagggaagatgtaaaataggattgtgataggaaacagcatttgaaagccattgctttacgaagggaagcgtagaacccttttccccttgttagccaattcaaatgaagacttttgggagctagtgaagagaaagacaggatttctagggagatgtttcagaagcagcctaactatacccgtgtcttcagaaagagcagtgtcgtctcagaagtaatcaccttcatcaaccagcaggtcagtgtgggtctcctgaagagcccgaacaaccacgggaagcgacatccactgttgtgcagtcaaaagaatctttgctctcattttctccactctctttccttcccaaatagggtatcataggaagatcctgcctttcttccagttccaacatttatgaagtgaaatttccatcagacagttgcttttgacaaacaaagattgactagaagcttctgtgagagagccttaggaagttctctggggaagccctgcctttgtttggttttctttgcctttggctctgtgatgtttgctgtaaaaaatgattactttcaggtgattaaaagtggggaagaatggtttcaagcttttcatgtagcaaataatatccctgtctgtaggattactttagttgaaaaaaaaacatggcttcagtgatgccttctcaatgtacaagattcagaggaatggaaagaaaatgaaataaggccggcctcggtggctcacacctgtaatcccgggaagccgaggcgggtggatcacgaggtcaggagttcaagaccagcctggccaagatggtgaaaccccatctctactaaaaatacaaaaattagccaggcatggtggcaggcgcctatactcccagctactcaggaggctaaggcagagaattgcttgaaccccggaggcggaggttgcagtgagccaagatcatgccactgcagtccagcctgggcgacagagcaagactccatctcaaaaacaaaacaaaacaaaacaaaggaaaagaaatctgcagttaatattttggcaagctttcttcacttgtatgcatttttaaaatgctaatgttaataacagttcgggacttctaacttctatatttaagcaacaaataaataaattgtcagatggtacttcatcatccttctctcccatcttcttagaaatataaattgctttaggtgggaatgctataattttagaccagaaaatacatgccagatgtctcttatatgaagccgtcccgcccaaggatatatatatgccttagtcattaggatgtgttctaaataatactgcaaagcccttggaaggatgggtctgaacactcacttatatttaactgctggcatgttgctttgtccctgtgtcttgtgctactatttccattgatgtaaaggaagcaccaattaaataacactccattattagagaaccaggcacaagtcagctgaggcaggagacccgccttcttttccagaaacaatgtaaagcctgggtgggtgagggtctctgggcttccgccgtgccttgcttttgacattctccagcacaccctataaacatgtctaaggctgtcctgtttagtctgattattcaaactatattgtccagggtagagcaaagggaaacctagctgaaccctggagatgacagcagggagagagagaggggcaaagaagggcaaaacgggaaaaacaggaaacaggctagtgagaagagtaaaaacgctcagggtgaggaagcagggtttctaagctctctaatctcccctgtgcagctggcttgctgtatggtttatacaaatccagtggtgatctctgtgcaacgtggtatcacctgtttaaagaggtctcatcttcattttcaaagaggaatacatgtttttttacttactcttctgcatggctgactccttttcatgctttaagtctcaatcttaatgccacctcctccttccagacgttcccagctaaagtggcacttcccagccccattactctctatgtttattgcctgcatagctcttatttgtaatgatttcgtaatagtttgatgatgatcatgatgaatattactttacctatttatggcctctcttttagtattaaattctgtaagccacatgagcatggggacacatctcctttgtcactgccccattgctggcatttagcacaagcatggtctataatagataccaaacaaatatgtattaatcatgtaaatgactaaatccatgaatgaatctatcagacagtgtagatagcagcacataaaggaaagggaatgtagtaaatttttcattttccttgaagatgtagctatgtattaggaatttgaaaaatacattatcaaacacaaagctaaattatgccagctaatgactactaaatataataaaatcggctgggcacggtggctcgcacctgtaatcccagcactttgggaggccgaggtgggtggatcacgaggtcaggagattgagaccatcctggcaaacatggtgaaacgctgtctctactaaaaatacaaaaaattagctgggcatggtagcaggcacctgtaatcccagctactcgggaggctgaggcaggagaatcgcttgaacccaggaggcagagcttgcagtgagcagagatcacaccactgcactccagcctgggcgacagagtgaacctctgtctcaaaaataaataaataaatataataaagtatgtagaaagtcagaaatcttggggattatattgcaaagaatttccactatattgataatggagaaaggcttttaatattatattttttgaatattaagaaattggcatctactcaccagtttggacatcgcttttaaaatacacactaaacgaaagccattttgtacttataagtgctagattaaattcctgcataggctgaaaaaggtctctttccatgcctttccaaatttacaattaacaaagagttaattattctcagagtcatttcttccaattcaccaattaggatgagggctatttgttacaatcataaaagaggaaatggtgcatgggcaagaagaaatttggaaaggaaatgtgattggaggaattatattgaaaggtgaaacaagggagaaaagataaagagaagaaaaattagaaattggaaacaaagttattccagcccctctctaataactactactctttggaacaagggaagcagtacctgacaagaaattttttttcttttattttttatttttattattatactttaagttttagggtacatgtgcacaatgtgcaggtttgttacatatgtatacatgtgccgtgctggtgtgctacacccattaactcgtcatttagcattagttatatctcccaatgctatccctcccccctccccccaccccacagcagtccccagagtgtgatgttccccttcctgtgtccatgtgttctcattgttcaattcccatctatgagtaagaacatgcagtgtttggttttttgtccttgggatagtttactgagaatgatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccacaataaatatacgtgtgcatgtgtctttatagcagcatgatttatagtcctttgggtatatacccagtaatgggatgtctgggtcaaatgatatttctagttctagatccctgaggaatcaccacactgacttccacaatggttgaactagtttacagtcccaccaacagtgtaaaagtgtccctatttctccacagcctctccagcacctgttgtttcctgactttttaatgattgccattctaactggtgtgagatggtatctcattgtggttttgatttgcatttctctgatggccagtgatggtgagcattttttcatgtgtcttttggctgcaaaaatgtcttcttttgagaagtgtctgttcatatcctccgcccactttttgatggggttgtttgtttttttcttgtaaatttgtttgagttcattgtagattctggatattagccctttgtcagatgagtaggttgcgaaaattttctcccattttgtaggttgcctgttcactctgatggtagtttcttttgctgtgcagaagctctttagtttaattagatcccatttgtcaattttgtcttttgttgccattgcttttggtgttttagacatgaagtccttgcccatgcctatgtcctgaatggtaatgcctaggttttcttctagggttcttatggttttaggtctaacatttaagtctttaatccatcttgaattaatttttgtataaggtgtaaggaagggatccagtttcagctttctccatatggctagccagttttcccagcaccatttattaaatagggaatcctttccccattgcttgtttttctcaggtttgtcaaagatcagatagttgtagatatggggcgttatttctgagggctctgttctgttccattgatctatatctctgttttggtaccagtaccatgctgtttgggttactgtagccttgtagtatagtttgaagtcaggtaatgtgatgcctccagttttgttcttttggcttaggattgacttggcgatgcgggctcttttttggtgccatatgaactttaaagtagttttttccaattctgtgaagaaagtcattggtagcttgatggggatggcattgaatctataaattaccttgggcagtatggccattttcacgatattgattcttcctacccatgagcatggaatgttcttccatttgtttgtatcctcttttattttattgagcagtggtttgtagttctccttgaagaggtccttcacgtcccttgtaagttggattcctaagtattttattctctttgaagcaattgtgaatgggagttcactcatgatttggctctctgtctgttattggtgtataagaatgcttgtgatttttgtacattgattttgtatcctgagactttgctgaagttgcttatcagcttaaggagatactggcaaaaaccacatgattatctcaatagatgcagaaaaggccttgacaaaattcaacaacccttcatgccaaaaactctcaataaattaggtattgatgggacatatctcaaaataataagagctatctatgacaaacccacagccaatatcatactgaatgggcaaaaactggaagcattccctttgaaaactggcacaagacagggatgccctctcttaccactcctattcaacatcgtgttggaagttctggccagggcaattaggcaggagaaggaaataaagggtattcagttaggaaaagaggaagtcaaattgtccctgtttgcagacgacatgattgtatatctagaaaacctcattgtctgacaagaaattttatagtctgatgaaagggattctaaagagtcaggggccacaggtctcaggcttcgactggatgtgatcatgtctgaggcctttcgatcctcactttccttatctggaaaacaagaatagctgaatctccttctaagggcgtttgtgatatgaactgagatcttgcatatgactgcaccaagtctagctcaattcgcattagttccctccattatacccctccctcgagctttacccagactcagaagaaagccaggcaacatttctacttctctatatgcaaaaacaaaagcaaacaagtggaaaacctcacaaaaacagttaacttcaacatttgggcttacacaaacaattcaaaaatctctttttatttcatccgccatgattatagttatttttctaaagtgaatgattctacttcccaaatgcagtaaacccactgttaaagatagttaattttcctctagatgattgtggcccttgaaagtcatcaaggtcatatttttaattatttccccagaatttttcctgaaacagtgtccttttgtctaaatcaatccaagtaggttttagcattagtcataaagagggtgctgtcaacaaagaaatcaactgagtggaagtgatattataatgtaaataacttgacataggaatacataacactcataaatatttattgatttatttaataaattaaaaattaatgcttatgatatgtaaacaagatataacaaggcagtcaagaataactctctttagttcatatgattttttcccatacgtaactgaatagcaagaaaacaagtaacccagtttgaaaatggacaaaaaactgaaatagatatttctcaaaagaagacatacaaatggccaataggatattttttaaatgttactagtcatcaaggaaatgcaaatcaaaatgacaatgaactatcaccttacacttgttagaatggttactagcaaaaaaagacaagggataacaaggttggcaatgatgtagagaaaagggaatccttgtacattgttggagggaatgtaaattagtatagtcactatggaaaactgcatggaggagcttcaaaaaactgaaaataagcctaccatgtgatcctaatactgggtatatatccaaaggattggaaatcaatatgttgaagagatatctgcattcccatgttcgctgcagccttattcacaattgccaagtatgaaattggcttgagtgtccatcaacagatgaatggctatagaaaacatatacacagtggaatactattcagccttaaaaaagaaggcaatcctgtcatttgcaacaacatgatgaacctgtaggacattgtgctgagtaaaataagcctgtcacagaaagacaaatactgtataatctcatatgcagaatcttcaaaaagttgaacttataaaggtagagagtagagtgatgtttaccagagggtggggtggagaggggtggggtacagggaatgggagaatgttggtcaaagagtacaaagttttagttagacatgacaaataagttttcaatgctattgcacagtgtggtgaccataattaacaataatgtcttttatatctcaaaattgctgaaagaataggccttaaatgttttcagtatagaaaagtatatgagatgatgcctaatttaattagcttgatataatcattccacaatgtatacatatatgaaaacatcacattgtatcccataaatatatacaattattatttgttaattacaagtaaaagtttaaaaaatggcaagtaaatcatgtagcccctgagatagatggatatgtgagcctagcttgaaacaaatgtcatcatatatccaccattacacaagactttgagcagactgaatgctcacagaacatattgggaggagatatttggcagctgaagtggcaaatagtcattttccaagggaacaacaacagtagagaggtttccagttaaagttgcagagtttctgcagagtctctagcagtgctggatccaagggtatgcggtgtatccaagtagctcttgaggaaaccacaggcacatcctggcatggggagcacctcaggagcacatcctgagtttcagggcatttgaaatggatgtgcagtcacatcccacccaactgcaagggatacccagcctacatgcagaggtcaggaaagctgcccacattaagacattgcatgcaataggcccctccccactaggagtctatggagacagaaatgcattttgaggagcaatttcatgcagtcatgggttaagtgaaccaagtgagctatgaagccagattttccctcctgggccacatatttcagaggcacataactcaagcttgcaacacgtattcaaaagagaccagctacacttggtagagacagccataggaaagtgaaatgaccctagggtttagtaaagccagctgtttccacttctgaaaataataaaatgaaataataaaataaatttaaaatgatacaaagttcaaagtttaacaaatacatttgaagccatttgcaacaaatacatctgaagctaattgctggctctagaaagtgtggggtctttgttgtggagcagtgttaatgatttagcattacttatctctggcaaatggtatttttgagataacatgttatggaagaaagtgaactgaacttggaagtttgaagatctcgattgaagtatcatttctgcctcaactacttgcattaacttgtacaagtcattcaaccgctctgaacataatggaaaaatgggatgagaatacatgttgtatactctccaaagacagggagactgctgatataagagggcacttttagtaactgatggagcaaaatgttgttatatgagtgtcagcatagggccctgggcttacaacggtgccatgagccttagaacagaggaaggacagctatagcaatgaaaggactagtgcagattcagaaaaataagaagacagaaaccaaggtgtagtaacatgttttagtatggaggggaaggcagttatagaaacttgaattacataatttgtacatttctgggagatagaaggtaaagatagcagctaatggagacaggacaggactggtacttgattatggaagaaaggaggtaaatagaagagacaaaaagggagagaagagatgtcaactgcctactctggtagcctctgtatccaaaaggttgactcaaacattcgctcataactttgtctggcttaatcctgctcatcccagcagacttatttcaagtgtctccacgttttgggaagtcatcactcacttctctgggctttcatatgggagagcatttaattctgttgaaaaactatttaatactacatctacctttctctatggactctgagcttcttgagggcatgtatcatgtatgttctattctgaagcacccatacctagaacaaagcttagcacatagtaggaacttaataaatatttcggagttgaataactagccttatgtaatcctcacaacaaccctaagctggagactcaaacaaggctggaaataagtaggtgccaagaagaactgagattcagacacatatttgcaggtaaaacataggaacactgaacattcactgagaactgacaacttgtggggttgttgtaggatatgtgaccagagactcttgaatgccagtctctgtacctgtaccatgttggctaacaagaatcgcatggaatccttgctgaaaatacagaccctagaagttttctcaaatctggagagactgtactatggtttgaatatggtttgtcgccaccagaactcatgttgaggcttggtcctcaatgcagctgtgttgggaggtgggacctagagggaagtgtttgggtccagtgggcagatccctcatgaacagataaatattgtcttgtgggagtggatgacttctgtcttgcaggactggatgaattaccacaagaatgagttgttgtgaagcttctcctcatgttttgctgtgtttgcacgctgtctcttgacatttttcttctttgctatgttgtaaggcagcacataaccctctgcaagctgagcagatgccagtgccatgctcttggattttctagccactagagttgtgagccaaataacattttttttctttataaattacccagatcaggtattctgttagagcaacactaaagggactaagacactcttattctcaccaaatctttattttggtaatgatttctcacacctattcatttgctccagaaaaggtagttattctccatagtctatcttcatcttccacttcatgcttattcaatccattaccaattcctgtcaatatatcttcctaaatatctctttcaaccaccaacttttctatcctcactattactatccttttacaagcacatagaccactgccaggaacccttgacttgccgacctgactctatactagttcttcttcctactgcagccggagcaatctttttaatcaaagctatgactcaacatttactctcttgatgaaatctcctagaagaccttttgtagctcttagaacaaagactgaaataaaaactctatactatagtctaaaagttccttgttggtctcacctctccagcttcctccctctgtgctccaaccacatgggcttgccttcaatgcttcacatatcacccagcttcaaattcccttctggctgcagggccttggcacaaactgttttctctgcctgatgttttccacccttccacctacattcatcagtttgacttctacttatcttttggagctcagctcagacaaggttagatcctgccattgacatactcattagcaccctgaaatatttcttaatcacagcgtatgattatatatttatttgtgtgattaactgataaatgactgtctgacccctcctccctgctttaagactataagttgtattaattcagggcctatgttagctttactcagtactctgtacccaatgcccaccccagcatcctcacaagtaagggtgttcagtacatgggtgttgaatagatgcatgaataatacaataagtcaacaattggtctcaggaatctcaataattttaatgctatcaaagtgatttgatgcagatttgggaaaaattatctaaaaaattcatcccaagctaagatcctatgattcttagctatcacagaatctgtgattctgtgctactcctctgcgcttctcatgtacacttacatgggtatacccatgaaaaatgtttgttggtttgtttgtttgttttgagacagagtttaactcttgttgtccaggctggagtgcaatggtgcaatctcggctcactgcaaactccacctcctggcctcaagggactctcctgccccagcctccagagtagctgagattacaggcgcatgtcactacactcagctaatttttatatttttagtagagacagagtttcaccatgttggccaggttggtgtcaaactcctgatctcaggtgatctactgcctgagcctcccaaagtgctgggattacaggcgtgagccaccgtgcccagccgaaaaatgttttaaagcatctaggatccttggcaggcctttagcacactgcacagaagggacattctgtgcctgtcactggaatgaccagcaactctggttccctgctttgcccagactgtttctatgtccccttcagtttagttcagttcaacaattatctagtgagcactttctctgagcggggcatctgctttgtgctaagtgtaagccctgcctccaagaactcattgcataaggagagacacacacatgaaaaccaactaattgtgattcagtgtaaaatacgtagtaattgacaaatgcatatagtttcatgacagccctgtagaaggagtaggcaagtgttctagtatggcttcaaggaggaaatgtagcttaaaacaggttcggagggatgagtaagagtttaccatacttttaagggggttgggaggaatatattgaaatgaaaattacattttgcaaatgcagtatgaggagccgtggtacagtttaatgtgtttagagaacaacgagtaactgtatggcagaagcagtaaatatgacgaaggaggaagctggtgtgtttggagaaggctgagggatcatgaggcatattaccttcctttaaaagccatgccctattctccctctcctgccacttcaaattcaggttcaccatttatatgtctattagtcctggtgtctttctcatgctctttgattagtccttaatccacaagcgcaacattgcaatacttgcctagcatatttcacaggcaggggacctaatgtccctgtgagaacccatcttgctgagattgtcgctggcagatttacttccagtgtgattgttgcaagaatttgtctaacagaatgaatgatcaaccttgagcagaagagattatgaaaaacttaatagcattgtagcaatgtggctgttaatgaaatacagttggctgctcccgctgtttggcaccaaccaacctgacactgtcaacatcacaatacgatatttattcccaattattttacggcaacaactgaaatacaatgtgttattaatcatatttattataagtatcaatttgagaaatttctgacatgccagaagataaataggtttattatgaaaagcagttctgcttggtgcatgctggctgctgctgtgtaataaatagcctctgtggggaaagttttttaaaagaaataaagcaaaaaaatagcactgaaaacagaaaggaagcatcaaaactcttcaaatacctgctgtgtccattggtcaagcacattcaggacatcgcatgcctttagaactccagcaggttccaacagctagtaggacattctagactctgagagagagcaagggaggttttatgactggggacaaagaaaagagacactgaaggcgaaggacaatctctgaaaatgcagtaccctccagactgctcctcctctcacaaaaacaccttcccagcatgcactgctttagggactatgattataccattgattctgtccagaaaacctgtgtcctgaatatattacagggctcattccttcacttctttcaggtgcctactcaggtatttccttatcagaacagtctttcgaacgaccccattaaaaaaatagtcctgtcaaccctatgttaacaattttatttatttttattatttgttaacaatacataataggtgcatatattttgggggtacatataataatttgatacattcatattgtgcataaagattgaatcggagtaattgggatatccattgccttaagtgttttaccttttctttatgctgtggacattcaaattactttctaactttttgaaatatacaatagaagaatgttaactataatcaccctattgatctatcaaatgctagatcttatttcttctaactatatattgtacctattaatctgtaattccacaactatatttacttcttatacttttccccttctaggctataaaccaaatgagagctgagcatctgtttggttcactgcccaacacatgcatgcctactacatggcagtcaaaatatttgtggaataaatgaatgaatgaaaaaaaaaagaaatagatgaatgaatcatggatgaatgaatcaaatcagtcagcaatgtctttctaaacaaaatttggatgattttggatgattacgcctcttaaaaatatttcttcatttcctaccccaatttagtttctactcaggactttttcaatatcttccaaacctattgttcttttttatttgtttgctttttgaggcaaggtcttgctctgttgccagggctagagtgcagtggtgtgatcacagctcactaaagcctccaactcttgggttcaagtgattctccacctcagcctcccaaatagctgggatgaaagtgtacaccacaatgcccggagaattatttcatttcttctttgtagagattgagtcttactctgttgaccaggttgctttcgaactcctggcctcaagccatccttccacctcagccttcccaagtgctaggattacaggcgcgagccaacttgcccagccctggaatttttgagcctgttcaattctaactattgtcaccaaaagtaaccttaagaaaaaaaatgcattatctccttgcttcattgcaccattaaaatctttcctaaattttccatgttaaagatgaagctcaaaatcctcagcatagcatacaaaacacttcataatcagatgcctcttcaaatacctcctatcagaatggtctctttgactacccctttaaaaaaattccccccaaccctatttttaaattatttacttatttttattatatttttgatacataatagatggacacattcaaacagtgcccccaaaactggggcagcagaaacaggtccttgcttattttctcagcttcacctcctgcctccaccccatctgtactgctggtccagacattcctacagaggtgtcctcctaagttggtctcttcctctcctgcttcagaggctttgccctgctcttctctgcctcttgaggctctgtcctgctcttctctgcgtcttgtggttggaatgcctgtctttttcctactgaagatctggatgcctaaaccataatgtaaaattgctgctttttacttccatttacagcagagaaattcctcctctggcctctcctcttctctgtgtttctttcttcataatttttatttatttatatatttatttatttatttatttatttatttattttcattgagatggagtctcgttctgtcgcccaggctagagtgcagtggtgtgatctcagctcactgcaatctccaccttccaggttcaagcgaacctcttgcttcagcctccctcctgtagctgggactacaggtgcccgccaccacccctggctagttttcatatttttagtagagacagggtttcaccatgttggccaggctattctcaaactcctgatctcaagtgatctgcccaccttggcctcccaaagtgctgggattacaggcgtgagtcaccgcacccagcctctctctttataattttcctactgttcacctgcatcaaactcctgaattctgtcatgcaactggaacagtaagagggaaaaacatggagctcaaagaaagatgttgagaaacgtagagttgcatagaatttactgtataagaatggaatctgtcaagtcagacaagcgacagagacctatttacaaagagacccagtgaaaattactggagaaataataaagagaaatgctgtgactttgaaataaataatgttcaaaagtcacctgcaatatttaggatagtgtctgaaacagatacaaatatttctcagcagtaaaagaattttgtatttagtctagtcatggaatagtagtcagttgtcactgaggaagcactttggggtagaagaagcatttgaatgtgtttgaagtctgaggcagcaggtgaggtttgattttatatttttgaaaatggatctatcagatggtggagctaccctcataaaagatttgtaatacgcctgtttacctacaagattaaatcaagtgtcatttcttcagggaagattgcccttcaccatgtgaaatatacataaagtatatgtcacaatgtgtgacagttctttgctcacatatatatttctccacttaaaggagaattttttttgagatatgatcttgttctgttatccaggatggggtgcaatggggcaatcacggctcattgcagccttcacctcctgggttcaagtggtcctcccacatcagcctcctgagtagctgagactacaagtgtgcactatcaagcctggctaatgtttaatttttagtagagacaaggtcttgctacatttcccaggctggtcttgaattcctggcctcaagtgatcctcccaccttagcccagaaggagtcttatttcattcatcgtatattcttagtatctacctgtcatcaggcctatagtagatactcagtagatgtagattgaggtttgaagaataagagatagctcaccaagtagaacactggatggtattggaactaatgtattcctttattgtcagcagaatggaccatgcacatagaaataataaaatgggagaattgattgccatggtctaaattttgtgccccctacaattcatatgttgagaccctgacccccaaggtgccggtactaagaagtggggcctttgggcagtgataaggttgtgagggtggagccctcacaaatgggatttatgccctcataaaagaaactgcagagaactagttaaacccttctactatctgaggacatggcaagaagctgctgttctctgaacctggatagaggacctcactagacgctgaccagcgctttcatcttggatttcccaggctccaaaactgtgagaaataagtttctgttgtttataagctaccaggtttaaggtattttgtacagcaacccaaagagtctgagaccataatgaagccattggaatggtgggaaggcaacttcatgtgagtaactacagtaaagccaggtgctggtaacagtcatgttgcccatagagcagatcctactattacagtgcctagcacattacctgcatatgatgatatgtgatcaattagttaactgattagtttatgaatcagtctgccaaaaactagggcagaaattgatagcacattaaaataaatatgccttaaagtttgcaaggagaccctattaactgcgcactgttttctttttattttctttttttttcttttgagacagggtctcactctgtcacccaggctggagtgcaatggcacagtcttggctcattgcaacctccacttcccgggttcaagcgattcttgtgcctcagcctcccaagtagttgggaatacaggtgtgcaccaccacacctggctaatttttatatttttagtagatagggggtttcccatattagccagcctggtcctgaactcctggcctcaagtgatctacctgccttggcctctcaaagtgctgggattacaggcgtgagccactgcacctggctagccactcactgttttcatgttaggctaagtaagcttttttgaagaccattaacataaatatacaacctaaatgtattttacctgaataattttactcatgtccacagcttgttctttcataggctgccatgatgaggaagaacagagattagtagtagcactattcatttctgatatttttgcagtagtggttctaattctcactccagtttagaaaagatctgtaggaaatcacaggtctacatttcgtcctctaaactactctgttgggtagaatttattttgcaaagacttatgtaggatcacttttttactacaggttttgtcatatgggatttttacaacctttttttctgctgaaacaaatggcttttaatccttaaaagggcagggctatattttccttcaaacatttttaaaataacttaagagaattaatttttagtaatagcaagtgaagaacattttaatcctagagcttaagaaaggggaggcccaataaccagatgctggaaatctattgaggttttttttaaattccagtatccagacaattggcatgaaaataaaggagcctagaaaaaatgttgaaaatgaaaacaataaaagtgacaactaaacatattattttgtatttgcaaagcacttccacaaatggccacaaatgtcatctctttaaattttattcctgagttgagtaggaaataaaccatttggagattcaaggtttagcttaagatatgggtgtttcttgaacctgggaggtggaggtttcagtgagctgagatcatgccactgcactccagcctagatgacagagcgagagtttgtctcaaaaaaaaaaaaaaaaaaaaaaaaagatatggatgttgtcaatacaatcgggggaaaggaatactttgaactactttgttggaaggagtttgaaatcgttgaggactcagcagcatgaagtagagaaattcacaattggtagaaaggactattgtccttcaaccttcattaaggttaactattcaaccttcattaaaaacagaaagtgacaatttcacagcaaattctagaactttagatcaaaagtcaactcaatatgggggatttatataagaaagagttaaaaaaaagacgaaatgtaatatctatgttattgcaagtgaaaggaaaacaggaagataaatatcacaagaagacaaaaatgtatctaacattttgggacaagattgtgggatccacagaaaattggaacttggaacttcctgttccacagagataagaaatacacttgcttttatctcacttctcaaaaaaagtaagatgaatggggttttaggccccagagagaaattgtagctgcaatcaattgtactatctgagtaaaaattgtcctcagaggaaagtgagtagggagctgtctgaagggacaggttattaacaaaagagagggataatggattgcgtttgcaagtgcagttggggctaacatcaatgccatcttcatagctggttcaaaaaaatattctggattcttttagtgtcttggttcttacctgttgtggttgcagaaggtataaatgtacccttaaaagagattagggagagaagtgcctcccacagcaccacgaccagaaagggaagaggaaggacaggcaatagccaaggactcctggcagtgaactcatgtccacatcaagatctaatgagcttgcactcaactcatttctagctctgccttggaagctggagctcctgcactgactatcaatgtgagcccctgagtaggagcagcttggtagagttgaaagaccattgatctgggtcaacagactctggttcctgtctcagcagtgctgtaatcaatccaagtcaaatatcatctctgggacttaatttgctaaatttaaaatgaaaagaaaaacaaaaatagaacaattagactagatcaggattcggcaaaccaaagcctgcttatcaaatctggcataccacctgtttctgtctatacaattgtattgaaactcagccacactcattcatttgcatattgtccatgaaagaagcttttgcgctgcctcaggagatctgagtagtggccacagagatgttgcagtggaccatgttgcaacattgtccaaaatacctaaaatatttacttcttgttttggagagtttgctgactggcaccagagaaatctatggtctaaaatcatctaaaaatttaagcacatatgtgtgaccacacatttcgaaatgccgtcttcccaatctagaacacagcacatgaatcagtaggtaaacctatccatgtcaattctcaaatttgaaattcattcacttgaaaatccagcaatttttcatgcttcatatcatatctgggtttggaataaagaagtgtgaggggagaaaaattccctgagcattttaaatctaatttcacctttattatgagactactgagtcttttcttgagcaaaggagagagtgtgaaatagaataaggtgctcaaaacaatagattaaatttattgaaaggatgagtaattggagtaatgttacagaatattaagcagattatttagatagcatatacatttccagtttgattaagtcaattcacaggccatcaaaaagtacacagaaaaatagagggtttgatccgatagccttctgcgttagaatgtagtcatttgctctctttagctatttaatctcttctgcatgttcagagggagaaaatgagtgatgagagagagaagtaataagatcatactgcaaatccttacatcgataagataacagaaggcttttcacactggctaatatttcatgttgtatttacagctcttgtccatgtacagatttggggtcattaacagaggtgttgatcagcaaagtcttagagtgagtctagggaaatggttgccaaatatatctgggtattggaaccatcaacagagcctgtgccccagacttactgaatcatggtctctggtggtggatcccaggtagtcacagctttaactgagtgattaggagattcactgtgcagccagttcgggaaccaccagtgaaatccaaccccttcatttattagtgttggaaccgtaactttctgaagatgtaccactgtcccagccacttcacctgtattcccagctcatttctctcagagaaaactgccaagtccccacagtggccttgcatgggccatttgtgatgtcacctctctgacctcatctcctactttccttcacccggttgtgtctagctattgccatgctctttctccaacacaccaagcacactcctgcatcagaggctgtgaattttccatttcctgctttgttcctccccaatatttgcatgaaccactttcccacttaatctaattttctgttcagaagtcactttcacagaaaagtcttcctcagccacatgtattttcttcttggtgcttattatcacctgacattttatatatctatgtttgtttcttgtttgtttcctactccattcggtaagctctaattgagaacagaaaagctctgtttattcactgttgtatccccaacatctatatctagcacatcattgattctcaacaactatttgtaaatgaatgaataaatggctctgctagatttttgtcacctggattgccagaccagtgcaataatggaaagctaagtaatgtaaagagctttgcaatcagacagctctggcttgcattcttcatgtgtgattttgagcaagttactgattcttgctgagcatcagttttctatgtgtaaagtggaggcatggacactggaggatcatggtgagaatttcatagagcaggccaataagacctccttggggaaagaaggctaaagtgggcctcgggtttaggtttcccccactgggagttccaaggggctatggagttgataggattccccatttgggctgcagaggagccagaaacatgctttagtttcttaatcctgaaggagtgggggattgtgtaagcctcattggtgcccttttgaaccacatttcatcaagatattgtgaaatggggagcagcaggcagccttctctggccaacatgtatcagggcctcgtgcttagtgggatgtgcccagagtagacagggaatccctggcagccttgcatagtccctctcatgcctttgctttcatggggctggctggaaacaggccggtaagccccatggaactaccgggacaaattaccaagcaatctcccatggccatgtagaacagccatgtagaacaggatgtctcccaaggtaagatttaaggtttgtggtttgcagaaaagagaagggaggaaataaaaaagcactgacaatcattgaaagcccacgttctaggtaactggctactcactttcacagcccagtaacaattttgcttaatcctatacatctataaataggattattgtcttcattttccagataaggaaacaagacttggcaaatgtgtagtgtagtgtttgacagtattatgtagttgtttagcatgcacatgaacttgcaagaccttgctcttccctaataattctgtcctttactaattctgtgatcttacaagctgtgccttatgtattttgtgttctattttgtaaaacctaatgacaacaacataataataccttctcatagaacaattattgtgataaattaagctgttgagagtcaggcactcaacccaacaacatgcccataatgtgtactcaataaatgcttgctagtatttttaataatattgttaataaaataacccagccagaggtagaacccaagattccgactctagttgacctgattctaagccaactcttctacagcaacacatcggcccattgatttagacagcatggaactcatagcccagggaattcacactcttaaagaacttaaacagcacagcttaatatggtcccttccaaacatacgactcag

SEQ ID NO: 2 - DNA, 97335 bp, Homo sapiens
aaatgaattaattttacagacattacactgagttaaagaagacagacacaatgaggacctattatatgaagttctacaacaagcaaagtgaatctgtggtctatggtgatagaaggaaaaaagggattacttttttgtggttgcagaaaaacttactggatagggccatgagagaactatctggagtgatggacgtattctatatcttgatccatatgtggttacatgattatatatatttgccaaaatgtattgaactaaattattaggggaaaaattcagggtacataaatgctttcttaaatatatagggtcaagataagaaactctggataagaatccaagttttctaatatcaggtcatatactcttgtcttgaagccataggtcttcggcctaaagctactagagaatccgggttgctgacccctagacaaatgccatgactctagtagccagtgtaacccttttccctttgaagttgtagctaataaagtctagagttcaaagtcatgtcctgcatgggcaaagtgaatgttctagaataagagaaactatgctgagaatgaaagagtttttttgagacaatagaataattttaggtcctgaaccatgtgagaaaagtgttctaagaaaagctctcaagatctagaatgaggcagataaccaagacagcttgaaataaaccctgtaaatggactggggtagcttatcaggatttagagtcattctaatgacacttttgacaacagtgtccacatgattgatgctataaaactgataacggaaggcgatgcccttacaggaatggcattttggcaaaactcaatatcttctcacagttcattttttataagattctattcatggactgcatacttatacaatctcattagtctgccattaatcatttctatgtctgtattctgtagttttcaagacttgagtttgcatacatgaaaacaatatgctccattcatataacattttggtttacaaggcatttgggatctgaatcatgagacattcaggagaggagaggtgatgacttctgtacctatctgtaggtcgttgggtgagctaaagccatgatggagagtcaggtcccatgaggtccatgctcaaatgaaacctgaacaactaaaggaatgggaaaaacagagatgctggctttgagggtgatgaatttgtgagcactcaggctctctgatgctgtgattttgttactcctcgtgaatcagcttatgcataataatgtcacacctctctttagaatacattagataatacatttagaatacattatacctccacacaggtataacgtatcctggaagacataaagtgtcctcttaccttgtcctgaagtatttgccttatgcacagcattccaagccaggaagctcttgctaagtttaaaaatggatggatatgggccgggcgtggtggctaacgcctgtaatcccagcactttgggaggccgaagagggcggatcatgaggtcaggagattgagaccatcctggctaacactgtgaaaccccgtctctactaaaaatacaaaacattagccaggcgggcacctgtagtcccagctacttgggaggctgaggcaggagaacaggagaatggtgtgaactcgggaggcggagcttgcagtgagccgagatcatgccactgcactccagcctgggcgacagagcaagactccgtctcaggaaaaaaaaaaaaaaaaaaaaatggatggatatgaaggtggaggcagagttgggttaaggaatcattacacatgaagtatcctagttaccagaaaaaggtcactggactgaagcaggaaacttcttgttttggttctttaaggtattctactcagcataacagctcattctaatactctgtattagtttgctaagggtgtcataacaacattccacaaacctagtggcttaaacaacagaaatgtattgtcttgtagttctggaggccagaagtcacgatgtcgctggtgttggctcctcctgagggctgtgagggaagggtctattcctgatctttctttctggtttgtggatgccctttttcctgtgtctcctcacaccatcttccctctgtacatgtcttatccaaatttcccctttgtataagaacactaatcatgttggattaaggtccaccctaataatttcattttaacttgactaccactgtacagactctatctccaaatacggtcacactctggggcagtgaaggttaggactacaacgtatttgttttgggtgacacaattcaaccggtaacatatcctgatccttggctccctcatctgcaaaacaagtatatttaccttacactgttatttaaaaattgaaaagcagccaatgtatttagaagaacttagtaaactgaagattctacataagaacaaggcagtaaaaacagtgagcgataacagcatagatatacataataataagagctctctcttactgagaacagccatgtgcctggcagttctaaatgtattcacttatgaatctatgagatctcctatgaagattgatggtagcattatctctactttccagatgaggaaactgaaggacagcaagtttgggcaatttgctaacgacacagcttgtaagtggtatggccacttgtaagccactccattagaagccagagcctgtaagcttaatcacgtcccctccccacatacagatactagtgagagaaccaactttaaatttccagaataaagattgtttcaaaaatagaaatagatattaagaaataaaaatagaaattatttctatagacagctaggtaaatggaaaagcaatagttaaaatgtgaacatattttaagtatgatatactgtagctaaagataacatgcagtacttttaagtaatactgtatatagagaagtagatcgaaataggtaaactattaacataaattttcctgtagtggaatcccaattaaagcaataatgtagagcaataagaagcaaaatctgcttggatccaaatcatttctgaactttgtaactttggtcgtgttgctttctgtgaatcagtttcttcatttaaaaaagttgttatgaggatagaacatgctagtatttataaaatgctcataaattcttggcacaaagtaagctatagactcaaaaatggtgattccagacctcaagaaataagaacttgaatttcagagaaatcttcccttattatccctgaggtccactccatagcttgatcaaagaagtgggatactatgtagtagactagtgtggcagcaccttaaacccacaaataagtttggctgttgtaaagagaagggaaaccaaaattatttcaggacctactatgtgcagagtttttatctgctatatacataggcatatgctattccatcagtggttcagagggcataaacagttaataaagctatctcagggatgattaaattgagagttgctcagggtcatacaatttttgagtcaggaattaaacccagggctatttcattccaaggcccatgttactttctggtaaaacaaataaccatgaaatataatatattttctttgaatccctaatagtgggtgctattatacccccaattacttaaattgatcctgtacattgccaagagattaaattataaattattgagttcaaaaagttaatttcttcctccaaagagatggcatcttacagtttaattactctgacagctttttttttttattttttctcctagtgaattatcaacagaaactacctagagaaacctgtaacttcagatcttctgattaattgattttctttgggtgctgcttgacagtaacagaatggtccatggggacagtttttcagtgaatgttttcaacaatgataggaacacagagctaagcagagtgtcaagagacatttagtgcagtagagacatctcatctgtttttctgcagaacttgcataaaattagcatctgattattatcacctcagaatgaaccggaacccgtaaagatgtctttgatcgtaaaagtggggaggagaccctgatggaagaagagaagacttcagcctcacaatagaaaagactgtgtttatgcacctaaatctaacttagacaccatctaattcaagaaacaggcacatttagggaaataaagaaaagaggcagaaagagatgaagcaccttggctgaatcagagaaattaggggttaatgttccagactgatgacttgaaggagggtgtgcattttcatgcaagggaccattctgtagaagaagcagcacattcacccttaaatgagcaaagagaatcccaatcactctccctctcaccctccccttttccttcatttctctagctctttatttccaagaaggccagcccttctcttcagccacatggtatttggacagcctaggctcaatccctgcagggaaagctcaatcagccagctgagccagtcactgaacaaccaagtctggtaagaacaggggtgaagtatttaagaagcctatcgtggctggagggctaaagccttgttctcaaatcagctttataggtaataaaactgacattaaagcaaaaacaaataagagtaataaggcttcttctgtgtgtttcttgggttttgtttctcatttgattccaaatggggagacaagggtatatttatcctacccttcccccagcctcaacaggttaaatgggttaatacatgaaaagcatcaatcacagtgtctggagcattatcagtatcagtaaatgttaactattactttgaggctggcccatgtggctcctggcattacaggcaccagaggaattccagggagaccaggaggattatagtctgggggagtatggaggatgtgctactgtgtggcaggtgctttgcaaactgcattaaaatagtcttgatattttcagaatctaacgggatcccatatactatgattctgaaattcacttgcgcttctttagattttgcatctacatgatgcatagtaacagttcttaatttggtttattcctagcttatctatggttgtcattatttcaagagcctagtaaagttgaaatcaattttagaactacaaaaaaaaaaaaaaaggaagagatgtgagtagggtctcactgtgttgcacgtaaatgcattgacatggaagatttaaaacacctgatgagttttattctgtttctttattctctattaaaggtagtactgtgcaacattccctaccctctatttttgacactgacgttgggcttattttgattgtctaagaggtcctgtgctacaagctggcagggcttgctgggatgaagtgacaagcaaagatttgctatatggtatagccaggggcagagacaggttttctgggggcatgagacttctacagtggggagacactattaagaaatagaatacaaaattatgactacaaaatgagaggccccaaagcttatgtttcattagcttcacagggagctcacttctgcccagagccaaggactgtggacatgacctggtaatggcatcaaatgattccctttattgcctcccctggggcagaaatggtggcagcaaagcaacttccaatcacaactatcttctttcatttgacacaggaaggggaagctccctctttaatctctaatagaaacattaaacagcaggccatcacaatttctagctaaagagttttcataggacctttgcaaaggttgcattcctcccttcccttgtggcagtatgcataatgtaccttttaggaatataaattcagtgtcttggaacacatgatttagaatcagtgttcagttaccaactatcaaataaaaaaataaatccagcatatgactaacggaaagaagtcaatctgaaatggttacatattatatgattccaactatatgacattttgggaaaggcaagactatggagacagtgaaaaggtcagtggttgccaggggttagaaggaagggagagatgaatcggggaagcacagaggatttttagggcagcaaaactgttctgtatgatactataatggttgatacatgtcattgtacatttgtcaaaacacatatgtcttggtccatttaggctgctctaagaaaatatcttagactgggtggcttataaacaacagcaattggtttcttacagttctgaagtccacgatgaaggcactggcagattcagtgtctggtgcaggcgatcctcttgctttaacttcactggtggaaggggcaaagcagctgtctagggcttcttacataaaggtattaatctcattcatgagggctctgccctcatgacctagtcacctctaaaaggtcccacctcttaaaaccatcacactgggctttaggtttagtggggatgaattttgtggggaacccaaataacagaccaaaattcactgggaattgttttggggagacataaacattcagaccacagaagcacagcacatacaacaccaagagtaaatgtttatgtaaactgcagatttttagtgatgtcacggagaaaaacttctcctcgactctcttaggtttagtgcttgggaaccagtaaattaaactaactaaagacaagttaacaagagaaaaagcacaagtttttattgatatttacatgcataggagttcatggaaaagaaatgaaattcaaagatgcaattagactcacaggggttatataccattttaacaaaggaaaggtttggggcttcaaatgatgctaaattgtggggaagtgactaggaaagatacagggaaacaaaggaaagataagggttattttactaaggtctgtttatgtaaacttgtctgactctcaatctccagtggtaagagtggctctcctcttcccagttcaggagagggagacaccttcacaagaggaaatgtatgccctgattttaatctgataagaagagggcagagaacccttcctacatttggttgttttcaattgccttcagctcaaaatggtccttacgtggaagtgacatatttttatggtggtggtatattctgatctctttcaataataattgtgtgtcaatgagtctcattgattataatagatataccgctctggtgcaggatttgatagtaagagaggttgtacttgcgtggaggcaggatgagtaggtgaatgaactttctatattctccactcaatttttgtttgaatttaatattgctctaaacataaaatctgttcaataaataaagtaaaaaacaaaacaataaaataaagtaaaaaaaacaaatctagactgaggtaagtagaagctttattggaaaagattattacagagggggaagggactccaactataaaacagcaagcttctcagagctcaggcagaaaaaggcttttcttttatacgaaggagagaacaaggctagaaagaaccagatgtaagtaagtgaggtggcctgattggatagtagatcagagaacgttttaccctcaggccagccgcttcctggaaggagccattgagaaggagctaaattctggctcgacttagaatgcatcagagttcaagagtctggggaaaagaaagaaccgtaaccaaaattttttcaggtcaggttaataggcattctgttctaatagatcactggggacaataaatttagctaatcatttatgaagccaagaacaggagtttggagcgtctgtatctggctttgtcataggtaaacaatgccggcaatccttgagcctcatctaactcatatcgggaagggtggtcctttggaataagctattctctgaaacagtttagtctaagtacaatgtatttcaccagcaaataccttgattgtgccttggaaggaaggggttggtgatggtaagtctggaaaaggccagacaaagagagggagaattctggtccctgaagagacatcccatatgctctcgggtgagattgtgagaaatgctggaagaatgaagtggatgactaccaagcccatcttatttcaatgtacatagataatacttcattttgcaaaaatgaaaagtatgggacaaatactaaatcagacagggcactgggataagaggcgcgaattaggatatgtagtcatttactgtactaatttgcatgaatagaaatgcagagagagctctataaaagaaaaatattttgtgtaatctattagtatagaaactcgtaattaatttactaagatataacgatttattcgtcatgttaggcaacgtattttcaatgtcatcttatggagaattgtaaagattttcttgtatgttaaaggtctttggtcaccatcattattcacacgattgttattcacctggttatcattattcctcatcattaaggacactagtaatcttttaaacctttctggctctttctggggaaaagggcaggcttaatacaggcattcctcattttagttagcttcactagcttcactttattgtgcctcacagatactatgtttttcacaaattgaaggtttgtggcaaccctgcctcaagcaaacctatcagcaccattcttccaacaacatgggctcatttcctgtttccacgtcacatttgggtaattctgacaatatttcaaaatgtattattattatagctgttatgatagtcagtgagcagtgatctttgatgttactcttgtacttgattggggaaccatgaaccacaaccatattaggcagcaagcttaattgataaatgttatgtgtagtctaaccgctcctccaaatggccattcccccatctctttctctctgctcaggcctccctattccctgagacacaaaaatatttaaattatgctaattaataacccaacaatagcctctaagtgttcaaataacaagaagagtcccacgtctctcactttaaatcaaaaccttggcataattaaggttactgaggagggcatatagaaagctgagacagaccaaaagctagacttcttatgcagaacagttaaccatgttgtgaatgcaaaagaaacgttcttgaaagaaataaaaaatagaagatgctaccctctataattctatgaaggttcagagaggtgaggaagctgcaaaggagaagttggaagctagcagaagttggttcatgagatttaaggaaataagccacctctataatataaaagtacaaggagaagcagcaagtgctgatgtagaaggtgcagcaagttatccagaagatgtagctaagatcattgatgaaggtggctacagtaagtaacagatttttaatgtagaccaaacaaccttatgttgaaagaagacaccatctaggactttcatacctatagaggagaagtcaatgcctggcttcaaagcttcaaaagataagctgactctcttgttaggggctattgtatctggtggctttaagtcaaagccaatgttcatttaccattctgaaaatcaaagagtccttcagaattatgctaaatctactctgcctgtgctctgtaagcagaacaaactggatagcagcacgtctgtttatagcatggcttactcaatcttttaagcccactgttgaaaattaatgctcagaaaaataagatttatttcaaaatattactgcttattgacaatgcacctagttgcccaagagctctgatggagttgtacaaggagattaatgttgttttcatgcctgctaatacaacattcattccaaagcctatggaccaatgagcaatttggaatttcaaatcttattatttaaaaaatacattttgtaaggctatagtttctatagatagtgattcctctgacggatctcagcaaagtacagaacaacatggctttgaactgtgctggtccacctatacatagatttttatctgcctctgccacccctgagacagcaaggcccacacctcctcctcctcagtctactcaacgtgaagatgatgaggatgaagacctttatgatgatccaccccacttaataaatagtaaatatatgttttcttccttatgattttcttaatacattttcttttctctagcttactttattctaaaaatccattatataatacatatgacataaaaaatatgttaattgacggtttatgttatccataaggcttctgttctacattaggccattagtttagtttttgggaagtcaaagttatatacaaattttcaactgtgcaggtggcccaaatccctaactcccacattactcaagggtccactgtaagttgaaaactcctggaaaggattcatcactgtagatattagggctttcatgattcatggggggaagtcaaaatttcaatattaacaggggtatgggagaagttgattccaacccttatcgatgactttgaagggttcaaagatttagtggtggaaggagctgcagatgtggtagaaatagcaagagaggcctggcgtggtagctcacgcctgtaatcccagcactttgggaggccaacgtggtggatcacaaggtcaggaaatcgagaccatcttggtcaacatggtgaaacctcatctttactaaaatacaaaaaattagccgggtatggtgacgcatgcctataatcccagctacttgggaggctgaggcaggggaatcgcttgaaaccaggaggtggaggttgcagtgagctgagatcacgccactgttctccagcctggccacagagcaagactccatccaaaaaaaaaaaaaaaaagaagaaagtaagaaagaaagagcaagagaactagaagtgggtcctgaagatgtgactgaattgctacaatctcatggtaaaacttgaatgaataaggagttgctttgtctgaatcagcaaagaaagtggttgcctgagatggaatctacttctggtgcacaggctttgaacattgttgaaataaagacaaaggatttagaatattgtacataaacttaattgataaagtggtagcagagttcaagaggattgactccaaatttgaaagaagttttattgtagataaaacgttaaatagcagcacattctacagagaaacctttcaggaaaggaagagtcaatttatgtggcaaacttcattgttgtcatattttcagaaattgccacagacacaccagctattagcaactaccaccctgttcagtcagcagccatcaacactgaggcaaaatcttccaccagcaaaatgaatatgagttgctgaagactcagacatctttagcattttttagcaataaagttttaactaagatagtacatttttaagacatatgctattgcacattttatacactacagtatagggtaaacataacttttatatgtattggaaacaaaaaaaatttatatgactcactttattgtaatattggctttaccgtggtgatctggacacagaatccacagtatctctgaggaatgcctgtagtttgaatatgagtgatgcaaagaaggatttttagctattgtagtatctctggatgtatttcccacaatctttttgggttatagttgctcttgtatctgttttgcctttcccaatagtgattgcacttagcagaatttgtgctaggaaacattgttattgttattgatacaaaagtaattttctgtaaagaatagatcattttgtctgattcattattttagtggctctctgaaaagtatttttcttcatttcacttctgaaacagaatctagaaaaaaaaaataccatcagctgagacatttagaaacatctgtactttcacacaatgcatagacaacaaacccttacagaattatttttaggaactctattgaaattagatattttctaaaagtatttgctaaaagagaaatgcattttagcttacatcatattgtttaattttaaatctatcatattgtgacaataacaagaataaaaacaatttcaccctcagtgtaaaacttacagattttgttgccgagaaacttcaaaacatacctctgaatagtaagaagtgtagtaaaatttaaatgaaatgttgggccaaatatagaatgattgaaaaatcacccttgaattataaaaatttgtgctcatcttgtgaatactttaaatgtcttgctcatggtaaggacttcgtgctatctttgtacaattaacatatagggcaaatttcaataccaatgacagtagatgtaatttcacatatcaagtagtttccctaataatttaggacttttttcactgacttcttgtcatatatgattctgtcattacttttttatatagctcaaaatatggctgatggagtgcttgtgcccttccatggttttttcatttggtatcactagtattatatttatttattctttttttccaggacatttgcaagacattcgtcaagttttgaggtttatttaaggtataactagataatgtattatgtaaatcagcctaaccagcacgcataaactaaaccatgtcacaggatgccaatagtgacagacggagtgctgatgcctctggaaatctcttcacattgtggagtttctcctcttctcttgggatatcttctctattttctgtgacagatgcctatttgctagatcaactgagagcatcagtgcctataggtaatgaaatacaaataaaactcaatttgtgttatggtctgaatgtttatattcctccaaaattcatatgtcgaaatccggtccccagtgtgttggtattaagagtcgaggcttttgggaagtgacagggtcatgagagtggagcacttgtgaacaggattagtggtcttatgaaagatggagcgtctgtttttcccttcccccatgggagtgggaaaatacggatagaagccactatccatggggaacaagcctccaccaaaaactgaatctgctggcaccttgatcttggacttcccagcctccagaacgctgaacaacacatttctgttgtttgtaaattacctaatctaagacagcagcctgagtggactaggacaattggtttcttttttgtttaggtagcaataactagaaacagaatttctacagaggcaacacacacaaaaataggtgtgattttatcaaattaaaatacttctgcacagtaaaagaaacaattaccaaagtggaaaaaaaataacctatggggtgggagaaaatatttgtaaaccatgtatatgataaagcgttaatatccaaaatgtatgagaaattcctccaagtcaatagcaaacaaaccaatagaaaaccagaagcaaaagaaaaaagaaaataagttaaccaatttgaaaaatagacaaagaaactgaatagacatctctcgaaagaagacatacaaatggtcaacagatatctgaaaaagtgtggatatcactaatcaggaaaatgcaagttaatatcacaagataccacctcacagctgttaggataactattagcaaaaaaaacaaaagcggccccttgctttgtgtctgccagaatccattatggctgccactatagttctccaggagtctggatagcagtcccggctgggaatgtctggagtctgcaggccccagtcactatggacagtttttccttcagtattggggttctaagaggcagaaattagaaataggaatgcagatggttgcagagtgtggttgggggttagagcggcgccctggaaaaaaaaaaaaaaaaaagatgagtgttggaaatgatgtgcagaaaagggaaccccagtatattattagtggaaatgtaaattggtgcagcctctatgaaataatgtatatttcaatacataataatgtatattccaaaattgtttttatagagtagattttagatgttctcaccacaaataaatgataagtatgtgaaatgatgcatgtgttagcctgttttaatcattccacaatgtacacaagtataacatttacattataccacataaatatatactctatatatatactatatatatatatactactgtttgtccatttaagtaattttttacaaataattgttctaaataataagattatcttataaaggagacagtaggtttgttttgctccagatggtagagttaatccctgaagctagaagatgcttagggtgaagtttgtgctcaaaatatttttctaaaaatagagcagactgcctcaaaggtgaggtatgttgcagaaaaaagtcactgtaaggtatgtgtgggatgctgtttggggataatcagggactcaggatagggaactaaggagtttaggttgtctgctgtatagaggaaactagaggagatagtttattcagccatcattaattcattcattcagtaaacattactgaacacctgtgtgccaggtgctgtgattgacactggagatttaaagttgaataatatctagtcactggggccctgatgtcaaggcacatgctctagtggaagagaggcacacgtagctaagagtgtgtggaaagtgttatgaggaaagtatgttcaggatagtgtagaggctgaggaagaggattcccatctcacctgctgcgggcaaagaaaggtggatggtattcattagggattatcacaggagggatcaactgaactaatccttaaaatataatacagtaggagccagcccacaaaatgctaatagaggcagtggggagggtggtggaaaaaaaaaataggtatgagtcaaaggagagaattccagacagatgaaagctgtaaaaatagtttagttttactgtaaaaaggtcaaagccagaagtaaggggctgcaaagtccatttaagctgagaactttattacagctcctgttccaagcccatgctctgtgcgactgcagatgggatattctaataccacttgtggaaatcaatctttttggacaaagaccaagagagacccatgttgtgattcactctctatggcaacccaaaaggaagggtgaatgcctgcagatgagagttgcctcttcatgccagccacttaataggctatggaaaagggaagagcagtccccatggtgggcctaataggaatcataatctaactgattatcacagcagtgactctttcaaatctggcctaactgaagctagcaccacaccaggatctctgctgggcacacaccaatcacccaggaggtcagtatcatccccattttacagatttgaaagctgaggcacaaggtaaataacagttatgcagaagtcttgcttcagtgtctaacttcctcatctcactttattctgtttttcaataggagaagagatataatctcatgatgaaaagtgccatctaaagtggcaccttagacacagtaagcaaactggattggaagtgaagaagtcagtctcaagatacttgacaatgtctcctatttgtagattgtttagcaaaatgctttcacctgagttatttcatttgttgctcacaaccaatctgtctggtaggaaaggcagatgatatgttcacctccagataagaaaaatgaaattcagagaggccaaggggcttgctcaagattccacagtgaagaatctacttgcaggaattttaacaaaggttttcaaattcagaagtccacaggatgactgcccttgaaatccagtggtaacatataataaggtgtctttgagcaggtaagagaggaccaaccttttctaaagcctttctcctcttgctctctgtgcgccaacctgagcagcctcctgagagattctggaacatgctagaatttttcttgctttagggccacagttcatgtcactccttcttcctggaatgttcttcacaagcactgtgtggctccttcttatccttcaaatatctgctgaaatgttcccttcttagaaaggacttgctgaccaccctgatggaagtgccctctccagctcacctctttactgactctgtttttattccttttgcaagcacattttataatctatatgtagtgtttaatgcaacagtttgctaaggactgaaatttgcattcctccagcaatcaaaacccactgcgatggtttgtggaggtgggacttttagggggtaattaggtttagatgaggttgtgagggtggggcccctcataatggaattagtgttcttataagaagagacagagagaacgaagctctctctctttgtgtgtgtgtgtctctctctctctaagacatgggaggacacaatgagaagacaactgtctacaagccaggaaaagggacgtcaccaggaaccgagcctgctgacatcttgatcttagactttccaacctcccaaactgttagaaatcattatctgttgtttaagccacccagactgtgatattttgccatgatgcctgaactgactaatatacagtttacctgtacattgtttgttcctctagattataagctctccaaggcagggaccatatagttctttaaaagataatgtcatggctataagtttactgctatttccccatcacatgggaaaaagaattgtcagagttgtcagccattagagtttatttaccttaagggtgtctgacgaaatgatctgattatctccttaacttcctaaatagctcaaacttcataatgtttgaatttggaaatatctgttgattcaagagagacagattacatggtctctcttgaactttggacatctcttccacaaacttccaaatattaggccatctattaccagaccttctgaacttaaacaaatcttactttcaaccccactttagccaccttcaccatggcctaacttttgcccttgttatttgttatccatagttttctaaacttagaatccttaatcactggcattatagttgtttacttcaacctccaacacttacaggtccctcaaatcctcatttattcaacccttgttttttgtcatcttataaactgcaaattcttgaaaatcacatgtcctctctataaatcagatcccttcatatcatgttttcaatctgagtttggctccatgattaatcttccaaatcaaaggtttacaaactgtggcctgtggaataaatctggtctgcacctgttttagtaaataaagttttactggaacacagtcatactcatccatttacatattttttatatccattttcatgttacaatggcacaggtgactagtggcaacagaatctgtgtggctcacaaagcctagaacattttctatctgtcctttatagaaagaagttaccaacccctaatcaaaatcaataatctatgaagtagaaagtaaaagatgatcttttggtgctgacatgaacatattatgattaaaaaaacactttattgaggtttgattagcatacagaagctgtacataacacatacaacttgaagggtttggtgatcagtatgcaatcatgaaaccatcactatgatgaatgtcataaacatatccatcacccccaaaggtttcctctgccttatttatttatctatttataagaatacttaacataagatttgctaccctcttagcaaatttagcaactctttgagtatgcaatataatatagttaactatagacactatgctgcacatagatttctaggacttatttaacttgcgtagcaaaaactttgttctctttgcccaatacctccctgtttccccctccttcatcccctggtaaccatcattctactgtctgcttctatgagtttgactgttttagatttctcatatgagtgggatcatttagtatttgtccttcgatgtctggcttatttcacttagcataatgttcaccaggtttatcaatgttgttgcaaatgacaagattcctttttttattattaaggctagataatattccattgcaaatatataccacattttctttgtctattcatctgtagatgaacattcaggttgctttcatgtcttgggtattgcgagtaatgttgcaatggacatagaagaacaggtatctctttgacatactaatttcatgcctttgggtaaatacccagaagtgaaattactaaatcacatggtagttctattttttggtttttgaggagcctccatacagttttccataatggctgtactaatttacattctcaccaacagtgtaaaaaggttcctttttctccacttcttcaccaacatttaaatttcatctttttgataatagccattctgacagatctgaggtgatatttaattgtggtttcaatttgcatttccccaatgattagtgatagggatattgggctttgttattaataacccactggccatttatgtgtcttcttttgagaaatatctgttcaagtcatttgactatttttaatgtaatcacttgttttcttattattgagttgtttaatttctctctgtattttggatattagagccccttaacagatgtattatttgcacatatttttctcttaacctatgggttgtctctttattttgtaaattgtttcctttgctgtgcgtaagctttttagtttgatgcaatacaataactaatagattaatggtctctttttgtttttgttgcctgtgcttttagggtcatggtcaaaaaaatctttgtccagatcagtgtgtggagctttcctcttacgttttttatttctaatagttttatagtttcagatcttatatttaagtcttcaacccactttgagttgattcttgtatatgggatgtgatgtgttcaatttcattcttcttcatgaggacatccagttttttcaacaccatttgttgagaattcaataaagttgcagtatataaaatcaacgtaaaaaatcagtaggtttttcatacactgacaatgaactatctggaaaagaaattaagaaaataatcccattttaataccatagcaaaatacttagtggtaaatttaaccaagcaagaatatggaaatatctatattctaaaaactataaaacattgatcaaagaaattgaagattacacaaataaatggaaatatgtctcatatccataagttgaaaaccgatattgttgaaatgtccatactaaatgtgatctaaagagtccatgtgatttctataaaaaatccaacgtcatcttttatcgaaattgaaaaaaaaatgctaaaatttatatggaaccagagaagacctgaataaacaaagcaatcttgagccataagaacaaagttggaggcatcacactacctgatttcaaaatatattgcaaaactatagtaatcaaaatggcatagtactggtgaacaaacagatacaaaaaccaatggaataggatagagagcccaggaataaatccacaaattaaaaaccaactgatttttgacaaagatgccaagaatggggaagggagagtttctttcattatttattctctgtattcttttcatctcaggaaaaatatccagtttcctcaattgtatatccattcactcacccgataatttcttcattcacttatttgtttattcatttaatctcaattgtttgttcattctgtaaatattcagatttctttttatgcattttctcagaattggaagcataatactgaacaaaataactataaatctcagcctcccactcccatatttacagtttgattagggaggcacatttagatatgcagtgataattgctttgcttagagaaattcaaggtgatagaatgcatggtgacacctaacccagactggtagagaaagggaattcttccactgggaatgacatgattatctaaataagtaggctcaatcaggtcaggaaagggcctgaaagactatttcaagcagagggaaggtatttgccaaggccagggtgtgtagtggagagaatgggcagtggcagagaattatgaagtgttccaatgactaaaagtaaagtaacaagttccttgtccaagagcttggattgtatcctaactgaaatgagtatacactaagtgtttgaagaagagggatgaaatggtcaagttttcattacacaaaaataacctgttcctttcattttatgtttatttatttttttaattttctgactgctcctttctggaaatctcaaatttatatttgccaaatattgtcacattttcgatggagaatacaaactaagaatgggttagggaactgagtcagaaagtccctgttgtacaattcaatcatgtttttctaaggatgtgcttttggacattatggaaactatcttaggctctcacttggatcctagaaaagaaggcacctgttaaaaggaatgtccagccccacctaatttggcagctgcccctccaagctaatgacattagggatgtagtggattcaagaggctgatgatgccatctggtcaactcatgtgactatttctatcagctttatttctgcaacatcctgtgcccacagaggggacacaaaatcgctttataattccttcaccatgtgaagatacagatacacccagaaccttaaaggcaagaatatgattgaaatgtcaaatggggacttggtgatctaaattatgtcccccaaaagccaatgtcttgccaccaccagtgccctatgggtggagtttctaaacagattactcaaaacacaaactttcaaaaagggaaagtcataaccctctagtcatcagggcaattacggaataacattgctggagtaaggttttctcaatgcccaagagatgagctggcaatgccacaacaatgtccaattcttagtgggtccaagaccatgtgttacatttccctcccatgattactcacagcttcacagttctgctgtcctcttcgcctctctgccacctcttaactgcacctttgacctcctacccctaagattcaaccctgtgagattacttgtcttttcatctacactctggtcactctgacccccattctttagattcaggattttctcttttcctggcttactctccagcacaagtagaaaaatattgtgcttcattggaaaatgcatgttgtttgaatcacactctttcagattatacaattgtagtctttcattatctttgagccattttataatgctgtaaactaatattaatgcatataattcttgtctatagacatgtaaattgtgtccaggggtgatttaattgacttttcttccccattgtggaaaaggctagtagttttgcctccatttgcccatttatttctatattcctgatctgtaaatgcatttctgggttttcccttggactcattctaacatctcttctttttcctcattaattaattaaattaaatctttaacacctcttcatatttttgtcagtatgagagtataaaaaaaaatctttttttgaaaattatcttttaataggtgacaaaaagaataaacactgaacaagaagtcctgagctcctactacaatccaaagtgaactagttgcagtgccataggatgatgtttctcctgagacttctttactaggatcatttagactgttataaatgcagattcctgggcctctccttagacccactgaatcagacactctaagaggagggtcagaaatctccatcttagcaagttaatgtccaggagagttagagactcactatgacgtatgataaaaagtagaaggaaggactttccagcaccttaaactacttggaaagatagattaatgtctagatgaaacttgatgagactttagactaatatgttacatatacatcaggagatgcatgtataaaccaaatccaaataacccaaagcaaaatttcttttagaaatagtgataaataaatgagtgaggagtttgtcactcacatcccccaggtaaaacatacctttttagcctaaataaatgctatagtttgaatgtgttccctccaaaattcaggtactgatagttaacagcagccgatatgatggtattacacaaaatagtaggttctttaggaggttgttaggccatgagtgcctcccccaagaatgggatgaaggacctcataaaagaggcttctcacagcattgtgacctctagccctcccaccttccataagtgaggacacagtgttcctcccctctggaggatgcagcaacaaagtgtcatcttggaagcagaggagccatcaccagacaacagaaccagccaacagcttgatcctggacatctcattttctccagaactgggataaaataaattcctgttttttaataaatttcccaatttcaggtattttgtgatagcagcaaaaacagactaagacaactagtatgaaaatatacattaacaaataaaattaatcataatatatgtttgcttttaaaagaaaataaataagccaatatgctttctgttgattgattgatttactaaacattgattggccatctccactggggatatggcatttaagagatctctttgatcttagtacttttactgctttttaaataggatcaaatacacccaaggtaaaaaatagaacacactatacgttacattttggaactgttagaaattcctttgaagctaaaattactgctatcatttgacaacttttacccctaaaataatgtggtgctcaccagcttgcttaagttacagcacttgctgtcttctcagatacaatatcagaaacttataatccaagaaaaatctaaatggcaagtgtgagttaatggaagcctcataaagcaagaggtgttttggaagtgtatggaagacatcaataaatgatatgtataacatcaagtgcaaaagtgtgtgctaggaaagttcaaaaaagaaaaaaaatatggtaaggtaagaccagagattggggagtatatagcttttgggaattcaggaaatgctaacgtctatggagaatttgcataggtgaaagatcagattggaagcctttctctgtggaagcattgtggatctaagtccagaagtgatcctgagccacctactatggaaaggtgtcagtgagcaagagactgtctgacaaaggtggaagctgagcagacttctactgcgcatcgcctatgtacaggccagattccaagggctgatattacactgctagtttgatctttctcagatagctgggtagagagggaaaagtttcaccccaaataccagatgcctccaaacatctagatgcttggtgttatgtatttcaaacagcaggtttgataaagctgtttttactctccagttggatgttggttgtcaaggctgtcattaacttctgggagttcccaaatcctcagagagagagaaataagcagttctgcctgcaatcagaagctggctttgagccccacatggcaacatgcctgtatttaattagaagtggcttagttcttgggatttactgtggattgagtctacgaagctggaaggttttatcctgagtattccactgtctcctcatgtggattttatcaaatttctcctttaaccatataagcgctttcaaagttgacatatcacacttagttatgaggggaacctagaagtatgattgtgacgtgcattgttgagagaccatgctttgtaagtgttaataattaaaattattttgaacactaattatgctgaagactggattttctactccttcagatgttttccagtggacatattttgcccttttaataaattgattgcgaacttatcttcatttcacctttatgatgttataccttttcatttttgtcttctcatagctagggtctcctggtccccaaatgtagacacacatcttacaccaatcccagagccattttgtataagagccaccatggatttaaccagctttagctccagtatttgaacataatgttcagcatcatcacctggccaccaaatcaaaactgagcaccctttaatccatcaacaagttctctgcagccatgcaaggttatgaaatgggcacagacatcaatatacagtctttgtgtttaagaggttcatggtctacctgagaaatgcatctttaaacctaaagtagacgctctgtttattccataaatgatttttaagcatcaatggtatatcaagcactgtactggcttctgggctataataaatatataaagaccacaagtttgaatttcatgacattgaactataatgtttaaatgttataataatcatagtaaatgtccttgaggagctacggaagattcctgcatgaagcagaaacaagaagctgaagaaaaaacaactggctttgggggctatataaatataaccctcaaattaaaaactcaatagtttgattgattaccaaatcaatacagccagaaaataaatgtattaccccaaaagcttgagtgaagaaagacttctgtaagttactagaaagcgctaaggaagaaataaaagaatgatattcctgagagccaggtgctttctgtaggacacacagatacagaatgagggaaaacagaaaattctatggttgtggattcaaaatagaggcatcaccatgtctatctcatcagggtttaaccagaggaaaccaaaccagtaagatatctgtattaagagatttcttgcaaagaattgacctatgtgattgtgggcactggctaggtaaatcctaagtccacagagcaggcaggaagaaaacaggctgggacttgtaggcacaggatgaagctgcaatactcatgtggatgctgctcttcttcagggaagacttggctctgctctcaaggactttcaggtgattaaatcaggcccactcacattatctaaaataatctcccttactcaaaaccaagtgattatggactttaatcacatctataaaatatcattatagtaacacctaaattagtgtttgaataactgagagttgtaactgatatggtttggctatgtccccacccaaatctcatcttgaattatagttcttataatccccatgtgttgtgggaggaaccaggtggagataattgaatcataggggcagtttctcccattctgttctcatgatagtgagttagttctcaggagatctcatggttttataaagggcttccctctttgctccactttcattctccttcttcctgctgccatgtgaagaaggacatgtttgcttccccttctgccatgattgtaagtttcctgcggcctccccagccacgctgaactgtgagtcaattaaatctcttttctgtataaattacccagtcttgggcagttctttatagcagcatgagaatggactaatacagtaactttaccaagtggacacataaaactgatcattacaatgtacagtgaatatttggtgagttaatagatatattcataactgaatgaaagaggatggtgattcctacttcagggtggtattatgagagttaaaagggttagcatagatagaacacttttctatgattgatctaaggttggcttttagggactacattatacatatgggttttgtcttcatgtttttgcttgcatttctctctctgcccactcttggggctatttggatgtcacctcttcttcttctgagctccagtctatgtattccctgttccatcatcacaaccactgaagtctacactcctcattagattcagggaacccagatgccagccaaagacatccctttccctcatgacccagttgaaaaaattctccacccttctgttaattctagaatatttaggagtaaagatcttttccctcagatatctgtcaattcccggccttatatctcaaagccccttttcatccaggtttcctagatcatttcttgtcgtccctgtgctataactcattaacccaggcataaaactcatcagctactaatgtcctcttccatcccagactcccctcacaccaataattcttgaatagagcaacccaaattgagaattttaagtaaaattgaaggtaaatttaattgcaggaggacatacaacattaacctttaaatagagatcctaattcttaaaaaaaaaagtcctcattgatctcggggccgtttcacgcaggtgcttttctaaatcatcaaggttatcttcagcttcactttcagtctccttcttcggctctggcgctgccactggctcttcctgtgctaatgtggttgtggcagctgcaatggctgcaggggtcacagcagctgcagcagctgcagccacagccttttccttcttgtgttttttgtgcttcttgtgcttcttatcctttttgtgtttcctgtccttctttttcttctttttctttccatctccttcttgatctgaatttcttggcagtgatgggctcggtgttgggcttttggcctttttgaccggtacagctggtgaccagtttgtagacggtgactgagactggatgggggatggagatgctgggggctttttagctgctggctcaggagacccagagacagatcgggaggatgagaccctccttacagactgtgggcttggggaagcagcctttttttatctttttaggttccggagtcctggagactctcctaatgggcctagtacttggagacggggactgccttctttggggtgatgacgacgctcctcttcgaacgggtggaggacttgaggtctgaggagctcgaggccgtgatgagggcgaatgccatttgtttggatgcggtgatcgggcctcccgggtagagcggcttggggaagaccctttcctatgcttggatgatagtgaaggtgaacgtctcttggtgactggggagcttctttggaaggtggagaatgggagacccgccgctttggtggtggaaatggtgatgctcttcgtttaggagggggaggaggtgaagccgttcttctctttggaggtggagaaggagagtatctcctctgtattggaggagagtatcttctaggagaaggtgagcgccgacgtaggggaggagaaggagtccttcgtcgtggtggtggtggtgtgggagtcctgagccgtcgaggatgaggggcgggagaaggagaccgtcgccttctggtgggtggtggggatggacttctcctccgtctaccatgaggggaagtctctttttggtgctttcgtggtgatggagaagcactccgggaaggggaatgtctccgccgcttgccaacctcaccattcttcacatgggatctcttgggtcgttcatcttctgaggagaaggaggaaccagagtcagatgaagactgctggttttgtcgtctgtattggcgtctctgctgagatcaatgaggaaggcccaagtgtccccacagtcttagggggaaatgtttgttatgatgtaaattttatttggtttgtacgcagttcaatttcaaaattgctaaaatgtgtttgagctttagactataacatttgttgtaataattgctaggttgaagttcaacatgtaaaaaaagggggcatggatttacattgcaaaaggtgtccacagtgtattagtgacattctttcattgacagctgacataattcattgagtgaaatattttaagccaaaaaaaattccctttttaaaaaagggggtttaaatactgttgacacttttatggttcctttaaatgctctggctattcccagaggggtttttttgtttgtttttttggttttgatttgctttttgtttttctttcttcttcttacatttttttccatttgagtcttagctcccatttaagttatgcttctgaccttgtatggtctgtaagcttgcccagaaataagaccactgttttgaactaccacaaaagtataaatgaatattttaatgccacagtctttcctgttgcctgtggagtctctgctgaaatgaatcaggattcgagctctaggataagacagaaaatgaaagcatgttgtttgccaggacactgtgggtttatattgatgtgtaacaacttgatttggaacactggactctcattctgttcttctggttttgtttttttgttttgtttttttcttttgtaaaggccatgaactagtcccagaaaggattccttcagttacatacaatttgtttaatgaaatgtcatggctctgttcatatttttgtcttgttcttccaattggtgtatacaactttcagagcctcttgtatttggaaggctggaagggcccagactttggaatagtgtctcggtttcactgtttttgttttgattttttttttttattttttttaaactaaagctatataaagcttgtggattaaacagaataaatttctaaatttaaaaatttaaaaaaaaaaaagtctattgtcttccctcccctaccctaagcaatatgcaatagtggctcttcaatagtcccagactcttcttctcttcctggactgcccatctcctgatcaacccttaatttctcttccttctctcacccttcttttcaggattgaattaatgaatcctttcttctcactcatgcagagtaagtttctgcctccctgggtctttctgtttactgaccgcaacaacttcagattatacctcttctactccaagtgctttcaaagaaagtcctctgccaagacaaattcattacgttttttccctctacctgtttgcctttattctcttttgtatttcatcttctcatctagattgaataatctttgagagcacagatgtttatttatatttttcctttccatttctactcagcatgaggtgtccattgaacaaacttgatgaatttttattgcttaatatcttgctagaggtggggagagaggttgggggcggttaaggaactatcagctagcctaggagatattagagctgcagagatttggctatcttgttcaacgttatatccctagggattagtacataggcttgcaaatagcaggtatgaataaaaaattattgaatgagtaaatgaatttaaaatataagttacttaggcggtatcttcaggcatatctgtgtttatgtggtattcaatggcccacaaatgtctacatcctaattcctaagatctgtaaacattaatttgcatgacaaaagagactttacagatgtgattaaatgaaaggattttgacatgcagataatatcctgtattcttcatgtggaaccaatgtatttacaagggtccttataagtaaaacagagaagcaggaaaatgagggtcgcaaaaaaaaaaaaaaaaacaaacatgaagacagagaagaggttagagtgatgttggctttagagatggaaggagtcacaagctgtcttaaaggaataagacaagctgtcttaaaggaattgttataaaggaatagctgaagctgggtaatttattttaaaaaggtttattttgctcactattctcatgtctggaaaagtttaatattgggtagctgcatctggcaagggcctcaggctgtttccactcatgtcagaacgtaaaggggagctggtgtgtttagagatcacgtggggagagaggaagcaagagagagggaggaggggccaggctttttttaaacaaccagctctttttttaaaaaaaaattatactttaagttctggtatacatgtgcagaatgtgcaggtttgctacataggaatacacatgccatagtggtttgctgcacccattaacctgtcttctacattaggtatttctcctaatgctatcccttccttaacccccaaaccctgacaggccctggtgtgtgatgttcccctccctgtgtccatgtgttgtcattgttcatctcccacttatgaatgataacatgcagtgttgggttttctgtccttgtgatagtttgctgagaatgatgatttccagcttcatccatgtccctgcaaaggacatgaacttatcctttttatggttgcatagtattccatggtgtatatgtgccacattttctttatccactctatcattgatgggcatttgggttggttccaagtctttgctattatgaacagtgctgcagtaaacatacgtgtgcatgtgtctttgtagtacagtgatttataatcctttgggtatatacacagtaatggaattgctgagtcaaatggtatttctggttctagatccttgaggaattgcaacattgtcttccacaatggaacaaccagttctcttaagaataaaagtgagaactcacttccctggccccagagagagcaaaagcaattcatccccatcacccaaacacctcccattaggccctacctccaacattgggatcaaatttcaacatgaggtttttaggggacaaacatccaaactatgtcacaagacaattcatgtaagcagcctctaaaagatggaagaggcaaggaaacagattctcccctaaagcccacagaaggaaagcagccctgccaacttcttgattttaacccagtaagacccgttttggactttggacaccaagagctataagatgattatgttgttttaagccattaagcttgaggcaatttgttacagcagcaattggaaactaatacagatcacattctaattcaattagtattgttcccagttctctggacctcagatttctttcctgaaaaacattaaaaataatacctgaaagttttgcacacgagtgcagagtgcctatttactagagagatcagcatttgtttaggctctgaatagatttgaggatgaaattaaatagcataaataaagttcctagtgatgcttctgataaaaaaatatctccttcaaaatgccagaggcaggtcctaaaaacccacaaagcaggtgaactggcaaaagactgtaaaaagcaaagtagaggttcctcttcaaagactttcctctccatctaattaggaataaatagtaacttatcttagaaacaaaatttattcaaagacctgtgctaacattctgaaatatctgctagccgtaataaataaatcgatgtactttatgttcttagctcccacaatttaacctaaatatttgccctggcatgcttatactggtccaagcaagcattaggtcatagcctgttcctcttctttattttaaggtgtttttacctttgtcagcatgccacaagttacttcctccttcctttgttctcctctgcctttgactcttttaaagagtcctaagttgctagccaatcaggacaaatacagaatgtgaggtcccgtttcagccaatggaaagtggacacagcaggaaggtggatgggtcaggttataaatgaccctgtctcctttgttcggtgtactcttgtggcaaaactgctggcaagtgtaccctttctgcaataggtaaaaactgccttgctgaggaaattaaatttatgttcaagtgctatttctttatggcaccggggaacaagcatttctaacaagactatgtaatttaatttcaggaacctaaaaaagtgggatgaagaactgaggttgctaataaatctatacaacttataagtaaatatttaatttactaacatataataataaagacatcattgtaagacaatgttaaaacattttacacattttaaatgtgcaatagtaaatccttcactattcagggattatttggaatcccttgtcaccagaagctcttaaggaaataacttctacttcgttgcaaatatgttcttggcttagttgaggtaatgcaaatactagaatacttgtttgtttaacagcttattcttccctgaagctgttcctccagtccctgccagtgggatcttatgtctccaggagtacttaacacccctaatagccccatcttttaagcctccctgggacctgccctcgcagtacctcttatacctactccacttcctcctcatggcctcctgcagaatgccattctaaaattaggttctattttcctcgcccgcattctcttttgcaaagcctccaaaaaatttactttgcttctctgcgcctgctttatctctattttctacactcgctccttctttttctaattatctataataggcgtcacaaaatttgcatttgttggaaccaaaatttccatggttgcctcaaaatatacagatgtaaatttgcatataattaaattttgcataagggaaactctcatttggggagatatgcaatgcccaataaatggcagtttccttcaatgtccccaggccagcctcccagtctgtgtgtttccccctggctgcagctaccaggactctgctctggggatttacggacaagggtatcaagttttaattaaactaaccctctcaaactgaatgagtggcttaaaatcttcctgtaaagaaaccgcaaaataataatgctggcattgagaagtaagaaaagagcgagccagcacccccaccccccaaatcctgtgacaaggtgtatttttgtgttttgttttttttctttggcagcattatgggggaaaagcaatgatgatctaatgagatctgataagaagttagcccaaaacaaggaaattgttgagggttctctttgaagtatggatttatacccaccaaccttagctgcgaaccttacctcaagtgttacctgtgccttgagatgtttcctggtcatagtactaagctatcataatgagcaagacattcaataagcaagtgtgatggctatgaggacagatcttaacaggtttttttttctggaaggcttaaaatcatgcattactcaatctaatacttcacgaaatttcagtaaaacctaatgataatatagaagcttgtgttgtagttttgtaatcaacagcaaaacataaaatttaaaaaaaacatacattactggggctgtatcctgctacaataataaggctgacataatagatggagaacaatatggtaacaagccaaaatgtattacttcatccacaaatagtatcgtgctatatatagacagacttgttaaaaatttaaagaaatacacaatcaattacacaatagaaaatttgctatatggtgcatggtggcatgcaactacaaatgtttctaacatgtttctcttcataggattttctgaattttcatttaatattcaagcacatcaaaaacaccttttcaggtgtgatcctatacagcaaagctgtcctcacaaacaatagttgactaaataaacacatggctttatggaagaaatgtgtaagtatagccattgttggagcagatgctctgcttaaaaagaaaaaaaataagttaaagttatagatctcaccatgcttatttactgcttaagtcatagccaatttattgcaccaaagttgaagttcaaagcataaagaatactatatataatgcaattaatgaggttgatgtccctaaaacaagagagaattagtaaatgttttacaatagttttcatgagatgggaaatgacaatagaaatctttgttacaatgcagattttattgtggaaatgatctcatggcaaagtttttaaagaggctgcagaacaataaagagagataacacattttgcttttatgaaaaagccgatgttccaaatttgctgaccttctctgtaaataagaagtgactgtcagtagccagcagatgtgtttataaaaataacccacttgatctgtccctgcaaggtaagagtggcattttcaaagtgcatgagagaactgctctcaagagaaaatcatactgtaaagacagcatttcaaaaacatttatttggaaatatttaaatgatgtgatctatttatttatttggtcaaaaatgcccaacttgcctaacattattttattccttaaaacacagatcaagcaacagatcctacagttagactcttctgatcaccctgagagttagttacccctttctttggtttcttctctttaacgtcctgcatatatgctgccattgtgtttatagcacaatatcagagtgtgttatttatacgtgcatctccctcattagattgggagtttcttgatgtcagaacacagcaattgtcccaccagaataaaaagaatattgccaaaagatgctctataaatgtttggccattggtttaaaaaacaaataatggaccaatgggctcaaaagcaaactggctaatataaaaataataataacaaccacaataataaaataagggctaacacttaaacggttgtgtactcactatgtactaggcactgatcaaagtactttgtacatattttcttatttaatattcgctaccatcatattacaatatactgttattaaccccaatgtatagatgtaggtgaagaaacttgtcacaaatcatacagctagttgtctgagatgcaatccatgtgatttgttcacagagctcaggttctgtgaagcgggtaaaaacaaaatttggcatccagtttcaaaaggagaattgcaaactaatagaacatatagcacaaaatgattatatcaatagaatgctaattgcatatcaaggatatttggtataatacaaattattctaccttaaacatatggaaatttgtggtccatgatgttgtagattctatcttcccactctgcattttcaaaggcatatggtattgactcattcgattaattgttggatagtctttattatagactaaatcatagaataaatacatggatacatgcacgaatattatatctcaagggctttacatagttcattatctcacttcatagtcaaaacaaacctactgatagttccaatgcaaagcctagaacgctttggcttagagaggcccaagtcttttctcagtgctgcactgctggtacgtggcgtggtcccctctcttctctcagtacacactacccatgcagactatcactctcagtcttgtttatctcaaatacagagggtataactaactggaatgtatccagaacagtgaggccaaagtgtggggaagctccttaaccatgctgctgcatgaggaacagctggagagactgagaacatgaggcctaaagaggagactcagggagatgggatcacaatcttcaaatatttaaaagacatcaaggggaaaagagattaaacaaggtaatgtagctctagagagcaaatccaagagtgttgagtggaagtgaaagggaggctggtttcagtcagatagtaggaagaactttctagtatttggtactacaatgggaaagactattttgtgagatttttttaaatttttttttaattatactttaagttctagggtacatgtgcacaacgtgcaggtttgttacatacgtatacatgtgccatgttggtgtgctgtacccattaactcatcatttaacattaggtatatctcctaatgctatccctccccgctccccccaccccacaacaggccccggtgtgtgatgttccccttcctgtgtccaagtgttctcactgttcaattcccacctatgagtgagaacatgcggtgtttggttttttgtccttgcaatagtttgctgagaatgatggtttccagcttcatccatgttcctacaaaggacatgaactcatcattttttatggctgcatagtattccatggtgtatatgtgccacattttcttaatccagtctatcattgttggacatttgggttggttccaagtctttgctattgtgaatagtgcctcaataaacatacatgtgcatgtgtctttatagcagcatgacttaaaatcctttgggtatatacccagtaatgggatggctgggtcaaatggtatttctagttctagatccctgaggaataaatgaccaactattgagaaattgcagggtagtccctacatgagggttaggtagaattgacctgctttctgcctcataaattttagaaaattaataagataatttattacggggtggtgtttgttccctcagtactttatcatctatgttgataatgttaataattaattgcataattaacaaatagcaaattattgtgggggtgtgtgtgtgtgtgtgtgtgtgtgtttagacagggtcttgctgtgtcacccaggctggagtgcagtggcgtgatctcggctcactgcaacctgtgccttccaggttcaagccatcatcctgcctcagcctccctagtagctgggattacaggcgcctgccaccatgcccagctaatttttgtatttttaatagaaatgggatttcaccatgttggctaggctagtcttgaactcctgacatcaggtgatccatccgactcatttcccaaagtgctgggattacaggcatgggccatcatgcctggcccgcaaattgttgttatttataactcttcaatccaaatcatcagtgtctatgttgtttccttaactatcaaatgatgataataatagtaccttcttcataagatagttgaaaggtttttaatatccatatggtactgagaatgatgcctgaaacatagtaactaccccatttttattatatttctgttaataataatacataccattattgctcttgcataccatattgctcttgcataccatatatgctcttgctatatgctacacacagtatttcatttaggcctcactatgtccctgatgtaggcattaatatctttattttgcaaatgagaaaacagtctgtaccttgtatgccatgctgctattgtttatctgtttgaatctcaagcaaatctgcttgataattggtaccaaaataagcctttttctgggtaaggaatctgatattgtgttttaaaaaacacacatttaatcctggggctgctgcattactcctgctgccccatcctactgtgatcaaaggcacatacatgagatggtgagttgtccccttgccaatgagggtttggtaagaaaggaaagtgcagtacttctttgtttctgaattgcaagtatgtgtgggttagagggggaggctgaatatgaaggtcctgggacagcccaccaggtatcccatgagactttgcaaaggaaaaggaggtgagtgacagcccagggtccaataggatagaaggaaaagccaggccatggagttcctcagacctgctttctaagggcaactctaccacctcagcaagccattgaacttctctgagctcagtcctttcatttataaaatggggtgacagtgctcacatgccaggaatacaaagggattgaaagataaaacacgtaattaagcacctgttgttacacatctgtcagggaccccaataaggtcagctgtcttcctgttgacttctgttcttggtggttctccaagatcataccttccatcaacatttaccgtcactcccccaccccatgcccaatactgaacagtggagggacgcttcacctacagttataatgttgaaacttcaacccaaagcaagtactgttaggatctctggaaactttccctcaaataagggatttgaatgggacaagaagaagttttacagatagccaatggagatgatttaatggggttatgatagaaacgagaaagtaaaacaaacccatgctttaaagtctaccatttcaggtccatattttcgcttgaaaattgagattcctattaaacaatgacatttacaccaaaaagtagaggagttggttgaaggacagggtaatgccaggaggaattgggaatttgagagtcaagtcaaaggactgaaatactcagaatactaagggcacctcagggctctaccaaggacacgtagaagctttgaatttgcagcaccaccctaatttaacgagctacctcagcacgtagtggagccttggaaaacagatgtcacaaactctcattagattgtcaaacattttccagcatttcctctcccatcatagctggttatcaagatatatagacacacacgtgcatacacataaataccttgataagttactagagaaagcagaaaaatgtctgacagtttaatgagatttgggtgaaagaaaattctatatttcattgttttccaggcactagaaataattcatcaatgtttctaagactcattcagcgtggctgcattttttaaaatattttcataaattttgaggagcaaataccattattaggcactaaaaaggttgaagtctaatagattagccgcttcatcctccttcactcagctcagcattcgttcaactggctcttactggttaacatccacacgcctcctgactggctactcagtgccgatgacatttccttcacacacagggctggttttaagatacattgaggtgacatcaggtggcctgtaaagtggtcattttaggatatcctattcaaagacatctgtggaagtgtggaccaatttattgatgaataacagtgaaggggtttccaccagcaagtaacataattttttacaatgatgatgctgaagtagaaagagtttctagtcagggactggacaaatcaatttgcagacgatttttaggaagaaaaacattgcaacagtaaattgtaattgataacttctagagccactttaagtactgctattttaggattctgaggggaagaaagtgttctgcaaagcaataagcaaagtgatttgttccaagccccaaatttaagcagtttgagaggtaaaaagagtcattaccaatgtgggtatagaacatgtgctaggcaaatctctttcacatacatgtggggaggtaataaaattataatttgaggccgggcacggtggctcatgcctgtaatcccagcactttgggaggctgaggtgggtggatcacgagatcaagagattgagaccatcctggccaacatggtaaaacctcgtctctactaaaaatacaaaaaattagctgggcatggtggcacacacctgtagtcccagctgctcgggaggctgaggtgggagaatcacttgaacctgggaggcagaggttgcattgagttgaaatcacgccactgcactccagcctggtgacagagcaagactccatctcaaaaacaaacaaaaattatagtatgaaataggcattaaaatattgtgtattttagaggagactgaggattggaggctgaagaattactctaaattaatcagcttgtgtacttcagagctaagatagctctttgggttctaaattctgtgatcttctttttgatttctcttggagcaataatgaaggcaaaacatcaataaacataacaaactgggtaagggagaccattgagaaggactaaggacaccttcaaagttctgagtgagtttaaaaagaagaatgatgaaaactttgatagaaataggaaaaaaagtagaggaacttgtttggcttgaaacttcttaatgtttaggctaattatattgaagatgacagtggtcattgagaaaacaaaatccccaaagcaattttggaataagagccaacatttaatacttaccagacaactattctaagtattttactatattcactcatagcaactctaaaaagcaggtagtattaacagagaaaatgaggcacagtgaggttaaatagctggtccgaggctacacagctaatcagtgggagagttgggacttagacccagaggtccagttttgaagtccacacttttagccattacactacaatggaaagaaatttagaagatatacacagaaaactataggcacatagattaggggttagtagaatgctctgggcagttaaaggaactcttcttaaaggaggtaaagcttgaatgagactgttagtaagctatttttcactcattggtgaatgatgttttgtgcagtgtgtttttttccccatagaaaaataagaaagaaaagaaaattgagaactctctctataaaaatgtgtaacatatctcatattccaagagatccttttggtagtattaatttttatctgctcacagtactggcttcattatttggagttaaaaattaactcaaccagataaaaaaatcagtgctgtgtatttgtttatctttcaaatctgtgttctaattttaaaaagttatttaacagaacgaagctatcagctaagacaatggcaaagccgtaaacaaacataggttgcgtttatgcgaatggtcaggtccaaagtagatgcagaatatgccaggttcactaattttaatccctattcagcccaggactatgtaccataagattactgctagtgttttctgaaaatgatgtatcaaggcattttctgtagaaatacgaaacagtgacatacagtagggagagctggattgaggcagagtagtatagatggaagtttcctgaaagcattttggggaaacatcttttgggtatggttcttggatgaagagttgatttattagtactggaagggtgtatgggagagaggaagtgagaggttatgagagaatgaccctcccgtgatggtgagtgggagaattattgcagtatgtacgttagcattgctatgtggtgaagttcttgggatttcctggggtccgtgctggacagcatgcttagccaccagtcacatgtgggcactgagcactgacaatgtgggtagtctgaactgggatatgctgtaagtgtaaaatacaaactggactccaaagatttagtatgaaaaaaagaatttgaaatatctcattaatgatgtgtatttggtttcatattgaaaacaactttggtattatatattgagttaaataaaatgtcattaaaattaaattttacttaaactaaaatttaaaattctatctttacctttttttttttttttttttttttttgaggtgccgtttcactcttgttgtccaggcttgagtgcaatggtgcgatcttggctcaccacaacctccgtctcctgggttcaagcgattctcctgcctcagcctccctggtagctgggattgcaggcacgcaccaccacgcctggctaattttttatttttagtagagacagggtttctccatgttggtcaggctggtctcaaactcccaacctcaggtgatccgtccacctcggcctcccaaagggctgggattacaggtgtgagccaccgtgcctggcctatctttacctttttaaggtagtgactagcaactttaagattcatatgtggctcatgctgtatttctattatggaactgccttatgactttcaatgggtaggatggacacatccttggtgggatggagaaatctatcatagcagctggtcttgaaggtgggtggggatatgataataacttaggtggggaggctcaggaggactcacagaataactggcaaccctgcccctgtctgtaaaaaccccatcctggaggaaataagttaggaaaaggttttgcattttgtggaatgaaaagtctgttgcatctagcttgagacagagcaaaaagagttgattgtcagcttcatgaagaccagggggtctaaaagacccagggatcaacaaccaatgagagcagcatggaggccaagaaccaggcaaaatgctgattccaggactgggattcaggatgatttccttctatgcaataatctgctccttgaaagggtatctaattgggcattgcttttacttgctgctttcaattcttttatgttctttcctagtaaatattttttcttaatttcattgcagctcgtatttatcctgggaacagagagaaatgtttcacaagcttaagccagtcttttaaaaggagaatggcaggactccaaaaacagacatgctgatatgtactggggaatttttaagtgctgaaacctccaagacaaaagagactgtgtctttattgttctctgaattactcgtacccagctcggtacctggaacatgatagggatcccatagtggtttgatgaataaattagtgactccaagagtaaagtaatcctcaggaggacaaaggcagatagcttcccttccctatcagaatgtacttctcttaaagcttttcttggtataattcttggagaattttgccttacagaagtcaaatcacataccaaagtgaaaactggatcttctacaaataatggaagaatcaactctatcaaaacaacaattatacatatgatcaatggaggggttgtcacgagccaggctaagagctttacatatattatctcattctgtctatgccagagaatcaactatgacatatgtaacattaaatctcattttatagatgcaaaaactggggtgtaaagaagtcaaagaatcagccagaatgtacagaattagcaaaggtggaactgggatttgaattcagacagtctgactccagacgccatctccgaattatgcataattatatttcaattattaacattcataaattgaaatatgagggataatgtaccttttcatgaaagctttgctcgttgtgtggatgagtgtgtgtacatgtaactgcttatgtgtgctatcactgaggtagaagacatctctctctctctctctctctctctctctctgttttggtctacttttagtaagacttgtatttgattgagttcagaagtttgattatctttttaactaacctgtttgttttaattatattaaaaattagtcactttcaacatatttgcataggtaattgttagggtgatcttttggatgatcagatgtaatatactactactacacacagacacccagacacacacacacacacacacacacacacgcatgcagacacatccttgagctcaaagagctttctcaggactacatactttacatttacaacaagtcatttagaaaactatcaatcctattcaaatctcagcaaaacagagataccaggctctgcctttctctttaactgctctttttttgctgtagacaaagctgtttcttgcactgctacatatataaaaaagtgacaaatccttaactgtcaaacaagagaaatagtttgataaatataataattccataagatggcacattatacactactaaattgtaaggacagtaaagttactgttaagtaccaaaaagtcatgattaaatgttaagtgaaaaaatagaatatagctagatttgaatttgaatattcaatctgtataccagtatgtatagaaggaagagtatataccaaatagtaagagtatctatctgttttataatttgatataatacaaattattctaccttaaacatatgagaatttgtggtccatgatgttgtagattctatcttctcaccctgcatttccgaagacatatggtattggctcattagactatttgttgaatagtctttattctattatcatagaaaaaataaatgagtgcatatatccatatacaaaatagaggtctgttcttcctgtatatatttatactaaaaaaactgagacttttttttacagttgtatatatacaaacatatttgtttatttatatacacatatataaatcaattttatgtacatgtgggtatacatacatccatgcatataactctgaagtgctgactctctaaagaaagcccaggtattggtcagaattcatgctcggctcaggagtatagaattaagagatacaaacctcaaaaaagagggaaccgaatcttcaaatctgagccaccttacaagaatttttaaggtaactgttttaagtgtaaacattatggcaatgtaatagtataatttgtatggcacaagatggagtcctggtggccagaactgagtatgggaatagtcagtgctaatctttgtgcaaagcacaaaggaaaattggtatacaaggcctgagagagaggtcaaggaagcaaatactataaagtcccagaaggcagttggataggacaaggggatcggaagagcaggatcaagatcagagagcagggaagatggggctagctaatctagggaatggagggagaagggatacccagaagcaggattcagggggttagagcacttacaccatgctcatggccatgcccatggacaggagaagcatatgatgggtgcacatggtggagctggtctaaagaagagggggattcatatcacagtaaaattaagtgcaatttatccacagagcagaggaggttgtgaagagcaggcataggctatttttcatgtctgaatgctctcaagacctcttagtgttgtaggtagatgacgcacaataaatattccttgatttgaattggcataaaaggtcaaatcagaaagccagaagttctttaaggtttcaaactagattctaagaagtccaaaggcatctcagaggtcatctcagggagaaaaggggaagcaatgaggtagaactctgaacctccactctacctaaacaaggcagccctgctgtgctccgagtcatgtatggcgacatcagggaagatgtaaaataggattgtgataggaaacagcatttgaaagccattgctttacgaagggaagcgtagaacccttttccccttgttagccaattcaaatgaagacttttgggagctagtgaagagaaagacaggatttctagggagatgtttcagaagcagcctaactatacccgtgtcttcagaaagagcagtgtcgtctcagaagtaatcaccttcatcaaccagcaggtcagtgtgggtctcctgaagagcccgaacaaccacgggaagcgacatccactgttgtgcagtcaaaagaatctttgctctcattttctccactctctttccttcccaaatagggtatcataggaagatcctgcctttcttccagttccaacatttatgaagtgaaatttccatcagacagttgcttttgacaaacaaagattgactagaagcttctgtgagagagccttaggaagttctctggggaagccctgcctttgtttggttttctttgcctttggctctgtgatgtttgctgtaaaaaatgattactttcaggtgattaaaagtggggaagaatggtttcaagcttttcatgtagcaaataatatccctgtctgtaggattactttagttgaaaaaaaaacatggcttcagtgatgccttctcaatgtacaagattcagaggaatggaaagaaaatgaaataaggccggcctcggtggctcacacctgtaatcccgggaagccgaggcgggtggatcacgaggtcaggagttcaagaccagcctggccaagatggtgaaaccccatctctactaaaaatacaaaaattagccaggcatggtggcaggcgcctatactcccagctactcaggaggctaaggcagagaattgcttgaaccccggaggcggaggttgcagtgagccaagatcatgccactgcagtccagcctgggcgacagagcaagactccatctcaaaaacaaaacaaaacaaaacaaaggaaaagaaatctgcagttaatattttggcaagctttcttcacttgtatgcatttttaaaatgctaatgttaataacagttcgggacttctaacttctatatttaagcaacaaataaataaattgtcagatggtacttcatcatccttctctcccatcttcttagaaatataaattgctttaggtgggaatgctataattttagaccagaaaatacatgccagatgtctcttatatgaagccgtcccgcccaaggatatatatatgccttagtcattaggatgtgttctaaataatactgcaaagcccttggaaggatgggtctgaacactcacttatatttaactgctggcatgttgctttgtccctgtgtcttgtgctactatttccattgatgtaaaggaagcaccaattaaataacactccattattagagaaccaggcacaagtcagctgaggcaggagacccgccttcttttccagaaacaatgtaaagcctgggtgggtgagggtctctgggcttccgccgtgccttgcttttgacattctccagcacaccctataaacatgtctaaggctgtcctgtttagtctgattattcaaactatattgtccagggtagagcaaagggaaacctagctgaaccctggagatgacagcagggagagagagaggggcaaagaagggcaaaacgggaaaaacaggaaacaggctagtgagaagagtaaaaacgctcagggtgaggaagcagggtttctaagctctctaatctcccctgtgcagctggcttgctgtatggtttatacaaatccagtggtgatctctgtgcaacgtggtatcacctgtttaaagaggtctcatcttcattttcaaagaggaatacatgtttttttacttactcttctgcatggctgactccttttcatgctttaagtctcaatcttaatgccacctcctccttccagacgttcccagctaaagtggcacttcccagccccattactctctatgtttattgcctgcatagctcttatttgtaatgatttcgtaatagtttgatgatgatcatgatgaatattactttacctatttatggcctctcttttagtattaaattctgtaagccacatgagcatggggacacatctcctttgtcactgccccattgctggcatttagcacaagcatggtctataatagataccaaacaaatatgtattaatcatgtaaatgactaaatccatgaatgaatctatcagacagtgtagatagcagcacataaaggaaagggaatgtagtaaatttttcattttccttgaagatgtagctatgtattaggaatttgaaaaatacattatcaaacacaaagctaaattatgccagctaatgactactaaatataataaaatcggctgggcacggtggctcgcacctgtaatcccagcactttgggaggccgaggtgggtggatcacgaggtcaggagattgagaccatcctggcaaacatggtgaaacgctgtctctactaaaaatacaaaaaattagctgggcatggtagcaggcacctgtaatcccagctactcgggaggctgaggcaggagaatcgcttgaacccaggaggcagagcttgcagtgagcagagatcacaccactgcactccagcctgggcgacagagtgaacctctgtctcaaaaataaataaataaatataataaagtatgtagaaagtcagaaatcttggggattatattgcaaagaatttccactatattgataatggagaaaggcttttaatattatattttttgaatattaagaaattggcatctactcaccagtttggacatcgcttttaaaatacacactaaacgaaagccattttgtacttataagtgctagattaaattcctgcataggctgaaaaaggtctctttccatgcctttccaaatttacaattaacaaagagttaattattctcagagtcatttcttccaattcaccaattaggatgagggctatttgttacaatcataaaagaggaaatggtgcatgggcaagaagaaatttggaaaggaaatgtgattggaggaattatattgaaaggtgaaacaagggagaaaagataaagagaagaaaaattagaaattggaaacaaagttattccagcccctctctaataactactactctttggaacaagggaagcagtacctgacaagaaattttttttcttttattttttatttttattattatactttaagttttagggtacatgtgcacaatgtgcaggtttgttacatatgtatacatgtgccgtgctggtgtgctacacccattaactcgtcatttagcattagttatatctcccaatgctatccctcccccctccccccaccccacagcagtccccagagtgtgatgttccccttcctgtgtccatgtgttctcattgttcaattcccatctatgagtaagaacatgcagtgtttggttttttgtccttgggatagtttactgagaatgatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccacaataaatatacgtgtgcatgtgtctttatagcagcatgatttatagtcctttgggtatatacccagtaatgggatgtctgggtcaaatgatatttctagttctagatccctgaggaatcaccacactgacttccacaatggttgaactagtttacagtcccaccaacagtgtaaaagtgtccctatttctccacagcctctccagcacctgttgtttcctgactttttaatgattgccattctaactggtgtgagatggtatctcattgtggttttgatttgcatttctctgatggccagtgatggtgagcattttttcatgtgtcttttggctgcaaaaatgtcttcttttgagaagtgtctgttcatatcctccgcccactttttgatggggttgtttgtttttttcttgtaaatttgtttgagttcattgtagattctggatattagccctttgtcagatgagtaggttgcgaaaattttctcccattttgtaggttgcctgttcactctgatggtagtttcttttgctgtgcagaagctctttagtttaattagatcccatttgtcaattttgtcttttgttgccattgcttttggtgttttagacatgaagtccttgcccatgcctatgtcctgaatggtaatgcctaggttttcttctagggttcttatggttttaggtctaacatttaagtctttaatccatcttgaattaatttttgtataaggtgtaaggaagggatccagtttcagctttctccatatggctagccagttttcccagcaccatttattaaatagggaatcctttccccattgcttgtttttctcaggtttgtcaaagatcagatagttgtagatatggggcgttatttctgagggctctgttctgttccattgatctatatctctgttttggtaccagtaccatgctgtttgggttactgtagccttgtagtatagtttgaagtcaggtaatgtgatgcctccagttttgttcttttggcttaggattgacttggcgatgcgggctcttttttggtgccatatgaactttaaagtagttttttccaattctgtgaagaaagtcattggtagcttgatggggatggcattgaatctataaattaccttgggcagtatggccattttcacgatattgattcttcctacccatgagcatggaatgttcttccatttgtttgtatcctcttttattttattgagcagtggtttgtagttctccttgaagaggtccttcacgtcccttgtaagttggattcctaagtattttattctctttgaagcaattgtgaatgggagttcactcatgatttggctctctgtctgttattggtgtataagaatgcttgtgatttttgtacattgattttgtatcctgagactttgctgaagttgcttatcagcttaaggagatactggcaaaaaccacatgattatctcaatagatgcagaaaaggccttgacaaaattcaacaacccttcatgccaaaaactctcaataaattaggtattgatgggacatatctcaaaataataagagctatctatgacaaacccacagccaatatcatactgaatgggcaaaaactggaagcattccctttgaaaactggcacaagacagggatgccctctcttaccactcctattcaacatcgtgttggaagttctggccagggcaattaggcaggagaaggaaataaagggtattcagttaggaaaagaggaagtcaaattgtccctgtttgcagacgacatgattgtatatctagaaaacctcattgtctgacaagaaattttatagtctgatgaaagggattctaaagagtcaggggccacaggtctcaggcttcgactggatgtgatcatgtctgaggcctttcgatcctcactttccttatctggaaaacaagaatagctgaatctccttctaagggcgtttgtgatatgaactgagatcttgcatatgactgcaccaagtctagctcaattcgcattagttccctccattatacccctccctcgagctttacccagactcagaagaaagccaggcaacatttctacttctctatatgcaaaaacaaaagcaaacaagtggaaaacctcacaaaaacagttaacttcaacatttgggcttacacaaacaattcaaaaatctctttttatttcatccgccatgattatagttatttttctaaagtgaatgattctacttcccaaatgcagtaaacccactgttaaagatagttaattttcctctagatgattgtggcccttgaaagtcatcaaggtcatatttttaattatttccccagaatttttcctgaaacagtgtccttttgtctaaatcaatccaagtaggttttagcattagtcataaagagggtgctgtcaacaaagaaatcaactgagtggaagtgatattataatgtaaataacttgacataggaatacataacactcataaatatttattgatttatttaataaattaaaaattaatgcttatgatatgtaaacaagatataacaaggcagtcaagaataactctctttagttcatatgattttttcccatacgtaactgaatagcaagaaaacaagtaacccagtttgaaaatggacaaaaaactgaaatagatatttctcaaaagaagacatacaaatggccaataggatattttttaaatgttactagtcatcaaggaaatgcaaatcaaaatgacaatgaactatcaccttacacttgttagaatggttactagcaaaaaaagacaagggataacaaggttggcaatgatgtagagaaaagggaatccttgtacattgttggagggaatgtaaattagtatagtcactatggaaaactgcatggaggagcttcaaaaaactgaaaataagcctaccatgtgatcctaatactgggtatatatccaaaggattggaaatcaatatgttgaagagatatctgcattcccatgttcgctgcagccttattcacaattgccaagtatgaaattggcttgagtgtccatcaacagatgaatggctatagaaaacatatacacagtggaatactattcagccttaaaaaagaaggcaatcctgtcatttgcaacaacatgatgaacctgtaggacattgtgctgagtaaaataagcctgtcacagaaagacaaatactgtataatctcatatgcagaatcttcaaaaagttgaacttataaaggtagagagtagagtgatgtttaccagagggtggggtggagaggggtggggtacagggaatgggagaatgttggtcaaagagtacaaagttttagttagacatgacaaataagttttcaatgctattgcacagtgtggtgaccataattaacaataatgtcttttatatctcaaaattgctgaaagaataggccttaaatgttttcagtatagaaaagtatatgagatgatgcctaatttaattagcttgatataatcattccacaatgtatacatatatgaaaacatcacattgtatcccataaatatatacaattattatttgttaattacaagtaaaagtttaaaaaatggcaagtaaatcatgtagcccctgagatagatggatatgtgagcctagcttgaaacaaatgtcatcatatatccaccattacacaagactttgagcagactgaatgctcacagaacatattgggaggagatatttggcagctgaagtggcaaatagtcattttccaagggaacaacaacagtagagaggtttccagttaaagttgcagagtttctgcagagtctctagcagtgctggatccaagggtatgcggtgtatccaagtagctcttgaggaaaccacaggcacatcctggcatggggagcacctcaggagcacatcctgagtttcagggcatttgaaatggatgtgcagtcacatcccacccaactgcaagggatacccagcctacatgcagaggtcaggaaagctgcccacattaagacattgcatgcaataggcccctccccactaggagtctatggagacagaaatgcattttgaggagcaatttcatgcagtcatgggttaagtgaaccaagtgagctatgaagccagattttccctcctgggccacatatttcagaggcacataactcaagcttgcaacacgtattcaaaagagaccagctacacttggtagagacagccataggaaagtgaaatgaccctagggtttagtaaagccagctgtttccacttctgaaaataataaaatgaaataataaaataaatttaaaatgatacaaagttcaaagtttaacaaatacatttgaagccatttgcaacaaatacatctgaagctaattgctggctctagaaagtgtggggtctttgttgtggagcagtgttaatgatttagcattacttatctctggcaaatggtatttttgagataacatgttatggaagaaagtgaactgaacttggaagtttgaagatctcgattgaagtatcatttctgcctcaactacttgcattaacttgtacaagtcattcaaccgctctgaacataatggaaaaatgggatgagaatacatgttgtatactctccaaagacagggagactgctgatataagagggcacttttagtaactgatggagcaaaatgttgttatatgagtgtcagcatagggccctgggcttacaacggtgccatgagccttagaacagaggaaggacagctatagcaatgaaaggactagtgcagattcagaaaaataagaagacagaaaccaaggtgtagtaacatgttttagtatggaggggaaggcagttatagaaacttgaattacataatttgtacatttctgggagatagaaggtaaagatagcagctaatggagacaggacaggactggtacttgattatggaagaaaggaggtaaatagaagagacaaaaagggagagaagagatgtcaactgcctactctggtagcctctgtatccaaaaggttgactcaaacattcgctcataactttgtctggcttaatcctgctcatcccagcagacttatttcaagtgtctccacgttttgggaagtcatcactcacttctctgggctttcatatgggagagcatttaattctgttgaaaaactatttaatactacatctacctttctctatggactctgagcttcttgagggcatgtatcatgtatgttctattctgaagcacccatacctagaacaaagcttagcacatagtaggaacttaataaatatttcggagttgaataactagccttatgtaatcctcacaacaaccctaagctggagactcaaacaaggctggaaataagtaggtgccaagaagaactgagattcagacacatatttgcaggtaaaacataggaacactgaacattcactgagaactgacaacttgtggggttgttgtaggatatgtgaccagagactcttgaatgccagtctctgtacctgtaccatgttggctaacaagaatcgcatggaatccttgctgaaaatacagaccctagaagttttctcaaatctggagagactgtactatggtttgaatatggtttgtcgccaccagaactcatgttgaggcttggtcctcaatgcagctgtgttgggaggtgggacctagagggaagtgtttgggtccagtgggcagatccctcatgaacagataaatattgtcttgtgggagtggatgacttctgtcttgcaggactggatgaattaccacaagaatgagttgttgtgaagcttctcctcatgttttgctgtgtttgcacgctgtctcttgacatttttcttctttgctatgttgtaaggcagcacataaccctctgcaagctgagcagatgccagtgccatgctcttggattttctagccactagagttgtgagccaaataacattttttttctttataaattacccagatcaggtattctgttagagcaacactaaagggactaagacactcttattctcaccaaatctttattttggtaatgatttctcacacctattcatttgctccagaaaaggtagttattctccatagtctatcttcatcttccacttcatgcttattcaatccattaccaattcctgtcaatatatcttcctaaatatctctttcaaccaccaacttttctatcctcactattactatccttttacaagcacatagaccactgccaggaacccttgacttgccgacctgactctatactagttcttcttcctactgcagccggagcaatctttttaatcaaagctatgactcaacatttactctcttgatgaaatctcctagaagaccttttgtagctcttagaacaaagactgaaataaaaactctatactatagtctaaaagttccttgttggtctcacctctccagcttcctccctctgtgctccaaccacatgggcttgccttcaatgcttcacatatcacccagcttcaaattcccttctggctgcagggccttggcacaaactgttttctctgcctgatgttttccacccttccacctacattcatcagtttgacttctacttatcttttggagctcagctcagacaaggttagatcctgccattgacatactcattagcaccctgaaatatttcttaatcacagcgtatgattatatatttatttgtgtgattaactgataaatgactgtctgacccctcctccctgctttaagactataagttgtattaattcagggcctatgttagctttactcagtactctgtacccaatgcccaccccagcatcctcacaagtaagggtgttcagtacatgggtgttgaatagatgcatgaataatacaataagtcaacaattggtctcaggaatctcaataattttaatgctatcaaagtgatttgatgcagatttgggaaaaattatctaaaaaattcatcccaagctaagatcctatgattcttagctatcacagaatctgtgattctgtgctactcctctgcgcttctcatgtacacttacatgggtatacccatgaaaaatgtttgttggtttgtttgtttgttttgagacagagtttaactcttgttgtccaggctggagtgcaatggtgcaatctcggctcactgcaaactccacctcctggcctcaagggactctcctgccccagcctccagagtagctgagattacaggcgcatgtcactacactcagctaatttttatatttttagtagagacagagtttcaccatgttggccaggttggtgtcaaactcctgatctcaggtgatctactgcctgagcctcccaaagtgctgggattacaggcgtgagccaccgtgcccagccgaaaaatgttttaaagcatctaggatccttggcaggcctttagcacactgcacagaagggacattctgtgcctgtcactggaatgaccagcaactctggttccctgctttgcccagactgtttctatgtccccttcagtttagttcagttcaacaattatctagtgagcactttctctgagcggggcatctgctttgtgctaagtgtaagccctgcctccaagaactcattgcataaggagagacacacacatgaaaaccaactaattgtgattcagtgtaaaatacgtagtaattgacaaatgcatatagtttcatgacagccctgtagaaggagtaggcaagtgttctagtatggcttcaaggaggaaatgtagcttaaaacaggttcggagggatgagtaagagtttaccatacttttaagggggttgggaggaatatattgaaatgaaaattacattttgcaaatgcagtatgaggagccgtggtacagtttaatgtgtttagagaacaacgagtaactgtatggcagaagcagtaaatatgacgaaggaggaagctggtgtgtttggagaaggctgagggatcatgaggcatattaccttcctttaaaagccatgccctattctccctctcctgccacttcaaattcaggttcaccatttatatgtctattagtcctggtgtctttctcatgctctttgattagtccttaatccacaagcgcaacattgcaatacttgcctagcatatttcacaggcaggggacctaatgtccctgtgagaacccatcttgctgagattgtcgctggcagatttacttccagtgtgattgttgcaagaatttgtctaacagaatgaatgatcaaccttgagcagaagagattatgaaaaacttaatagcattgtagcaatgtggctgttaatgaaatacagttggctgctcccgctgtttggcaccaaccaacctgacactgtcaacatcacaatacgatatttattcccaattattttacggcaacaactgaaatacaatgtgttattaatcatatttattataagtatcaatttgagaaatttctgacatgccagaagataaataggtttattatgaaaagcagttctgcttggtgcatgctggctgctgctgtgtaataaatagcctctgtggggaaagttttttaaaagaaataaagcaaaaaaatagcactgaaaacagaaaggaagcatcaaaactcttcaaatacctgctgtgtccattggtcaagcacattcaggacatcgcatgcctttagaactccagcaggttccaacagctagtaggacattctagactctgagagagagcaagggaggttttatgactggggacaaagaaaagagacactgaaggcgaaggacaatctctgaaaatgcagtaccctccagactgctcctcctctcacaaaaacaccttcccagcatgcactgctttagggactatgattataccattgattctgtccagaaaacctgtgtcctgaatatattacagggctcattccttcacttctttcaggtgcctactcaggtatttccttatcagaacagtctttcgaacgaccccattaaaaaaatagtcctgtcaaccctatgttaacaattttatttatttttattatttgttaacaatacataataggtgcatatattttgggggtacatataataatttgatacattcatattgtgcataaagattgaatcggagtaattgggatatccattgccttaagtgttttaccttttctttatgctgtggacattcaaattactttctaactttttgaaatatacaatagaagaatgttaactataatcaccctattgatctatcaaatgctagatcttatttcttctaactatatattgtacctattaatctgtaattccacaactatatttacttcttatacttttccccttctaggctataaaccaaatgagagctgagcatctgtttggttcactgcccaacacatgcatgcctactacatggcagtcaaaatatttgtggaataaatgaatgaatgaaaaaaaaaagaaatagatgaatgaatcatggatgaatgaatcaaatcagtcagcaatgtctttctaaacaaaatttggatgattttggatgattacgcctcttaaaaatatttcttcatttcctaccccaatttagtttctactcaggactttttcaatatcttccaaacctattgttcttttttatttgtttgctttttgaggcaaggtcttgctctgttgccagggctagagtgcagtggtgtgatcacagctcactaaagcctccaactcttgggttcaagtgattctccacctcagcctcccaaatagctgggatgaaagtgtacaccacaatgcccggagaattatttcatttcttctttgtagagattgagtcttactctgttgaccaggttgctttcgaactcctggcctcaagccatccttccacctcagccttcccaagtgctaggattacaggcgcgagccaacttgcccagccctggaatttttgagcctgttcaattctaactattgtcaccaaaagtaaccttaagaaaaaaaatgcattatctccttgcttcattgcaccattaaaatctttcctaaattttccatgttaaagatgaagctcaaaatcctcagcatagcatacaaaacacttcataatcagatgcctcttcaaatacctcctatcagaatggtctctttgactacccctttaaaaaaattccccccaaccctatttttaaattatttacttatttttattatatttttgatacataatagatggacacattcaaacagtgcccccaaaactggggcagcagaaacaggtccttgcttattttctcagcttcacctcctgcctccaccccatctgtactgctggtccagacattcctacagaggtgtcctcctaagttggtctcttcctctcctgcttcagaggctttgccctgctcttctctgcctcttgaggctctgtcctgctcttctctgcgtcttgtggttggaatgcctgtctttttcctactgaagatctggatgcctaaaccataatgtaaaattgctgctttttacttccatttacagcagagaaattcctcctctggcctctcctcttctctgtgtttctttcttcataatttttatttatttatatatttatttatttatttatttatttatttattttcattgagatggagtctcgttctgtcgcccaggctagagtgcagtggtgtgatctcagctcactgcaatctccaccttccaggttcaagcgaacctcttgcttcagcctccctcctgtagctgggactacaggtgcccgccaccacccctggctagttttcatatttttagtagagacagggtttcaccatgttggccaggctattctcaaactcctgatctcaagtgatctgcccaccttggcctcccaaagtgctgggattacaggcgtgagtcaccgcacccagcctctctctttataattttcctactgttcacctgcatcaaactcctgaattctgtcatgcaactggaacagtaagagggaaaaacatggagctcaaagaaagatgttgagaaacgtagagttgcatagaatttactgtataagaatggaatctgtcaagtcagacaagcgacagagacctatttacaaagagacccagtgaaaattactggagaaataataaagagaaatgctgtgactttgaaataaataatgttcaaaagtcacctgcaatatttaggatagtgtctgaaacagatacaaatatttctcagcagtaaaagaattttgtatttagtctagtcatggaatagtagtcagttgtcactgaggaagcactttggggtagaagaagcatttgaatgtgtttgaagtctgaggcagcaggtgaggtttgattttatatttttgaaaatggatctatcagatggtggagctaccctcataaaagatttgtaatacgcctgtttacctacaagattaaatcaagtgtcatttcttcagggaagattgcccttcaccatgtgaaatatacataaagtatatgtcacaatgtgtgacagttctttgctcacatatatatttctccacttaaaggagaattttttttgagatatgatcttgttctgttatccaggatggggtgcaatggggcaatcacggctcattgcagccttcacctcctgggttcaagtggtcctcccacatcagcctcctgagtagctgagactacaagtgtgcactatcaagcctggctaatgtttaatttttagtagagacaaggtcttgctacatttcccaggctggtcttgaattcctggcctcaagtgatcctcccaccttagcccagaaggagtcttatttcattcatcgtatattcttagtatctacctgtcatcaggcctatagtagatactcagtagatgtagattgaggtttgaagaataagagatagctcaccaagtagaacactggatggtattggaactaatgtattcctttattgtcagcagaatggaccatgcacatagaaataataaaatgggagaattgattgccatggtctaaattttgtgccccctacaattcatatgttgagaccctgacccccaaggtgccggtactaagaagtggggcctttgggcagtgataaggttgtgagggtggagccctcacaaatgggatttatgccctcataaaagaaactgcagagaactagttaaacccttctactatctgaggacatggcaagaagctgctgttctctgaacctggatagaggacctcactagacgctgaccagcgctttcatcttggatttcccaggctccaaaactgtgagaaataagtttctgttgtttataagctaccaggtttaaggtattttgtacagcaacccaaagagtctgagaccataatgaagccattggaatggtgggaaggcaacttcatgtgagtaactacagtaaagccaggtgctggtaacagtcatgttgcccatagagcagatcctactattacagtgcctagcacattacctgcatatgatgatatgtgatcaattagttaactgattagtttatgaatcagtctgccaaaaactagggcagaaattgatagcacattaaaataaatatgccttaaagtttgcaaggagaccctattaactgcgcactgttttctttttattttctttttttttcttttgagacagggtctcactctgtcacccaggctggagtgcaatggcacagtcttggctcattgcaacctccacttcccgggttcaagcgattcttgtgcctcagcctcccaagtagttgggaatacaggtgtgcaccaccacacctggctaatttttatatttttagtagatagggggtttcccatattagccagcctggtcctgaactcctggcctcaagtgatctacctgccttggcctctcaaagtgctgggattacaggcgtgagccactgcacctggctagccactcactgttttcatgttaggctaagtaagcttttttgaagaccattaacataaatatacaacctaaatgtattttacctgaataattttactcatgtccacagcttgttctttcataggctgccatgatgaggaagaacagagattagtagtagcactattcatttctgatatttttgcagtagtggttctaattctcactccagtttagaaaagatctgtaggaaatcacaggtctacatttcgtcctctaaactactctgttgggtagaatttattttgcaaagacttatgtaggatcacttttttactacaggttttgtcatatgggatttttacaacctttttttctgctgaaacaaatggcttttaatccttaaaagggcagggctatattttccttcaaacatttttaaaataacttaagagaattaatttttagtaatagcaagtgaagaacattttaatcctagagcttaagaaaggggaggcccaataaccagatgctggaaatctattgaggttttttttaaattccagtatccagacaattggcatgaaaataaaggagcctagaaaaaatgttgaaaatgaaaacaataaaagtgacaactaaacatattattttgtatttgcaaagcacttccacaaatggccacaaatgtcatctctttaaattttattcctgagttgagtaggaaataaaccatttggagattcaaggtttagcttaagatatgggtgtttcttgaacctgggaggtggaggtttcagtgagctgagatcatgccactgcactccagcctagatgacagagcgagagtttgtctcaaaaaaaaaaaaaaaaaaaaaaaaagatatggatgttgtcaatacaatcgggggaaaggaatactttgaactactttgttggaaggagtttgaaatcgttgaggactcagcagcatgaagtagagaaattcacaattggtagaaaggactattgtccttcaaccttcattaaggttaactattcaaccttcattaaaaacagaaagtgacaatttcacagcaaattctagaactttagatcaaaagtcaactcaatatgggggatttatataagaaagagttaaaaaaaagacgaaatgtaatatctatgttattgcaagtgaaaggaaaacaggaagataaatatcacaagaagacaaaaatgtatctaacattttgggacaagattgtgggatccacagaaaattggaacttggaacttcctgttccacagagataagaaatacacttgcttttatctcacttctcaaaaaaagtaagatgaatggggttttaggccccagagagaaattgtagctgcaatcaattgtactatctgagtaaaaattgtcctcagaggaaagtgagtagggagctgtctgaagggacaggttattaacaaaagagagggataatggattgcgtttgcaagtgcagttggggctaacatcaatgccatcttcatagctggttcaaaaaaatattctggattcttttagtgtcttggttcttacctgttgtggttgcagaaggtataaatgtacccttaaaagagattagggagagaagtgcctcccacagcaccacgaccagaaagggaagaggaaggacaggcaatagccaaggactcctggcagtgaactcatgtccacatcaagatctaatgagcttgcactcaactcatttctagctctgccttggaagctggagctcctgcactgactatcaatgtgagcccctgagtaggagcagcttggtagagttgaaagaccattgatctgggtcaacagactctggttcctgtctcagcagtgctgtaatcaatccaagtcaaatatcatctctgggacttaatttgctaaatttaaaatgaaaagaaaaacaaaaatagaacaattagactagatcaggattcggcaaaccaaagcctgcttatcaaatctggcataccacctgtttctgtctatacaattgtattgaaactcagccacactcattcatttgcatattgtccatgaaagaagcttttgcgctgcctcaggagatctgagtagtggccacagagatgttgcagtggaccatgttgcaacattgtccaaaatacctaaaatatttacttcttgttttggagagtttgctgactggcaccagagaaatctatggtctaaaatcatctaaaaatttaagcacatatgtgtgaccacacatttcgaaatgccgtcttcccaatctagaacacagcacatgaatcagtaggtaaacctatccatgtcaattctcaaatttgaaattcattcacttgaaaatccagcaatttttcatgcttcatatcatatctgggtttggaataaagaagtgtgaggggagaaaaattccctgagcattttaaatctaatttcacctttattatgagactactgagtcttttcttgagcaaaggagagagtgtgaaatagaataaggtgctcaaaacaatagattaaatttattgaaaggatgagtaattggagtaatgttacagaatattaagcagattatttagatagcatatacatttccagtttgattaagtcaattcacaggccatcaaaaagtacacagaaaaatagagggtttgatccgatagccttctgcgttagaatgtagtcatttgctctctttagctatttaatctcttctgcatgttcagagggagaaaatgagtgatgagagagagaagtaataagatcatactgcaaatccttacatcgataagataacagaaggcttttcacactggctaatatttcatgttgtatttacagctcttgtccatgtacagatttggggtcattaacagaggtgttgatcagcaaagtcttagagtgagtctagggaaatggttgccaaatatatctgggtattggaaccatcaacagagcctgtgccccagacttactgaatcatggtctctggtggtggatcccaggtagtcacagctttaactgagtgattaggagattcactgtgcagccagttcgggaaccaccagtgaaatccaaccccttcatttattagtgttggaaccgtaactttctgaagatgtaccactgtcccagccacttcacctgtattcccagctcatttctctcagagaaaactgccaagtccccacagtggccttgcatgggccatttgtgatgtcacctctctgacctcatctcctactttccttcacccggttgtgtctagctattgccatgctctttctccaacacaccaagcacactcctgcatcagaggctgtgaattttccatttcctgctttgttcctccccaatatttgcatgaaccactttcccacttaatctaattttctgttcagaagtcactttcacagaaaagtcttcctcagccacatgtattttcttcttggtgcttattatcacctgacattttatatatctatgtttgtttcttgtttgtttcctactccattcggtaagctctaattgagaacagaaaagctctgtttattcactgttgtatccccaacatctatatctagcacatcattgattctcaacaactatttgtaaatgaatgaataaatggctctgctagatttttgtcacctggattgccagaccagtgcaataatggaaagctaagtaatgtaaagagctttgcaatcagacagctctggcttgcattcttcatgtgtgattttgagcaagttactgattcttgctgagcatcagttttctatgtgtaaagtggaggcatggacactggaggatcatggtgagaatttcatagagcaggccaataagacctccttggggaaagaaggctaaagtgggcctcgggtttaggtttcccccactgggagttccaaggggctatggagttgataggattccccatttgggctgcagaggagccagaaacatgctttagtttcttaatcctgaaggagtgggggattgtgtaagcctcattggtgcccttttgaaccacatttcatcaagatattgtgaaatggggagcagcaggcagccttctctggccaacatgtatcagggcctcgtgcttagtgggatgtgcccagagtagacagggaatccctggcagccttgcatagtccctctcatgcctttgctttcatggggctggctggaaacaggccggtaagccccatggaactaccgggacaaattaccaagcaatctcccatggccatgtagaacagccatgtagaacaggatgtctcccaaggtaagatttaaggtttgtggtttgcagaaaagagaagggaggaaataaaaaagcactgacaatcattgaaagcccacgttctaggtaactggctactcactttcacagcccagtaacaattttgcttaatcctatacatctataaataggattattgtcttcattttccagataaggaaacaagacttggcaaatgtgtagtgtagtgtttgacagtattatgtagttgtttagcatgcacatgaacttgcaagaccttgctcttccctaataattctgtcctttactaattctgtgatcttacaagctgtgccttatgtattttgtgttctattttgtaaaacctaatgacaacaacataataataccttctcatagaacaattattgtgataaattaagctgttgagagtcaggcactcaacccaacaacatgcccataatgtgtactcaataaatgcttgctagtatttttaataatattgttaataaaataacccagccagaggtagaacccaagattccgactctagttgacctgattctaagccaactcttctacagcaacacatcggcccattgatttagacagcatggaactcatagcccagggaattcacactcttaaagaacttaaacagcacagcttaatatggtcccttccaaacatacgactcagtaattccagtaggagggctaaacaacctattctgagaggtctccaggaaaggaagcctggagtcttccctggaaacatgttcccttatcgcacctcccttctcttcaggatgctgaggacctctggctccatggacttcatcctcccatacaattggttcccctcacaattagtttctggcactgtcatcaaagtgatccctctccaaggacatggaacaggatgaaaaacaaattgtaattgtaaactcgaagtacgatcagggcaggcacagcaaaggaggaaggttacgttgtgggaatcaggggtcaacaataaaagccagcctgctgcctgtccccaccaggcaattttctgggctgttcatgtccccagttatgcccccaaacattcccatgacagcaatgcctaaggaaaataacctctgaagattgagcctaaaactagaatcatttttctctctcattctaattcatagcagggaagtggactggctccttaaatccagaaagtatcagacttatctgatcaaatcattagggcaactgtgttttccctcatctccagacagcatggatgaggagaaggagccaaaagacacagcatctctcttcaaatgaatactcaacttcaaagaagaaagtgaccactgttaaggaggtgaagtcatagcctcggcaaacctttgaagctgggggagaagatgtaaattaagcaccgacaagcagggtataaaaataaaatccttgaaggaaaagcaaaactgctttgcaatgggtgtgaaagtaaaggaagctggccaaggccagaggaggtaaacagggttgcatgatgtaaccacactaggaaaaggcaaactagttgagaaatatagagaaaatacatggaagttgtaaattgtctattttatctgagaacttataagaatcaaagtggaaacaaggtcaattttcttaatattcaggccattataaacccttgtggggaacaggaaacttgcttttatttgctgtttctgcaatagtgttgtttgggtacacgacaggaccatggaactctataatcttgtatagggatacatgccaagtgccattcctgattcctttgtagttttttttttttcttatgtctcctattagttgaataattcattttttgtttatttattcattcattcaataaactgcttttgtgcttacagtataccagacactctactagatgctaaataagcaaagatgaacatggccttgtccttacccttaaagaaccataactctatataaaaacacatttaatttgtaaattagaggacaggatagttagtttgtgatagagaaaaccaaatggtcccaaaggaagacaaagtataaagttttgaaaactatcaccccaaaattcacatctaccttgaagtgcaaaatggcaacttgtttggaaatacggtctttgcagatgtaattagttaaggtcatagcagattaggatgtgttctaaatgcaatgactgatattcttaaaaaggagagagcatgcactcagacacacacagggaaaaaatccatgtgatgatggaagcagagactgaagccaagaaaagacaaggattgacaggagccaccagaaactaaaaagaggcaaggaagcattttccctagaaaggagcatggctccactggcactgtaatttcaggttgctagcctctagaactataaaacaataaatttattggttttcttaaactacccaattggtgttactttgtcatagcagccctaggaagctaacatacaggtgaaggacatttaaagctatagtagtaagagaagagatgtcctaagatgtttctttggtgacatcatgtgatttttaatatggattttgctctagggaattatctctgtcactgccaatcaaggtaccttgctccactcatcaaggggtggcccatatccaagctggagctttttgattctctctttctgcagtttggatatttatgctgatcatagctcccagaaaaaaaaaaataaattggccgttttgcttccttgattccaggagctgtcccagctcttgtgcatcctacaactcagttctttggcttttccttctttgtttgcttaagtgaaccaaagatggcatttattgcttgcagcaaagaagtctaactgatataggacccatgattaatgttctggttcttcttttacagagaacatgatagtgttctctaatttcatctttttttctattttttttgttctgttgcaagaacaaatctgctatatttatggcaacttgtggcaataatggaagattctaagatacatttttataaacatgttttagccctggataaaataacaccttaattcatcatggtctcttcatctggaatcccctttcttttaaccaatttcacataccaaaaattattttttgaccacagaaatgccttaagggataaataaataatacagtcatcagagtttaactaaactgggctttaactgaggtgcctgcaatcaattgtatatggtctaaatttggacttagccttcctgagccatgggcttcccaggtcatatccaccaatgggtcattcttgaaatcagaaaagtattatatggcaggtactcagcccagtacccgacatatagagatgctaaaaatattgtttttctctctttctttcttcttccaatcctgaccctccaaaggtcatcctgactcttctcaaagcctgtccttttttctcaagtggacctgtagctacagaaagttgagaatgataattggataaatgaggacagatgtctctttagcccccttcctctttagcttgctggggtctcctgactggattactctcagctcaaggaacccctccctcaacctcaaaaagagaagacaagattcatttttatacaatgttgtttgtcaattcaatccaatctcaaagtacgttttgccttctgaaatctaaatgttcacgtttctttttccaaggtatctatattcatttaatggggaaaaaacaatacctggagtcattatatgtgcatctatgtctttctaaatatgcacaagcattttacttgaatatttccaggtcctgcaagaaattctgaaattgattttcatttgtgtggcttcaggattcatttcaatggcagcccaaacattcttccagcagaatggccattcagacctgcttaatgttgtgaatccattgatatttttacccttccaaaagcatacactattgattctgcctctctcttaattgcacagaagaaaaactgacttctcatgaacacaacttgatatctcatttgatatcttcattttcataccagtgtaactgagttatcaatgtttgcttggaccagactattaaaacagccacttggatagtttgggtttttaaaaagtctggcaaggtatttgcctcattgttattatttggaaatttctagaaatatgtcatctgtaatatcagtgtactggcccaccaactgggttagaagtttctgtttgatgttcttcccccaggactcccatcctattctatcttacctctagggtggcaatgatcacattgtcccagaaaaatgaccatataactttatttttcaaactggaactttcaagagtgaaagggaggatggttattaattaagctaggaaaagggaataaactcagaatgtcctaagtaaaaataggtcactctgattttcaccattagttcagtgctgtgtctcttcctctagacattaagcccctggccatatttagcttgttctctgctctctctctagcatctgacatagtacacagggaaggatctgcacttcagtcatggtcctgtgtcatggcaagaacagataaaacaatctccctcaaacttacaactcttttgttgtaatcactagaatatattatatgttggtatcactcaaagtagcaattggccaatcatctcttcttttctggctaattgcttatccttcaagacacctgatgtcaactgcattgaggtacattcttatatttgtgtagcattctgtcttgatatctttgtgcttgatcatgttctattataattgtttctttaatagacagctgagaactgctgtgaactccatttcttctctataccagtatttagctcggagcctgtcacagaataaggaaatagtacataacaactgaatgaatgtatgtgcagattcgtgaatgaaaaaatgaaatatccagattggacattagaatattctatctgtcaactgctgtttggtttgcctgatactgattatttgcctacttggtaatatttatatagctagaatcatggacccacagactactataaactaagagacaattccagacattagtaagacagaaaagttttctagtatatcataaaatattaaatctgatagtagcactaccaatggcatgatatatttaaaggtgaacatagagctaatgttcatataaaaatcatcctgtgatatttgttgctgcttaacatttttaattcaatttcatgggagaacaaaagagtgattaagagctagaaaatagaattcatgagaaaaggttagaggaattgaaattatttagcccagaggaaaaaagtatatgtagccattcagtgattcagtgaaacctcattatccaatatcagtttattcaaacgatggcataagttaaactgacagctatgtttccaatgaacaaatgacctaccatttcttaaaaaataaaaaaaaaagataatcagaaaccatttaacttaaatatcttttttttttctgtctgatctcgtctattttggattataaaggatttattttacatgaaatcttctgggtcacactttgcatggcagtagaggtagtagatcaggtcaaagtgggttgtgccccaatttttttttttctcttaagtttctattttctaaaatgctgttgtgttttcattcattcaacaagtatttattgaggaactatgcatgtcagcgtccatgctaagcacgagggacatagaagctatgacacacaagctatttgcattattataagatgtttcagttaagttcctcccttaccgaacttaagtgtgttgggaaacattaaaaagtcatacccctgacacagtgagatggtatctcactgtgattttgattttcatttctctaatgaccagagatgatgagctttttttcatatgtttgttggctgcataaatatcttcttttgagaagtgtctgttcatatcatttgcccactttttgatggggttgtttgttttttttcttgtaaatttgtttaagttctttgtagattctggatagtagccctttgtcagatggatagattgcaagaattttctcccattatgtaggttgcctgtatactctgatgatagtttcttttgctgtgcagaagctctttagtttaattaggtcattaaaacgtcaggaaaaaaccgatgttggaaaggatgtggagaaataggaatgcttttacactgttggtaggagtgtaaattagttcagccattgtggaagacagtgtggcaattcctcaaggatctagaaccagaaataccatttgacccagcaattccattactgggtatataccgaaaggattataaatcattctactataaagacacatgcacaggtatgtttcttgcagcactgttcacaatagcaaagactttgaaccagcccaaatgcccatcagtgatagatgggataaagaaaatgtggcaaatatacaccatggaatactatgcagccataaaaaaggatatgttcatgtcattcacagggacatggatgaagctggaaaccatcatcttcagcaaacttacacaggaacagaaaacaaaacaccacatgttctcactcataagtgggagctgaagaatgagaacacatggatacagggaggagaacatcacacactggggcctgtcggagggtggggggttagggaagggatagcattaggagaaatacctgatgtagatgacaggttgatgggtgcagcaaacgaccatggcatatgtataactatgtaacctgcacgttctgcgcatgtatcccagaacttaaagtataataataataatagtaaagatatcaccccccaaaaaagtcatacccgtaggttaaggcatctctaaagtacacgtattcttatgttgggggcaaggaagtagcaaattaagctattatctgtattgtttatggaattatttttcagatgggttcccattgaagagaaaatgggatatagatcaaatgagctttgaaggataagaaaaaagaaatgagctttgaaggatgaaaaaaggaattttttttaaaaagacaatcatgttgggaaaactggatatccatgtacagaagaataataatagactcttatctaaccacttttacaaaaatcgactcaaaatggattaacagcttaaatgtaacacctgaatctataaagctactagaagaaaatgtagggggaaagcttcattatattggttttaggtatgacttcaaaagcacaggcagcaaaagcaaaaataaagaaatggaattgcatcaaactaaaaagcttttgcatagcacaagaaacaattacagagtgaagagatcacccacagattaggagaaaatatttgcaaatcatattgcaagtcataactgttaaagagctaatatcccaattagacagggaactcaaaactattcaattacaaggaaacagagaacccaattttaaaaacgggcaaagaaccggaatagacatctctcaaaagatgaaatacaaatggccaacagctatatgaaaaaaatccttaaaatttttaatcatagaaatgcaaactaaaaccatactaagatatcaactattatattggctattatcaaactgatgaaagataagtgttggcgaggatgtggagaaaagtgaatccatacacactgttggtggcattgtaaattagtacaggcattttggaaaatagtatggaggttcctcaaaaaactaaaaacagaattatccatatgatccagcaatcccacaactggatatgtacccaagtaaatagaagtcagtataccaaaggagatacctgcactcccatgttcattgcagcattattcacaatagccaagatatgaaaaaaatctgtgtccaaaaatgaatgaatggatttttaaaatgcagtacatctacacaatggaatactattctgcctgaaaaaataaaacaggaaattctatcatttacaacagtcacacttgaaggatattacgttaagtgaaataagccaggcacacttacatgtggaagctaaaaaaacgggactcatagaagtagagagtagaactgtggttatcagaggcaggtgggggctaggagggtagtgtatggggagatgatggtcagcaggagatgttggtcaacaggtccaaagttacaatgttaggtttgtgcaacagtaattgcatttttgccattaaaagtaatgacaaaacgcatttatccacttgcttcaaatcataattgctaggaggaataagttctagagttctattgcaatgcgagatgactataattaataataatgtatatttcaaaatagctaagagatatgactttcgatgttctcaccctaaagaaatgattaatctttgagatggtgaatatgctaattaccttgatttatcttgctatcttgcagtgcttacatgcgtcaaaaatcacattgtactttataaatatatagttgtcaactaaaaattaatttttaaaaatattattaaaaataaaggttattaaaaataaatattttattatataaaatgttattttaagaatttagtattaaattcttaaaatttaaatttctaatttaggattaaaaaagttagagctataaagttcataatattaagctgagaaatataaaattcacagaagtcagttgacaagttaacacaataagttagtaattactgtgactatacttccggtctcagatatcttactatctagtactcttttcttcttccaaattagtatttgcatttatttactaatagtaactatttgtatatggttgtgaataacagaacccaactatgatggcttaagcacataatatttatccacctacatcaaatccacagctaagcagtccatagctgctataacagtgccacaaagtcatcagagacctagactcctgtcattatgctttaccctttttgttatgtagctgtaatcttttttttttttttttttttgagacggagtttcgctctgtcgcccaggctggagtgcagtggcgtgatctcggctcattgcaagctccgcctcccgggttcacgctattctcctgcctcagcctcctgagtagctgggactacaggcgcctgccaacacgcccggctaattttttgtatttttagtagagacggggtttcaccgcattagccagaatggtctcgatctcctgacctcgtgatctgcccgcctcggcctcacaaagtgctgggattacaggcgtgagccaccgcgcctggcctatgtagctatactgtttaaggctacaagaaggctgcttgacatatatttagttttgttgctaagtgaaaaaaaaaatattgggtaggcaacttgttatctttaccacatcgttgttaagagacaattttctatgggtctccagaatttctgcacatcttgtagacagaggcactgcctctctttgttccgaagtgattttccaaggttgtgtataagccttggaagctagagatcatgtgtcccttcaaagcagagagaagtttctttactgtccaatataataaaggtaatgtctgtctctctggggcgaagattaggcatgagcactgccagttataaaagattcaaatttgctaaactcagtatttctttcctgtaacacgatgcatcatgtgtgcaggcatcacatggccctattcatgtcaccctatgggaattgaggcccagggactggcttgagaaaatgaagttactggttactgttattgttgtaagtaaccagatgtcttttgtctctgactctgaaatatcatgccatgtgccgatacctatgacactgcagcaggctaccttgttagcttgcaagtaagtttcagacccttcacagttcttgacagtcatccataactcgctttgtatgctataatatctaaggtgtctaactccaaggtgtctagaacagagtaggtgactaatacatacttaaataaatagagagtcccaaaaggcattttaatgagtcagaccagttgacataaagtcccagatgataccactgaggagccttattaccttccacaaaatcacttaccctctctaacctcagcttcttaaattagtagtctgcattttctccttccccttgctcaactgttgtttatttcctaacttactgctaattggctatgggcctcatcccttcagagaaactgttcttccagggtctctagtgacccactaactggcacagcctgctgaccctttccactctgtatctcactccctgctgttctgttaacatttaactccttaaaactctttcttttcttgccttccatgaaacttttctctcctggtttctcttatatctccaggaaactatttatccgtctcttcttccattatttactcttaaacattgatatgccataaatgcccaccctccctccagccctcttctcactctgtagaataaagtttataccttttttttaaaagaaaaaattggttttgcttaccatgtttgtgcttataacttttgaatttctatctccagtccagatttttctcctaagcttcaaatctatatttttaacccccaattcgttacctctacctggatgtgttattggaatttcaaatgaaaacatgtttgaagttacacttattatttattttcgcacctcccaaactggtagagctggcatccctttctcagtgaatggtatcaccattcatgattttttttttttattttataaacctatcgactctaaggccaataacattcccatccagtactatggctgtctcatcaatacttctcaaaacccttgtctttcttcccactgcctccccattaatttaggccctcactatctattacgctggccccatgttcaaaccgtgtctccagaatctgtcttactggatcataaaagcccccagaataaactttcagaaccaaactcacataatgctgctgccctgattaaaatctttctttggcttttctcggtcagaagaatgggagaaagttcaaactctttagtatgatacacacaaccctccttaatctgacagttcctgcctcttcagcttcacttcctatttctctccatctcacagcctgtgccccagccataataaaccactcacattttccagcacgtgccctgttaatatagaccttgcaccgctaaatagactgattcttctacctgctatttgacatcacccccaaatccagtctagggaatctttcatccttcaaaagctatagcttaactttttctagccctgggaagtttacccttaactatcaaacccctcactcttaagactggtggattactgcctacctggtgcttctctctaccttgaaggatttttttttttatcacatttatcattgctgtaccatactaactcgttaacatacctgaaaccactaataaaagttgtcttatttatcgttatatgtccaacacagagcacaaatccagacgcagagtggaggctcggtaaaattatttaaagagtaagaaagctggagaatttgagtaataccagtaaaaaggcagttactgaagtggaatgaaaggaagaaagcacagaatttacctttttcatagtttgccttaagtaaactctggaatgttcttaggcaatgatcaccctcctgggccccaaaatgagaaatcaagttcaccaggaagtagccaattgggaatggcacatacaaggacagtgtccttcacctcccagtcggtccaaagataagccagtggggaaaagggcagctacagggcaatggtttgtcttgaactacaaacctaaagaacaaactatgaccattgggaaactttctagttaaggagcacacagtctggcccagactgagtttgccagattaggagagagagagcactgggcaggagtaaaggtcagaaggcttgggtgcaggtgcctctgctaacctcaccactcctgggtccttggacagtttctttcacttctctaaacctctgtgtccccatatgcaaaatggaaatgcaaaatacacattttaccgaattcatgagtttgttaagagaattaaaaaagagaatgtgtatggaatgttttcacagtgcccagaacagggtaagtgctcagtaaatgttaattattgtttgtaatgaaatacagaaaacactcaagtaggactcagctctgttccagctattcttcaagaggtgtgacgaccaaaatgtgtgtaaaattattaattatttggctcaaacacctttcctcttcaatgtgtagtggagttcctttatagatttattacaaaaaaaataagtttaaacaactaaatacaccctgtggaatgaaaatcaaaaatagttactctggtattaaatagctactgtagcattaattgctaaagaaattacatagagaacaacacacaggcataatgtaccatagcaataccgttaaattccaggccctccttcatgaaatgtctcttttctggcctcccttttaatcttcctactttcttcatgcacatcctaaaagtccactcctgtcttctatacccccaacactttcatctcagattgtttatactcatgtagttgcttcttttattactagtttttttctaactgcaatgaactctcggtttttttttttttttttttgtaatcattttcttagagtttgcactttaggtgcagtttttattaattttattataaatattcacttgcctaattgctattcccactggggtattccacagagaacttaaatttcacaagtctcaatttcaactcattttcttcttcttaatgcaaattcttctcctatgacttctaatggcaaacacaaacctcatacccaggatagtacacaccaaagccagattatttgaatttaactcccagatctaatagtcagttgctgtgtgacctggaataaattattaaccctctgtgcctccttcttctcatctgatattgatattacttaactcatagggtctttatgagggttaaatgaattatcacgtgtaaacactcagaatagctccaaaaacatggcaattgttacatatgcaagtagcaactcaccaagaaaaactaatctgaaagccatctctgaactcctacttccatttagtcactttttttttttttttggagagagtcttgctctgtcgcccgcactggagtgcagtggtgtgatcttggctcactgcaacctccatctcccaggttcaagtgattctcctgcctcagcctcccgagtaactggggttacaggtgtgcaccaccacgcctggctaatttttgtatttttagtagagacagggcttcaccatattggccaggctggtcttgaactcctgacctcaagtgatctgcctgcttcagccttccaaagtgctgggattacaggcatgagccaccatgcctagccacttcttttctaaatattttaaatagatccatatcaggatatctctactttcattaccattgtttaaacccaatcttctttaacctaaatagtgtaacaggctctgaaatggttcactcatttgttcatccatcatgtaacaggtaaatattcacttcttacttttaacctgacggtgggaagtattaatgtatgggatagtcatgctctcctccttctggtcttgcttcccctaatccagaataatcagacagtgccattgccccatgatccccaatgccttcatgtcaaagtcaaaacatttactgtgccatgatctgacccttatttaccactccatcctctctgctttccattccttgtttgaatctcagctgtaagcactcgtctccttgctttgtagagaaggagccatgttgctactttcctctgggactttgcacctgtcatttactctgatttcctcccaagtccaccctatccacacacttttgaggagctagttcctattggcggtttcagcaagacgtcttccctggttcccagacctatgttctgataacatgtagaactcagtcctatcatgacacaaaattgcattgagttgagttttctggtttattcatctgcccggcatactgtaaggggtatttgttaaggaaattctaaccaaaggaacaaataaactctgaaacttcagtggcttaatataggttttatatatatgtatatacaaaatgtatagattatatattttatatatgtacagtatgtaatattttagaaataatacactatacaaaaataggttatatacatattctatagtaatatattaacatatttatatattatatatgagatatacatctagaatatatgttatatatttatagattgtatattatacataaaatggtagaatatttttataaaatagaaaatatataatcatatataatatatatttatatattatatattatatttacacataatatataaatacatttataaataatatatttatatattattatatttatatattatatataaatattatatatttcctcgattcatttattataatatgtacatgtgcatgtccctttaagagaagagaggtatttctggaaaggagttgtccaagcagagatgacttttcctagcctccctgataatcatatggagtcagggactgtgtcttgcttattgtttgttttctctgcctaacatagtacacagctcagaggatgttctcaataaataagtatgagataaattgatactttacctctctggttgcagcttgtctttttgctaattacacagatatgggaaggttgtaggattcaaataaattttacaataagctgtttcctttttaaacttagagagcatctctcaagtctaggggcatgtcacttttccatcagcttccctgctatgaacactttgtgaatcatcctaaattacatcaaaagaaaagacatcatgttctcttctgcttggtgatacaagagggcttttccactggaaaaaatactaaattttctatttcctgtgtgttaccagcaaaaacttttttcacaagctgctgctttctgggttagagtgaataagtaactaaccaattagctcgttcatttccaattcaaggacatcaacaatcctctcatctattggtggcaaattcttgtcctatgtgatgtcaacactttaatctgtctatggcaagcattactaattattcatacttaccttttagccctgtctgtggcttcagaataatttccaacgcaaccatccaaattttgacccacagcgtgttacatttggcattgcggtgactcagttcctcatctttagtgtactccttcatcgtttttgcagatataaagaagttaaaggaaaggaaggtaaagtaactacaaagaacatgcaagatgttggtagcaaagcaaaacgcaaaattttgggattctgatcctcagccactattttttttttaaccacttggaaatagacatctaaagcagaaagaaatgttccagaagccttggtgtgtggggggtgagtggatgtggctgtatgccctaaagacaaattttaagtctcacaacttggctgcgggcaagctctggtgtttagcaaggtcttgtattttgggtttacaagatgctcatttgtttctcaatggttccagcttgtcctttcacctggaatgttgtcaggaacctggggcctagaaacagctgataaagataagctttgagccactgagtgtccttgggcaagttggcatgctgctccgggcctcagttgcctgctcgacaaaatatggtattgggtttttgaggtaaatttagattaacttgtgtgaagcactgaatccaatatatggtgtctttaggtccaacctcaggtctccctacatgttgtcagaaaaagacatttgagcattttaagagtgaaatcaaaagtgcactaccaaggtttctaacagcccagttctgtcctggcttttcttcctgcaactgttttcagcttgggtgagtcacttttctctctggacctccattttctcacctgtaaagtaaggagattggaaaatgatcttaaggccctttcatctttgaatatctatgattctgtgcctctctgtatctgagtccccagaattctttttcttggaatatctccctcagttgagagaaaattataagaagggctgcttcccaggtttattttgggtgcagtggccagtctcccttgaacttggcatttgtaactaatctctataagcaaagtgtgctccaattatgacttcaccctactggcccagcagagctattgggtgctgccagtagggataaaggagtgttccagccatgaggcaactattttgttacaattaatggacatggggttgagagacatgtccctcaggcattctcttgtgccaccttgaagaggtcatcctaggtaaaagttctctgaagagaaatattggcagatacatatggcaaaacacagcctccaactccagtcaaaccaccaccacaagcacagtgaaaagcagaaaagaactgttgggtgtagtagggtggaggtgaagaccttggtcctgcctttatgaagcatacatcacttcgcaccaagcatcacgcttggtgctggacacaaagagatgatgaagatgcaaaatgtggtccctgtttttaaggagatcacagttttctcaggcagtcttacagtaactatggcagtcaagatattaataataatatatatcttggacattcacctagagattaaaatgatgattctagcaagataagtcatcaggtaatttagattataggtgggaataatgaatctttcagaattactaagtggggccttccatagaagagtataattcaagacacattggtttcaccagtggtgacctcccctcaaagtcagatacaggggagcagacttaatattgttggaggaaaagagagtcaaaacctaccagaaggagcatcacaagggagacaaagaattgaattgtttctgagttggtaatgtgaaagttgctagaaataatccttgctcaggtgctctgggactagtttatgtgccagctgtgtgcacaaagtgaaaggcttaattgctagaactgtattacttttggctctagtctgctggaccttccatctcccaaactgtagctccctggagctttctggaagtgcttttgtattcctggacttacctatagcatacttttaaaggcaagggaagcttcttaactcctcttaaatcagctaagctatgctggggtagtaataaacacctatatctcagggacttaacacagatttctcacccacatcacagtccagtgagggcttctctccatcctaaaactttccccctggaactcaagtctctctatgttgccttgaaagggaaaagagagccgcagggtcaggatttaaggaccatgcctagaatggcttacataatttccacccacattgcattgtcccaaattcagtcatatgatcccaacttattgtcaccaaaatgctagggatttggtctaggccctgctgctcacagcacagaatgccaatcaaagagacaatgagtattgccatggaagaaggctttaattggatgctgcagctgaggagttaggagatcagtctcaaatttgtctttcaaattagctaaaattaggggtttacatagcagggaagaaatgtaaccatgtataagaaaacaggaattagggaggggtaaggaagagaagttggttgacaggaagtaggtggaaagttaggcaatcctcatgggtgaggggtctggcatctctttgtccagatgcagtgatctggtaagtttcacctccttgatactgtctgggaggtgtgatggttggtttcctgagaaagaaactgagataagacaaatgtaactttcttgagtttcaagactggggggatcaatttgtatgtttattcaatagaaataatagacattagttctgtagaaaacttggaccagtttcagtctcccctttctattttcagttcctcgatcatgggaaatttggtcattgatctttctggctgcttcatgctgaggaggggcattacaagcagctccatactaagggtgatgaatcaaatgttaatctaatactgcagtctccttctatgacacaatctttctctctctagtctcccacttccaccaaagacaaatcatagcaggaccaacctacctgcaaaattagcttcagtcccatatacttggcccgattacccacagaaagtacagcaagcatcatcatccacatagggtctccaaaattggctttcctggaaccattcacaaggccatttcagtcaaagccctgggaaaataaccagttcctccaactgtgtctcattgtaaaagaaaacagattattattgaacttatgtaagcaaccatattgccataaattaagaatattcacaaatagtttacaaattctacagaaatcaggcagagagagaaatgtgcttcaaattctattgacaagagtacactctactcaattgctaaaggttgtaaacagctcaaaagaaaaagtgttctccagactctgaaaaacaaaacaaaaagaatcagcaatgtttcaaagaaccaaaaaaaaaaaaaaaaaaaaaccataaaaattatttctgtcctccattagctcagtccatgcaatcaactcccactctgcttcatattgggttagtaatctttaagaacacatcagcttttcaattaatatcctggaagttttctctctagcccaatggcaaaatctccaaagttatcagaaacctgcattcaaggattcttttaatgaattccccaaaagaagcaagttccagactgtagctgatgataagcctgtttttaaaaacaattgaagcaaaataattatggatgagaaaagtctcaacagacacaattgacaagaaaatttggttatttctgtgacatacaaaaatttaacataatcgtaattattactgataacgtatattaagacatcagaattttaggaatctcatgcattaatgttaaagctaattttaacaaaaccttataaacaaatctaatttgaatcagtttgatcataaggtaagaagtcttttataaccttttacaatgttttttattaaagataaaatcattgctctaagaaaactgtgttattccatcacaatggcccagatactggtattgcatcagtgtgcttttgatattaacatttaatttatagaaaaccttgaactaatttgtcccttaaaattagcccttaaaatctcacatgcgcccacatcttccacaatagttcctgggcctacagggattgaatagtttcaatttctagccctgtgtctcgggcaagttgttcattttgattgtcaccttctcctgggtctgaaaacaaggctttgactactgtcaatgttcaagatttggcatgagtctgtgccttttttagacccaagagtcaaagtcctaacttaacagtacaaggactagttaataggacatttatattatagaaagtcctgtcattctccctcacatgtcacaaattaaagcactgtgaattggtgtctagtagttaatgcctgcagcactttaaatcactgtattaaagtggctagattattccttgcatatatctaatttctaacattgtagtgacagaactgtgacccaaagcatcaaaaatgtgataggtccaatgccaaacttatccaagtaagacaattaactttattctccatcatttaagaaaatggtaaatgcaaatatcggttttggaaattcaatatgaagataaataatctctttttgcttaaatactatacagtgaaacagggacaaagtaaaaacaagcacagaatatttttttctgctattttaaaagagcatcatcatacatttccaagactggtttctagatacggtactgacaactattaggtagatttcaactccagaatcttcaaaacagaataatactagaatatattttgttttcaagtacacacattaaggcctaatagtgataacagatttggaataaaaaagaatcactagaaagcctgacatttttactactacttaatccaagtgaatgtcacttagttttgatagtggtaaatacaactaacatagtctgagagaaatcccagtcaatataatttcttttaaggacaaagctagtctttcctgaacattaaaactttgtacccctatcacaatttttcctcattacctaaagaaaaagatctgaaaccgattcaaattattgattgaattgaatttccttagggaaaaaaactgtctaaacatttcttctcttacctgcttttccagataaataagtaatctactatttctgctcagaacttatttaaagaaacaaggttttttgttttttaggggttcttttttgacccatagcttaaagctcttgtagctctctagatcatcagagtcagcaaaaccaatcaaatttttaatggctggtgtcccctatccatttttgcaggcctgacaaagatagcttaagaactttagataaatagagcaaataatgaattgttggaaatgcataggaaacaaaatgactattcatagaagaaaatacaagccttccattaaaaactaaaactatcaatggttttttatatgtgtatataccagtaaaacccaaaggagaacaaatagcaaataattaaaaattaaaagcaaaaacaaactggaaaccaacccccaatttttcacctactcagtttactttgaaggttacagtataatccagagcctaaaacaaacatatgttggatattttgttcctattatacaaattaatgtctctaagtccagcaacatcactataccttctgtgcaattaagaaatttacttcaagcacgtgaccagtaagtactttagtgctagtactgtctatgcagaatagcaaatacagtgtgaaacaaagcaatgcaagcctgtatgtaaaatttggctccatgctaaatctggattcatgcttaagtatgttaaaaaaaaaaaaagaaaagaattgccaagctgccaatatatttctttacagtatttcttaatttaccttcatcaatactaagatctttagctatgagcaatgttaattagccaaatgtctccaattttctatcagattttaaagagtattttactacttatttacacaaaccattcaacatgcttggacttcgtttttgtcctagattttctttctttctttgttttttttttttttaaaatgaccaattattttattttaggacaaaagtttaccatggtccataatttgtatcaacctttacataacttatgaattagacaaaataatttgtttttctctgtaaaaacacatcttctctcacacattttatatacagaataatatattaattgccattctgattcttagtaactttaaattttagtgaaaacctaggaagtaagacattctgaactagctgtcagatattaacatttcagagatgagaacattccacaattttaaaaaacatgcttcccaacgtcataaccctttctttctctcctttctctttctttcttttccttccttccttccttcctttctttctttctttctttctttctttctttctttctttgtccctccctccctccccccccttctttctttcttttctttttttcttttctttttttttgacggagttttgctcttgtcactcaggctggaatgcaatggcatggtcttggctcactgcaacctccgcctcctgggttcaagtgattttcctgcctcagccttccaagaaactgggattacaggtgcctgccaccgtgcccagctaatttttgtatttttagcagagatggggtttcaccatgttggccaggctggtcttgaactccggatctcaggtgatctgcctgccttggcctcccaaagtgctgagattacaggcctgagccaccacatccggccccataatcctttcttaattggaagtgacccagatggccaaaaagcatctattattcaatcctaaataacttcaagatttcaaattacattaaaaagtgcagctacaagcatttatcccatttacatgtacatgattctttcattttttcaaacgatttatctagattacttctgaaaactgaaatattagacaaagctaatcatcatttcaagttatttccttgttaaccttttttatagcctgcgaagatcagatgttcacctaagtaagaacacttttaaagttaaatacatgggtattgtgtcaataattcagaaaattcagctattttcattaaactaacattgaattagtcttacttattgaaaaaagtcacacaaacaaagactatttagttttggctgggtatattgttttataaccttttatgctaaaccccgacaccccaaaatatctagcagagacaaatataaaacccagacaaaaaggtatgctggcaattccaaagacatttctatttttaccttaccaataattttaaagctagtttttaaaattaacaatttacttaatcaagtgaatttaaaaaattcttgaacttatttacttaatttacaagcattcttttgcttataagcaaagttggtagacacaacatataataaatgtacatacacataaacacatctaaatatgtatacacacacagacacaaagatccaatagcatttacctcaaaactttagctatgaaatagcaatagaaactcaccagtttacaaacaggttcacatggctaaactatttttgccccaatacataatcaaatgaaggctgtgaaccaaaatttggggtagagcagttctcatggcagtttgctttttaaaggccataccttcccagatgccaaagagcactaggtccagatagcaccacagaaaaacatcatctataacctactaatcaggcccaaccctgcttagaacagcagcgtaggagtctgactacatggaatttcatcttgccttctcattcaacagcaaactccagatcccaaagaatactggggccaggccaagtgcagtggctaacacctgcaatctcagcactttgagaggctgatgtgggaggaccacttgagtccaagagttagagaccagcctgggcaacatgatgggaacttatctctccaaaaattaaaaaaaaaaaaagctaggcatgatggcatgtacctttactcctagctacttgggaggctgagctgggaggatcccttgagcccagcagttccaggctacagtgagccatgatgacactattgcactccagcctgggcaacagcgtaagatcctgtcttaaaaaaaaaatggaatattgaagtcaaacagcattacagaagaatatcagttatatcagtttatcaaactctaatttcccatgactatatcaacacacacaaccacaaaaatataaaccaactgctgtaacaacaagctctaagagtatccaaactgaggcagtcagggtgcttccctctctcagttgggcttgttcaacctataaatggaaattctttaaaaaatttcccagccaggtgtggtagctcatgcctgtaatgccagcactttgggaggccgaggcagggggatcacgaggtcaggagatcgagaccatcctggctaacacggtgaaaccccgtttctactaaaaatacaaaaaaattagccgggcatggtggcaggctcctgtagtcccagctactcaggaggctgaggcaggagaatggagtgaacccgggaggcggagcttgcagtgagccgagatcacgccattgcactgtagcctgggcaacagagccagactctgtctcaaaaaaaaaaaaaaaaaaattcccaaattgagagaagcagataccgtctaggcccacaaaggacacttttacctatccagatgcagatgtctaatttctaaggctgtttttcctaggtaatcaggaacatggttggggccagcagtggtggggctggacagagagagaaactgagactcacctctggccaaaaaagggtctggcacctgcttaggagggcttccaaaactttttcagcctgtggaaacaaacccacaagcaatgtgttactggtcagggaactaaaatctgttacctaaatgccaggggtttattctaggttctgctgcttgcagcgcagaaagccggtcactgagacagtgaagatagccagagaaggctttaatcaggtgctacagccaaggagatgacagataagtttcaaatccatctccccaagcaaccaaaattaggggtttatatagtaggaaagaaatgtacccatgtataggaaaatagaaactagggaggggcaagaaagagaagttggtcaacaggaagcaggtggttggttacgcaatcatgatgggtgagggggtcttatgtttcattgtcaagatgcagtgatctgctaagtttcagctccttgatactatctggaaggcctgatggttgttttcctgagaaaggaactcagataagacaaacataactttcttgagttttaagactggagcatcaatttctatgtttatcccaaagaaaccatacacgttagttctatgagacaactgggacaatttcataacctgcattagaggttgggaaatgtcttcccatatttccaagataagaaaatggtgcgggtgccaaaaggcatttgtcccagccatactcccctgtaatgtacacttttctcaaacattttccaaattcactgctacaatgctctgaaccctattttctgcagatgcaactttaacactgtatattcccgatctgatatgaaacaaacaacagcaccacattttccctggatattgttttgccaaggctttgatcgccactgcaatgtgccagtaaggcaagaggaaaatgagagatctttgagttcaatgatcttgtctaattaagctggagctcttccttggaaccccaagccatcatgtttgtggtcagcattggagctgtgaacatccttgcaactggtgtttggcacaacctactttgatcagcttctgttcatccaccagcattcctatgccatagagagtcaatgaggtaagactaagccagatggaccacctacaaatcacctggagacctctgaatccatagcaaagaaattaagacaaagttgaggaaatctgaactgctggttctccagctactgtagtcaaatgaagaggacacagaagccctgaatttttgaattcttaggatgaaaagggacacattggttgtatgactaactttatagggaaagaccttcaatacttataatgtgaactcaataaatagtaatgtagcaaagagaagtttccatggggaaaataacatgtaaagtcaaaacttaaaaaaaaaaaaaagtaagatttttccaagttgcatttcagaagagggaggagaaaaagcaaaggaacatgaaacgatgaaaaacaaactataacatattcctcacccttcaacaataatctggaggtcaaaagctaaattctttatcaccaaagaatcttttgaccttcacattggcttattgggtgtaattgtatttccagagtcactctctgattcctgatactcccttataaagtggccatcaatgagctgaagaaagaaagagtggagcatctggatcactgaacaacgggctggtttgacctcagaaagattgtaaaggtggccttgtgttaaatgagtcactaagacaagcccacacttagttgcaaagagccagaaggaaagcttgtctctgttggtgtgaaatggtgcagtcttctcatccattcatgaaaggaggaaaagactttgaccattggtgcagagcaaagctaaagaggaagaccaagataatgacatattttctccacatgcctttcaaaatggtattttatttactcttctttcaagtaacgtgaaataagggcaccacttttcacaaaatgcaaaatactttatggtttaatcctctcagccattctcaaaatgggtaccaatctctatcttacctataaggaaaccagggctcagaacaagtgaagggtttgttcaaaatctcccagttagtctgaagcagagcaaaaatcacacacaagatgatctgagttcaaaacacaagctgtggtcactaaatggtcagaatgagctcctctgtgctctgagagcctggcaaagactgggaatgaaatggacttccttgccttaaatggaaggtgttgaggaagaaagagttaaggtctcacagagtttaactaaaggcaagttttctcttccctcaggaaggaatcgtggatcattctccagctttttctctagaccatgttctaataacctttctctccacttggctcctgatcttatttcaatttctgagtgtaggggcttttcattctgagcacattatagtattccagcttgcccatcacccatctgtcccaggagtgtatcttgagggcttaaatgctcagtgtggtggaggaaagtttagcactagtttcctttggtaagggaataatttgcttatcaaatattaaacagaaatgttttagcaggttactagttcctctgcagaatggaacactgcatagtacttcattaccagctaatttcttctgcaaatgaatgactactttctgaatttaattaaactatgtaaacattattgtgaggggtccactccgcaacagagctgtgctctgctaggagatacaaaaatgagtaactcatagtctctgcctttgagacctctcaattcatttgagtaaagtcagtaagtgagtagcattccatggatgggggttctcataaaggtgttttcgttcaccttagatgtcattgaggaatatcagcaagtcctaaattatctgatttttaaataatagagtcttttacacatgtagctatttttcatgcattgcatgtaattttctttggtatctatatggatatgctttgtgttagctttgatactggtcacagttctttcctgaggacagaatagcatataactattaataataatgccagcatttgttgggtatttattatgcatcagatgttagagctcttctagtagagaatctgtggccagtgaatgtcatgtactactgtggagctttattctccaagcaacacagatgataaatatagctattactccaattttgcagataaagaaactgagactcagaaagcttattaacatgcccaaagtcacacaagaagaactgtggatccgtttgactttttctaagcttatttttaaactttattaagactaatccagagatgttagttttgcatccctttaagtgtcatcttgcatctgtaacaaaatccttgaacattcagcaagttctctatcctgtaggtggcataaactcaaccaattccccaggtctgtgtgagccctggcaatttttcagcttagagttccctgataattcttctttccacaaagtttttctttgcccagcctcttggaatttggagcagtttatcttctccggtactttgctccacaaatttgagctatcgctgcttctgccccaatggtgtcttcatcctcaactcagattgccaagctgttttgatctctgttgtttgccccacagttcatagattgtctctaggcaaaaagccagaataattgtggggcttgtctcatttgcttcccttctttgggggtctcattcatgctctgtctgctgtcctgtattggaaaactgttgtcaaatatttaattccattttctagtcatttgtgataagaaagtatgctgctagcagcagacattctcccaagatgtaaaaatgttcgtggtgttttaagtgaaaaaaaaaatcacactttaatgcgacatatacagtttatgcttattatggaaaaatatgttaatattctattgaatggatctaaaaatacagacataaaatgtttgagatgcttgatatcccaattatcatgatttgatcactacacattatatacacgtatcaaaatatcacatgtactccataatatgtacaactattatttatcgattaaaaaatgtcaatagtagtttctaggtgggtaagattgttgccatttttttctttttttttttttttctgagaggaaacctacattgcttagataacatgaaaaggaaagcttcaggaaaaattaaatgacaaaatatacagtccttaaaagcagaattacaactaaaatatggcatctattttcatcattagagctgagtgatgtcaatcagaatcagagctatcctactagaaaacttaatggtactatttaagcattcaaagatcaatcactgctattttacttaaccccaatttctctctatctcagactaaaaaaaaaaatgtatccaaggtgtttctttattgttcccaataatttccccaaatggtagcaatgcctccactttacaaagggtttctgcctaaagtttttcttaacacctttgtccctctgagctgtgttggtaatttgtttaaaaattgctattttatttttctctttctctggtctgttttatcctgagaggaagaaggaagttgacgtttaccactatggatccagcactgtgttctcattcatatacttgattttatttgattttcaaaataatcctgcaaagtattttgaattcactttaggttgaaagtggggagacagtctcaaaagacaaagcacttgcccaagattataaaactagtaagcaacaaatctagaattgagctctggtatctagatgtattttccaatttcttgaaagactattatttaggaattaggtagaatttcccctccctttatgtcctctcctcaatttccatcaaagttttataatatgcaagttagttcacaaaaaacatgtggctctctcctcttccaaccatgtgcctgggcacaactctgcatagtatatataccaaaatagtctctgagcatatatatgcagatttgtgaggtcattttcatttcagttatcatcttctctctacttccatagcactcagttaattcatttatatattctttcctttaactgatatgcctgggacccaggttatgtagtaataagtaagactcgatttctcctttcagggtgctcagagtctaatgtgaacacacatccactcagtgtagtaagtatagtgttagaagtggccatggagtcctacagatgcataaagaaagggctctttatttaatttgcagaggagaggagatcagaaatggcttcttggagaatatcataactgagtcaaatatcaaaagctatgtaaaagtgaggcaggtgggtaacataggaaagaggattcaggtgctgggaatattacacaaaaaatacttgtaagcatggaagaacacagtgtaagtgaaaactcatgaacatttaaagctagttgaagaacaagtaagatggcggaatggtgagaaggaagatggaaacttagacacaattcagatcttaagatggaaagtttacatcttaaaccaaagattgaatagtcattgaagattttcaggaaacaaatgatgtaatcagatctcctcttagtgaaatcatgtaggataccaatataaagaatgctcacatattttactaaaagtattccaaaagataaagagggaatactccttaaattattctataaggccagtatcatcctaataccaaaaccaggaaaggatataacaaaaatagaaaaatacagaccaatatctctgatgaacatagatgcaaaaatcctcaacaaaatgctagctaactgagtccaacagcatatcaaaaagctaacacaccatgatcgagtgggtttcataccaggaatgcagggatggttgaacatttgcaagtcaataaatgtgatacatcataaacagaattaaaaacaaaattcatcttaatagatgcataaaaaggcatttgacaaaatttaacatccctttataattaaaagcctcagcaaaatcagcttagaagggacatgccttaaggtaataaaagccatctatgacaaacccacagccaacattataccgaacaggaaaaaggtgaaaacattcccctgagaactggaacaaaacaaggatacccactttcaccacttctttttaacatagtactagaagtcctagctagagcaatcagacaagagaaagaaataaagggcatccaaatcagtaaagaggaagtcaaactgctgctattcactgatgatatgatcatatacatagcaaaccctaaagactcatccacaaagctcctagatttgttaaatgaattcagcaaatttccgggatacaaaatcagtgtacacatatcagtagcactgctatatgccaacaaccaagctgagaataaaatcgagtactcaacgcatttcataacagctgcaaaaaaataaaataaaatacttaggaatatactttaccaaggaggcaaaagagttctgcaaggaaaactacaaaacgttgctgaaagaaatcatccatgacacaaacagatggaaacacatcccatgttcatggatgggtagaatcaaaattgtgaaaatgaccatgctgccaaaaacagtctataaattcagtacaattcccatcaaaataccatcatcattcttcacagaactataacaaaaaatactaaaattcatatggaatcaacacagagcccatgtagccaaagcctgactaagcgaaaagaacaaatctgatggcatcacattacccgacttcaaactatactacaaggctatcattaccagaacagcatggtactggtataaaaataagcaggtagaccaatggaacagaatagagaaccctgaaataaagccaaatacttacagccaactgatctttgacaaagcaaacaaaaacataaagtgaggaaaggacaccctattcaacaaatggtgctgggataattggcaagccacatgtagaagaatgaagctggatcctcatctctcaccttatacacaaatcaactaaagatgaatcaaatacttaaatctaagacctgaaactataaaaattctagaaaataacattggaaaaactcttctagacattggcttaggcaaagagtacatgaccaagaacccaaaagcaaatgcaacaaaaacaaaaaataaatagaggggacctgattaaactgaaaaccttctgcacagcagaataaataaacagcagagtaaaaagacaacccgcagagtaaaaagacaacccacggagtgggagaaaatattcacaaactatgcatccgacaagggactaacatccagaatctacaaggaactcataggaatcagcaagaaaaaaaaacaaataatcccatcaagaagtgggcaaaggacgtgaatagacaattttcaaaagcagatatacaaatggccaatgagcatatgaaaaaaatacttaacatccctaattatcagggaaatacaaattaaaaccacgatgagataccaccttcctcctgcaagaataaccataattaaaaaattgaaaaataacagatgatggcatggatgtgaggcaaaaggaacacttttacatggctgaagggaatttgaactagtacaaccactatgggaaacagtatggagattccaaatagaactactatttgatccagcaatcccatatatatggcattcctatatataccatggaatactacacagccataaaaaggaatgaaataacggcattcacagcaacctgaatggagttggagaccattattctaagtgaaataactcaagaatggaaaaccaagcatcgtatattctcacttataagtgggagctaagctgtgaggatgcaaaggcgtaagaatgatataatggactttgaagacttagggggaagggtggaagtgaggtgagggataaaagactacacattgggtacagtgtacactgctcaggtgatgggtgcaccaaaatctcagaat

SEQ ID NO: 3 - DNA, 163 bp, Homo sapiens
caagtttagctgtgatgtacaggtttcttaactattagatttctcagattctaatatgaaacaatgccaattttcctgtgtgtgtgtgtgtgtgtgtgtgtgtgtgtgattggaggtttgttggcaaattgatgctaaagcatatttggctttggttctggaa

SEQ ID NO: 4 - DNA, 26 bp, Homo sapiens
caagtttagctgtgatgtacaggttt

SEQ ID NO: 5 - DNA, 20 bp, Homo sapiens
ttccagaaccaaagccaaat

SEQ ID NO: 6 - DNA, 599 bp, Homo sapiens
aacaaacataggttgcgtttatgcgaatggtcaggtccaaagtagatgcagaatatgccaggttcactaattttaatccctattcagcccaggactatgtaccataagattactgctagtgttttctgaaaatgatgtatcaaggcattttctgtagaaatacgaaacagtgacatacagtagggagagctggattgaggcagagtagtatagatggaagtttcctgaaagcattttggggaaacatcttttgggtatggttcttggatgaagagttgatttattagtactggaagggtrtatgggagagaggaagtgagaggttatgagagaatgaccctcccgtgatggtgagtgggagaattattgcagtatgtacgttagcattgctatgtggtgaagttcttgggatttcctggggtccgtgctggacagcatgcttagccaccagtcacatgtgggcactgagcactgacaatgtgggtagtctgaactgggatatgctgtaagtgtaaaatacaaactggactccaaagatttagtatgaaaaaaagaatttgaaatatctcattaatgatgtgtatttggtttcatattga

SEQ ID NO: 7 - DNA, 599 bp, Homo sapiens
ggatacagaggctaccagagtaggcagttgacatctcttctctccctttttgtctcttctatttacctcctttcttccataatcaagtaccagtcctgtcctgtctccattagctgctatctttaccttctatctcccagaaatgtacaaattatgtaattcaagtttctataactgccttcccctccatactaaaacatgttactacaccttggtttctgtcttcttatttttctgaatctgcactagtcctttcattgctatagctgtccttcctctgttctaaggctcatggcaccrttgtaagcccagggccctatgctgacactcatataacaacattttgctccatcagttactaaaagtgccctcttatatcagcagtctccctgtctttggagagtatacaacatgtattctcatcccatttttccattatgttcagagcggttgaatgacttgtacaagttaatgcaagtagttgaggcagaaatgatacttcaatcgagatcttcaaacttccaagttcagttcactttcttccataacatgttatctcaaaaataccatttgccagagataagtaatgctaaatcatt

SEQ ID NO: 8 - DNA, 599 bp, Homo sapiens
gatgtgactgcacatccatttcaaatgccctgaaactcaggatgtgctcctgaggtgctccccatgccaggatgtgcctgtggtttcctcaagagctacttggatacaccgcatacccttggatccagcactgctagagactctgcagaaactctgcaactttaactggaaacctctctactgttgttgttcccttggaaaatgactatttgccacttcagctgccaaatatctcctcccaatatgttctgtgagcattcagtctgctcaaagtcttgtgtaatggtggatatatgatgmcatttgtttcaagctaggctcacatatccatctatctcaggggctacatgatttacttgccattttttaaacttttacttgtaattaacaaataataattgtatatatttatgggatacaatgtgatgttttcatatatgtatacattgtggaatgattatatcaagctaattaaattaggcatcatctcatatacttttctatactgaaaacatttaaggcctattctttcagcaattttgagatataaaagacattattgttaattatggtcaccacactgtgcaatagcattgaaa

SEQ ID NO: 9 - DNA, 599 bp, Homo sapiens
gttctgtgaagcgggtaaaaacaaaatttggcatccagtttcaaaaggagaattgcaaactaatagaacatatagcacaaaatgattatatcaatagaatgctaattgcatatcaaggatatttggtataatacaaattattctaccttaaacatatggaaatttgtggtccatgatgttgtagattctatcttcccactctgcattttcaaaggcatatggtattgactcattcgattaattgttggatagtctttattatagactaaatcatagaataaatacatggatacatgcacraatattatatctcaagggctttacatagttcattatctcacttcatagtcaaaacaaacctactgatagttccaatgcaaagcctagaacgctttggcttagagaggcccaagtcttttctcagtgctgcactgctggtacgtggcgtggtcccctctcttctctcagtacacactacccatgcagactatcactctcagtcttgtttatctcaaatacagagggtataactaactggaatgtatccagaacagtgaggccaaagtgtggggaagctccttaaccatgctgctgcatga

SEQ ID NO: 10 - DNA, 599 bp, Homo sapiens
ttgcaaactaatagaacatatagcacaaaatgattatatcaatagaatgctaattgcatatcaaggatatttggtataatacaaattattctaccttaaacatatggaaatttgtggtccatgatgttgtagattctatcttcccactctgcattttcaaaggcatatggtattgactcattcgattaattgttggatagtctttattatagactaaatcatagaataaatacatggatacatgcacgaatattatatctcaagggctttacatagttcattatctcacttcatagtcamaacaaacctactgatagttccaatgcaaagcctagaacgctttggcttagagaggcccaagtcttttctcagtgctgcactgctggtacgtggcgtggtcccctctcttctctcagtacacactacccatgcagactatcactctcagtcttgtttatctcaaatacagagggtataactaactggaatgtatccagaacagtgaggccaaagtgtggggaagctccttaaccatgctgctgcatgaggaacagctggagagactgagaacatgaggcctaaagaggagactcagggag

SEQ ID NO: 11 - DNA, 599 bp, Homo sapiens
ctaccttaaacatatggaaatttgtggtccatgatgttgtagattctatcttcccactctgcattttcaaaggcatatggtattgactcattcgattaattgttggatagtctttattatagactaaatcatagaataaatacatggatacatgcacgaatattatatctcaagggctttacatagttcattatctcacttcatagtcaaaacaaacctactgatagttccaatgcaaagcctagaacgctttggcttagagaggcccaagtcttttctcagtgctgcactgctggtacrtggcgtggtcccctctcttctctcagtacacactacccatgcagactatcactctcagtcttgtttatctcaaatacagagggtataactaactggaatgtatccagaacagtgaggccaaagtgtggggaagctccttaaccatgctgctgcatgaggaacagctggagagactgagaacatgaggcctaaagaggagactcagggagatgggatcacaatcttcaaatatttaaaagacatcaaggggaaaagagattaaacaaggtaatgtagctctagagagcaaatccaagagt

SEQ ID NO: 12 - DNA, 599 bp, Homo sapiens
tccatgatgttgtagattctatcttcccactctgcattttcaaaggcatatggtattgactcattcgattaattgttggatagtctttattatagactaaatcatagaataaatacatggatacatgcacgaatattatatctcaagggctttacatagttcattatctcacttcatagtcaaaacaaacctactgatagttccaatgcaaagcctagaacgctttggcttagagaggcccaagtcttttctcagtgctgcactgctggtacgtggcgtggtcccctctcttctctcagyacacactacccatgcagactatcactctcagtcttgtttatctcaaatacagagggtataactaactggaatgtatccagaacagtgaggccaaagtgtggggaagctccttaaccatgctgctgcatgaggaacagctggagagactgagaacatgaggcctaaagaggagactcagggagatgggatcacaatcttcaaatatttaaaagacatcaaggggaaaagagattaaacaaggtaatgtagctctagagagcaaatccaagagtgttgagtggaagtgaaagggaggctgg

SEQ ID NO: 13 - DNA, 599 bp, Homo sapiens
atgatggtttccagcttcatccatgttcctacaaaggacatgaactcatcattttttatggctgcatagtattccatggtgtatatgtgccacattttcttaatccagtctatcattgttggacatttgggttggttccaagtctttgctattgtgaatagtgcctcaataaacatacatgtgcatgtgtctttatagcagcatgacttaaaatcctttgggtatatacccagtaatgggatggctgggtcaaatggtatttctagttctagatccctgaggaataaatgaccaactatygagaaattgcagggtagtccctacatgagggttaggtagaattgacctgctttctgcctcataaattttagaaaattaataagataatttattacggggtggtgtttgttccctcagtactttatcatctatgttgataatgttaataattaattgcataattaacaaatagcaaattattgtgggggtgtgtgtgtgtgtgtgtgtgtgtgtttagacagggtcttgctgtgtcacccaggctggagtgcagtggcgtgatctcggctcactgcaacctgtgccttccaggttcaagc

SEQ ID NO: 14 - DNA, 599 bp, Homo sapiens
atcctactgtgatcaaaggcacatacatgagatggtgagttgtccccttgccaatgagggtttggtaagaaaggaaagtgcagtacttctttgtttctgaattgcaagtatgtgtgggttagagggggaggctgaatatgaaggtcctgggacagcccaccaggtatcccatgagactttgcaaaggaaaaggaggtgagtgacagcccagggtccaataggatagaaggaaaagccaggccatggagttcctcagacctgctttctaagggcaactctaccacctcagcaagccattgracttctctgagctcagtcctttcatttataaaatggggtgacagtgctcacatgccaggaatacaaagggattgaaagataaaacacgtaattaagcacctgttgttacacatctgtcagggaccccaataaggtcagctgtcttcctgttgacttctgttcttggtggttctccaagatcataccttccatcaacatttaccgtcactcccccaccccatgcccaatactgaacagtggagggacgcttcacctacagttataatgttgaaacttcaacccaaagcaagtactgttag

SEQ ID NO: 15 - DNA, 599 bp, Homo sapiens
tttaaagtctaccatttcaggtccatattttcgcttgaaaattgagattcctattaaacaatgacatttacaccaaaaagtagaggagttggttgaaggacagggtaatgccaggaggaattgggaatttgagagtcaagtcaaaggactgaaatactcagaatactaagggcacctcagggctctaccaaggacacgtagaagctttgaatttgcagcaccaccctaatttaacgagctacctcagcacgtagtggagccttggaaaacagatgtcacaaactctcattagattgtcaracattttccagcatttcctctcccatcatagctggttatcaagatatatagacacacacgtgcatacacataaataccttgataagttactagagaaagcagaaaaatgtctgacagtttaatgagatttgggtgaaagaaaattctatatttcattgttttccaggcactagaaataattcatcaatgtttctaagactcattcagcgtggctgcattttttaaaatattttcataaattttgaggagcaaataccattattaggcactaaaaaggttgaagtctaatagattagccg

SEQ ID NO: 16 - DNA, 549 bp, Homo sapiens
tattttcgcttgaaaattgagattcctattaaacaatgacatttacaccaaaaagtagaggagttggttgaaggacagggtaatgccaggaggaattgggaatttgagagtcaagtcaaaggactgaaatactcagaatactaagggcacctcagggctctaccaaggacacgtagaagctttgaatttgcagcaccaccctaatttaacgagctacctcagcacgtagtggagccttggaaaacagatgtcacaaactctcattagattgtcaaacattttccagcatttcctctcccrtcatagctggttatcaagatatatagacacacacgtgcatacacataaataccttgataagttactagagaaagcagaaaaatgtctgacagtttaatgagatttgggtgaaagaaaattctatatttcattgttttccaggcactagaaataattcatcaatgtttctaagactcattcagcgtggctgcattttttaaaatattttcataaattttgaggagcaaataccattattaggcactaaaa

SEQ ID NO: 17 - DNA, 599 bp, Homo sapiens
acaaaatccccaaagcaattttggaataagagccaacatttaatacttaccagacaactattctaagtattttactatattcactcatagcaactctaaaaagcaggtagtattaacagagaaaatgaggcacagtgaggttaaatagctggtccgaggctacacagctaatcagtgggagagttgggacttagacccagaggtccagttttgaagtccacacttttagccattacactacaatggaaagaaatttagaagatatacacagaaaactataggcacatagattaggggttmgtagaatgctctgggcagttaaaggaactcttcttaaaggaggtaaagcttgaatgagactgttagtaagctatttttcactcattggtgaatgatgttttgtgcagtgtgtttttttccccatagaaaaataagaaagaaaagaaaattgagaactctctctataaaaatgtgtaacatatctcatattccaagagatccttttggtagtattaatttttatctgctcacagtactggcttcattatttggagttaaaaattaactcaaccagataaaaaaatcagtgctgtgtattt

SEQ ID NO: 18 - DNA, 599 bp, Homo sapiens
gttctaattttaaaaagttatttaacagaacgaagctatcagctaagacaatggcaaagccgtaaacaaacataggttgcgtttatgcgaatggtcaggtccaaagtagatgcagaatatgccaggttcactaattttaatccctattcagcccaggactatgtaccataagattactgctagtgttttctgaaaatgatgtatcaaggcattttctgtagaaatacgaaacagtgacatacagtagggagagctggattgaggcagagtagtatagatggaagtttcctgaaagcattktggggaaacatcttttgggtatggttcttggatgaagagttgatttattagtactggaagggtgtatgggagagaggaagtgagaggttatgagagaatgaccctcccgtgatggtgagtgggagaattattgcagtatgtacgttagcattgctatgtggtgaagttcttgggatttcctggggtccgtgctggacagcatgcttagccaccagtcacatgtgggcactgagcactgacaatgtgggtagtctgaactgggatatgctgtaagtgtaaaatacaaactggactccaaa

SEQ ID NO: 19 - DNA, 599 bp, Homo sapiens
ccccccaaccctatttttaaattatttacttatttttattatatttttgatacataatagatggacacattcaaacagtgcccccaaaactggggcagcagaaacaggtccttgcttattttctcagcttcacctcctgcctccaccccatctgtactgctggtccagacattcctacagaggtgtcctcctaagttggtctcttcctctcctgcttcagaggctttgccctgctcttctctgcctcttgaggctctgtcctgctcttctctgcgtcttgtggttggaatgcctgtcttyttcctactgaagatctggatgcctaaaccataatgtaaaattgctgctttttacttccatttacagcagagaaattcctcctctggcctctcctcttctctgtgtttctttcttcataatttttatttatttatatatttatttatttatttatttatttatttattttcattgagatggagtctcgttctgtcgcccaggctagagtgcagtggtgtgatctcagctcactgcaatctccaccttccaggttcaagcgaacctcttgcttcagcctccctcctgtagctgggactaca

SEQ ID NO: 20 - DNA, 599 bp, Homo sapiens
gtcatgattaaatgttaagtgaaaaaatagaatatagctagatttgaatttgaatattcaatctgtataccagtatgtatagaaggaagagtatataccaaatagtaagagtatctatctgttttataatttgatataatacaaattattctaccttaaacatatgagaatttgtggtccatgatgttgtagattctatcttctcaccctgcatttccgaagacatatggtattggctcattagactatttgttgaatagtctttattctattatcatagaaaaaataaatgagtgcatrtatccatatacaaaatagaggtctgttcttcctgtatatatttatactaaaaaaactgagacttttttttacagttgtatatatacaaacatatttgtttatttatatacacatatataaatcaattttatgtacatgtgggtatacatacatccatgcatataactctgaagtgctgactctctaaagaaagcccaggtattggtcagaattcatgctcggctcaggagtatagaattaagagatacaaacctcaaaaaagagggaaccgaatcttcaaatctgagccaccttacaag

SEQ ID NO: 21 - DNA, 599 bp, Homo sapiens
ataataaaataagggctaacacttaaacggttgtgtactcactatgtactaggcactgatcaaagtactttgtacatattttcttatttaatattcgctaccatcatattacaatatactgttattaaccccaatgtatagatgtaggtgaagaaacttgtcacaaatcatacagctagttgtctgagatgcaatccatgtgatttgttcacagagctcaggttctgtgaagcgggtaaaaacaaaatttggcatccagtttcaaaaggagaattgcaaactaatagaacatatagcacmaaatgattatatcaatagaatgctaattgcatatcaaggatatttggtataatacaaattattctaccttaaacatatggaaatttgtggtccatgatgttgtagattctatcttcccactctgcattttcaaaggcatatggtattgactcattcgattaattgttggatagtctttattatagactaaatcatagaataaatacatggatacatgcacgaatattatatctcaagggctttacatagttcattatctcacttcatagtcaaaacaaacctactgatagttccaatgc

SEQ ID NO: 22 - DNA, 599 bp, Homo sapiens
tttgggtatggttcttggatgaagagttgatttattagtactggaagggtgtatgggagagaggaagtgagaggttatgagagaatgaccctcccgtgatggtgagtgggagaattattgcagtatgtacgttagcattgctatgtggtgaagttcttgggatttcctggggtccgtgctggacagcatgcttagccaccagtcacatgtgggcactgagcactgacaatgtgggtagtctgaactgggatatgctgtaagtgtaaaatacaaactggactccaaagatttagtatgaawaaaagaatttgaaatatctcattaatgatgtgtatttggtttcatattgaaaacaactttggtattatatattgagttaaataaaatgtcattaaaattaaattttacttaaactaaaatttaaaattctatctttacctttttttttttttttttttttttttgaggtgccgtttcactcttgttgtccaggcttgagtgcaatggtgcgatcttggctcaccacaacctccgtctcctgggttcaagcgattctcctgcctcagcctccctggtagctgggattgcaggcacgcacc

SEQ ID NO: 23 - DNA, 599 bp, Homo sapiens
tattcagcccaggactatgtaccataagattactgctagtgttttctgaaaatgatgtatcaaggcattttctgtagaaatacgaaacagtgacatacagtagggagagctggattgaggcagagtagtatagatggaagtttcctgaaagcattttggggaaacatcttttgggtatggttcttggatgaagagttgatttattagtactggaagggtgtatgggagagaggaagtgagaggttatgagagaatgaccctcccgtgatggtgagtgggagaattattgcagtatgtacrttagcattgctatgtggtgaagttcttgggatttcctggggtccgtgctggacagcatgcttagccaccagtcacatgtgggcactgagcactgacaatgtgggtagtctgaactgggatatgctgtaagtgtaaaatacaaactggactccaaagatttagtatgaaaaaaagaatttgaaatatctcattaatgatgtgtatttggtttcatattgaaaacaactttggtattatatattgagttaaataaaatgtcattaaaattaaattttacttaaactaaaatttaaaattct

SEQ ID NO: 24 - DNA, 599 bp, Homo sapiens
tcccagcactttgggaggctgaggtgggtggatcacgagatcaagagattgagaccatcctggccaacatggtaaaacctcgtctctactaaaaatacaaaaaattagctgggcatggtggcacacacctgtagtcccagctgctcgggaggctgaggtgggagaatcacttgaacctgggaggcagaggttgcattgagttgaaatcacgccactgcactccagcctggtgacagagcaagactccatctcaaaaacaaacaaaaattatagtatgaaataggcattaaaatattgtgyattttagaggagactgaggattggaggctgaagaattactctaaattaatcagcttgtgtacttcagagctaagatagctctttgggttctaaattctgtgatcttctttttgatttctcttggagcaataatgaaggcaaaacatcaataaacataacaaactgggtaagggagaccattgagaaggactaaggacaccttcaaagttctgagtgagtttaaaaagaagaatgatgaaaactttgatagaaataggaaaaaaagtagaggaacttgtttggcttgaaacttcttaatg

SEQ ID NO: 25 - DNA, 599 bp, Homo sapiens
aaaaaatagcactgaaaacagaaaggaagcatcaaaactcttcaaatacctgctgtgtccattggtcaagcacattcaggacatcgcatgcctttagaactccagcaggttccaacagctagtaggacattctagactctgagagagagcaagggaggttttatgactggggacaaagaaaagagacactgaaggcgaaggacaatctctgaaaatgcagtaccctccagactgctcctcctctcacaaaaacaccttcccagcatgcactgctttagggactatgattataccattgaytctgtccagaaaacctgtgtcctgaatatattacagggctcattccttcacttctttcaggtgcctactcaggtatttccttatcagaacagtctttcgaacgaccccattaaaaaaatagtcctgtcaaccctatgttaacaattttatttatttttattatttgttaacaatacataataggtgcatatattttgggggtacatataataatttgatacattcatattgtgcataaagattgaatcggagtaattgggatatccattgccttaagtgttttaccttttctttatgct

SEQ ID NO: 26 - DNA, 599 bp, Homo sapiens
tgtgtgtgtgtgtgtgtttagacagggtcttgctgtgtcacccaggctggagtgcagtggcgtgatctcggctcactgcaacctgtgccttccaggttcaagccatcatcctgcctcagcctccctagtagctgggattacaggcgcctgccaccatgcccagctaatttttgtatttttaatagaaatgggatttcaccatgttggctaggctagtcttgaactcctgacatcaggtgatccatccgactcatttcccaaagtgctgggattacaggcatgggccatcatgcctggccygcaaattgttgttatttataactcttcaatccaaatcatcagtgtctatgttgtttccttaactatcaaatgatgataataatagtaccttcttcataagatagttgaaaggtttttaatatccatatggtactgagaatgatgcctgaaacatagtaactaccccatttttattatatttctgttaataataatacataccattattgctcttgcataccatattgctcttgcataccatatatgctcttgctatatgctacacacagtatttcatttaggcctcactatgtccctga

SEQ ID NO: 27 - DNA, 599 bp, Homo sapiens
gaaagcagaaaaatgtctgacagtttaatgagatttgggtgaaagaaaattctatatttcattgttttccaggcactagaaataattcatcaatgtttctaagactcattcagcgtggctgcattttttaaaatattttcataaattttgaggagcaaataccattattaggcactaaaaaggttgaagtctaatagattagccgcttcatcctccttcactcagctcagcattcgttcaactggctcttactggttaacatccacacgcctcctgactggctactcagtgccgatgacrtttccttcacacacagggctggttttaagatacattgaggtgacatcaggtggcctgtaaagtggtcattttaggatatcctattcaaagacatctgtggaagtgtggaccaatttattgatgaataacagtgaaggggtttccaccagcaagtaacataattttttacaatgatgatgctgaagtagaaagagtttctagtcagggactggacaaatcaatttgcagacgatttttaggaagaaaaacattgcaacagtaaattgtaattgataacttctagagccactttaagtact

SEQ ID NO: 28 - DNA, 599 bp, Homo sapiens
taattgcataattaacaaatagcaaattattgtgggggtgtgtgtgtgtgtgtgtgtgtgtgtttagacagggtcttgctgtgtcacccaggctggagtgcagtggcgtgatctcggctcactgcaacctgtgccttccaggttcaagccatcatcctgcctcagcctccctagtagctgggattacaggcgcctgccaccatgcccagctaatttttgtatttttaatagaaatgggatttcaccatgttggctaggctagtcttgaactcctgacatcaggtgatccatccgactcakttcccaaagtgctgggattacaggcatgggccatcatgcctggcccgcaaattgttgttatttataactcttcaatccaaatcatcagtgtctatgttgtttccttaactatcaaatgatgataataatagtaccttcttcataagatagttgaaaggtttttaatatccatatggtactgagaatgatgcctgaaacatagtaactaccccatttttattatatttctgttaataataatacataccattattgctcttgcataccatattgctcttgcataccatatatgctcttgc

SEQ ID NO: 29 - DNA, 599 bp, Homo sapiens
ggtgtgatcacagctcactaaagcctccaactcttgggttcaagtgattctccacctcagcctcccaaatagctgggatgaaagtgtacaccacaatgcccggagaattatttcatttcttctttgtagagattgagtcttactctgttgaccaggttgctttcgaactcctggcctcaagccatccttccacctcagccttcccaagtgctaggattacaggcgcgagccaacttgcccagccctggaatttttgagcctgttcaattctaactattgtcaccaaaagtaaccttaagraaaaaaatgcattatctccttgcttcattgcaccattaaaatctttcctaaattttccatgttaaagatgaagctcaaaatcctcagcatagcatacaaaacacttcataatcagatgcctcttcaaatacctcctatcagaatggtctctttgactacccctttaaaaaaattccccccaaccctatttttaaattatttacttatttttattatatttttgatacataatagatggacacattcaaacagtgcccccaaaactggggcagcagaaacaggtccttgcttattttctc

SEQ ID NO: 30 - DNA, 599 bp, Homo sapiens
agccttagaacagaggaaggacagctatagcaatgaaaggactagtgcagattcagaaaaataagaagacagaaaccaaggtgtagtaacatgttttagtatggaggggaaggcagttatagaaacttgaattacataatttgtacatttctgggagatagaaggtaaagatagcagctaatggagacaggacaggactggtacttgattatggaagaaaggaggtaaatagaagagacaaaaagggagagaagagatgtcaactgcctactctggtagcctctgtatccaaaaggttgrctcaaacattcgctcataactttgtctggcttaatcctgctcatcccagcagacttatttcaagtgtctccacgttttgggaagtcatcactcacttctctgggctttcatatgggagagcatttaattctgttgaaaaactatttaatactacatctacctttctctatggactctgagcttcttgagggcatgtatcatgtatgttctattctgaagcacccatacctagaacaaagcttagcacatagtaggaacttaataaatatttcggagttgaataactagccttatgtaat

SEQ ID NO: 31 - DNA, 599 bp, Homo sapiens
tttaagtgctgaaacctccaagacaaaagagactgtgtctttattgttctctgaattactcgtacccagctcggtacctggaacatgatagggatcccatagtggtttgatgaataaattagtgactccaagagtaaagtaatcctcaggaggacaaaggcagatagcttcccttccctatcagaatgtacttctcttaaagcttttcttggtataattcttggagaattttgccttacagaagtcaaatcacataccaaagtgaaaactggatcttctacaaataatggaagaatcaastctatcaaaacaacaattatacatatgatcaatggaggggttgtcacgagccaggctaagagctttacatatattatctcattctgtctatgccagagaatcaactatgacatatgtaacattaaatctcattttatagatgcaaaaactggggtgtaaagaagtcaaagaatcagccagaatgtacagaattagcaaaggtggaactgggatttgaattcagacagtctgactccagacgccatctccgaattatgcataattatatttcaattattaacattcataaattgaaatat

SEQ ID NO: 32 - DNA, 599 bp, Homo sapiens
tcacataccaaagtgaaaactggatcttctacaaataatggaagaatcaactctatcaaaacaacaattatacatatgatcaatggaggggttgtcacgagccaggctaagagctttacatatattatctcattctgtctatgccagagaatcaactatgacatatgtaacattaaatctcattttatagatgcaaaaactggggtgtaaagaagtcaaagaatcagccagaatgtacagaattagcaaaggtggaactgggatttgaattcagacagtctgactccagacgccatctcygaattatgcataattatatttcaattattaacattcataaattgaaatatgagggataatgtaccttttcatgaaagctttgctcgttgtgtggatgagtgtgtgtacatgtaactgcttatgtgtgctatcactgaggtagaagacatctctctctctctctctctctctctctctctgttttggtctacttttagtaagacttgtatttgattgagttcagaagtttgattatctttttaactaacctgtttgttttaattatattaaaaattagtcactttcaacatatttgcata

SEQ ID NO: 33 - DNA, 599 bp, Homo sapiens
agttcctcagacctgctttctaagggcaactctaccacctcagcaagccattgaacttctctgagctcagtcctttcatttataaaatggggtgacagtgctcacatgccaggaatacaaagggattgaaagataaaacacgtaattaagcacctgttgttacacatctgtcagggaccccaataaggtcagctgtcttcctgttgacttctgttcttggtggttctccaagatcataccttccatcaacatttaccgtcactcccccaccccatgcccaatactgaacagtggagggaygcttcacctacagttataatgttgaaacttcaacccaaagcaagtactgttaggatctctggaaactttccctcaaataagggatttgaatgggacaagaagaagttttacagatagccaatggagatgatttaatggggttatgatagaaacgagaaagtaaaacaaacccatgctttaaagtctaccatttcaggtccatattttcgcttgaaaattgagattcctattaaacaatgacatttacaccaaaaagtagaggagttggttgaaggacagggtaatgccaggaggaattg

SEQ ID NO: 34 - DNA, 599 bp, Homo sapiens
tgttgttacacatctgtcagggaccccaataaggtcagctgtcttcctgttgacttctgttcttggtggttctccaagatcataccttccatcaacatttaccgtcactcccccaccccatgcccaatactgaacagtggagggacgcttcacctacagttataatgttgaaacttcaacccaaagcaagtactgttaggatctctggaaactttccctcaaataagggatttgaatgggacaagaagaagttttacagatagccaatggagatgatttaatggggttatgatagaaacragaaagtaaaacaaacccatgctttaaagtctaccatttcaggtccatattttcgcttgaaaattgagattcctattaaacaatgacatttacaccaaaaagtagaggagttggttgaaggacagggtaatgccaggaggaattgggaatttgagagtcaagtcaaaggactgaaatactcagaatactaagggcacctcagggctctaccaaggacacgtagaagctttgaatttgcagcaccaccctaatttaacgagctacctcagcacgtagtggagccttggaaaacagatgtc

SEQ ID NO: 35 - DNA, 599 bp, Homo sapiens
gggtttctaagctctctaatctcccctgtgcagctggcttgctgtatggtttatacaaatccagtggtgatctctgtgcaacgtggtatcacctgtttaaagaggtctcatcttcattttcaaagaggaatacatgtttttttacttactcttctgcatggctgactccttttcatgctttaagtctcaatcttaatgccacctcctccttccagacgttcccagctaaagtggcacttcccagccccattactctctatgtttattgcctgcatagctcttatttgtaatgatttcgtratagtttgatgatgatcatgatgaatattactttacctatttatggcctctcttttagtattaaattctgtaagccacatgagcatggggacacatctcctttgtcactgccccattgctggcatttagcacaagcatggtctataatagataccaaacaaatatgtattaatcatgtaaatgactaaatccatgaatgaatctatcagacagtgtagatagcagcacataaaggaaagggaatgtagtaaatttttcattttccttgaagatgtagctatgtattaggaatttgaaaa

SEQ ID NO: 36 - DNA, 599 bp, Homo sapiens
tagggaggcacatttagatatgcagtgataattgctttgcttagagaaattcaaggtgatagaatgcatggtgacacctaacccagactggtagagaaagggaattcttccactgggaatgacatgattatctaaataagtaggctcaatcaggtcaggaaagggcctgaaagactatttcaagcagagggaaggtatttgccaaggccagggtgtgtagtggagagaatgggcagtggcagagaattatgaagtgttccaatgactaaaagtaaagtaacaagttccttgtccaagagyttggattgtatcctaactgaaatgagtatacactaagtgtttgaagaagagggatgaaatggtcaagttttcattacacaaaaataacctgttcctttcattttatgtttatttatttttttaattttctgactgctcctttctggaaatctcaaatttatatttgccaaatattgtcacattttcgatggagaatacaaactaagaatgggttagggaactgagtcagaaagtccctgttgtacaattcaatcatgtttttctaaggatgtgcttttggacattatggaaactatctt

SEQ ID NO: 37 - DNA, 599 bp, Homo sapiens
acccagaaccttaaaggcaagaatatgattgaaatgtcaaatggggacttggtgatctaaattatgtcccccaaaagccaatgtcttgccaccaccagtgccctatgggtggagtttctaaacagattactcaaaacacaaactttcaaaaagggaaagtcataaccctctagtcatcagggcaattacggaataacattgctggagtaaggttttctcaatgcccaagagatgagctggcaatgccacaacaatgtccaattcttagtgggtccaagaccatgtgttacatttccctcycatgattactcacagcttcacagttctgctgtcctcttcgcctctctgccacctcttaactgcacctttgacctcctacccctaagattcaaccctgtgagattacttgtcttttcatctacactctggtcactctgacccccattctttagattcaggattttctcttttcctggcttactctccagcacaagtagaaaaatattgtgcttcattggaaaatgcatgttgtttgaatcacactctttcagattatacaattgtagtctttcattatctttgagccattttataatgct

SEQ ID NO: 38 - DNA, 599 bp, Homo sapiens
aatagactgttgcattttgaattttatatttgccataattttgattttttttcttctctgactactttcttttgtagtctcatcagctcagaaggtcaaagttgagatacttgtcaagacagtcaaagtgtagaacaatgagcctatgacagaagtgaggcatgatattaattgtacattaagctttataggtttttggatcatccatccctctgacagtggtaataggaatcctgagtgctattatatttccttcacagcaaagattataaaaagcaagaggaattacatgtgcttctrccgcatcttatcagcactcaggcaaggtttactaatatgtataatcacaatgacttccaaaggttccaccttggtacttctgttaatttaattcaatgtaacagacacttagtgagcatctaataagtgtcaggcattaggcctagtgctagggatagagaaatgaatacacagatgtggactttgccagtaaaaataagaagtagagttgtgtgcagtggatttatatgttttaggctaaatatggacttttcctagaagagggattcagatggcatttcagttcacatttagtacta

SEQ ID NO: 39 - DNA, 599 bp, Homo sapiens
agtccaagggaaaacccagaaatgcatttacagatcaggaatatagaaataaatgggcaaatggaggcaaaactactagccttttccacaatggggaagaaaagtcaattaaatcacccctggacacaatttacatgtctatagacaagaattatatgcattaatattagtttacagcattataaaatggctcaaagataatgaaagactacaattgtataatctgaaagagtgtgattcaaacaacatgcattttccaatgaagcacaatatttttctacttgtgctggagagtaagcyaggaaaagagaaaatcctgaatctaaagaatgggggtcagagtgaccagagtgtagatgaaaagacaagtaatctcacagggttgaatcttaggggtaggaggtcaaaggtgcagttaagaggtggcagagaggcgaagaggacagcagaactgtgaagctgtgagtaatcatgggagggaaatgtaacacatggtcttggacccactaagaattggacattgttgtggcattgccagctcatctcttgggcattgagaaaaccttactccagcaatgttattccgtaattgccctgat

SEQ ID NO: 40 - DNA, 599 bp, Homo sapiens
catggtcttggacccactaagaattggacattgttgtggcattgccagctcatctcttgggcattgagaaaaccttactccagcaatgttattccgtaattgccctgatgactagagggttatgactttccctttttgaaagtttgtgttttgagtaatctgtttagaaactccacccatagggcactggtggtggcaagacattggcttttgggggacataatttagatcaccaagtccccatttgacatttcaatcatattcttgcctttaaggttctgggtgtatctgtatcttcayatggtgaaggaattataaagcgattttgtgtcccctctgtgggcacaggatgttgcagaaataaagctgatagaaatagtcacatgagttgaccagatggcatcatcagcctcttgaatccactacatccctaatgtcattagcttggaggggcagctgccaaattaggtggggctggacattccttttaacaggtgccttcttttctaggatccaagtgagagcctaagatagtttccataatgtccaaaagcacatccttagaaaaacatgattgaattgtacaacagggactttct

SEQ ID NO: 41 - DNA, 599 bp, Homo sapiens
ggcagcattatgggggaaaagcaatgatgatctaatgagatctgataagaagttagcccaaaacaaggaaattgttgagggttctctttgaagtatggatttatacccaccaaccttagctgcgaaccttacctcaagtgttacctgtgccttgagatgtttcctggtcatagtactaagctatcataatgagcaagacattcaataagcaagtgtgatggctatgaggacagatcttaacaggtttttttttctggaaggcttaaaatcatgcattactcaatctaatacttcacgaamtttcagtaaaacctaatgataatatagaagcttgtgttgtagttttgtaatcaacagcaaaacataaaatttaaaaaaaacatacattactggggctgtatcctgctacaataataaggctgacataatagatggagaacaatatggtaacaagccaaaatgtattacttcatccacaaatagtatcgtgctatatatagacagacttgttaaaaatttaaagaaatacacaatcaattacacaatagaaaatttgctatatggtgcatggtggcatgcaactacaaatgtttctaaca

SEQ ID NO: 42 - DNA, 599 bp, Homo sapiens
cacacagtatttcatttaggcctcactatgtccctgatgtaggcattaatatctttattttgcaaatgagaaaacagtctgtaccttgtatgccatgctgctattgtttatctgtttgaatctcaagcaaatctgcttgataattggtaccaaaataagcctttttctgggtaaggaatctgatattgtgttttaaaaaacacacatttaatcctggggctgctgcattactcctgctgccccatcctactgtgatcaaaggcacatacatgagatggtgagttgtccccttgccaatgrgggtttggtaagaaaggaaagtgcagtacttctttgtttctgaattgcaagtatgtgtgggttagagggggaggctgaatatgaaggtcctgggacagcccaccaggtatcccatgagactttgcaaaggaaaaggaggtgagtgacagcccagggtccaataggatagaaggaaaagccaggccatggagttcctcagacctgctttctaagggcaactctaccacctcagcaagccattgaacttctctgagctcagtcctttcatttataaaatggggtgacagtgctcacatgcc

SEQ ID NO: 43 - DNA, 599 bp, Homo sapiens
ctctataaatgtttggccattggtttaaaaaacaaataatggaccaatgggctcaaaagcaaactggctaatataaaaataataataacaaccacaataataaaataagggctaacacttaaacggttgtgtactcactatgtactaggcactgatcaaagtactttgtacatattttcttatttaatattcgctaccatcatattacaatatactgttattaaccccaatgtatagatgtaggtgaagaaacttgtcacaaatcatacagctagttgtctgagatgcaatccatgtgakttgttcacagagctcaggttctgtgaagcgggtaaaaacaaaatttggcatccagtttcaaaaggagaattgcaaactaatagaacatatagcacaaaatgattatatcaatagaatgctaattgcatatcaaggatatttggtataatacaaattattctaccttaaacatatggaaatttgtggtccatgatgttgtagattctatcttcccactctgcattttcaaaggcatatggtattgactcattcgattaattgttggatagtctttattatagactaaatcatagaataaa

SEQ ID NO: 44 - DNA, 599 bp, Homo sapiens
taaataagccaatatgctttctgttgattgattgatttactaaacattgattggccatctccactggggatatggcatttaagagatctctttgatcttagtacttttactgctttttaaataggatcaaatacacccaaggtaaaaaatagaacacactatacgttacattttggaactgttagaaattcctttgaagctaaaattactgctatcatttgacaacttttacccctaaaataatgtggtgctcaccagcttgcttaagttacagcacttgctgtcttctcagatacaatrtcagaaacttataatccaagaaaaatctaaatggcaagtgtgagttaatggaagcctcataaagcaagaggtgttttggaagtgtatggaagacatcaataaatgatatgtataacatcaagtgcaaaagtgtgtgctaggaaagttcaaaaaagaaaaaaaatatggtaaggtaagaccagagattggggagtatatagcttttgggaattcaggaaatgctaacgtctatggagaatttgcataggtgaaagatcagattggaagcctttctctgtggaagcattgtggatctaagt

SEQ ID NO: 45 - DNA, 599 bp, Homo sapiens
ttcactattcagggattatttggaatcccttgtcaccagaagctcttaaggaaataacttctacttcgttgcaaatatgttcttggcttagttgaggtaatgcaaatactagaatacttgtttgtttaacagcttattcttccctgaagctgttcctccagtccctgccagtgggatcttatgtctccaggagtacttaacacccctaatagccccatcttttaagcctccctgggacctgccctcgcagtacctcttatacctactccacttcctcctcatggcctcctgcagaatgcmattctaaaattaggttctattttcctcgcccgcattctcttttgcaaagcctccaaaaaatttactttgcttctctgcgcctgctttatctctattttctacactcgctccttctttttctaattatctataataggcgtcacaaaatttgcatttgttggaaccaaaatttccatggttgcctcaaaatatacagatgtaaatttgcatataattaaattttgcataagggaaactctcatttggggagatatgcaatgcccaataaatggcagtttccttcaatgtccccaggccag

SEQ ID NO: 46 - DNA, 599 bp, Homo sapiens
ctcctggagacataagatcccactggcagggactggaggaacagcttcagggaagaataagctgttaaacaaacaagtattctagtatttgcattacctcaactaagccaagaacatatttgcaacgaagtagaagttatttccttaagagcttctggtgacaagggattccaaataatccctgaatagtgaaggatttactattgcacatttaaaatgtgtaaaatgttttaacattgtcttacaatgatgtctttattattatatgttagtaaattaaatatttacttataagttgtrtagatttattagcaacctcagttcttcatcccacttttttaggttcctgaaattaaattacatagtcttgttagaaatgcttgttccccggtgccataaagaaatagcacttgaacataaatttaatttcctcagcaaggcagtttttacctattgcagaaagggtacacttgccagcagttttgccacaagagtacaccgaacaaaggagacagggtcatttataacctgacccatccaccttcctgctgtgtccactttccattggctgaaacgggacctcacattctgtatttgtc

SEQ ID NO: 47 - DNA, 599 bp, Homo sapiens
agaatgagtccaagggaaaacccagaaatgcatttacagatcaggaatatagaaataaatgggcaaatggaggcaaaactactagccttttccacaatggggaagaaaagtcaattaaatcacccctggacacaatttacatgtctatagacaagaattatatgcattaatattagtttacagcattataaaatggctcaaagataatgaaagactacaattgtataatctgaaagagtgtgattcaaacaacatgcattttccaatgaagcacaatatttttctacttgtgctggagartaagccaggaaaagagaaaatcctgaatctaaagaatgggggtcagagtgaccagagtgtagatgaaaagacaagtaatctcacagggttgaatcttaggggtaggaggtcaaaggtgcagttaagaggtggcagagaggcgaagaggacagcagaactgtgaagctgtgagtaatcatgggagggaaatgtaacacatggtcttggacccactaagaattggacattgttgtggcattgccagctcatctcttgggcattgagaaaaccttactccagcaatgttattccgtaattgc

SEQ ID NO: 48 - DNA, 599 bp, Homo sapiens
tgctgacatcccaatttataaaaataaaaaggaaacaatatcagcaacattaacttgtcactgccctcccattacctcatgaaagccaaacccacttttctaacaaaccccaaaacttcgcaccacttccttccctacagggacctcccatctgccagtcacccaccctctgtacagctggctaacatacttttcagttatcatttctggattgttatttaatagataatttccagtcttctctcccttatttgagatcttaaataagcttctggaatgcaaagtgcaggtctctttttycatagtgaccacaaatacaccgttttatttttgagcatataattcttatatatttttgttataaaacagtcacaaatgtccacaatgcatcgtagtatatgaaatgttccctgtccatattcattaatccaactaataattttagaacaatttctgggtgccaagcatgattctctacctagttgaaggggtcaacgagggcctctatgagaagggaacatttaagctgagacataaatggaagaacaagcagacctgcagtaaagaggggaacttgtcaggccccatcacaggaataa

SEQ ID NO: 49 - DNA, 599 bp, Homo sapiens
acagtttaattactctgacagctttttttttttattttttctcctagtgaattatcaacagaaactacctagagaaacctgtaacttcagatcttctgattaattgattttctttgggtgctgcttgacagtaacagaatggtccatggggacagtttttcagtgaatgttttcaacaatgataggaacacagagctaagcagagtgtcaagagacatttagtgcagtagagacatctcatctgtttttctgcagaacttgcataaaattagcatctgattattatcacctcagaatgarccggaacccgtaaagatgtctttgatcgtaaaagtggggaggagaccctgatggaagaagagaagacttcagcctcacaatagaaaagactgtgtttatgcacctaaatctaacttagacaccatctaattcaagaaacaggcacatttagggaaataaagaaaagaggcagaaagagatgaagcaccttggctgaatcagagaaattaggggttaatgttccagactgatgacttgaaggagggtgtgcattttcatgcaagggaccattctgtagaagaagcagcacattcaccctt

SEQ ID NO: 50 - DNA, 599 bp, Homo sapiens
tggcccaatttagaaaaccattttgtgggggtcccattaggttcagctttcagttgctggaagagcatggactgacatatgccacaaagcaggtcagaaaatgactttctgtcaacattcatccctactccacgctccccacctctacaacattaacaagaaaatgagaacgcctgcccagatgtcctgttggatgtcactgggtaaattatcttttattaagaatctgtaattgttcttcactgagccgtgacctaaatttagcggtgctgaagagtgagccccagccaacaggatttycctgagaacacgatgaagttcctgtatcaattcgattcattaactagaaagagggaacaagcagccagctttttttttctgcccccattcttcagtgaatcatttgttagtttgaccagaatttttgtgagatccagtctacaattctgtaagaaaaaaagacagcaaacctcattcaaatacttcttgcgacattaattccgaggcagaaaccaaattacattaagcagatctgattgtcttgctattaaagaccttgggtttactcactgagtattttcagcctgaggagaagaaat

SEQ ID NO: 51 - DNA, 599 bp, Homo sapiens
tctgactgctcaaaaatgcctttggagctctaaaaatttatacattcttgggcccgacctgattcagtagttctagtgcaaagctcaggaatctgcataaaactttcagagaattctgatatgttagctcagttgaggaacctctactcttgtctcccatagtccagttaccctgttttgttgtcatatttagccttgtagggtgtgcgcttcacctgacagtaagctggtgaaggctgtgttgcagaggatgagcctggagaacactggcttttcagttggctgatgtttaaattccamctttggtgcttgctaggaggtgggactagggcatcttggctgtttcattttcctcatctttaaatggggagaataatattttctataattaccactttactactaaatcaaggctaccagttactaaatttttgtaatggttctaggataagatagtaagggtctgaaagagaaggaaagaacagagccattagatgaaagaatggaaaggttatgctgtcctctcctcacttttattgaatgctgactgtcgtaggtcttatgagtagcggtaacactatattatctcatttattctt

SEQ ID NO: 52 - DNA, 599 bp, Homo sapiens
tgtcttctggggaaggcttaacacaagagagcaggtggcttcacgggcctttcatttgggtaatgagttggaacacttacccaaagccaagcccaaggaactcccagctgctccacactggaagttgacacacagtaacatatatgcatatgtgtgtgtgttgagatggtggggggatcatttttttgtaacaatcaacaatcttgtaactgtttcctgcctatgttataagccaggataagagcttcctggatcacttgatctctttttgtgttggtaaaaataatcaagcctgtcttyaggcaattagccacctcttcaagccatcaaccttatgcacaatatccaaggaactcctccttgccttttctgaactactgtgggaagtgattggaaggagacagccaaaggccaacaatatagggcgaggcccctgggcatccttctcagctctggggaaggttaatgaattgtacttggggaacaaagagaactttgacaacagagattctgacagtttcataaggcaggaaatgtggccactatcaacccagagctgggtgtgcaaacacaatctatgtgtttatataccttctcct

SEQ ID NO: 53 - DNA, 599 bp, Homo sapiens
tcatcttgctgttacataataaggcactgattagatgctttaggttttgaattttttccttacccgattgaccaactctctaagtctcagtttctccatccataaaatagaagtaataatgcctgcatgtagagttgtaaaaatatataatgagaaaaatattgtgaggtacccagcatagtagccaccacacagtagacaataacctattttacttatctaccttccaatccttaagctaactatctcttagtcccaagaataaaatctcaggattgggaaaaggcttcttcatttctstttaggggccccagatcattgagattcttctttcctattaactaccaggttatggatttgaaagaatctcagttagaaatataatagcatcattctagacagaagaatggaaaactagtgttctaaactggatcaaagggtgtcttgtgaaataatgcagtcattgttctctatagttttgctccaataggtcaaggatatactagtatatcaggggtagctatggatcctgggatttcccctttaccacaaaggagatgatgtaggtgtgaaattccaggcaccttgcccatcttttg

SEQ ID NO: 54 - DNA, 599 bp, Homo sapiens
gccacaacaatgtccaattcttagtgggtccaagaccatgtgttacatttccctcccatgattactcacagcttcacagttctgctgtcctcttcgcctctctgccacctcttaactgcacctttgacctcctacccctaagattcaaccctgtgagattacttgtcttttcatctacactctggtcactctgacccccattctttagattcaggattttctcttttcctggcttactctccagcacaagtagaaaaatattgtgcttcattggaaaatgcatgttgtttgaatcacacyctttcagattatacaattgtagtctttcattatctttgagccattttataatgctgtaaactaatattaatgcatataattcttgtctatagacatgtaaattgtgtccaggggtgatttaattgacttttcttccccattgtggaaaaggctagtagttttgcctccatttgcccatttatttctatattcctgatctgtaaatgcatttctgggttttcccttggactcattctaacatctcttctttttcctcattaattaattaaattaaatctttaacacctcttcatattttt

SEQ ID NO: 55 - DNA, 599 bp, Homo sapiens
atcacagcagtgactctttcaaatctggcctaactgaagctagcaccacaccaggatctctgctgggcacacaccaatcacccaggaggtcagtatcatccccattttacagatttgaaagctgaggcacaaggtaaataacagttatgcagaagtcttgcttcagtgtctaacttcctcatctcactttattctgtttttcaataggagaagagatataatctcatgatgaaaagtgccatctaaagtggcaccttagacacagtaagcaaactggattggaagtgaagaagtcagtcycaagatacttgacaatgtctcctatttgtagattgtttagcaaaatgctttcacctgagttatttcatttgttgctcacaaccaatctgtctggtaggaaaggcagatgatatgttcacctccagataagaaaaatgaaattcagagaggccaaggggcttgctcaagattccacagtgaagaatctacttgcaggaattttaacaaaggttttcaaattcagaagtccacaggatgactgcccttgaaatccagtggtaacatataataaggtgtctttgagcaggtaagagaggacc

SEQ ID NO: 56 - DNA, 599 bp, Homo sapiens
cagtttgaaaatggacaaaaaactgaaatagatatttctcaaaagaagacatacaaatggccaataggatattttttaaatgttactagtcatcaaggaaatgcaaatcaaaatgacaatgaactatcaccttacacttgttagaatggttactagcaaaaaaagacaagggataacaaggttggcaatgatgtagagaaaagggaatccttgtacattgttggagggaatgtaaattagtatagtcactatggaaaactgcatggaggagcttcaaaaaactgaaaataagcctaccaygtgatcctaatactgggtatatatccaaaggattggaaatcaatatgttgaagagatatctgcattcccatgttcgctgcagccttattcacaattgccaagtatgaaattggcttgagtgtccatcaacagatgaatggctatagaaaacatatacacagtggaatactattcagccttaaaaaagaaggcaatcctgtcatttgcaacaacatgatgaacctgtaggacattgtgctgagtaaaataagcctgtcacagaaagacaaatactgtataatctcatatgcagaatcttc

SEQ ID NO: 57 - DNA, 599 bp, Homo sapiens
gactttccagcaccttaaactacttggaaagatagattaatgtctagatgaaacttgatgagactttagactaatatgttacatatacatcaggagatgcatgtataaaccaaatccaaataacccaaagcaaaatttcttttagaaatagtgataaataaatgagtgaggagtttgtcactcacatcccccaggtaaaacatacctttttagcctaaataaatgctatagtttgaatgtgttccctccaaaattcaggtactgatagttaacagcagccgatatgatggtattacacamaatagtaggttctttaggaggttgttaggccatgagtgcctcccccaagaatgggatgaaggacctcataaaagaggcttctcacagcattgtgacctctagccctcccaccttccataagtgaggacacagtgttcctcccctctggaggatgcagcaacaaagtgtcatcttggaagcagaggagccatcaccagacaacagaaccagccaacagcttgatcctggacatctcattttctccagaactgggataaaataaattcctgttttttaataaatttcccaatttcaggtat

SEQ ID NO: 58 - DNA, 599 bp, Homo sapiens
gatgtggaaagcatagacagaaggcactgcagaagaggagagtaaatgactttaggtcattgaactggtttttgatagaaatcaagttaagaaaaatagaagtcaatagtactaagtttgaactttatctttctaaaaactgcttctgtgggcagagttcatatgctggaggaatgaatcataatgacaccctttggtatgaccctggggagaataaatgtgacttagttgaggtttctgaggatgacattcagaaagaaaacaagagagggaaataccctttctctctgtcacactctyaggttagaaactcactttaggggccacagcctatgacaaatacactcatgcaaattctgtgcttgcttccatgctccatttctttgccaactatgtagctaagcaaaatggaacaagaaatagtgtcttgggagaaataaaattgctggattagatacaggccacctttatataagaaaaaacgttaaattatgctactggtgaaataagtccatttgggttattgtggaattttatgtgagactgagatagctgattaagatggtgatgcttgagtattatcattttagtaataaata

SEQ ID NO: 59 - DNA, 599 bp, Homo sapiens
ccctgggaaaatcaccgctctttgtgcctaaatgtcttcatctacaaaaggtggataaataatatttatctcatgaggtgtttggagatttaattaacacttgcaaagcactttgaataggagatgaaaggccctgttaaaggaactattagtgtcagcacctagcgctctttggccactgaggaaatttaatcaggatggggaagtcggtgcaggcccctcctaggccatcctctcctccaccagaagccatattggcctttcattatcctctaagcctggaattctctccaccaagcygctggttatgtcatttccctgcctacaattccctctcttcaattttctccctcctcttttccacgtattaaatcctccaggaccgcttcaagccccatctgctctgcaaagtcttccctaaccattcattgactgattcattcatttacttattcatttgacaaggctttggtgagagcctcctctgtcccaggctccacgaagagccctgaggttgctgtaaattgcatattcttgacctcgtgtgccggaaggggagactggagctcagacaggctacaacgcgtccaaggtcacac

SEQ ID NO: 60 - DNA, 599 bp, Homo sapiens
aataatctcccttactcaaaaccaagtgattatggactttaatcacatctataaaatatcattatagtaacacctaaattagtgtttgaataactgagagttgtaactgatatggtttggctatgtccccacccaaatctcatcttgaattatagttcttataatccccatgtgttgtgggaggaaccaggtggagataattgaatcataggggcagtttctcccattctgttctcatgatagtgagttagttctcaggagatctcatggttttataaagggcttccctctttgctccaytttcattctccttcttcctgctgccatgtgaagaaggacatgtttgcttccccttctgccatgattgtaagtttcctgcggcctccccagccacgctgaactgtgagtcaattaaatctcttttctgtataaattacccagtcttgggcagttctttatagcagcatgagaatggactaatacagtaactttaccaagtggacacataaaactgatcattacaatgtacagtgaatatttggtgagttaatagatatattcataactgaatgaaagaggatggtgattcctacttcagg

SEQ ID NO: 61 - DNA, 599 bp, Homo sapiens
ccaggctccaaaactgtgagaaataagtttctgttgtttataagctaccaggtttaaggtattttgtacagcaacccaaagagtctgagaccataatgaagccattggaatggtgggaaggcaacttcatgtgagtaactacagtaaagccaggtgctggtaacagtcatgttgcccatagagcagatcctactattacagtgcctagcacattacctgcatatgatgatatgtgatcaattagttaactgattagtttatgaatcagtctgccaaaaactagggcagaaattgatagcrcattaaaataaatatgccttaaagtttgcaaggagaccctattaactgcgcactgttttctttttattttctttttttttcttttgagacagggtctcactctgtcacccaggctggagtgcaatggcacagtcttggctcattgcaacctccacttcccgggttcaagcgattcttgtgcctcagcctcccaagtagttgggaatacaggtgtgcaccaccacacctggctaatttttatatttttagtagatagggggtttcccatattagccagcctggtcctgaactcctggcct

SEQ ID NO: 62 - DNA, 599 bp, Homo sapiens
acacacgtgcatacacataaataccttgataagttactagagaaagcagaaaaatgtctgacagtttaatgagatttgggtgaaagaaaattctatatttcattgttttccaggcactagaaataattcatcaatgtttctaagactcattcagcgtggctgcattttttaaaatattttcataaattttgaggagcaaataccattattaggcactaaaaaggttgaagtctaatagattagccgcttcatcctccttcactcagctcagcattcgttcaactggctcttactggttawcatccacacgcctcctgactggctactcagtgccgatgacatttccttcacacacagggctggttttaagatacattgaggtgacatcaggtggcctgtaaagtggtcattttaggatatcctattcaaagacatctgtggaagtgtggaccaatttattgatgaataacagtgaaggggtttccaccagcaagtaacataattttttacaatgatgatgctgaagtagaaagagtttctagtcagggactggacaaatcaatttgcagacgatttttaggaagaaaaacattgcaaca

SEQ ID NO: 63 - DNA, 599 bp, Homo sapiens
atcccttgtcaccagaagctcttaaggaaataacttctacttcgttgcaaatatgttcttggcttagttgaggtaatgcaaatactagaatacttgtttgtttaacagcttattcttccctgaagctgttcctccagtccctgccagtgggatcttatgtctccaggagtacttaacacccctaatagccccatcttttaagcctccctgggacctgccctcgcagtacctcttatacctactccacttcctcctcatggcctcctgcagaatgccattctaaaattaggttctattttyctcgcccgcattctcttttgcaaagcctccaaaaaatttactttgcttctctgcgcctgctttatctctattttctacactcgctccttctttttctaattatctataataggcgtcacaaaatttgcatttgttggaaccaaaatttccatggttgcctcaaaatatacagatgtaaatttgcatataattaaattttgcataagggaaactctcatttggggagatatgcaatgcccaataaatggcagtttccttcaatgtccccaggccagcctcccagtctgtgtgtttccccc

SEQ ID NO: 64 - DNA, 599 bp, Homo sapiens
gccgggcacggtggctcatgcctgtaatcccagcactttgggaggctgaggtgggtggatcacgagatcaagagattgagaccatcctggccaacatggtaaaacctcgtctctactaaaaatacaaaaaattagctgggcatggtggcacacacctgtagtcccagctgctcgggaggctgaggtgggagaatcacttgaacctgggaggcagaggttgcattgagttgaaatcacgccactgcactccagcctggtgacagagcaagactccatctcaaaaacaaacaaaaattatartatgaaataggcattaaaatattgtgtattttagaggagactgaggattggaggctgaagaattactctaaattaatcagcttgtgtacttcagagctaagatagctctttgggttctaaattctgtgatcttctttttgatttctcttggagcaataatgaaggcaaaacatcaataaacataacaaactgggtaagggagaccattgagaaggactaaggacaccttcaaagttctgagtgagtttaaaaagaagaatgatgaaaactttgatagaaataggaaaaaaagtagagga

SEQ ID NO: 65 - DNA, 599 bp, Homo sapiens
atagaaaatttgctatatggtgcatggtggcatgcaactacaaatgtttctaacatgtttctcttcataggattttctgaattttcatttaatattcaagcacatcaaaaacaccttttcaggtgtgatcctatacagcaaagctgtcctcacaaacaatagttgactaaataaacacatggctttatggaagaaatgtgtaagtatagccattgttggagcagatgctctgcttaaaaagaaaaaaaataagttaaagttatagatctcaccatgcttatttactgcttaagtcatagscaatttattgcaccaaagttgaagttcaaagcataaagaatactatatataatgcaattaatgaggttgatgtccctaaaacaagagagaattagtaaatgttttacaatagttttcatgagatgggaaatgacaatagaaatctttgttacaatgcagattttattgtggaaatgatctcatggcaaagtttttaaagaggctgcagaacaataaagagagataacacattttgcttttatgaaaaagccgatgttccaaatttgctgaccttctctgtaaataagaagtgactgtca

SEQ ID NO: 66 - DNA, 599 bp, Homo sapiens
tcaaatctgccaatcagataccagtaataagccatctccttagcataatgtaagacacattcactgattatctaacttgtggcaggcattgtcctgggtcctgaggaagaaaaattacaaatttacaaatctcatagtttacaaagcattttactgaagccagatagacctcaggataaatagcaaacttcatctgagttggccatagggagtctgggcaaaagaaacactcttgatacagaaagaaagaaaagatgggttttctgaaagaaagaagaagcttgtgcagtatttggcagmcccagccaacagcacaagagggaagggagaaagagcacctcattcccaattgtagggcaagaacagattcaatacagctacataaggagccaagactgaattagatgtgtagggaggatgtcctgttacctgagcccaaagtttaacaccctttgatcactttccgtctagagagatcgctcagcaaaggtcattggttatgataattaaagtgtttttctaaatagaagcaatttcaagaaggcaggttgacctttgggagaatttctagcagcaatcacttcatgattgattggaaa

SEQ ID NO: 67 - DNA, 599 bp, Homo sapiens
ataggattccccatttgggctgcagaggagccagaaacatgctttagtttcttaatcctgaaggagtgggggattgtgtaagcctcattggtgcccttttgaaccacatttcatcaagatattgtgaaatggggagcagcaggcagccttctctggccaacatgtatcagggcctcgtgcttagtgggatgtgcccagagtagacagggaatccctggcagccttgcatagtccctctcatgcctttgctttcatggggctggctggaaacaggccggtaagccccatggaactaccggracaaattaccaagcaatctcccatggccatgtagaacagccatgtagaacaggatgtctcccaaggtaagatttaaggtttgtggtttgcagaaaagagaagggaggaaataaaaaagcactgacaatcattgaaagcccacgttctaggtaactggctactcactttcacagcccagtaacaattttgcttaatcctatacatctataaataggattattgtcttcattttccagataaggaaacaagacttggcaaatgtgtagtgtagtgtttgacagtattatgtagttgtttag

SEQ ID NO: 68 - DNA, 599 bp, Homo sapiens
ccagaaagggaagaggaaggacaggcaatagccaaggactcctggcagtgaactcatgtccacatcaagatctaatgagcttgcactcaactcatttctagctctgccttggaagctggagctcctgcactgactatcaatgtgagcccctgagtaggagcagcttggtagagttgaaagaccattgatctgggtcaacagactctggttcctgtctcagcagtgctgtaatcaatccaagtcaaatatcatctctgggacttaatttgctaaatttaaaatgaaaagaaaaacaaaaayagaacaattagactagatcaggattcggcaaaccaaagcctgcttatcaaatctggcataccacctgtttctgtctatacaattgtattgaaactcagccacactcattcatttgcatattgtccatgaaagaagcttttgcgctgcctcaggagatctgagtagtggccacagagatgttgcagtggaccatgttgcaacattgtccaaaatacctaaaatatttacttcttgttttggagagtttgctgactggcaccagagaaatctatggtctaaaatcatctaaaaatttaagc

SEQ ID NO: 69 - DNA, 599 bp, Homo sapiens
cccttgccaatgagggtttggtaagaaaggaaagtgcagtacttctttgtttctgaattgcaagtatgtgtgggttagagggggaggctgaatatgaaggtcctgggacagcccaccaggtatcccatgagactttgcaaaggaaaaggaggtgagtgacagcccagggtccaataggatagaaggaaaagccaggccatggagttcctcagacctgctttctaagggcaactctaccacctcagcaagccattgaacttctctgagctcagtcctttcatttataaaatggggtgacartgctcacatgccaggaatacaaagggattgaaagataaaacacgtaattaagcacctgttgttacacatctgtcagggaccccaataaggtcagctgtcttcctgttgacttctgttcttggtggttctccaagatcataccttccatcaacatttaccgtcactcccccaccccatgcccaatactgaacagtggagggacgcttcacctacagttataatgttgaaacttcaacccaaagcaagtactgttaggatctctggaaactttccctcaaataagggatttgaatgggaca

SEQ ID NO: 70 - DNA, 599 bp, Homo sapiens
cacttgtatgcatttttaaaatgctaatgttaataacagttcgggacttctaacttctatatttaagcaacaaataaataaattgtcagatggtacttcatcatccttctctcccatcttcttagaaatataaattgctttaggtgggaatgctataattttagaccagaaaatacatgccagatgtctcttatatgaagccgtcccgcccaaggatatatatatgccttagtcattaggatgtgttctaaataatactgcaaagcccttggaaggatgggtctgaacactcacttatayttaactgctggcatgttgctttgtccctgtgtcttgtgctactatttccattgatgtaaaggaagcaccaattaaataacactccattattagagaaccaggcacaagtcagctgaggcaggagacccgccttcttttccagaaacaatgtaaagcctgggtgggtgagggtctctgggcttccgccgtgccttgcttttgacattctccagcacaccctataaacatgtctaaggctgtcctgtttagtctgattattcaaactatattgtccagggtagagcaaagggaaacctagc

SEQ ID NO: 71 - DNA, 599 bp, Homo sapiens
agggagagaagtgcctcccacagcaccacgaccagaaagggaagaggaaggacaggcaatagccaaggactcctggcagtgaactcatgtccacatcaagatctaatgagcttgcactcaactcatttctagctctgccttggaagctggagctcctgcactgactatcaatgtgagcccctgagtaggagcagcttggtagagttgaaagaccattgatctgggtcaacagactctggttcctgtctcagcagtgctgtaatcaatccaagtcaaatatcatctctgggacttaatttyctaaatttaaaatgaaaagaaaaacaaaaatagaacaattagactagatcaggattcggcaaaccaaagcctgcttatcaaatctggcataccacctgtttctgtctatacaattgtattgaaactcagccacactcattcatttgcatattgtccatgaaagaagcttttgcgctgcctcaggagatctgagtagtggccacagagatgttgcagtggaccatgttgcaacattgtccaaaatacctaaaatatttacttcttgttttggagagtttgctgactggcaccagagaaat

SEQ ID NO: 72 - DNA, 599 bp, Homo sapiens
gattgcgaacttatcttcatttcacctttatgatgttataccttttcatttttgtcttctcatagctagggtctcctggtccccaaatgtagacacacatcttacaccaatcccagagccattttgtataagagccaccatggatttaaccagctttagctccagtatttgaacataatgttcagcatcatcacctggccaccaaatcaaaactgagcaccctttaatccatcaacaagttctctgcagccatgcaaggttatgaaatgggcacagacatcaatatacagtctttgtgtktaagaggttcatggtctacctgagaaatgcatctttaaacctaaagtagacgctctgtttattccataaatgatttttaagcatcaatggtatatcaagcactgtactggcttctgggctataataaatatataaagaccacaagtttgaatttcatgacattgaactataatgtttaaatgttataataatcatagtaaatgtccttgaggagctacggaagattcctgcatgaagcagaaacaagaagctgaagaaaaaacaactggctttgggggctatataaatataaccctcaa

SEQ ID NO: 73 - DNA, 599 bp, Homo sapiens
ccacatatttcagaggcacataactcaagcttgcaacacgtattcaaaagagaccagctacacttggtagagacagccataggaaagtgaaatgaccctagggtttagtaaagccagctgtttccacttctgaaaataataaaatgaaataataaaataaatttaaaatgatacaaagttcaaagtttaacaaatacatttgaagccatttgcaacaaatacatctgaagctaattgctggctctagaaagtgtggggtctttgttgtggagcagtgttaatgatttagcattacttatmtctggcaaatggtatttttgagataacatgttatggaagaaagtgaactgaacttggaagtttgaagatctcgattgaagtatcatttctgcctcaactacttgcattaacttgtacaagtcattcaaccgctctgaacataatggaaaaatgggatgagaatacatgttgtatactctccaaagacagggagactgctgatataagagggcacttttagtaactgatggagcaaaatgttgttatatgagtgtcagcatagggccctgggcttacaacggtgccatgagccttagaac